SEQUENCE LISTING (66 sequences)

SEQ ID NO: 1 - DNA, 25 bp, synthetic construct
  misc_feature 1..25: note = synthetic oligonucleotide
  source 1..25: mol_type = other DNA
cagtttaagagcaaagtcgtttttc

SEQ ID NO: 2 - DNA, 25 bp, synthetic construct
  misc_feature 1..25: note = synthetic oligonucleotide
  source 1..25: mol_type = other DNA
aatatgtttacattacaggtggcaa

SEQ ID NO: 3 - DNA, 25 bp, synthetic construct
  misc_feature 1..25: note = synthetic oligonucleotide
  source 1..25: mol_type = other DNA
taaagaccaagcaaagatacttgtc

SEQ ID NO: 4 - DNA, 25 bp, synthetic construct
  misc_feature 1..25: note = synthetic oligonucleotide
  source 1..25: mol_type = other DNA
atgcttcatatattcagtggttcac

SEQ ID NO: 5 - DNA, 25 bp, synthetic construct
  misc_feature 1..25: note = synthetic oligonucleotide
  source 1..25: mol_type = other DNA
tgtattaagtgaaattccatgaccc

SEQ ID NO: 6 - DNA, 25 bp, synthetic construct
  misc_feature 1..25: note = synthetic oligonucleotide
  source 1..25: mol_type = other DNA
aacttagcaattaattctgggactc

SEQ ID NO: 7 - DNA, 25 bp, synthetic construct
  misc_feature 1..25: note = synthetic oligonucleotide
  source 1..25: mol_type = other DNA
atgcatatctgtatgcatgcttatt

SEQ ID NO: 8 - DNA, 25 bp, synthetic construct
  misc_feature 1..25: note = synthetic oligonucleotide
  source 1..25: mol_type = other DNA
catattacttggggactaaggacta

SEQ ID NO: 9 - DNA, 23 bp, synthetic construct
  misc_feature 1..23: note = synthetic oligonucleotide
  source 1..23: mol_type = other DNA
atgggcactgcattttagcaata

SEQ ID NO: 10 - DNA, 21 bp, synthetic construct
  misc_feature 1..21: note = synthetic oligonucleotide
  source 1..21: mol_type = other DNA
ccagggcgaggcttatccatt

SEQ ID NO: 11 - DNA, 22 bp, synthetic construct
  misc_feature 1..22: note = synthetic oligonucleotide
  source 1..22: mol_type = other DNA
gcagtcccccactaccacaaat

SEQ ID NO: 12 - DNA, 20 bp, synthetic construct
  misc_feature 1..20: note = synthetic oligonucleotide
  source 1..20: mol_type = other DNA
gacgtaaacggccacaagtt

SEQ ID NO: 13 - DNA, 20 bp, synthetic construct
  misc_feature 1..20: note = synthetic oligonucleotide
  source 1..20: mol_type = other DNA
aagtcgtgctgcttcatgtg

SEQ ID NO: 14 - DNA, 17 bp, synthetic construct
  misc_feature 1..17: note = synthetic oligonucleotide
  source 1..17: mol_type = other DNA
ctcgcttcggcagcaca

SEQ ID NO: 15 - DNA, 20 bp, synthetic construct
  misc_feature 1..20: note = synthetic oligonucleotide
  source 1..20: mol_type = other DNA
aacgcttcacgaatttgcgt

SEQ ID NO: 16 - DNA, 23 bp, synthetic construct
  misc_feature 1..23: note = synthetic oligonucleotide
  source 1..23: mol_type = other DNA
tcggaccatttcagaggtttacc

SEQ ID NO: 17 - DNA, 21 bp, synthetic construct
  misc_feature 1..21: note = synthetic oligonucleotide
  source 1..21: mol_type = other DNA
caggtgctccatgtatcaggt

SEQ ID NO: 18 - DNA, 21 bp, synthetic construct
  misc_feature 1..21: note = synthetic oligonucleotide
  source 1..21: mol_type = other DNA
cgaagttaccgagaccaaaca

SEQ ID NO: 19 - DNA, 23 bp, synthetic construct
  misc_feature 1..23: note = synthetic oligonucleotide
  source 1..23: mol_type = other DNA
tcactgagaacaaactggattgc

SEQ ID NO: 20 - DNA, 23 bp, synthetic construct
  misc_feature 1..23: note = synthetic oligonucleotide
  source 1..23: mol_type = other DNA
atgttctacgcacattttgtcct

SEQ ID NO: 21 - DNA, 22 bp, synthetic construct
  misc_feature 1..22: note = synthetic oligonucleotide
  source 1..22: mol_type = other DNA
tgcactcaaatacatgggcttt

SEQ ID NO: 22 - DNA, 21 bp, synthetic construct
  misc_feature 1..21: note = synthetic oligonucleotide
  source 1..21: mol_type = other DNA
aggtgaaggggattcccgtaa

SEQ ID NO: 23 - DNA, 22 bp, synthetic construct
  misc_feature 1..22: note = synthetic oligonucleotide
  source 1..22: mol_type = other DNA
aaacacgccttttatgagtgga

SEQ ID NO: 24 - DNA, 22 bp, synthetic construct
  misc_feature 1..22: note = synthetic oligonucleotide
  source 1..22: mol_type = other DNA
ttgggaaactgatgaccatagc

SEQ ID NO: 25 - DNA, 20 bp, synthetic construct
  misc_feature 1..20: note = synthetic oligonucleotide
  source 1..20: mol_type = other DNA
acacaaacgtcagcctgctt

SEQ ID NO: 26 - DNA, 20 bp, synthetic construct
  misc_feature 1..20: note = synthetic oligonucleotide
  source 1..20: mol_type = other DNA
cagaaggagaacgcctaccc

SEQ ID NO: 27 - DNA, 19 bp, synthetic construct
  misc_feature 1..19: note = synthetic oligonucleotide
  source 1..19: mol_type = other DNA
gagagcaagcgcagatgtc

SEQ ID NO: 28 - DNA, 21 bp, synthetic construct
  misc_feature 1..21: note = synthetic oligonucleotide
  source 1..21: mol_type = other DNA
ctgacctgggtgaacaatgct

SEQ ID NO: 29 - DNA, 22 bp, synthetic construct
  misc_feature 1..22: note = synthetic oligonucleotide
  source 1..22: mol_type = other DNA
tggctccactgatccaatgtat

SEQ ID NO: 30 - DNA, 20 bp, synthetic construct
  misc_feature 1..20: note = synthetic oligonucleotide
  source 1..20: mol_type = other DNA
gagaggctacgactctgacc

SEQ ID NO: 31 - DNA, 20 bp, synthetic construct
  misc_feature 1..20: note = synthetic oligonucleotide
  source 1..20: mol_type = other DNA
ctccaggtagggggatgttg

SEQ ID NO: 32 - DNA, 21 bp, synthetic construct
  misc_feature 1..21: note = synthetic oligonucleotide
  source 1..21: mol_type = other DNA
aagatcgagaacaccggcata

SEQ ID NO: 33 - DNA, 22 bp, synthetic construct
  misc_feature 1..22: note = synthetic oligonucleotide
  source 1..22: mol_type = other DNA
cttttcctccttcggtctttcc

SEQ ID NO: 34 - DNA, 23 bp, synthetic construct
  misc_feature 1..23: note = synthetic oligonucleotide
  source 1..23: mol_type = other DNA
gatcctacccttctccagatgaa

SEQ ID NO: 35 - DNA, 20 bp, synthetic construct
  misc_feature 1..20: note = synthetic oligonucleotide
  source 1..20: mol_type = other DNA
gtaccgtcacaggaacaggt

SEQ ID NO: 36 - DNA, 23 bp, synthetic construct
  misc_feature 1..23: note = synthetic oligonucleotide
  source 1..23: mol_type = other DNA
caaagacaagcatatcctagcca

SEQ ID NO: 37 - DNA, 22 bp, synthetic construct
  misc_feature 1..22: note = synthetic oligonucleotide
  source 1..22: mol_type = other DNA
cacgtagtgtgtgttaaggacc

SEQ ID NO: 38 - DNA, 21 bp, synthetic construct
  misc_feature 1..21: note = synthetic oligonucleotide
  source 1..21: mol_type = other DNA
gacaccgagatggaggaagta

SEQ ID NO: 39 - DNA, 21 bp, synthetic construct
  misc_feature 1..21: note = synthetic oligonucleotide
  source 1..21: mol_type = other DNA
cgaacagctctgtctgcttta

SEQ ID NO: 40 - DNA, 21 bp, synthetic construct
  misc_feature 1..21: note = synthetic oligonucleotide
  source 1..21: mol_type = other DNA
agctagattcggtgcgagtct

SEQ ID NO: 41 - DNA, 22 bp, synthetic construct
  misc_feature 1..22: note = synthetic oligonucleotide
  source 1..22: mol_type = other DNA
ccaccagtccagctagtgtttt

SEQ ID NO: 42 - DNA, 23 bp, synthetic construct
  misc_feature 1..23: note = synthetic oligonucleotide
  source 1..23: mol_type = other DNA
gctgcctacaaattcaagagtga

SEQ ID NO: 43 - DNA, 23 bp, synthetic construct
  misc_feature 1..23: note = synthetic oligonucleotide
  source 1..23: mol_type = other DNA
aggaaaatgttaggtcgtgacag

SEQ ID NO: 44 - DNA, 21 bp, synthetic construct
  misc_feature 1..21: note = synthetic oligonucleotide
  source 1..21: mol_type = other DNA
tccgaggtgggaaactacctg

SEQ ID NO: 45 - DNA, 22 bp, synthetic construct
  misc_feature 1..22: note = synthetic oligonucleotide
  source 1..22: mol_type = other DNA
cagagtgaggggtatctcttgt

SEQ ID NO: 46 - DNA, 23 bp, synthetic construct
  misc_feature 1..23: note = synthetic oligonucleotide
  source 1..23: mol_type = other DNA
atccagaccttctatttccaggc

SEQ ID NO: 47 - DNA, 20 bp, synthetic construct
  misc_feature 1..20: note = synthetic oligonucleotide
  source 1..20: mol_type = other DNA
cccggaagcggtagatacac

SEQ ID NO: 48 - DNA, 21 bp, synthetic construct
  misc_feature 1..21: note = synthetic oligonucleotide
  source 1..21: mol_type = other DNA
atggtagctgggatgttaggg

SEQ ID NO: 49 - DNA, 21 bp, synthetic construct
  misc_feature 1..21: note = synthetic oligonucleotide
  source 1..21: mol_type = other DNA
gagcgaaaagcttttccctgg

SEQ ID NO: 50 - DNA, 16 bp, synthetic construct
  misc_feature 1..16: note = synthetic oligonucleotide
  source 1..16: mol_type = other DNA
tcttggttactaacag

SEQ ID NO: 51 - DNA, 16 bp, synthetic construct
  misc_feature 1..16: note = synthetic oligonucleotide
  source 1..16: mol_type = other DNA
gaagaagcagagaaca

SEQ ID NO: 52 - DNA, 15 bp, synthetic construct
  misc_feature 1..15: note = synthetic oligonucleotide
  source 1..15: mol_type = other DNA
agtagctcggtggat

SEQ ID NO: 53 - DNA, 16 bp, synthetic construct
  misc_feature 1..16: note = synthetic oligonucleotide
  source 1..16: mol_type = other DNA
tgagtcttgaggagaa

SEQ ID NO: 54 - DNA, 22 bp, synthetic construct
  misc_feature 1..22: note = synthetic oligonucleotide
  source 1..22: mol_type = other DNA
gctggttcgtctatcttgtggg

SEQ ID NO: 55 - DNA, 23 bp, synthetic construct
  misc_feature 1..23: note = synthetic oligonucleotide
  source 1..23: mol_type = other DNA
cagagtagcgaggacttgaagag

SEQ ID NO: 56 - DNA, 23 bp, synthetic construct
  misc_feature 1..23: note = synthetic oligonucleotide
  source 1..23: mol_type = other DNA
atgaatacggctacagcaacagg

SEQ ID NO: 57 - DNA, 22 bp, synthetic construct
  misc_feature 1..22: note = synthetic oligonucleotide
  source 1..22: mol_type = other DNA
ctcttgctcagtgtccttgctg

SEQ ID NO: 58 - DNA, 20 bp, synthetic construct
  misc_feature 1..20: note = synthetic oligonucleotide
  source 1..20: mol_type = other DNA
tctaaggaagtcggggaagc

SEQ ID NO: 59 - DNA, 20 bp, synthetic construct
  misc_feature 1..20: note = synthetic oligonucleotide
  source 1..20: mol_type = other DNA
ccctcgggtgtaatcagaat

SEQ ID NO: 60 - DNA, 21 bp, synthetic construct
  misc_feature 1..21: note = synthetic oligonucleotide
  source 1..21: mol_type = other DNA
acggacaactgcgttgatttt

SEQ ID NO: 61 - DNA, 20 bp, synthetic construct
  misc_feature 1..20: note = synthetic oligonucleotide
  source 1..20: mol_type = other DNA
acttagctgggaagcccaac

SEQ ID NO: 62 - DNA, 20 bp, synthetic construct
  misc_feature 1..20: note = synthetic oligonucleotide
  source 1..20: mol_type = other DNA
accatcttcttcaaggacga

SEQ ID NO: 63 - DNA, 20 bp, synthetic construct
  misc_feature 1..20: note = synthetic oligonucleotide
  source 1..20: mol_type = other DNA
ggctgttgtagttgtactcc

SEQ ID NO: 64 - DNA, 19 bp, synthetic construct
  misc_feature 1..19: note = synthetic oligonucleotide
  source 1..19: mol_type = other DNA
taggctcctcttggacatt

SEQ ID NO: 65 - DNA, 20 bp, synthetic construct
  misc_feature 1..20: note = synthetic oligonucleotide
  source 1..20: mol_type = other DNA
gcaacccatccaagtagatt

SEQ ID NO: 66 - DNA, 33303 bp, Homo sapiens
  source 1..33303: mol_type = genomic DNA
atcatggtgtgtgacctcgggcacatgatgtaacctctataggactctattttaatgtataaaacaggaataatcctttattattatctatgcaatacatattccattatctattacatgggataatggaatgggaagtcccttgaagatggtactaacctcaatgtattactcctttctagcttctttggttcaaaagtttggtggaggagttacaaattctggtttgaatgatatatttggatactttatcaacacatcaaagctctacctatcccttcccccattctcaaaaccaagctgaattaacatctttacatttattatgcagtttatggaggattttagcattaattattgcttgatttactcaatgtccccatgttatagatgagaactggaaaacccattgaagttgtgactcctggtctagaaatgaagtctacttccagttaatgttctttctggtatgtctttgctttcttgaaatttcccttttttgtccttactgggtaaattttgaaccaaccaaatcacaaagatgtccggctttcaatcttctaggccacgcctcttatgctctctccgccctcagcccccccttcagttcttaaagcgctgcaattcgctgctgcagccatatttcttactctctcggggctggaagcttcctgactgaagatctctctgcacttggggttctttctagaacattttctagtcccccaacaccctttatggcgtatttctttaaaaaaatcacctaaattccataaaatatttttttaaattctatactttctcctagtgtcttcttgacacgtcctccatatttttttaaagaaagtatttggaatattttgaggcaatttttaatatttaaggaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttggttttgtgggttgttgcactctctggaatatctacacttttttttgctgctgatcatttggtggtgtgtgagtgtacctaccgctttggcagagaatgactctgcagttaagctaagggcgtgttcagattgtggaggaaaagtggccgccattttagacttgccgcataactcggcttagggctagtcgtttgtgctaagttaaactagggaggcaagatggatgatagcaggtcaggcagaggaagtcatgtgcattgcatgagctaaacctatctgaatgaattgatttggggcttgttaggagctttgcgtgattgttgtatcgggaggcagtaagaatcatcttttatcagtacaagggactagttaaaaatggaaggttaggaaagactaaggtgcagggcttaaaatggcgattttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtgccacgcagagcgggagaaaaggtgggatggacagtgctggattgctgcataacccaaccaattagaaatgggggtggaattgatcacagccaattagagcagaagatggaattagactgatgacacactgtccagctactcagcgaagacctgggtgaattagcatggcacttcgcagctgtctttagccagtcaggagaaagaagtggaggggccacgtgtatgtctcccagtgggcggtacaccaggtgttttcaaggtcttttcaaggacatttagcctttccacctctgtcccctcttatttgtcccctcctgtccagtgctgcctcttgcagtgctggatatctggctgtgtggtctgaacctccctccattcctctgtattggtgcctcacctaaggctaagtatacctccccccccaccccccaacccccccaactccccacccccaccccccaccccccacctccccacccccctacccccctacccccctacccccctctggtctgccctgcactgcactgttgccatgggcagtgctccaggcctgcttggtgtggacatggtggtgagccgtggcaaggaccagaatggatcacagatgatcgttggccaacaggtggcagaagaggaattcctgccttcctcaagaggaacacctaccccttggctaatgctggggtcggattttgatttatatttatcttttggatgtcagtcatacagtctgattttgtggtttgctagtgtttgaatttaagtcttaagtgactattatagaaatgtattaagaggctttatttgtagaattcactttaattacatttaatgagtttttgttttgagttccttaaaattccttaaagtttttagcttctcattacaaattccttaacctttttttggcagtagatagtcaaagtcaaatcatttctaatgttttaaaaatgtgctggtcattttctttgaaattgacttaactattttcctttgaagagtctgtagcacagaaacagtaaaaaatttaacttcatgacctaatgtaaaaaagagtgtttgaaggtttacacaggtccaggccttgctttgttcccatccttgatgctgcactaattgactaatcacctacttatcagacaggaaacttgaattgctgtggtctggtgtcctctattcagacttattatattggagtatttcaatttttcgttgtatcctgcctgcctagcatccagttcctccccagccctgctcccagcaaacccctagtctagccccagccctactcccaccccgccccagccctgccccagccccagtcccctaaccccccagccctagccccagtcccagtcctagttcctcagtcccgcccagcttctctcgaaagtcactctaattttcattgattcagtgctcaaaataagttgtccattgcttatcctattatactgggatattccgtttacccttggcattgctgatcttcagtactgactccttgaccattttcagttaatgcatacaatcccatttgtctgtgatctcaggacaaagaatttccttactcggtacgttgaagttagggaatgtcaattgagagctttctatcagagcattattgcccacaatttgagttacttatcattttctcgatcccctgcccttaaaggagaaaccatttctctgtcattgcttctgtagtcacagtcccaattttgagtagtgatcttttcttgtgtactgtgttggccacctaaaactctttgcattgagtaaaattctaattgccaataatcctacccattggattagacagcactctgaaccccatttgcattcagcagggggtcgcagacaacccgtcttttgttggacagttaaaatgctcagtcccaattgtcatagctttgcctattaaacaaaggcaccctactgcgctttttgctgtgcttctggagaatcctgctgttcttggacaattaaagaacaaagtagtaattgctaattgtctcacccattaatcatgaagactaccagtcgcccttgcatttgccttgaggcagcgctgactacctgagatttaagagtttcttaaattattgagtaaaatcccaattatccatagttctgttagttacactatggcctttgcaaacatctttgcataacagcagtgggactgactcattcttagagccccttcccttggaatattaatggatacaatagtaattattcatggttctgcgtaacagagaagacccacttatgtgtatgcctttatcattgctcctagatagtgtgaactacctaccaccttgcattaatatgtaaaacactaattgcccatagtcccactcattagtctaggatgtcctctttgccattgctgctgagttctgactacccaagtttccttctcttaaacagttgatatgcataattgcatatattcatggttctgtgcaataaaaatggattctcaccccatcccaccttctgtgggatgttgctaacgagtgcagattattcaataacagctcttgaacagttaatttgcacagttgcaattgtccagagtcctgtccattagaaagggactctgtatcctatttgcacgctacaatgtgggctgatcacccaaggactcttcttgtgcattgatgttcataattgtatttgtccacgatcttgtgcactaacccttccactccctttgtattccagcaggggacccttactactcaagacctctgtactaggacagtttatgtgcacaatcctaattgattagaactgagtcttttatatcaaggtccctgcatcatctttgctttacatcaagagggtgctggttacctaatgcccctcctccagaaattattgatgtgcaaaatgcaatttccctatctgctgttagtctggggtctcatcccctcatattccttttgtcttacagcagggggtacttgggactgttaatgcgcataattgcaattatggtcttttccattaaattaagatcccaactgctcacaccctcttagcattacagtagagggtgctaatcacaaggacatttcttttgtactgttaatgtgctacttgcatttgtccctcttcctgtgcactaaagaccccactcacttccctagtgttcagcagtggatgacctctagtcaagacctttgcactaggatagttaatgtgaaccatggcaactgatcacaacaatgtctttcagatcagatccattttatcctccttgttttacagcaagggatattaattacctatgttacctttccctgggactatgaatgtgcaaaattccaatgttcatggtctctccctttaaacctatattctaccccttttacattatagaaagggatgctggaaacccagagtccttctcttgggactcttaatgtgtatttctaattatccatgactcttaatgtgcatattttcaattgcctaattgatttcaattgtctaagacatttcaaatgtctaattgattagaactgagtcttttatatcaagctaatatctagcttttatatcaagctaatatcttgacttctcagcatcatagaagggggtactgatttcctaaagtctttcttgaatttctattatgcaaaattgccctgaggccgggtgtggtggctcacacctgtaatcccagcactttgggaggctgaggtgggaagatcccttactgccaggagtttgagaccagcctggccaacattaaaaaaaaaaaaaagtaagacaattgccctggaatcccatccccctcacacctccttggcaaagcagcaggagtgctaactagctagtgcttcttctcttatactgcttaaatgcgcataattagcagtagttgatgtgcccctatgttagagtagaatcccgcttccttgctccatttgcattactgcaggagcttctaactagcctgaattcactctcttggactgttaatgtgcatacttatatttgctgctgtacttttttaccatgtaaggaccccacccactgtatttacatcccagctggaagtacctactacttaagacccttagactagtaaagttagcgtgcataatcttaggtgttatatacacattttcagttgcatacagttgtgccttttatcaggactcctgtacttatcaaagcagagagtgctaatcaatattaagcccttctcttcgaactgtagatggcatgtaattgcagttgtcaatggtccttcaattagacttgggtttctgacctatcacaccctctttgctttattgcatggggtactattcacttaaggcccctttctcaaactgttaatgtgcctaatgacaattacatcagtatccttccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttgacatgcataattgcatttatgttggttcttgtgccctagacaaggatgccccacctcttttcaatagtgggtgcccactccttatgatctttacatttgaacagttaatgtgaataattgcagttgtccacaaccctatcacttctaggaccattatacctcttttgcattactgtggggtatactgtttccctccaaggccccttctggtggactatcaacatataattgaaattttcttttgtctttgtcagtagattaaggtcataccccatcacctttcctttgtagtacaacagggtgtcctgatcaaccaaagtcctgttgttttggactgttaatatgtgcaattacatttgctcctgatctgtgcactagataaggatcctacctactttcttagtgtttttagcaggtagtgcccactactcaagactgtcacttggaatgttcatgtgcacaaactcaattctctaagcatgttcctgtaccacctttgctttagagcagggggatgatattcactaagtgccccttcttttggacttaatatgcattaatgcaattgtccacctcttcttttagactaagagttgatctccacatattccccttgcatcaggggcatgttaattatgaatgaacccttttcttttaatattaatgtcataattgtatttgtggacctgtgtaggagaaaaagaccctatgttcctcccattaccctttggattgctgctgagaagtgttaactactcataatctcagctcttggacaattaatagcattaataacaattatcaagggcactgatcattagataagactcctgcttcctcgttgcttacatcgggggtactgacccactaaggccccttgtactgttaatgtgaatatttgcaattatatatgtctccttctggtagagtgggatattatgccctagtatcccctttgcattactgcaggggctgctgactactcaaaacttctcctgggactgttaataggcacaatggcagttatcaatggttttctccctccctgaccttgttaagcaagcgccccaccccacccttagtttcccatggcataataaagtataagcattggagtattccatgcacttgtctatcaaacagtggtccatactcccaacccttttgcattgcgccagtgtgtaaaatcacaggtagccatggtgtcatgctttatatacgaagtcttccctctctctgccccttgtgtgcccttggcccctttttacagactattgctcacaatctcaggtgtccatatttgcagctattaggtaagattgtgctgtctccctcttcccttccctctgccctgccccttttgcctctttgctgggtaatgttgaccagacaaggccctttctcttggacttaaacaattctcagttgcactttccttggtcccacccattatacatgaacccctctacttcctttcgcattgcttctgagtatgctgactacccaaagccccttctgtgttattaataaacacagtactgattgtcccatttttcagcccatcagtccaagatctccctaccactttggtgtgttggtgcagtgttgactatgaaaagcaggcctgaactaggtggataagccttcactcattttctttcatttattaatgatcctagtttcaattattgtcagattctggggacaagaaccattcttgcccacctgtgttactgctttactgtgcaaaatactgaaggcaagtcagacccagggagctggattgccatcctttattttgtgtttccagtgtacactataaaattgtctccccaggaaggaaggttggcactttctctgcattcttctttccagagcagattgcctggttaagaatctcttgttgtcccctttgtatattgttattgtaaagtgccaaatgccaggatacagccagaaaaattgcttattattattaaaaaaatttttttaagaaagacatctggattgtagggtggactcgataacctggtcattatttttttgaagccaaaatatccatttatactatgtacctggtgaccagtgtctctcattttaactgagggtggtgggtctgtggatagaacactgactcttgctattttaatatcaaagatattctagagtggaactcttaagaccagtatctttgtgtgggctttaccagcattcacttttagaaaaactacctaaattttataatcctttaatttcttcatctggagcacctgcccctacttatttcaagaagattgcagtaaaacgattaaatgagggaacatatgcagaggtgcttttaaaaagcatatgccaccttttttattaattattatataaaatgaagcatttaattatagtaataatttgaagtagtttgaagtaccacactgaggtgaggacttaaaaatgataagacgagttccctattttataagaaaaataagccaaaattaaatattcttttggatataaatttcaacagtgagatagctgcctagtggaaatgaataatatcccagccactagtgtacagggtgttttgtggcacaggattatgtaatatggaactgctcaagcaaataactagtcatcacaacagcagttctttgtaataactgaaaaagaatattgtttctcggagaaggatgtcaaaagatcggcccagctcagggagcagtttgccctactagctcctcggacagctgtaaagaagagtctctggctctttagaatactgtaagtactacttcgtagctattaagtaatctttttcctattctattttctttctcttagatgccacctatagaaaagtcagagggtccagtaagtttctttccttcttcccacctcatctgcaatatatatatatagagagagaaatagatacatacatacatgcataaatacacatatgtgagttaaccagcagaactgtagaattaatattgtggacccagctctatgctaggttacactgataacctgggtaggaatgatatcatcctatataatttcattcctgagatgattttatcgttgaggagctaatgtgagcacatttgaaataactttagaaaataataagtgctgttttgtgtgaatcataagtagtagttttaggaagggaacccacaaggatttgaagttgatagaataaacttaaggaagtgggtttgctttttctctttaagccaagataggattaatattgcagccatctggatagtccagttggtttattttaatttcatttgttttttacctcttttggagccatggaaagagatgaaagggatagagcatagccattgtgtttggctatttgcgaaggttggcaaattagtgattgctaaatctcataagcttgagtattttaaagttcagagattgagggcataaatctaatacttcggctccttccacaattttactacatttctgcccaagaacagatgaccatggataatgcatatcgtagatactttttaagtttggaacctttttgccaagagggtagtggagaagtgaagtcaaaaccttgaccttccttgcctactttatgctgtagtttatataccttctttcctcccacctttcgtaaagctaaaagaagcttagcctccttaatgttttccagctgacaaaatattgtttaacataacattcgaaactttttttctggtgcacattcatgcatcacagcaggagcaacaagaaccatataagtgaactggcttcacttatagcccgttttaattcatatccatatttcctcagggcttgtttccatgcctcccagccccactccatatgcttaacaacattgtctggctgactgagggttatatacatcatggtcttgaaccttcttggaaacatggtctgtgccattgtttctcaaacccaagtaatgcttcatgatgaaacaccttctaaaggaacaaaattttctgagatcctaaaaaaatgtgttttgaggaacactgacttaacaaagatatttgaaatgtaaatatgttttccaatttcacgttgtctttgtcaaagatgtgttttatataacttatgtagaacttggggatccattagaatatattcacaaatccccagggttatcaccccaatttgagaaaccctggtctatgcttatgaaatcttctattggtaattaaattgtcattcattgtcaacatacaattataattattattggaatttgttttaaatgaatgaatttggaggtgattctgtaccttaagtcaagaggaaggatggcttgattttaggtggattgattatactagatagcatccaaaggtgaatcttgaagctgtatttaaattcattgcttgaaataatttccacccttaagaaaaatctctagcaattgtaaaaagggatgctctggaaatgtgggcatcttcaaaatagagataattcttgtgttagttcaacaaatattattgtaccaggtgctggaataaatagcaaaaccaaagacaggatttatatcaaggaatttgctttcttatggaggatgcagaaggaaatcattatggttttgggcagaaatgcttagactttagtcctggctctgagtttggttcagatcaccatcaatctgaccatctcgagactgctagtgaaataagataggggcttatatcaaatacctaaatccctgaaaatgacattttgtgatttggaaaattttcaaaagtctaatgaaggaaacttttttggcatttctttaaatgattattgtcatttcttttctgacttttccctttataaaaccttaacatgtaggattggaggaagttttctgaccattttctcatatcctctttcagctttatctttctgtaacttccatttctctagccacctccctaaattacagaagactgtgagacccagggctgctgtgattaggcattcataatttcttttcagggtgtttgtgccctgattatcaaatgtacagcttgaagggagttcatgtcttaaagtaatgaattaagagttgacctttgttgactgctaaaatattcttatatgtgaaagcatcctggaaaaatacgttaccagcttaaagagaaagaaactaatgattatatctgaactgagctaatgcctcttctcttcccccaaaccttatcagtttggatggcaaagagtaatgatgtgtcagttaaacagagctaatgccttcctctgccttgtcttaaagactggattgggagaaaattgatattctcactaccatattttgggctgtaggcaagtagcattttacacaggtttccttcaaaaatccaactcaagttggagctcatgtatttaagacatagctggcctgctgaatttaacaagttaaacttcagtggccatgtacagttatatatcactatatatatgtgtattaggctgtcgagttggtcatgtttttgttggtgacttaggctttacttgatagctcttccttgacctttccaaattgagtactgatacatggagcttgggcttcttctgcatcttatacaaatgagtttggtaaagaagcctctcctttactgttttgatgtttatattagaaataacttttgattattttttttcatgttaggatgagaaactgaaacaaaatgtaaatttgaccggtgctagacttcttaaattatgggtagacttaaagtattattttccttaaccaattagaatgctagtcttctagtgttcccggaaacatgagaggttatgcagtagacccaagcaataccctcttattacataatcaagtgcgtataagaatttaaaaatagggatatgactggaacatcactgtactttaccaggtcccattataaaattatctatgttactttacccatagctttgaaaactagtggcatagtatattttatagtatgctgttagtgtgattggcattgaacagtgatgggatataatcactctacaatctatatgttattaaagttttccagccttatagatctcccttgactgaaaattagctactaacttacgacttattttttacagcagattgactaggtctttccaggaaatctgttgatgtacaaaaacaaagtttaattgctaatgtttttttaaaaaataactttttgatattacggatacctggttatttgggccttgtatattttaacatcaaaattacctattataaatccatataaacagaaaagaaagagagtaagtctttagatcagatctgcaaacaatgatggtacgtactgtagaaaaatctggaacatagacttaccagttcttaggttccattttgcttgctttttaaaaactgtgtcttataagtcttcagcaactggttgggagatttttagaaaaaataaccttttaatgttagaacagtgtagagatttacagaatgattctgaagatagagtttctgtgtacttcacacccagtttttcccagtgttaacattttacattagtttggtacatttgtcacaacaaaccaatattgatacattattattaactagagtccatattttattcagatttccttagtttttccttaatgttctttttgtgttccaggatcccattgaagataccacgctgcatgtgtccttagtagtcatgtctccttaggctcctcttggtaatgacagtttctcagactctttgtttttgatgaacttcacagttttgaggactaatggtccagtattctatagaatgtctctctattggaatttgtctgatgttcttctcatgactagattgggtttatgagtgtttaggaggaagaccacaaaggtagagtgccattcttatcacttatcaagagtacatactatcaacatgacttatcactgtttatgttatccttaatcacctgtctgaggtactatttgtcaggtttctccagcgtaaaattagtctttatttctccatttccctactatactgttcacataggaagtcactatgtgcagccagcacttaaggaatgggaaattaccttccacctcattgagggcagagtatttacataaattatttggaattcttttgcacaggatgtcttttctccacaatgtattgtgtttattcagtcatttatatcagtatgatctcagggatattttatactctgggttataatacagtattactttattctgttgttcaaattgttccagctttggccattgggaggtctttcatttggctttgatataaccccatgaatgtgggttttttgtttgagcactttcttatttttggaactacaacatgcttcagactcatttgcatatctcctgcctggacctaaaatgatgtatttctgcaaggagccttgatactttttattggagagtaatattagaaatcaagaagtgaatgctaggtgcgctcattactactggagtgtcattccttcaagaccttttcagttgacaagagcaaggagatatatatttgcattctaacgtgtgtatatgcacatagctataaatatatataaccatctgtatctatattaaactaaatgtgtttatacctacgtctccaactctaatcattgccacatggatcattatagtctcacctccttgcttatctgttacctcccatttctacagtgagaaacctggcttggttgggaaatttttctgttaatattacggtagtgagtgtttgacatttgcttctatggttaagtttagggagagtttagctgtagggtattcttgaaactagaaatgacccttctgccctaaatgtttctgccagttttgaaacgtaaaataggttgcagaaacaaactttatcttaagaaccagaatttacttcaatccacattttgacattgattttcagattaaattattctgatatcgccaggtaagctgttccttgggtatgcatttcttctttccgtttttttctaagagctaaaggaccctgagaacactggaggtgggaaaggaagggaaaggcatgttcacacgtgggataggaaaggttcatttactgacctccagctagccttccaaagtgcctatttaagacccaaggagtagatgtcttccttggcaattgtaacccaaatataatttttaacctttcaattttagtcaagaaagttggtgtgctgttacaaaaagtgccctgattaacagcattgtcatgtgcattgcatattaatcagcaatttaaaataacatgaaattatgttgagtataattttaatattttatattagatattagtttgagacagtgtttctcaagtctgtataataagtttgatagtagggaggttttctctcaagaaaagaattattcagtgtgcacctacataatcactgcttagattctacaattaatattttgctatatttgattaaacgttttctgtaaaagaaaaatattattatgtactatttaggtttatgggaataattgttaagttaaagtgtatgaacaaacctggaatgaaatctgtttgcctacatctataatacaactataaaacatagcagatgtacaaattagtagttaatagataactaaaatgcaaatatggcactactattatagtattatagtttcttttgagtggcgtgtctgtaatatcacatgctgtgttgatgcacttcaccaaactgctgttttcaaactgctttaaatcctgccattatagcacatagcaatgctatttcactttcatttggcacaaaacacatttatatattgtttgcttctcttcttttctgtaatccccaggcaacaaaactagaacatttgccactaatctggcaacgtggtcctatattatgaagtagtcatatagctgatctaaactatccttacagtgaaatgagagtattgtgaaagttttgtagaaagctccccatatgtcctgagaatctatgcacagaccccacagttaaaagacctttgaattgtgggaagacatgggtttaagtatcacttggttaccttctatttgtgtaacattgaggtagtttcatcttctgggttcccagtttccttagagaatgaaaatgttgaattatgtgatttttttttttttttgagacggagttttgctctttcgcccaggctggagtgaagtagcacgatctcgactcactgcaacctccttcccccatgatcaagcaattctcctgcctcagcctcccaagtagctgggattacaggcacccgccccccaccccccgcccccagctaatgtttgtatttttagtacagatggagttttgccgtgttggccaggctggtctcgaacttctgacctcaggtgatccactcgccttggcctcccaaagtgctaggattacaggcatgagccactgcgcctggcctatgtgattattaatatcacgtctagctgtgacaattctgtctgatgctggagtatttgaaccagatggctggctgtgccactcagttattctctccataagactttgatattttgttggtctgcaagatgacggattctcaaaattcttgtcagtgaatattgaaccctagtgaaatgtatggttctgtatcagttccaaaatgtaaccactttctctagccttagattcccagttccaaaatgtaaccattttctctagccttagattcccgttaagggaaagggaatgctctttgagtatgtcatcaccatagtaacaggcaaaactagagggctttgatgctaaagcaagatactccataaatatgcttaagaagacttggggagactggaatagttgttcccttttagatgccagtgtataaatgaatttgagctaggatccgtttatttaaaatttctttaggtgtatttgcttgcatatggagtgcacatttactctcattaatggagttttaggaagcagtagagtaaatgcataaacatgtatgaaccgccatgtttaactggaagcctgcatttggaagtcaagtatctaatcttagattaaattaggatggggaaggatgttggcaagagattttgaagcttgttctgcttatattgagaacatcatagaacagtttggcctttttaaagctagagaatagtgttgaataagtgatgttccatatattcctgtttgacattgacataaaggtttcctcatgatacagtaatccctgatcagggatctggaagcctgtattcatttaaggtactcaggtttaacatactgggtgcttttcacaccatactatacagtaccatgcaaagtgctttcaagactgcaaatttggcttagatcccctttagtgagctcctatgctatagtaaaggtagatagccaattattaaaaacagtcaagacaattgcacctctaagcagtagtagcagttgccacaccaccttgaatcttgaagtattttcagcaacaggatgaccattagccacaaatttagtgtcagcccttaaggtcggtattggtttgacccatattttcatgtagttctttttcttcacttgtctaatcttcccgtgtactgccagggcttgtcattagaggactttagggagaccaagcaggctagaaagtagagacaggagatacctatgtctaatgcttcagtttatacttcctaggtttttttcattggggtttttgtaactcttttggtatcctaccggtgctttggtagcctactgaaccctgtctttcttcttaaggacattctgagcatgtgagacctgaggactgcaaacagctataagaggctccaaattaatcatatctttccctttgagaatctggccaagctccagctaatctacttggatgggttgccagctatctggagaaaaaggtagtttggggaatttattgttgtagtgcttctgtctttggattgaacttcccacaactctcctttttaaagcagaacacagctgggcatggtggctcctgcttgtaattccagggctttgggaggttgaggtggggggatcacttgaggccaggagttgaagacccatgtctctacaataaaataaaattagttgggcatggtggtacgtgcctgtagtcctacctactctggaggctgaggcagcaggattgcttgagcccaggagttcaaggctgcagtgagccatcattagccactgcactccagcctaggtggcagagcgggacccagtctcttaaaaagaaagaaaagcagaacgtgagccagttttcatcaattcctatactttttcttttgcatgtacacatacattttaactttacataatgagttcggcctgtttcatttatccctcagagctgggctccagtgaggtctgtaagggcaagcatacttgatccccaatgaagaatgagagatgcaaagcactaaattatttcttttctcaccacacagcaagatagatttaatgaacttaacaccttttgattagtggccttttaaattattcccactttcctttggcagatgggtattaagttctcaggatttgtttacaaataagactaacttcatctgtattagctcagttttggtaggcctaattccattatcactgccatttccttgttttaagaaatcaaaatttcttagcttgaaaaacaattgaaattgttaaaaagtggaataggagagccccgggggcctgtataaggaatttactgaatccctggttttctgtaccttgtttttccttctgcatagatttgcttaactgtttttgtggcgtgtattttttttttttcgcagtttcgctcttgttgcccaggctggagtgcaatggcgcaatctcagctcactgcaacctctgtctcctgggttcaagttattctcctgcctcagcctctcgagtagctgagattacaggcatgcgcgaccacgccaggctaattttgtatttttagtagagacggggtttctccatgttggtcaggctggtctcaaactcctgacctcaggtgattcacccgcctcgacctcccaaactgctgggattacaggcgtgagccaccacgcctggccagctgttgttataactggagttctatgtgcttgtgaccattcttggtttctccgaatatcctagaactttggtggcgccctattatacaggttgttgaagaaatgttaccatgtggattgagtaggaaacaattctctttatcttggcaatattatggcatggcactacttaaagtacaaattaaaagagggggatgctacagaactagctgacaggcactttgatagaggtggatttctcagttcttaaaatagctctttataaaggaagccagaggcattgtggaggagaattcttacataactcatagggttagaccacatccgaccttttctgtgtggcttcatggctctcttggttgagaaagcattagtttctccttccattagtttcaacctcttgatttcttgacccccctactatattttgtgctgagaacacaagggtattaacaacccacattgtagaggatcgctcagtaataaagactggagaataaaatgcagcatgggaatattggcaattactcagttctaaatttctcttggaaatgagggaaagcatacagaatagagctggaatgaataggataattttttttttttttgctaagttggtagccagaatataacagctccgcacaactgtaaatgtccactcttcaatccacatgaagaaaagggtaaaaatatggttgaactcaaccactagttgcccattagaacagactttcccagtgtactgcatttcaatactttttcttttatctcttttcagatcttcctcagaagaataggcttgttgttttacagtgttagtgatccattccctttgacgatccctaggtggagatggggcatgaggatcctccaggggaaaagctcactaccactgggcaacaaccctaggtcaggaggttctgtcaagatactttcctggtcccagataggaagataaagtctcaaaaacaaccaccacacgtcaaggtgcgtaagctgtccctaaaagcataataagtagtcttaattttgattttgttttccagtatacattgcacttagtgtttcactgaggtcgtattcatcattattctgcatatgatttggtaaaaacagcttcctaactaacctgggaagcaactgggtgtgagattaactggttaaagtgatgatgtaaagagggtagcgggttgcatgtgttcgggtgtttggagtgggactatagcacgtggcagaggcttacagctaagttgttcttttaggagaacatggacaactgtcacatcagtgacattgatcacatgggcaaatcattctgttccatgtggtccccaaagtctctcttaaagccttacagaagaactttgccaatcatttacatacttcaggatggcttgggatgccatggtgtataatacaacaagtgagaggtgtgtctttttatgctatggttgctgattgatggaagccgcataaatacaaatggaaacctgactaaaaatggcacaaagttatctgtcatcaggcaggagctaaagaaccaggaccctacattctctaggtcagtgttgggagaggctgattagcgagtgagaattggcagataaaggtgaccattcggtgcaataaatcctgaacgtataggctttgcccagcattcttcgtaaatagtgggtagctataaatttcatgaaatattttcatgggtaagaactcttgaaatgttataattgactagaaatctctgtagatttagaaatagagagttactaacaaattgttagaaagtctaggaactagaaagctaagttgagagttatctaggaagatctatctattgtactcataatctttagataaattctcctagggccagtagtctatgtgaattttctttttcttcttcttcttcttctttttttttgtattttagctgcaatgttaaacaacctatgtgaattttcttattgtgagaatatttgccttccagagtgactcacctttatctcaaagagcaatattgtgagttttgaaaatgctgctctaaggctgtgttttgttagtcctgagccaggagacttaaagcaaacttgaggggtcttaaaacatcgaagtgagccttaaacattgggaagaccttatgtttttccctctcatatctattatttttgtgatctcagttattaatcatttaaagggactctttcctagctgattggcacttaaaacaggatggaagtctttttttttttttttttttttgagatggagttttgctcttgttgcccaggctggagtgcaatggtgcaatctcagctcactgcaacctctgcctcccgggttcaagcgattctcctgcctcagcctcccaagtagctgggattacagtcatgcaccaccacgcccggctaattttgtatttttaatagagacggtgtttctccatgttggtcaggctggtctcaaactcctgacctcaggtgatccgcccacctcaacctcccaaagtgctgggattatgggcgtgagccaccgcgcccggcagttctggtctttaactaaggtataaggctatgactggtagtggtgtctctagtgactcatcaagtgatatttggcaagacattttcccatttatgccagtttcctattctgttgaatgaggaaattttctctctaaagacctaaaagttttgactttataggtttcaaagttctgtggaaacattttctattgcttattaatttgaatcttatgtaactctagcacagtactcaatatttatggcatttacatggtttatctcatgtttttttatagctcttcattgttcctatctgccaaatcattatacttcctacaagcagtgcagagagctgagtcttcagcaggtccaagaaatttgaacacactgaaggaagtcagccttcccacctgaagatcaacatgcctggcactctagcacttgaggatagctgaatgaagtaagttgttgatgttgcagtcctgtgaggatcacttcagaactgttataacagctgttttttgggagctggtgttggatggggtgtgttggtctaatgtgaagtggggctaaatgtgagatggaaagatgaccagtcttccatattactgactgggttcactgaagcaactcaaagacattatggtcttcttaccagttgtatcacagaagaatttagcctttgcttgtgtgttctatgtcttcactgtataggccctctgtcattcttagagccttaaacgttgagaagcttaaaacaccatttctgctttctgctgaaagggtaaccctttctcatctccgtttgtgagagactctgtcgtcagttaagattagtgtaaaaagaaaactaaactctgaagtagccattataaaagtgtgagaatgaagtcagttttctaaagagttggggaaaggtgatgctaaaggaggggattgagcaagtcctatcaaagagccttttatgaaaatacttagtcatctgtgacatcccatttggctcttccagaaatcctagtaaatagttgtaacaggatgttaagaggcatacattgtgtgttttaaatcctctgctactcattaggtatatgacctttgacaacttaaagtctctagacttctctgtttgtgagggttaaatgaaatcatgtatgtaaagtgctcacctattgcagtgcctggcacatgtcaagtaaaaggtaacccaagaagactcataagttcatttcccacaatataagtgaccactagcactatcaggtagcaggcagagttggcatgctttggttctatgtaagaaatccctaaggtaaaagtttataaatagaagagcatctgtgttggtattggtggttgttattattgtagtactataagtagtattcgtagtaacaatagtttattataattactaatgacactttttgattttttttatctttctgtgatgcttttcatgcctcttgtgcccctcactgtatcttgcctcttctactacttacttcctctgaatgtctgcctttgcttatctcttgcactcaagtgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctagagaaaaatgtgaagagatgctccaggccaatgagaagaattagacaagaaatacacagatgtgccagacttctgagaagcacctgccagcaacagcttccttctttgagcttaggtgagcaggattctggggtttgggatttctagtgatggttatggaaagggtgactgtgcctgggacaaagcgaggtcccaaggggacagcctgaactccctgctcatagtagtggccaaataatttggtggactgtgccaacgctactcctgggtttaatacccatctctaggcttaaagatgagagaacctgggactgttgagcatgtttaatactttccttgatttttttcttcctgtttatgtgggaagttgatttaaatgactgataatgtgtatgaaagcactgtaaaacataagagaaaaaccaattagtgtattggcaatcatgcagttaacatttgaaagtgcagtgtaaattgtgaagcattatgtaaatcaggggtccacagtttttctgtaaggggtcaaatcataaatactttagactgtgggccatatggtttctgttacatatttgttttttaaacaacgtttttataaggtcaaaatcattcttagtttttgagccaattggatttggcctgctgttcatagcttaccaccccctgatgtattatttgttattcagagaaaatttctgaatactactagtttccttttctgtgcctgtccctgtgctaggcactaaaaatgcaatgattattgatatctaggtgacctgaaaaaaaatagtgaatgtgctttgtaaactgtaaagcacttgtattctactgtgataagcgttgtggatacaaagaaaggagcaagcataaaaaagtgctctttcaaaaggatatagtactatgcagacacaaggaattgtttgataaatgaataaattatatgtatatttgaggccaatttgtgtttgctgctctggtaattttgagtaaaaatgcagtattccaggtatcagaaacgaaaacacatggaaactgcttttaaactttaaaatatactgaaaacataagggactaagcttgttgtggtcacctataatgtgccagataccatgctgggtgctagagctaccaaagggggaaaagtattctcatagaacaaaaaatttcagaaaggtgcatattaaagtgctttgtaaactaaagcatgatacaaatgtcaatgggctacatatttatgaatgaatgaatggatgaatgaatattaagtgcctcttacataccagctattttgggtactgtaaaatacaagattaattctcctatgtaataagaggaaagtttatcctctatactattcagatgtaaggaatgatatattgcttaattttaaacaatcaagactttactggtgaggttaagttaaattattactgatacatttttccaggtaaccaggaaagagctagtatgaggaaatgaagtaatagatgtgagatccagaccgaaagtcacttaattcagcttgcgaatgtgctttctaaattataaagcacttgtaaatgaaaaatttgatgctttctgtatgaataaaactttctgtaagctaggtattgtctctacaaaattctcattgtatagttaaaccacagtgagaagggttctataagtagttatacaaaccaagggtttaaatacctgttaaatagatcaattttgattgcctactatgtgaactcactgttaaaggcactgaaaatttatcatatttcatttagccacagccaaaaataaggcaatacctatgttagcattttgtgaactctaaggcaccatataaatgtaactgttgattttctcacttggtgctgggtactaggtttataaaattgtatgatagttattatattgtgcaaataaagtaggaaaatttgaataacaatgattatcttttgaatacgcatacgcaagggattggttgtctgaagaatgccactatagtagttatctattgtgtgccaatctcattgctaggcattggggatgcaaagataaaccatctttattgtgtcttgggtagcagaagaaaatatgtgtaaaatcaatttataatttgtaaactgccacccatatataagctatatctgctgaatgatcattgattactcttatccttagagataacaactgggggcacaaacatttattatcattattgaacctacaacagagatctatgtgtagatttacaaagcctacagttctatacagataggaatgaactattggcttactgaatggtgattactttctgtggggctcggaactacatgccctaggatataaaaatgatgttatcattatagagtgctcacagaaggaaatgaagtaatataggtgtgagatccagaccaaaagtcatttaacaagtttattcagtgatgaaaacatgggacaaatggactaatataaggcagtgtactaagctgagtagagagataaagtcctgtccagaagatacatgcttcctggcctgattgaggagatggaaaatttttgcaaaaaacaaggtgttgtggtcttccatccagtttcttaagtgctgatgataaaagtgaattagacccaccttgacctggcctacagaagtaaaggagtaaaaataaatgcctcaggcgtgctttttgattcatttgataaacaaagcatcttttatgtggaatataccattctgggtcctgaggataagagagatgagggcattagatcactgacagctgaagatagaagaacatctttggtttgattgtttaaataatatttcaatgcctattctctgcaaggtactatgtttcgtaaattaaataggtctggcccagaagacccactcaattgcctttgagattaaaaaaaaaaaaaaaaagaaagaaaaatgcaagtttctttcaaaataaagagacatttttcctagtttcaggaatcccccaaatcacttcctcattggcttagtttaaagccaggagactgataaaagggctcagggtttgttctttaattcattaactaaacattctgcttttattacagttaaatggttcaagatgtaacaactagttttaaaggtatttgctcattggtctggcttagagacaggaagacatatgagcaataaaaaaaagattcttttgcatttaccaatttagtaaaaatttattaaaactgaataaagtgctgttcttaagtgcttgaaagacgtaaaccaaagtgcactttatctcatttatcttatggtggaaacacaggaacaaattctctaagagactgtgtttctttagttgagaagaaacttcattgagtagctgtgatatgttcgatactaaggaaaaactaaacagatcacctttgacatgcgttgtagagtgggaataagagagggctttttattttttcgttcatacgagtattgatgaagatgatactaaatgctaaatgaaatatatctgctccaaaaggcatttattctgacttggagatgcaacaaaaacacaaaaatggaatgaagtgatactcttcatcaaacagaagtgactgttatctcaaccattttgttaaatcctaaacagaaaacaaaaaaaatcatgacgaaaagacacttgcttattaattggcttggaaagtagaatataggagaaaggttactgtttattttttttcatgtattcattcattctacaaatatattcgggtgccaataggtacttggtataaggtttttggccccagagacatgggaaaaaaatgcatgccttcccagagaatgcctaatactttccttttggcttgttttcttgttaggggcatggcttagtccctaaataacattgtgtggtttaattcctactccgtatctcttctaccactctggccactacgataagcaggtagctgggttttgtagtgagcttgctccttaagttacaggaactctccttataatagacacttcattttcctagtccatccctcatgaaaaatgactgaccactgctgggcagcaggagggatgatgaccaactaattcccaaaccccagtctcattggtaccagccttggggaaccacctacacttgagccacaattggttttgaagtgcatttacaaggtttgtctattttcagttctttactttttacatgctgacacatacatacactgcctaaatagatctctttcagaaacaatcctcagataacgcatagcaaaatggagatggagacatgatttctcatgcaacagcttctctaattataccttagaaatgttctcctttttatcatcaaatctgctcaagaagggctttttatagtagaataatatcagtggatgaaaacagcttaacattttaccatgcttaagttttaagaataaaataaaaattggaaataattggccaaaattgaaaggaaaaatttttttaaaatttctctaaatgtaggcctggctgggctttgaccttttccgtttttaaatcactcacagagggtgggacaggaggaagagtgaaggaaaaggtcaaacctgttttaagggcaacctgcctttgttctgaattggtcttaagaacattaccagctccaggtttaaattgttcagtttcatgcagttccaatagctgatcattgttgagatgaggacaaaatcctttgtcctcactagtttgctttacatttttgaaaagtattatttttgtccaagtgcttatcaactaaaccttgtgttaggtaagaatggaatttattaagtgaatcagtgtgacccttcttgtcataagattatcttaaagctgaagccaaaatatgcttcaaaagaagaggactttattgttcattgtagttcatacattcaaagcatctgaactgtagtttctatagcaagccaattacatccataagtggagaaggaaatagataaatgtcaaagtatgattggtggagggagcaaggttgaagataatctggggttgaaattttctagttttcattctgtacatttttagttagacatcagatttgaaatattaatgtttacctttcaatgtgtggtatcagctggactcagtaacacccctttcttcagctggggatggggaatggattattggaaaatggaaagaagaaagtaactaaaagccttcctttcacagtttctggcatcactaccactactgattaaacaagaataagagaacattttatcatcatctgctttattcacataaatgaagttgtgatgaataaatctgcttttatgcagacacaaggaattaagtggcttcgtcattgtccttctacctcaaagataatttattccaaaagctaagataaatggaagactcttgaacttgtgaactgatgtgaaatgcagaatctcttttgagtctttgctgtttggaagattgaaaaatattgttcagcatgggtgaccaccagaaagtaatcttaagccatctagatgtcacaattgaaacaaactggggagttggttgctattgtaaaataaaatatactgttttgaaaactttgtattttgatgtgacaatttctaactcactgtccctaaccacactaattaccgtaagttcttcatttcttttttctttttctttttaaattaataaattaatttattttttggagatggagtctcgctttgttgcccaggctggagtgcagtggtgccatcttggctcactgtaacctctgcctcctgggttcaagcgattctcctgcctcagcctcaccagtagctgggattacagtcatgagccaccatgcctggctaatggttgtatttttagtagagacggggttttgccatgttggccaggctggtcacgaactcctgacttaaagtgaaacacctgcctcggcctcccaaagttctgggattacaggcgggagccaccgtgcctggcctcatttctttttttaaactaccttaatcacccagtgtgggtaaatggtttgaagtctcatttcttacagcatcagttgctttggtcaaggatactgagaagcacaaacccagtaaaaaatgtgagaagcaaaaaacttgaggatcaaaagtgggagaggagaagggtataaactgtaaggctgtgggaattaagcattcatgaaaga

(19) United States
(12) Patent Application Publication
(10) Pub. No.: US 2024/0327835 A1
(43) Pub. Date: Oct. 3, 2024

(54) METHODS FOR REACTIVATING GENES ON THE INACTIVE X CHROMOSOME

(71) Applicant: The General Hospital Corporation, Boston, MA (US)

(72) Inventors: Jeannie T. Lee, Boston, MA (US); Anand Minajigi, Boston, MA (US); Lieselot Carrette, Beernem (BE)

(21) Appl. No.: 18/417,527

(22) Filed: Jan. 19, 2024

Related U.S. Application Data
(63) Continuation of application No. 17/214,320, filed on Mar. 26, 2021, now Pat. No. 11,912,994.
Application No. 17/214,320 is a division of application No. 15/565,060, filed on Oct. 6, 2017, now Pat. No. 10,961,532.
(60) Provisional application No. 62/181,083, filed on Jun. 17, 2015.
(60) Provisional application No. 62/168,528, filed on May 29, 2015.
(60) Provisional application No. 62/144,219, filed on Apr. 7, 2015.

(51) Int. Cl. C12N 15/113; C12N 9/10; C12N 9/90; C12Q 1/6883

(52) U.S. Cl. CPC C12N 15/113; C12N 9/1007; C12N 9/90; C12Q 1/6883; C12Y 201/01037; C12Y 599/01002; C12N 2310/113; C12N 2310/14; C12N 2310/20; C12N 2310/3231; C12N 2310/531; C12N 2320/34; C12Q 2600/156

(57) ABSTRACT

Methods for reactivating genes on the inactive X chromosome that include administering one or both of a DNA methyltransferase (DNMT) Inhibitor and/or a topoisomerase inhibitor, e.g., etoposide and/or 5′-azacytidine (aza), optionally in combination with an inhibitor of XIST RNA and/or an Xist-interacting protein, e.g., a chromatin-modifying protein, e.g., a small molecule or an inhibitory nucleic acid (such as a small inhibitory RNA (siRNAs) or antisense oligonucleotide (ASO)) that targets XIST RNA and/or a gene encoding an Xist-interacting protein, e.g., a chromatin-modifying protein.

### CLAIM OF PRIORITY

This application is a continuation of U.S. patent application Ser. No. 17/214,320, filed Mar. 26, 2021, which is a divisional application of U.S. patent application Ser. No. 15/565,060, filed Oct. 6, 2017, now U.S. Pat. No. 10,961,532, which is a U.S. National Phase Application under 35 U.S.C. § 371 of International Patent Application No. PCT/US2016/026218, filed on Apr. 6, 2016, which claims the benefit of U.S. Patent Applications Serial No. 62/144,219, filed on Apr. 7, 2015; 62/168,528, filed on May 29, 2015; and 62/181,083, filed on Jun. 17, 2015. The entire contents of the foregoing are hereby incorporated by reference.

### FEDERALLY SPONSORED RESEARCH OR DEVELOPMENT

This invention was made with Government support under Grant Nos. DA-38695 and MH97478 awarded by the National Institutes of Health. The Government has certain rights in the invention.

### SEQUENCE LISTING

This application contains a Sequence Listing that has been submitted electronically as an XML file named “29539-0169003_SL_ST26.xml” The XML file, created on Jan. 18, 2024, is 117,804 bytes in size. The material in the ASCII text file is hereby incorporated by reference in its entirety.

### TECHNICAL FIELD

Described herein are methods for reactivating genes on the inactive X chromosome that include administering one or both of a DNA methyltransferase (DNMT) Inhibitor and/or a topoisomerase inhibitor, e.g., etoposide and/or 5′-azacytidine (aza), optionally in combination with an inhibitor of Xist RNA and/or an Xist-interacting protein, e.g., a chromatin-modifying protein, e.g., a small molecule or a nucleic acid such as a small inhibitory RNA (siRNAs), e.g., an antisense oligonucleotide (ASO), e.g., locked nucleic acid (LNA), that targets Xist RNA and/or a gene encoding an Xist-interacting protein, e.g., a chromatin-modifying protein.

### BACKGROUND

X chromosome inactivation (XCI) achieves dosage balance in mammals by repressing one of two X chromosomes in females. X-linked diseases occur in both males and females. In males, X-linked mutations result in disease because males carry only one X-chromosome. In females, disease occurs when a defective gene is present on the active X chromosome (Xa). In some cases, a normal, wild type copy of the gene is present on the inactive X chromosome (Xi), and the severity of the disease may depend on the prevalence (skewing) of inactivation of the X chromosome carrying the wild type gene. The invention described herein may be utilized to treat both male and female X-linked disease. In both females and males, upregulation of a hypomorphic or epigenetically silenced allele may alleviate disease phenotype, such as in Fragile X Syndrome. In females, reactivating a non-disease silent allele on the Xi would be therapeutic in many cases of X-linked disease, such as Rett Syndrome.

### SUMMARY

Provided herein are methods and compositions for reactivating genes on the inactive or active X chromosome.

Provided herein are compositions comprising a DNMT Inhibitor and/or topoisomerase inhibitor, and optionally an inhibitor of Xist RNA and/or an Xist-interacting protein.

Also provided herein are methods for activating an inactive X-linked allele in a cell, preferably a cell of a female heterozygous subject or a male hemizygous subject. The methods include administering to the cell (i) one or both of a DNA methyltransferase (DNMT) Inhibitor and/or a topoisomerase inhibitor; and optionally (ii) an inhibitor of Xist RNA and/or an Xist-interacting protein. As used herein, “an inhibitor of an Xist-interacting protein” can include one or more inhibitors, e.g., one or more small molecules or inhibitory nucleic acids. As used herein, “an inhibitor of Xist RNA” can include one or more inhibitors, e.g., one or more small molecules or inhibitory nucleic acids, e.g., an antisense oligonucleotide (ASO), e.g., locked nucleic acid (LNA), that target XIST RNA or a gene encoding XIST RNA.

In addition, provided herein are methods for activating an epigenetically silenced or hypomorphic allele on the active X-chromosome, e.g., FMRI, in a cell, e.g., in a cell of a male or female heterozygous subject. The methods include administering to the cell (i) one or both of a DNA methyltransferase (DNMT) Inhibitor and/or a topoisomerase inhibitor; and optionally (ii) an inhibitor of Xist RNA and/or an Xist-interacting protein.

Also provided here are a DNMT Inhibitor and/or topoisomerase inhibitor, and optionally an inhibitor of Xist and/or an Xist-interacting protein, for use in activating an inactive X-linked allele in a cell, preferably a cell of a female heterozygous subject, preferably wherein the inactive X-linked allele is associated with an X-linked disorder.

Also provided here are a DNMT Inhibitor and/or topoisomerase inhibitor, and optionally an inhibitor of Xist RNA and/or an Xist-interacting protein, for use in activating an epigenetically silenced or hypomorphic allele on the active X chromosome in a cell, either in a female heterozygous or male hemizygous subject, preferably wherein the active X-linked allele is associated with an X-linked disorder.

Also provided here are a DNMT Inhibitor and/or topoisomerase inhibitor, and optionally an inhibitor of Xist RNA and/or an Xist-interacting protein, for use in treating an X-linked disorder in a female heterozygous or male hemizygous subject.

In some embodiments of the methods or compositions described herein, the inhibitor of Xist RNA is an inhibitory nucleic acid that targets the Xist lncRNA, e.g., e.g., an antisense oligonucleotide (ASO), e.g., locked nucleic acid (LNA), or that targets a gene encoding XIST.

In some embodiments of the methods or compositions described herein, the inhibitor of an Xist-interacting protein inhibits a protein described herein, e.g., shown in Tables 5 or 6 or 7, e.g., SMC1a; SMC3; WAPL, RAD21; KIF4; PDS5a/b; CTCF; TOP1; TOP2a; TOP2b; SMARCA4 (BRG1); SMARCA5; SMARCC1; SMARCC2; SMARCB1; RING1a/b (PRC1); PRC2 (EZH2, SUZ12, RBBP7, RBBP4, EED); AURKB; SPEN/MINT/SHARP; DNMT1; SmcHD1; CTCF; MYEF2; ELAV1; SUN2; Lamin-B Receptor (LBR); LAP; hnRPU/SAF-A; hnRPK; hnRPC; PTBP2; RALY; MATRIN3; MacroH2A; and ATRX.

In some embodiments of the methods or compositions described herein, the inhibitor of an Xist-interacting protein is a small molecule inhibitor or an inhibitory nucleic acid that targets a gene encoding the Xist-interacting protein. In some embodiments, the inhibitor of an Xist-interacting protein is a small molecule inhibitor of cohesin or a cohesin subunit, e.g., a small molecule inhibitor of ECO-I or HDAC6, e.g., PCI34051, tubacin, apicidin, MS275, TSA, or saha.

In some embodiments of the methods or compositions described herein, the inactive X-linked allele is associated with an X-linked disorder, and the DNMT Inhibitor and/or topoisomerase inhibitor, and the optional inhibitor of Xist RNA and/or Xist-interacting protein, are administered in a therapeutically effective amount.

In some embodiments of the methods or compositions described herein, the active X-linked allele is associated with an X-linked disorder, and the DNMT Inhibitor and/or topoisomerase inhibitor, and the optional inhibitor of Xist RNA and/or Xist-interacting protein, are administered in a therapeutically effective amount.

In some embodiments of the methods described herein, the cell is in a living subject.

In some embodiments, the methods described herein optionally include administering (iii) one or more of an inhibitory nucleic acid targeting a strong or moderate RNA-binding protein binding site on the X chromosome, i.e., complementary or identical to a region within a strong or moderate RNA-binding protein site, and/or an inhibitory nucleic acid targeting (i.e., complementary to) a suppressive RNA (supRNA) associated with the X-linked allele.

In some embodiments, the compositions described herein optionally include (iii) one or more of: an inhibitory nucleic acid targeting a strong or moderate RNA-binding protein binding site on the X chromosome, i.e., complementary or identical to a region within a strong or moderate RNA-binding protein site, and/or an inhibitory nucleic acid targeting (i.e., complementary to) a suppressive RNA (supRNA) associated with the X-linked allele.

In some embodiments of the methods or compositions described herein, the inhibitory nucleic acid is identical or complementary to at least 8 consecutive nucleotides of a strong or moderate binding site nucleotide sequence as set forth in Tables A, IVA-C, or XIII-XV of WO 2014/025887 or Table 1 of U.S. Ser. No. 62/010,342, or complementary to at least 8 consecutive nucleotides of a supRNAs as set forth in Tables VI-IX or XVI-XVIII of WO 2014/025887.

In some embodiments of the methods or compositions described herein, the inhibitory nucleic acid does not comprise three or more consecutive guanosine nucleotides or does not comprise four or more consecutive guanosine nucleotides.

In some embodiments of the methods or compositions described herein, the inhibitory nucleic acid is 8 to 30 nucleotides in length.

In some embodiments of the methods or compositions described herein, at least one nucleotide of the inhibitory nucleic acid is a nucleotide analogue.

In some embodiments of the methods or compositions described herein, at least one nucleotide of the inhibitory nucleic acid comprises a 2′ O-methyl, e.g., wherein each nucleotide of the inhibitory nucleic acid comprises a 2′ O-methyl.

In some embodiments of the methods or compositions described herein, the inhibitory nucleic acid comprises at least one ribonucleotide, at least one deoxyribonucleotide, or at least one bridged nucleotide.

In some embodiments of the methods or compositions described herein, the bridged nucleotide is a LNA nucleotide, a cEt nucleotide or a ENA modified nucleotide.

In some embodiments of the methods or compositions described herein, each nucleotide of the inhibitory nucleic acid is a LNA nucleotide.

In some embodiments of the methods or compositions described herein, one or more of the nucleotides of the inhibitory nucleic acid comprise 2′-fluoro-deoxyribonucleotides and/or 2′-O-methyl nucleotides.

In some embodiments of the methods or compositions described herein, one or more of the nucleotides of the inhibitory nucleic acid comprise one of both of ENA nucleotide analogues or LNA nucleotides.

In some embodiments of the methods or compositions described herein, the nucleotides of the inhibitory nucleic acid comprise comprising phosphorothioate internucleotide linkages between at least two nucleotides, or between all nucleotides.

In some embodiments of the methods or compositions described herein, the inhibitory nucleic acid is a gapmer or a mixmer.

Also provided herein are methods for identifying proteins that interact with a selected nucleic acid, e.g., an RNA such as an supRNA. The methods include providing a sample comprising a living cell expressing the selected nucleic acid; exposing the living cell to ultraviolet radiation sufficient to crosslink proteins to DNA, to provide protein-DNA complexes; optionally isolating a nucleus from the cell; treating the isolated nucleus with DNase, e.g., DNase I; solubilizing chromatin in the nucleus; contacting the DNA-protein complexes with capture probes specific for the selected nucleic acid, treating the DNA-protein complexes with DNase, e.g., DNase I, and isolating the DNA-protein complexes from the sample using the capture probes.

In some embodiments, the capture probes comprise a sequence that hybridizes specifically to the selected nucleic acid, and an isolation moiety. In some embodiments, the isolation moiety is biotin, and isolating the DNA-protein complexes comprises contacting the sample with streptavidin or avidin, e.g., bound to a surface, e.g., bound to a bead (e.g., a magnetic bead). In some embodiments, the methods include washing the sample comprising DNA-protein complexes to eliminate protein factors covalently linked by UV to the selected nucleic acid.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E: iDRiP-MS reveals a large Xist interactome.

(A) Exemplary iDRiP schematic. UV-irradiated MEF cells (male, female) were subjected to in vivo capture of Xist RNA-bound proteins. Washes were performed under stringent denaturing conditions to eliminate non-covalently linked proteins. Quantitative mass spectrometry revealed the identity of bound proteins.

(B) RT-qPCR demonstrated the specificity of Xist pulldown by iDRiP. Xist and control luciferase probes were used for pulldown from UV-crosslinked female and control male fibroblasts. Efficiency of Xist pulldown was calculated by comparing to a standard curve generated using 10-fold dilutions of input. Data are shown as Mean±standard error (SE) of twothree independent experiments shown. P values determined by the Student t-test.

(C) Select high-confidence candidates from three biological replicates grouped into multiple functional classes. Additional candidates are shown in Tables 5-6.

(D) UV-RIP-qPCR validation of candidate interactors. The enrichment is calculated as % input for corresponding transcripts, as in (1B). P values determined by the Student t-test.

(E) RNA immunoFISH to examine localization of candidate interactors (green) in relation to Xist RNA (red). Immortalized MEF cells are tetraploid and harbor two Xi.

FIGS. 2A-C: Impact of depleting Xist interactors on H3K27 trimethylation.

(A) RNA immunoFISH of Xist (red) and H3K27me3 (green) after shRNA KD of interactors in fibroblasts (tetraploid; 2 Xist clouds). KD efficiencies (fraction remaining): SMC1a-0.48, SMC3-0.39, RAD21-0.15, AURKB-0.27, TOP2b-0.20, TOP2a-0.42, TOP1-0.34, CTCF-0.62, SMARCA4-0.52, SMARCA5-0.18, SMARCC1-0.25, SMARCC2-0.32, SMARCB1-0.52 and SUN2-0.72. Some factors are essential; therefore, high percentage KD may be inviable. All images presented at the same photographic exposure and contrast.

(B) Quantitation of RNA immunoFISH results from Panel A. n, sample size. % aberrant, percentage of nuclei with aberrant Xist/H3K27me3 associations.

(C) RT-qPCR of Xist RNA levels in fibroblasts after indicated KD. Data are normalized to shControl cells. Mean±SD of two independent experiments shown.

FIGS. 3A-E: De-repression of Xi genes by targeting Xist interactors.

(A) Relative GFP levels determined by RT-qPCR analysis in female fibroblasts stably knocked down for indicated Xist interactors, with or without 0.3 μM 5′-azacytidine (aza) and/or etoposide (eto). Xa-GFP, control X-linked GFP expression from male fibroblasts. Mean±SE of two independent experiments shown. P, determined by Student t-test.

(B) Allele-specific RNA-seq analysis: Number of upregulated Xi genes (range: 2×-250×)(Log 2 fold-change 2-8) for each indicated triple-drug treatment (aza+eto+shRNA). Blue, genes specifically reactivated on Xi (fold-change, FC>2); red, genes also unregulated on Xa (FC>1.3).

(C) RNA-seq heat map indicating that a large number of genes on the Xi were reactivated. X-linked genes reactivated in at least one of the triple-drug treatment (aza+eto+shRNA) were shown in the heat map. Color key, Log 2 fold-change (FC). Cluster analysis performed based on similarity of KD profiles (across) and on the sensitivity and selectivity of various genes to reactivation (down).

(D) Chromosomal locations of Xi reactivated genes for each triple-drug treatment (aza+eto+indicated shRNA). Positions of representative Refseq genes shown at the top. Reactivated genes shown as ticks in each track.

(E) Read coverage of 4 representative reactivated Xi genes after various triple-drug treatments. Xi, mus reads (scale: 0-2). Comp, total reads (scale: 0-6). Reactivation can be appreciated when comparing shControl to various shRNA KDs (Red tags appear only in exons with SNPs).

FIGS. 4A-H: Ablating Xist in cis restores cohesin binding on the Xi.

(A) Allele-specific ChIP-seq results: Violin plots of allelic skew for CTCF, RAD21, SMC1a in wild-type (WT) and Xi^{ΔXist}/Xa^{WT} (ΔXist) fibroblasts. Fraction of mus reads [mus/(mus+cas)] is plotted for every peak with ≥10 allelic reads. P values determined by the Kolmogorov-Smirnov (KS) test.

(B) Differences between SMC1a or RAD21 peaks on the Xi^{WT} versus Xa^{WT} Black diagonal, 1:1 ratio. Plotted are read counts for all SMC1a or RAD21 peaks. Allele-specific skewing is defined as ≥3-fold skew towards either Xa (cas, blue dots) or Xi (mus, red dots). Biallelic peaks, grey dots.

(C) Table of total, Xa-specific, and Xi-specific cohesin binding sites in WT versus ΔXist (Xi^{ΔXist}/Xa^{WT}) cells. Significant SMC1a and RAD21 allelic peaks with ≥5 reads were analyzed. Allele-specific skewing is defined as ≥3-fold skew towards Xa or Xi. Sites were considered “restored” if Xi^{ΔXist}'s read counts were ≥50% of Xa's. X-total, all X-linked binding sites. Allelic peaks, sites with allelic information. Xa-total, all Xa sites. Xi-total, all sites. Xa-spec, Xa-specific. Xi-spec, Xi-specific. Xi-invariant, Xi-specific in both WT and X^{ΔXist}/Xa^{WT} cells. Note: There is a net gain of 96 sites on the Xi in the mutant, a number different from the number of restored sites (106). This difference is due to defining restored peaks separately from calling ChIP peaks (macs2). Allele-specific skewing is defined as ≥3-fold skew towards either Xa or Xi.

(D) Partial restoration of SMC1a or RAD21 peaks on the Xi^{ΔXist} to an Xa-like pattern. Plotted are peaks with read counts with ≥3-fold skew to Xa^{WT} (“Xa-specific”). x-axis, normalized Xa^{WT} read counts. y-axis, normalized X^{ΔXist} read counts. Black diagonal, 1:1 X^{ΔXist}/Xa^{WT} ratio; red diagonal, 1:2 ratio.

(E) Xi-specific SMC1a or RAD21 peaks remained on X^{ΔXist}. Black diagonal, 1:1 ratio. Plotted are read counts for SMC1a or RAD21 peaks with ≥3-fold skew to Xi^{WT} (“Xi-specific peaks).

(F) Comparison of fold-changes for CTCF, RAD21, and SMC1 binding in X^{ΔXist} cells relative to WT cells. Shown are fold-changes for Xi versus Xa. The Xi showed significant gains in RAD21 and SMC1a binding, but not in CTCF binding. Method: X^{WT} and X^{ΔXist} ChIP samples were normalized by scaling to equal read counts. Fold-changes for Xi were computed by dividing the normalized mus read count in X^{ΔXist} by the mus read count X^{WT}; fold-changes for Xa were computed by dividing the normalized cas read count in Xi^{ΔXist} by the cas read count X^{WT}. To eliminate noise, peaks with <10 allelic reads were eliminated from analysis. P values determined by a paired Wilcoxon signed rank test.

(G) The representative examples of cohesion restoration on X^{ΔXist}. ChIP-seq peaks were called by MACS2 software with default settings. Arrowheads, restored peaks.

(H) Allelic-specific cohesin binding profiles of Xa, Xi^{WT}, and X^{ΔXist}. Shown below restored sites are regions of Xi-reactivation following shSMC1a and shRAD21 combination-drug treatments, as defined in FIG. 3.

FIGS. 5A-E: Ablating XIST results in Xi reversion to an Xa-like chromosome conformation.

(A) Chr13 and ChrX contact maps showing triangular domains representative of TADs. Purple shades correspond to varying interaction frequencies (dark, greater interactions). TADs called from our composite (non-allelic) HiC data at 40-kb resolution (blue bars) are highly similar to those (gray bars) called previously by Dixon et al. (27). Representative regions from ChrX and Chr13 are shown.

(B) Allele-specific HiC-seq analysis: Contact maps for three different ChrX regions at 100-kb resolution comparing Xi^{ΔXist} (red) to the Xi of WT cells (Xi^{WT}; orange), and Xi^{ΔXist} (red) versus the Xa (blue) of the mutant cell line. Our Xa TAD calls are shown with RefSeq genes.

(C) Fraction of interaction frequency per TAD on the Xi (mus) chromosome. The positions of TAD borders were rounded to the nearest 100 kb and submatrices were generated from all pixels between the two endpoints of the TAD border for each TAD. We calculated the average interaction score for each TAD by summing the interaction scores for all pixels in the submatrix defined by a TAD and dividing by the total number of pixels in the TAD. We then averaged the normalized interaction scores across all bins in a TAD in the Xi (mus) and Xa (cas) contact maps, and computed the fraction of averaged interaction scores from mus chromosomes. ChrX and a representative autosome, Chr5, are shown for the WT cell line and the X^{ΔXist}/+ cell line. P value determined by paired Wilcoxon signed rank test.

(D) Violin plots showing that TADs overlapping restored peaks have larger increases in interaction scores relative to all other TADs. We calculated the fold-change in average interaction scores on the Xi for all X-linked TADs and intersected the TADs with SMC1a sites (Xi^{ΔXist}/Xi^{WT}) 32 TADs occurred at restored cohesin sites; 80 TADs did not overlap restored cohesin sites. Violin plot shows distributions of fold-change average interaction scores between Xi WT and Xi^{ΔXist} p-value determined by Wilcoxon ranked sum test.

(E) Restored TADs overlap regions with restored cohesions on across Xi^{ΔXist} Several datasets were used to call restored TADs, each producing similar results. Restored TADs were called in two separate replicates (Rep1, Rep2) where the average interaction score was significantly higher on Xi^{ΔXist} than on Xi^{WT}. We also called restored TADs based on merged Rep1+Rep2 datasets. Finally, a consensus between Rep1 and Rep2 was derived. Method: We calculated the fold-change in mus or cas for all TADs on ChrX and on a control, Chr5; then defined a threshold for significant changes based on either the autosomes or the Xa. We treated Chr5 as a null distribution (few changes expected on autosomes) and found the fraction of TADs that crossed the threshold for several thresholds. These fractions corresponded to a false discovery rate (FDR) for each given threshold. An FDR of 0.05 was used.

FIG. 6: The Xi is suppressed by multiple synergistic mechanisms.

Xist RNA (red) suppresses the Xi by either recruiting repressive factors (e.g., Polycomb complexes PRC1, PRC2) or expelling architectural factors (e.g., cohesins).

FIG. 7. Xist knockdown with LNA. Knockdown of XIST was achieved using one of three gapmers, or a combination of all three. No=no LNA control, Scr=Scramble, K=mixmer, A1-A3=3 gapmers, Amix=3 gapmers combined, all at 20 nM FIGS. 8A-B. Luciferase and GFP Controls. Bar graphs showing reactivation of Mecp2 on the Xi, measured by luciferase or GFP reporter levels, after treatment with Aza plus a control LNA or Aza plus a LNA targeting XIST. The MEF cells carried either an Mecp2:luciferase fusion or an Mecp2:GFP fusion.

FIG. 9. The microscopic images of knock down day 7 ESCs.

The stable knock down embryonic stem cells (ESCs) were differentiated after the withdrawal of LIF for seven days. On day 4, the cells were plated on the gelatin coated coverslips until day 7 of differentiation. The coverslips were prepared for immunoFISH, as described in methods, followed by imaging for Xi markers, Xist (Red) and H3K27me3 (Green).

FIGS. 10A-B. Confirmation that the GFP transgene of Xi-TgGFP cells is on the inactive X.

(A) Fluorescent In Situ Hybridization (FISH) indicates the location of the GFP transgene (DNA FISH, red) relative to the inactive X (characterized by a cloud of Xist RNA, identified by RNA FISH in green). In primary fibroblasts selected for high GFP expression (top panels), the transgene is on the active X and does not colocalize with the inactive X (examples indicated by white arrowheads). However, in Xi-TgGFP cells the GPF transgene does colocalize with the inactive X (bottom panels, arrowheads indicate one cell as an example. Xi-TgGFP cells are tetraploid; thus two inactive X chromosomes are seen per cell).

(B) Allele-specific expression of the X-linked gene Mecp2 shown by RT-PCR. Hybrid Xi-TgGFP cells have one M. musculus (mus) X chromosome with the GFP transgene, and one M. castaneus (cas) X. A mus-cas single nucleotide polymorphism is detected by Dde I digest, yielding a 179-bp band for expression from the cas allele, or a 140-bp band for expression from the mus allele. A 200-bp band is common to both alleles. Only the expected cas allele of Mecp2 is expressed in Xi-TgGFP cells (lanes 1, 2, 5), as for purely cas cells (lanes 3, 4, 6), and in contrast to cells of a pure mus background (lane 8), or from a non-clonal hybrid cell population with expression from both alleles (lane 7).

FIGS. 11A-B. Xi reactivation by inhibiting single versus multiple Xist interactors.

(A) Quantitative RT-PCR demonstrated that shRNA knockdown of single Xist interactors resulted in a maximum of 4-fold GFP upregulation.

(B) Biological replicates for allele-specific RNA-seq analysis: Number of upregulated Xi genes for triple-drug treated cells (aza+eto+shRNA). Blue, genes specifically reactivated on Xi; red, genes also upregulated on Xa. There was a net increase in expression level (ΔFPKM) from the Xi in the triple-drug treated samples relative to the shControl+aza+eto, whereas the Xa and autosomes showed no obvious net increase, thereby suggesting direct effects on the Xi as a result of disrupting the Xist interactome. X-reactivation can be observed in various cell types, including proliferating fibroblasts and post-mitotic neurons.

FIG. 12. Correlations between biological replicates for allelic-specific RNA-seq analysis.

Shown are allelic (mus) FPKM values for replicate 1 (Rep1) and replicate 2 (Rep2) for indicated triple-drug treatment (orange text) for all genes, Xi genes, and Chr13 genes.

FIG. 13. Correlations between biological replicates for allelic-specific RNA-seq analysis.

Shown are allelic (mus) FPKM values for replicate 1 (Rep1) and replicate 2 (Rep2) for indicated triple-drug treatment (orange text) for all genes, Xi genes, and Chr13 genes.

FIG. 14. Correlations between biological replicates for allelic-specific RNA-seq analysis.

Shown are allelic (mus) FPKM values for replicate 1 (Rep1) and replicate 2 (Rep2) for indicated triple-drug treatment (orange text) for all genes, Xi genes, and Chr13 genes.

FIGS. 15A-B. Allelic expression of autosomal genes, including imprinted genes, is not affected by the triple-drug treatments.

Read coverages of three representative autosomal genes (A) and four representative imprinted genes (B) after triple-drug treatment. Mus, Mus musculus allele. Comp, total reads. Tracks are shown at the same scale within each grouping. Red tags appear only in exons with SNPs.

FIGS. 16A-D. Analysis of CTCF and cohesin ChIP-seq replicates demonstrates similar allelic trends on ChrX.

(A) Allele-specific ChIP-seq results of biological replicates: Violin plots of allelic skew for CTCF, RAD21, SMC1a in wild-type (WT) and X^{ΔXist}/Xa^{WT} (ΔXist) fibroblasts. Fraction of mus reads [mus/(mus+cas)] is plotted for every peak with ≥10 allelic reads. P values determined by the Kolmogorov-Smirnov (KS) test.

(B) Table of total, Xa-specific, and Xi-specific cohesin binding sites in WT versus ΔXist (Xi^{ΔXist}/Xa^{WT}) cells. Significant SMC1a and RAD21 allelic peaks with ≥5 reads were analyzed. Allele-specific skewing is defined as ≥3-fold skew towards Xa or Xi. Sites were considered “restored” if Xi^{Δxst}'s read counts were ≥50% of Xa's. X-total, all X-linked binding sites. Allelic peaks, sites with allelic information. Xa-total, all Xa sites. Xi-total, all sites. Xa-spec, Xa-specific. Xi-spec, Xi-specific. Xi-invariant, Xi-specific in both WT and Xi^{ΔXist}/Xa^{WT} cells. Note: The net gain of sites on the Xi in the mutant does not equal the number of restored sites. This difference is due to defining restored peaks separately from calling ChIP peaks (macs2). Allele-specific skewing is defined as ≥3-fold skew towards either Xa or Xi.

(C) Correlation analysis showing Log 2 Xi^{ΔXist} to Xa^{WT} ratios of SMC1a coverage in replicates 1 and 2 (Rep1, Rep2). Rep1, blue dots. Rep2, red dots. Both, purple dots. Consensus, upper right quadrant.

(D) Correlation analysis showing Log 2 Xi^{ΔXist} to Xa^{WT} ratios of RAD21 coverage in replicates 1 and 2 (Rep1, Rep2). Rep1, blue dots. Rep2, red dots. Both, purple dots. Consensus, upper right quadrant.

FIG. 17. Analysis of biological replicates for cohesin ChIP-seq confirms cohesin restoration in cis when Xist is ablated.

Allele-specific ChIP-seq analysis of SMC1a and RAD21 biological replicates. Top panels: Differences between SMC1a or RAD21 peaks on the Xi^{WT} versus Xa^{WT} Black diagonal, 1:1 ratio. Plotted are read counts for all SMC1a or RAD21 peaks. Allele-specific skewing is defined as ≥3-fold skew towards either Xa (cas, blue dots) or Xi (mus, red dots). Biallelic peaks, grey dots. Middle panels: Partial restoration of SMC1a or RAD21 peaks on the Xi^{Δxist} to an Xa pattern. Plotted are peaks with read counts with ≥3-fold skew to Xa^{WT} (“Xa-specific”). x-axis, normalized Xa^{WT} read counts. y-axis, normalized Xi^{ΔXist} read counts. Black diagonal, 1:1 Xi^{ΔXist}/Xa^{WT} ratio; red diagonal, 1:2 ratio. Bottom panels: Xi-specific SMC1a or RAD21 peaks remained on Xi^{ΔXist} Plotted are read counts for SMC1a or RAD21 peaks with ≥3-fold skew to Xi^{WT} (“Xi-specific”).

FIG. 18. Restored SMC1a peaks are reproducible in biological replicates and occur throughout X^{ΔXist} (Example set 1).

The representative examples of SMC1a restoration on Xi^{ΔXist} “Restored” peaks shown as ticks under each biological replicate (Rep1, Rep2). The “consensus” restored peaks are shown in the last track of each grouping.

FIG. 19. Restored SMC1a peaks are reproducible in biological replicates and occur throughout X^{ΔXist} (Example set 2).

The representative examples of SMC1a restoration on Xi^{Δxist} “Restored” peaks shown as ticks under each biological replicate (Rep1, Rep2). The “consensus” restored peaks are shown in the last track of each grouping.

FIG. 20. Restored RAD21 peaks are reproducible in biological replicates and occur throughout X^{ΔXist}.

The representative examples of RAD21 restoration on X^{ΔXist}. “Restored” peaks shown as ticks under each biological replicate (Rep1, Rep2). The “consensus” restored peaks are shown in the last track of each grouping.

FIG. 21. Cohesin restored in X^{ΔXist}/Xa^{WT} fibroblasts was Xi-specific and did not occur on autosomes.

Correlation plots comparing SMC1a or RAD21 coverages on the mus versus cas alleles in wildtype fibroblasts (WT) versus Xi^{ΔXist}/Xa^{WT} fibroblasts (ΔXist). Representative autosome, Chr5, is shown. Equation shows the slope and y-intercepts for the black diagonals as a measure of correlation. Pearson's r also shown.

FIGS. 22A-B. Biological replicates of HiC-seq analysis yield similar findings.

(A) Allele-specific contact map for the X-chromosome in wild-type fibroblasts at 100 kb resolution. Orange, Xi. Blue, Xa. DXZ4 location is indicated. The Xi appears to be partitioned into megadomains at DXZ4.

(B) Contact maps for various ChrX regions at 40-kb resolution comparing X^{ΔXist} (red) to Xi^{WT} (orange), and X^{ΔXist} (red) versus Xa (blue) of the mutant cell line. Our TAD calls are shown with RefSeq genes. Rep1 contact maps are shown above Rep2 contact maps.

FIG. 23A-C. Restored TADs identified in X^{ΔXist} using Xa TADs of Dixon et al. (28) as reference.

(A) Using TADs called by Dixon et al. (Dixon et al., Nature 485, 376 (May 17, 2012)) (rather than our own called TADs, as shown in FIG. 5C) as a basis for identifying restored TADs, we calculated the fraction of interaction frequency per TAD on the Xi (mus) chromosome. Highly similar results were obtained. The positions of our Xa TAD borders were rounded to the nearest 100 kb and submatrices were generated from all pixels between the two endpoints of the TAD border for each TAD. We calculated the average interaction score for each TAD by summing the interaction scores for all pixels in the submatrix defined by a TAD and dividing by the total number of pixels in the TAD. We then averaged the normalized interaction scores across all bins in a TAD in the Xi (mus) and Xa (cas) contact maps, and computed the fraction of averaged interaction scores from mus chromosomes. ChrX and a representative autosome, Chr5, are shown for the WT cell line and the X^{ΔXist}/+ cell line. P value determined by KS test. P-value determined by paired Wilcoxon signed rank test.

(B) Using TADs called by Dixon et al. (28) (rather than our own called TADs, as shown in FIG. 5C) as a basis for identifying restored TADs, violin plots also showed that TADs overlapping restored peaks have larger increases in interaction scores relative to all other TADs. We calculated the fold-change in average interaction scores on the Xi for all X-linked TADs and intersected the TADs with SMC1a sites (Xi^{ΔXist}/Xi^{WT}). 32 TADs occurred at restored cohesin sites; 80 TADs did not overlap restored cohesin sites. Violin plot shows distributions of fold-change average interaction scores between Xi^{WT} and Xi^{ΔXist} P-value determined by Wilcoxon ranked sum test.

(C) Using TADs called by Dixon et al. (28) (rather than our own called TADs, as shown in FIG. 5C) as a basis for identifying restored TADs, we also found that restored TADs overlapped regions with restored cohesins on across Xi^{ΔXist} Note highly similar results obtained here relative to FIG. 5E. Several datasets were used to identify restored TADs, each producing similar results. Restored TADs were called in two separate replicates (Rep1, Rep2) where the average interaction score was significantly higher on Xi^{ΔXist} than on Xi^{WT}. We also called restored TADs based on merged Rep1+Rep2 datasets. Finally, a consensus between Rep1 and Rep2 was derived. Method: We calculated the fold-change in mus or cas for all TADs on ChrX and on a control, Chr5; then defined a threshold for significant changes based on either the autosomes or the Xa. We treated Chr5 as a null distribution (few changes expected on autosomes) and found the fraction of TADs that crossed the threshold for several thresholds. These fractions corresponded to a false discovery rate (FDR) for each given threshold. An FDR of 0.05 was used.

### DETAILED DESCRIPTION

The mammalian X chromosome is unique in its ability to undergo whole-chromosome silencing. In the early female embryo, X-chromosome inactivation (XCI) enables mammals to achieve gene dosage equivalence between the XX female and the XY male (1-3). XCI depends on Xist RNA, a 17-kb long noncoding RNA (lncRNA) expressed only from the inactive X-chromosome (Xi)(4) and that implements whole-chromosome silencing by recruiting repressive complexes (5-8). While XCI initiates only once during development, the female mammal stably maintains the Xi through her lifetime. In mice, a germline deletion of Xist results in pen-implantation lethality due to a failure of Xi establishment (9), whereas a lineage-specific deletion of Xist causes a lethal blood cancer due to a failure of Xi maintenance (10). Thus, both the de novo establishment and proper maintenance of the Xi are crucial for viability and homeostasis. There are therefore two critical phases to XCI: (i) A one-time initiation/establishment phase that occurs in pen-implantation embryonic development that is recapitulated by differentiating embryonic stem (ES) cells in culture, and (ii) a life-long maintenance phase that persists in all somatic lineages.

Once established, the Xi is extremely stable and difficult to disrupt genetically and pharmacologically (11-13). In mice, X-reactivation is programmed to occur only twice—once in the blastocyst to erase the imprinted XCI pattern and a second time in the germline prior to meiosis (14, 15). Although the Xi's epigenetic stability is a homeostatic asset, an ability to unlock this epigenetic state is of great current interest. The X-chromosome is home to nearly 1000 genes, at least 50 of which have been implicated in X-linked diseases, such as Rett syndrome and Fragile X syndrome. The Xi is therefore a reservoir of functional genes that could be tapped to replace expression of a disease allele on the active X (Xa). A better understanding of repression would inform both basic biological mechanisms and treatment of X-linked diseases.

It is believed that Xist RNA silences the Xi through conjugate protein partners. A major gap in current understanding is the lack of a comprehensive Xist interactome. In spite of multiple attempts to define the complete interactome, only four directly interacting partners have been identified over the past two decades, including PRC2, ATRX, YY1, and HNRPU: Polycomb repressive complex 2 (PRC2) is targeted by Xist RNA to the Xi; the ATRX RNA helicase is required for the specific association between Xist and PRC2 (16, 17); YY1 tethers the Xist-PRC2 complex to the Xi nucleation center (18); and the nuclear matrix factor, HNRPU/SAF-A, enables stable association of Xist with the chromosomal territory (19). Many additional interacting partners are expected, given the large size of Xist RNA and its numerous conserved modular domains. Here, we develop a new RNA-based proteomic method and implement an unbiased screen for Xist's comprehensive interactome. We identify a large number of high-confidence candidates, demonstrate that it is possible to destabilize Xi repression by inhibiting multiple interacting components, and then delve into a focused set of interactors with the cohesins.

Using iDRiP, we have identified a comprehensive Xist interactome and revealed multiple synergistic pathways to Xi repression (FIG. 6). With Xist physically contacting 80-250 proteins at any given time, the Xist ribonucleoprotein particle may be as large as the ribosome. Our study supports a model in which Xist RNA simultaneously acts as (i) scaffold for the recruitment of repressive complexes (such as PRC1, PRC2, ATRX, mH2A, and SmcHD1) to establish and maintain the inactive state; and as (ii) a repulsion mechanism to extrude architectural factors such as cohesins in order to avoid acquisition of a transcription-favorable chromatin conformation. Without Xist, cohesins return to their default Xa binding state. Repulsion could be based on eviction, with Xist releasing cohesins as it extrudes them, or on sequestration, with Xist sheltering cohesins to prevent Xi binding. Our study shows that the Xi harbors three types of cohesin sites: (i) Xi-specific sites that do not depend on Xist; (ii) biallelic sites that are also Xist-independent; and (iii) Xa-specific sites, many of which cannot be established on the Xi because of active repulsion by Xist. The type i and type iii sites likely explain the paradoxical observations that, on the one hand, depleting cohesins leads to Xi reactivation but, on the other, loss of Xist-mediated cohesin recruitment leads to an Xa-like chromosome conformation that is permissive for transcription. In essence, modulating the Type i and Type iii sites both have the effect of destabilizing the Xi, rendering the Xi more accessible to transcription. Disrupting Type i sites by cohesin knockdown would change the repressive Xi structure, while ablating Xist would restore the Type iii sites that promote an Xa-like conformation. Our study has focused on cohesins, but RNA-mediated repulsion may be an outcome for other Xist interactors and may be as prevalent an epigenetic mechanism as RNA-mediated recruitment (47).

The robustness of Xi silencing is demonstrated by the observation that we destabilized the Xi only after pharmacologically targeting two or three distinct pathways. The fact that the triple-drug treatments varied with respect to reactivated loci and depth of de-repression creates the possibility of treating X-linked disease in a locus-specific manner by administering unique drug combinations. Given the existence of many other disease-associated lncRNAs, the iDRiP technique could be applied systematically towards identifying new drug targets for other diseases and generally for elucidating mechanisms of epigenetic regulation by lncRNA.

Based on the perturbation experiments, it is proposed that Xist interacting factors act synergistically to repress the Xi, possibly explaining why it has been difficult historically to achieve X reactivation by disrupting single genes (11-13). The present data show that drug combinations that hit three distinct pathways are required to achieve reactivation levels that approximate half to full levels of the Xa (FIG. 3). The combinations vary with respect to affected loci and depth of de-repression, thereby creating possibilities with respect to therapies for specific X-linked diseases. In conclusion, the Xist interactome unveiled by iDRiP contains a wealth of new factors to advance understanding of XCI and general lncRNA mechanisms, and to implement new strategies of tackling X-linked disease.

### Methods of Reactivating Genes on the Inactive X Chromosome (Xi)

The present disclosure provides methods for reactivating genes on Xi by combining inhibitors for two or three Xist-interacting factors (listed in Tables 5 and 6). The methods include co-administering a DNA methyltransferase (DNMT) Inhibitor and/or a topoisomerase inhibitor, optionally with an inhibitor of another Xist-interacting factor (listed in Tables 5-6), e.g., a small molecule or a nucleic acid such as a small inhibitory RNA (siRNAs) that targets Xist RNA and/or a gene encoding Xist or an Xist-interacting protein, e.g., a chromatin-modifying protein or a small molecule. These methods can be used, e.g., to reactivate genes in single cells, e.g., isolated cells in culture, or in tissues, organs, or whole animals. In some embodiments, the methods are used to reactivate genes on Xi in a cell or subject that has an X-linked disease. X-reactivation can be achieved in various cell types, including proliferating fibroblasts and post-mitotic neurons.

The methods described herein can be also be used to specifically reactivate one or more genes on Xi, by co-administering an inhibitory nucleic acid targeting a suppressive RNA or genomic DNA at strong and/or moderate binding sites as described in WO 2012/065143, WO 2012/087983, and WO 2014/025887 or in U.S. Ser. No. 62/010,342 (which are incorporated herein in their entirety), to disrupt RNA-mediated silencing in cis on the inactive X-chromosome. The suppressive RNAs can be noncoding (long noncoding RNA, lncRNA) or occasionally part of a coding mRNA; for simplicity, we will refer to them together as suppressive RNAs (supRNAs) henceforth. supRNAs that mediate silencing of genes on the X chromosome are known in the art; see, e.g., WO 2012/065143, WO 2012/087983, WO 2014/025887 and U.S. Ser. No. 62/010,342, and inhibitory nucleic acids and small molecules targeting (e.g., complementary to) the sRNAs, or complementary or identical to a region within a strong or moderate binding site in the genome, e.g., as described in WO 2014/025887, can be used to modulate gene expression in a cell, e.g., a cancer cell, a stem cell, or other normal cell types for gene or epigenetic therapy. The nucleic acids targeting supRNAs that are used in the methods described herein are termed “inhibitory” (though they increase gene expression) because they inhibit the supRNAs-mediated repression of a specified gene, either by binding to the supRNAs itself (e.g., an antisense oligo that is complementary to the supRNAs) or by binding to a strong or moderate binding site for an RNA-binding protein (e.g., PRC2—also termed an EZH2 or SUZ12 binding site- or CTCF) in the genome, and (without wishing to be bound by theory) preventing binding of the RNA-binding protein complex and thus disrupting silencing in the region of the strong or moderate binding site. The inhibitory nucleic acids that bind to a strong or moderate RNA-binding protein binding site can bind to either strand of the DNA, but preferably bind to the same strand to which the supRNAs binds. See, e.g., WO 2012/065143, WO 2012/087983, WO 2014/025887 and U.S. Ser. No. 62/010,342.

The cells can be in vitro, including ex vivo, or in vivo (e.g., in a subject who has cancer, e.g., a tumor).

In some embodiments, the methods include introducing into the cell (or administering to a subject) a DNA methyltransferase (DNMT) Inhibitor and/or a topoisomerase inhibitor, optionally with an inhibitor of XIST RNA or an Xist-interacting protein, e.g., a chromatin-modifying protein, e.g., a small molecule inhibitor of Xist or an Xist-interacting protein.

In some embodiments, the methods include introducing into the cell (or administering to a subject) a DNA methyltransferase (DNMT) Inhibitor and/or a topoisomerase inhibitor, optionally with an inhibitory nucleic acid (e.g., targeting Xist RNA or a gene encoding Xist or an Xist-interacting protein, e.g., a chromatin-modifying protein as described herein) that is modified in some way, e.g., an inhibitory nucleic acid that differs from the endogenous nucleic acids at least by including one or more modifications to the backbone or bases as described herein for inhibitory nucleic acids. Such modified nucleic acids are also within the scope of the present invention.

In some embodiments, the methods include introducing into the cell (or administering to a subject) a DNA methyltransferase (DNMT) Inhibitor and/or a topoisomerase inhibitor, optionally with an inhibitor of Xist RNA or an Xist-interacting protein, e.g., a chromatin-modifying protein, e.g., a small molecule inhibitor or an inhibitory nucleic acid such as a small inhibitory RNA (siRNA) or LNA that targets XIST or a gene encoding XIST or an Xist-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342. A nucleic acid that binds “specifically” binds primarily to the target, i.e., to the target DNA, mRNA, or supRNA to inhibit regulatory function or binding of the DNA, mRNA, or supRNA, but does not substantially inhibit function of other non-target nucleic acids. The specificity of the nucleic acid interaction thus refers to its function (e.g., inhibiting gene expression) rather than its hybridization capacity. Inhibitory nucleic acids may exhibit nonspecific binding to other sites in the genome or other RNAs without interfering with binding of other regulatory proteins and without causing degradation of the non-specifically-bound RNA. Thus this nonspecific binding does not significantly affect function of other non-target RNAs and results in no significant adverse effects. These methods can be used to treat an X-linked condition in a subject by administering to the subject a composition or compositions (e.g., as described herein) comprising a DNA methyltransferase (DNMT) Inhibitor and/or a topoisomerase inhibitor, optionally with an inhibitor of Xist RNA or an Xist-interacting protein, e.g., a chromatin-modifying protein, e.g., a small molecule inhibitor or an inhibitory nucleic acid such as a small inhibitory RNA (siRNA) or LNA that targets a gene encoding Xist or an Xist-interacting protein, e.g., a chromatin-modifying protein, and optionally an inhibitory nucleic acid that specifically binds, or is complementary, to a strong or moderate binding site or a supRNA (e.g., as described in WO 2012/065143, WO 2012/087983, WO 2014/025887 and U.S. Ser. No. 62/010,342) that is associated with an X-linked disease gene. Examples of genes involved in X-linked diseases are shown in Table 8.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, in a patient diagnosed with the disease.

In some embodiments, the methods described herein include administering a DNA methyltransferase (DNMT) Inhibitor and/or a topoisomerase inhibitor, and optionally a composition, e.g., a sterile composition, comprising an inhibitory nucleic acid that is complementary to Xist or a gene encoding Xist RNA or an Xist-interacting protein, e.g., a chromatin-modifying protein, and optionally an inhibitory nucleic acid that is complementary to a supRNA as known in the art, e.g., as described in WO 2012/065143, WO 2012/087983, and/or WO 2014/025887.

Inhibitory nucleic acids for use in practicing the methods described herein can be an antisense or small interfering RNA, including but not limited to an shRNA or siRNA. In some embodiments, the inhibitory nucleic acid is a modified nucleic acid polymer (e.g., a locked nucleic acid (LNA) molecule).

Inhibitory nucleic acids have been employed as therapeutic moieties in the treatment of disease states in animals, including humans. Inhibitory nucleic acids can be useful therapeutic modalities that can be configured to be useful in treatment regimens for the treatment of cells, tissues and animals, especially humans.

For therapeutics, an animal, preferably a human, who has an X-linked disorder is treated by administering a DNA methyltransferase (DNMT) Inhibitor and/or a topoisomerase inhibitor, an optionally inhibitor of XIST RNA and/or an Xist-interacting protein, e.g., a chromatin-modifying protein, e.g., a small molecule inhibitor or an inhibitory nucleic acid such as a small inhibitory RNA (siRNA) or LNA that targets a gene encoding Xist RNA and/or an Xist-interacting protein, e.g., a chromatin-modifying protein, and optionally an inhibitory nucleic acid that is complementary to a supRNA. For example, in some embodiments, the methods comprise the step of administering to the animal in need of treatment, a therapeutically effective amount of a DNA methyltransferase (DNMT) Inhibitor and/or a topoisomerase inhibitor and optionally an inhibitory nucleic acid that is complementary to XIST RNA or a gene encoding XIST and/or an Xist-interacting protein, e.g., a chromatin-modifying protein as described herein.

DNA methyltransferase (DNMT) Inhibitors

A number of DNMT inhibitors (against DNMT1, DNMT2, DNMT3a/b, as several examples) are known in the art, including 5-azacytidine (azacytidine, Azacitidine, 4-amino-1-beta-D-ribofuranosyl-s-triazin-2(1H)-one, Vidaza), decitabine (5-aza-2′-deoxycytidine, Dacogen), Zebularine (pyrimidin-2-one beta-ribofuranoside), procainamide, procaine, hydralazine, NSC14778, Olsalazine, Nanaomycin, SID 49645275, Δ²-isoxazoline, epigallocatechin-3-gallate (EGCG), MG98, SGI-110 (2′-deoxy-5-azacytidylyl-(3′-5′)-2′-deoxyguanosine), RG108 (N-phthalyl-L-tryptophan), SGI-1027, SW155246, SW15524601, SW155246-2, and DZNep (SGI-1036, 3-deazaneplanocin A). See also Medina-Franco et al., Int. J. Mol. Sci. 2014, 15(2), 3253-3261; Yoo et al., Computations Molecular Bioscience, 1(1):7-16 (2011)

### Topoisomerase Inhibitors

A number of topoisomerase inhibitors (against TOP1, TOP2a/b, as examples) are known in the art; in some embodiments, the topoisomerase inhibitor is an inhibitor of topoisomerase II. Exemplary inhibitors of topoisomerase I include camptothecin and its derivatives such as topotecan, irinotecan, lurtotecan, exatecan, diflometecan, S39625, CPT 11, SN38, gimatecan and belotecan; stibogluconate; indenoisoquinolines (e.g., 2,3-dimethoxy-12h-[1,3]dioxolo[5,6]indeno[1,2-c]isoquinolin-6-ium and 4-(5,11-dioxo-5h-indeno[1,2-c]isoquinolin-6(11h)-yl)butanoate) and indolocarbazoles. See, e.g., Pommier, Chem Rev. 2009 July; 109(7): 2894-2902; Pommier, Nat Rev Cancer. 2006 October; 6(10):789-802.; Sheng et al., Curr Med Chem. 2011; 18(28):4389-409. Exemplary inhibitors of topoisomerase II include etoposide, teniposide, mitoxantrone, amsacrine, saintopin, ICRF-193, genistein, CP-115,953, ellipticine, banoxantrone, Celastrol, NU 2058, Dexrazoxane, and anthracyclines (e.g., doxorubicin, daunorubicin, epirubicin, and idarubicin). See, e.g., Froelich-Ammon and Osheroff, Journal of Biological Chemistry, 270:21429-21432 (1995); Hande, Update on Cancer Therapeutics 3:13-26 (2008).

### Inhibitor of XIST RNA

The methods can optionally include administering an inhibitor of an XIST RNA itself, e.g., an inhibitory nucleic acid targeting XIST RNA. (Although in typical usage XIST refers to the human sequence and Xist to the mouse sequence, in the present application the terms are used interchangeably). The human XIST sequence is available in the ensemble database at ENSG00000229807; it is present on Chromosome X at 73,820,651-73,852,753 reverse strand (Human GRCh38.p2). The full sequence is shown in SEQ ID NO:66; XIST exons correspond to 601-11972 (exon 1); 15851-15914 (exon 2); 19593-20116 (exon 3); 21957-21984 (exon 4); 22080-22288 (exon 5); and 23887-33304 (exon 6). Alternatively, see NCBI Reference Sequence: NR_001564.2, Homo sapiens X inactive specific transcript (non-protein coding) (XIST), long non-coding RNA, wherein the exons correspond to 1-11372, 11373-11436, 11437-11573, 11574-11782, 11783-11946, and 11947-19280. The inhibitory nucleic acid targeting XIST RNA can be any inhibitory nucleic acid as described herein, and can include modifications described herein or known in the art. In some embodiments, the inhibitory nucleic acid is an antisense oligonucleotide (ASO) that targets a sequence in XIST RNA, e.g., a sequence within an XIST exon as shown in SEQ ID NO:66 or within the RNA sequence as set forth in NR 001564.2. In some embodiments, the inhibitory nucleic includes at least one locked nucleotide, e.g., is a locked nucleic acid (LNA).

### Xist Interacting Proteins

The methods can optionally include administering an inhibitor of an Xist-interacting protein. Tables 5 and 6 list Xist-interacting proteins, e.g., chromatin-modifying proteins that can be targeted in the methods described herein.

Small molecule inhibitors of many of these Xist interactors are known in the art; see, e.g., Table 7, for strong examples. In addition, small molecule inhibitors of PRc1 or PRC2 components can be used; for example, inhibitors of EZH2 include UNC1999, E7438, N-[(4,6-dimethyl-2-oxo-1,2-dihydro-3-pyridinyl)methyl]-3-methyl-1-[(1S)-1-methylpropyl]-6-[6-(1-piperazinyl)-3-pyridinyl]-1H-indole-4-carboxamide, EPZ-6438 (N-((4,6-dimethyl-2-oxo-1,2-dihydropyridin-3-yl)methyl)-5-(ethyl(tetrahyd-ro-2H-pyran-4-yl)amino)-4-methyl-4′-(morpholinomethyl)-[1,1′-biphenyl]-3-c-arboxamide), GSK-126 ((S)-1-(sec-butyl)-N-(4,6-dimethyl-2-oxo-1,2-dihydropyridin-3-yl)methyl)-3-methyl-6-(6-(piperazin-1-yl)pyridin-3-yl)-1H-indole-4-carboxamide), GSK-343 (1-Isopropyl-N-((6-methyl-2-oxo-4-propyl-1,2-dihydropyridin-3-yl)-methyl)-6-(2-(4-methylpiperazin-1-yl)pyridine-4-yl)-1H-indazole-4-carboxamide), Ell, 3-deazaneplanocin A (DNNep, 5R-(4-amino-1H-imidazo[4,5-c]pyridin-1-yl)-3-(hydroxymethyl)-3-cyclopente-ne-1S,2R-diol), isoliquiritigenin, and those provided in, for example, U.S. Publication Nos. 2009/0012031, 2009/0203010, 2010/0222420, 2011/0251216, 2011/0286990, 2012/0014962, 2012/0071418, 2013/0040906, US20140378470, US20140275081, US20140357688, and 2013/0195843; see also PCT/US2011/035336, PCT/US2011/035340, PCT/US2011/035344.

Cohesin is a multisubunit chromosome-associated protein complex that is highly conserved in eukaryotes; subunits include SMC1, SMC1b, SMC3, Scc1/RAD21, Rec8, SA-1/STAG-1, SA-2/STAG-2, SA-3/STAG-3, Pds5A, Pds5B, Wapl, and Sororin. See, e.g., Peters et al., Genes & Dev. 22:3089-3114 (2008); Lyons and Morgan, Mol Cell. 2011 May 6; 42(3):378-89; Jahnke et al., Nucleic Acids Res. 2008 November; 36(20): 6450-6458. In some embodiments, inhibitors of a cohesin are used, e.g., small molecule inhibitors of ECO-I and HDAC6, which in are a part of a cycle of acetylation-deacetylation that regulates the cohesins; inhibitors include, e.g., PCI34051, tubacin, apicidin, MS275, TSA, or saha. In some embodiments, of the methods described herein, an inhibitor of cohesin is used alone, e.g., without the DNA methyltransferase (DNMT) Inhibitor and/or a topoisomerase inhibitor, or in combination with one or both of them.

Tables 5 and 6, at the end of the Examples, provide the full list of possible Xist-interacting targets.

TABLE 7
Exemplary Xist-Interacting Proteins and Chromatin-Modifying
Proteins

| Xist-Interacting | |
|---|---|
| Protein | Small molecule inhibitor |
| WAPL | — |
| SNC1a | See above |
| SMC3 | See above |
| RAD21 | See above |
| KIF4 | — |
| PDS5a/b | See above |
| CTCF | 3-aminobenzamide |
| TOP1 | See above |
| TOP2a | See above |
| TOP2b | See above |
| SMARCA4 (BRG1) | PFI3 ((E)-1-(2-Hydroxyphenyl)-3-((1R,4R)-5-(pyridin-2-yl)-2,5- |
| | diazabicyclo[2.2.1]heptan-2-yl)prop-2-en-1-one); JQ1(+); AGN-PC- |
| | 0DAUWN |
| SMARCA5 | — |
| SMARCC1 | — |
| SMARCC2 | — |
| SMARCB1 | — |
| CBX2 | — |
| CBX4 | — |
| CBX5 | — |
| CBX6 | — |
| CBX7 | MS37452 |
| CBX8 | — |
| RINB1a | PRT4165 (2-pyridine-3-yl-methylene-indan-1,3-dione) |
| RING1b | — |
| AURKB | ZM447439, Hesperadin, VX-680/MK-0457 (4,6-diaminopyrimidine), |
| | AT9283, AZD1152, AKI-001, PHA-680632, VE-465, JNJ-7706621, |
| | CCT129202, MLN8237, ENMD-2076, MK-5108, PHA-739358, |
| | CYC116, SNS-314, R763, PF-03814375, GSK1070916, AMG-900 |
| | (see Kollareddy et al., Invest New Drugs. 2012 Dec; 30(6): 2411- |
| | 2432) |
| SPEN/MINT/SHARP | MG132 |
| DNMT1 | See above |
| SmcHD1 | — |
| CTCF | — |
| MYEF2 | — |
| ELAVL1 | — |
| SUN2 | mevinolin |
| Lamin-B Receptor | — |
| (LBR) | |
| LAP | bestatin |
| hnRPU/SAF-A | -DPQ |
| hnPRK | — |
| hnRPC | — |
| PTBP2 | — |
| RALY | — |
| MATRIN3 | plumbagin |
| MacroH2A | |
| ATRX | Berberine, Inhibitors of histone deacteylases (HDAC) such as |
| | trichostatin A (TSA), depsipeptide, vorinostat, |
| RYBP | — |
| YY1 | — |
| EZH2 | See above |
| SUZ12 | — |
| EED | Astemizole (inhibits EZH2-EED interaction) |
| RBBP7 | — |
| RBBP4 | — |
| JARID2 | — |

### Inhibitory Nucleic Acids

The methods and compositions described herein can include nucleic acids such as a small inhibitory RNA (siRNA) or LNA that targets (specifically binds, or is complementary to) XIST RNA or to a gene encoding XIST or an XIST-interacting protein, e.g., a chromatin-modifying protein, and optionally an inhibitory nucleic acid that targets a strong or moderate binding site or a supRNA described in WO 2012/065143, WO 2012/087983, WO 2014/025887 and U.S. Ser. No. 62/010,342. Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., U.S. Ser. No. 62/010,342, WO 2012/065143, WO 2012/087983, and WO 2014/025887. However, in some embodiments the inhibitory nucleic acid is not an miRNA, an stRNA, an shRNA, an siRNA, an RNAi, or a dsRNA.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Nat. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids), as well as WO 2012/065143, WO 2012/087983, and WO 2014/025887 (inhibitory nucleic acids targeting non-coding RNAs/supRNAss), all of which are incorporated herein by reference in their entirety.

### Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to an target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

### Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 mM) concentrations of Mg⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

### Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the inhibitory nucleic acid into which they are incorporated more resistant to nuclease digestion than the native oligodeoxynucleotide; these modified oligos survive intact for a longer time than unmodified inhibitory nucleic acids. Specific examples of modified inhibitory nucleic acids include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are inhibitory nucleic acids with phosphorothioate backbones and those with heteroatom backbones, particularly CH2—NH—O—CH2, CH, ˜N(CH3)˜O˜CH2 (known as a methylene(methylimino) or MMI backbone], CH2—O—N(CH3)—CH2, CH2—N(CH3)—N(CH3)—CH2 and O—N(CH3)—CH2—CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the inhibitory nucleic acid is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid inhibitory nucleic acid mimetics are described in Wang et al., J. Am. Chem. Soc., 2000, 122, 8595-8602.

Modified inhibitory nucleic acid backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH2)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an inhibitory nucleic acid; or a group for improving the pharmacodynamic properties of an inhibitory nucleic acid and other substituents having similar properties. A preferred modification includes 2′-methoxyethoxy [2′-O—CH₂CH₂OCH₃, also known as 2′-O-(2-methoxyethyl)](Martin et al, Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the inhibitory nucleic acid, particularly the 3′ position of the sugar on the 3′ terminal nucleotide and the 5′ position of 5′ terminal nucleotide. Inhibitory nucleic acids may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given inhibitory nucleic acid to be uniformly modified, and in fact more than one of the aforementioned modifications may be incorporated in a single inhibitory nucleic acid or even at within a single nucleoside within an inhibitory nucleic acid.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an inhibitory nucleic acid mimetic that has been shown to have excellent hybridization properties, is referred to as a peptide nucleic acid (PNA). In PNA compounds, the sugar-backbone of an inhibitory nucleic acid is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition‘, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. Nos. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the inhibitory nucleic acid. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S— tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., inhibitory nucleic acids containing at least one LNA monomer, that is, one 2′-0,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of inhibitory nucleic acids of 10-30 nucleotides spanning the length of a target RNA can be prepared, followed by testing for activity. Optionally, gaps, e.g., of 5-10 nucleotides or more, can be left between the LNAs to reduce the number of inhibitory nucleic acids synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) inhibitory nucleic acids). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Pouting et al., Cell 136(4):629-641 (2009), and references cited therein.

### Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2-O-methyl, 2′-2′—O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2-O-methyl-modified nucleotide, nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### Pharmaceutical Compositions

The methods described herein can include the administration of pharmaceutical compositions and formulations comprising a DNMT inhibitor and/or topoisomerase inhibitor, and optionally an inhibitor of XIST RNA and/or an Xist-interacting protein, e.g., a chromatin-modifying protein, e.g., a small molecule inhibitor or an inhibitory nucleic acid such as a small inhibitory RNA (siRNA) or LNA that targets XIST RNA and/or a gene encoding Xist or an Xist-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342. The methods can include administration of a single composition comprising a DNMT inhibitor and/or topoisomerase inhibitor, and an optional inhibitor of Xist or an Xist-interacting protein, e.g., a chromatin-modifying protein, or multiple compositions, e.g., each comprising one, two, or all three of a DNMT inhibitor, a topoisomerase inhibitor, and an optional inhibitor of Xist or an Xist-interacting protein, e.g., a chromatin-modifying protein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; A1-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krutzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Disorders Associated with X-Inactivation

The present disclosure provides methods for treating X-linked diseases formulated by administering a DNA methyltransferase (DNMT) Inhibitor and/or a topoisomerase inhibitor, optionally with an inhibitor of an Xist interacting protein, e.g., a small molecule inhibitor or an inhibitory nucleic acid such as a small inhibitory RNA (siRNA) or LNA that targets XIST or a gene encoding XIST or an Xist-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342, to disrupt silencing of genes controlled by the PRC2 sites (e.g., all of the genes within a cluster), or to disrupt silencing of one specific gene. This methodology is useful in X-linked disorders, e.g., in heterozygous women who retain a wildtype copy of a gene on the Xi (See, e.g., Lyon, Acta Paediatr Suppl. 2002; 91(439):107-12; Carrell and Willard, Nature. 434(7031):400-4 (2005); den Veyver, Semin Reprod Med. 19(2):183-91 (2001)). In females, reactivating a non-disease silent allele on the Xi would be therapeutic in many cases of X-linked disease, such as Rett Syndrome (caused by MECP2 mutations), Fabry's Disease (caused by GLA mutations), or X-linked hypophosphatemia (caused by mutation of PHEX). The methodology may also be utilized to treat male X-linked disease. In both females and males, upregulation of a hypomorphic or epigenetically silenced allele may alleviate disease phenotype, such as in Fragile X Syndrome, where the mechanism of epigenetic silencing of FMRI may be similar to epigenetic silencing of a whole Xi in having many different types of heterochromatic marks.

As a result of X-inactivation, heterozygous females are mosaic for X-linked gene expression; some cells express genes from the maternal X and other cells express genes from the paternal X. The relative ratio of these two cell populations in a given female is frequently referred to as the “X-inactivation pattern.” One cell population may be at a selective growth disadvantage, resulting in clonal outgrowth of cells with one or the other parental X chromosome active; this can cause significant deviation or skewing from an expected mean X-inactivation pattern (i.e., 50:50). See, e.g., Plenge et al., Am. J. Hum. Genet. 71:168-173 (2002) and references cited therein.

The present methods can be used to treat disorders associated with X-inactivation, which includes those listed in Table 8. The methods include administering a DNA methyltransferase (DNMT) Inhibitor and/or a topoisomerase inhibitor, optionally with an inhibitor of XIST RNA an Xist-interacting protein, e.g., a chromatin-modifying protein, e.g., a small molecule inhibitor or an inhibitory nucleic acid such as a small inhibitory RNA (siRNA) or LNA that targets Xist or a gene encoding Xist or an Xist-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342, i.e., a supRNA associated with the gene that causes the disorder, as shown in Table 8 and WO 2012/065143, WO 2012/087983, and WO 2014/025887.

TABLE 8
X Linked Disorders and Associated Genes

| Disorder | OMIM # | Locus | Gene |
|---|---|---|---|
| Dent's disease 1 | 300009 | Xp11.22 | CLCN5 |
| Testicular feminization syndrome | 300068 | Xq11-q12 | AR |
| Addison's disease with cerebral | 300100 | Xq28 | ABCD1 |
| sclerosis | | | |
| Adrenal hypoplasia | 300200 | XP21.3-p21.2 | DAX1 |
| siderius X-linked mental | 300263 | Xp11.22 | PHF8 |
| retardation syndrome | | | |
| Agammaglobulinaemia, Bruton | 300300 | Xq21.3-q22 | BTK |
| type | | | |
| Choroidoretinal degeneration | 300389 | Xp21.1 | RPGR |
| Choroidoaemia | 300390 | Xq21.2 | CHM |
| Albinism, ocular | 300500 | Xp22.3 | OA1 |
| Dent's disease 2 | 300555 | Xq25-q26 | OCRL |
| fragile X syndrome | 300624 | Xq27.3 | FMR1 |
| Rett/Epileptic encephalopathy, | 300672 | Xp22.13 | CDKL5 |
| early infantile, 2 | | | |
| Albinism-deafness syndrome | 300700 | Xq26.3-q27.1 | ADFN |
| paroxysmal nocturnal | 300818 | Xp22.2 | PIGA |
| hemoglobulinuria | | | |
| Aldrich syndrome | 301000 | Xp11.23-p11.22 | WAS |
| Alport syndrome | 301050 | Xq22.3 | COL4A5 |
| Anaemia, hereditary hypochromic | 301300 | Xp11.21 | ALAS2 |
| Anemia, sideroblastic, with ataxia | 301310 | Xq13.3 | ABCB7 |
| Fabry disease | 301500 | Xq22 | GLA |
| Spinal muscular atrophy 2 | 301830 | Xp11.23 | UBA1 |
| Cataract, congenital | 302200 | Xp | CCT |
| Charcot-Marie-Tooth, peroneal | 302800 | Xq13.1 | GJB1 |
| Spastic paraplegia | 303350 | Xq28 | L1CAM |
| Colour blindness | 303800 | Xq28 | OPN1MW |
| Diabetes insipidus, nephrogenic | 304800 | Xq28 | AVPR2 |
| Dyskeratosis congenita | 305000 | Xq28 | DKC1 |
| Ectodermal dysplasia, anhidrotic | 305100 | Xq12-q13.1 | ED1 |
| Faciagenital dysplasia (Aarskog | 305400 | Xp11.21 | FGD1 |
| syndrome) | | | |
| Glucose-6-phosphate | 305900 | Xq28 | G6PD |
| dehydrogenase deficiency | | | |
| Glycogen storage disease type | 306000 | Xp22.2-p22.1 | PHKA2 |
| VIII | | | |
| Gonadal dysgenesis (XY female | 306100 | Xp22.11-p21.2 | GDXY |
| type) | | | |
| Granulomatous disease (chronic) | 306400 | Xp21.1 | CYBB |
| Haemophilia A | 306700 | Xq28 | F8 |
| Haemophilia B | 306900 | Xq27.1-q27.2 | F9 |
| Hydrocephalus (aqueduct stenosis) | 307000 | Xq28 | L1CAM |
| Hydrophosphataemic rickets | 307800 | Xp22.2-p22.1 | PHEX |
| Lesch-Nyhan syndrome | 308000 | Xq26-q27.2 | HPRT1 |
| (hypoxanthine-guanine- | | | |
| phosphoribosyl transferase | | | |
| deficiency) | | | |
| Incontinentia pigmenti | 308300 | Xq28 | IBKBG |
| Kallmann syndrome | 308700 | Xp22.3 | KAL1 |
| Keratosis follicularis spinulosa | 308800 | Xp22.1 | SAT |
| Lowe (oculocerebrorenal) | 309000 | Xq26.1 | OCRL |
| syndrome | | | |
| Menkes syndrome | 309400 | Xq12-q13 | ATP7A |
| Renpenning syndrome | 309500 | Xp11.23 | PQBP1 |
| Mental retardation, with or | 309530 | Xp11.3-q21.1 | MRX1 |
| without fragile site (numerous | | | |
| specific types) | | | |
| Coffin-Lowry syndrome | 309580 | Xq13 | ATRX |
| Microphthalmia with multiple | 309800 | Xq27-q28 | MAA |
| anomalies (Lenz syndrome) | | | |
| Muscular dystrophy (Becker, | 310300 | Xq28 | EMD |
| Duchenne and Emery-Dreifuss | | | |
| types) | | | |
| Myotubular myopathy | 310400 | Xq28 | MTM1 |
| Night blindness, cogenital | 310500 | Xp11.4 | CSNB1 |
| stationary | | | |
| Norrie's disease (pseudoglioma) | 310600 | Xp11.4 | NDP |
| Nystagmus, oculomotor or ‘jerky’ | 310700 | Xq26-q27 | NYS1 |
| Orofaciodigital syndrome (type I) | 311200 | Xp22.2-p22.2 | OFD1 |
| Ornithine transcarbamylase | 311250 | Xp21.1 | OTC |
| deficiency (type I | | | |
| hyperammonaemia) | | | |
| Phosphoglycerate kinase | 311800 | Xq13 | PGK1 |
| deficiency | | | |
| Phosphoribosylpyrophosphate | 311850 | Xq22-q24 | PRPS1 |
| synthetase deficiency | | | |
| Retinitis pigmentosa | 312610 | Xp21.1 | RPGR |
| Retinoschisis | 312700 | Xp22.2-p22.1 | RS1 |
| Rett syndrome | 312750 | Xq28, Xp22 | MECP2 |
| Muscular atrophy/ | 313200 | Xq11-q12 | AR |
| Dihydrotestosterone | | | |
| receptor deficiency | | | |
| Spinal muscular atrophy | 313200 | Xq11-q12 | AR |
| Spondyloepiphyseal dysplasia | 313400 | Xp22.2-p22.1 | SEDL |
| tarda | | | |
| Thrombocytopenia, hereditary | 313900 | Xp11.23-p11.22 | WAS |
| Throxine-binding globulin, | 314200 | Xq22.2 | TBG |
| absence | | | |
| McLeod syndrome | 314850 | Xp21.1 | XK |

Table 8 was adapted in part from Germain, “Chapter 7: General aspects of X-linked diseases” in Fabry Disease: Perspectives from 5 Years of FOS. Mehta A, Beck M, Sunder-Plassmann G, editors. (OXford: Oxford PharmaGenesis; 2006).

Identification of Direct Rna Interacting Proteins (iDRIP)

Also described herein is a method for identifying proteins that interact with a selected nucleic acid, e.g., an RNA such as an supRNA. The methods include in vivo UV crosslinking the proteins to the DNA in a living cell, preparing the nuclei, solubilizing the chromatin (e.g., by DNase I digestion), creating protein-RNA complexes through hybridization to capture probes specific for the selected RNA, treating the protein-RNA complexes with DNase, isolating the protein-RNA complexes using the capture probes (e.g., capture probes bound to beads) and washing, preferably under denaturing conditions to eliminate protein factors that were not covalently linked by UV to the selected RNA. To minimize background due to DNA-bound proteins, a critical DNase I treatment can be performed prior to elution. These methods can be used to identify proteins bound to any nucleic acid, e.g., RNA, e.g., any non-coding or coding RNA.

### Examples

The invention is further described in the following examples, which do not limit the scope of the invention described in the claims.

### Materials and Methods

The following materials and methods were used in the Examples, below.

Identification of Direct RNA interacting Proteins (iDRiP)

Mouse Embryonic Fibroblasts (MEFs) were irradiated with UV light at 200 mJ energy (Stratagene 2400) after rinsing with PBS. The pellets were resuspended in CSKT-0.5% (10 mM PIPES, pH 6.8, 100 mM NaCl, 3 mM MgCl₂, 0.3 M sucrose, 0.5% Triton X-100, 1 mM PMSF) for 10 min at 4° C. followed by a spin. The pellets were again resuspended in Nuclear Isolation Buffer (10 mM Tris pH 7.5, 10 mM KCl, 0.5% Nonidet-P 40, 1× protease inhibitors, 1 mM PMSF), and rotated at 4° C. for 10 min. The pellets were collected after a spin, weighed, flash frozen in liquid nitrogen, and stored at −80° C. until use.

Approximately, equal amounts of female and male UV cross linked pellets were thawed and resuspended for treatment with Turbo DNase I in the DNase I digestion buffer (50 mM Tris pH 7.5, 0.5% Nonidet-P 40, 0.1% sodium lauroyl sarcosine, 1×protease inhibitors, SuperaseIn). The tubes were rotated at 37° C. for 45 min. The nuclear lysates were further solubilized by adding 1% sodium lauroyl sarcosine, 0.3 M lithium chloride, 25 mM EDTA and 25 mM EGTA to final concentrations and continued incubation at 37° C. for 15 min. The lysates were mixed with biotinylated DNA probes (Table IA) prebound to the streptavidin magnetic beads (MyOne streptavidin Cl Dyna beads, Invitrogen) and incubated at 55° C. for 1 hr before overnight incubation at 37° C. in the hybridization chamber. The beads were washed three times in Wash Buffer (10 mM Tris, pH 7.5, 0.3 M LiCl, 1% LDS, 0.5% Nonidet-P 40, 1×protease inhibitor) at room temperature followed by treatment with Turbo DNase I in DNase I digestion buffer with the addition of 0.3 M LiCl, protease inhibitors, and superaseIn at 37° C. for 20 min. Then, beads were washed two more times in the Wash Buffer. For MS analysis, elution was done in Elution Buffer (10 mM Tris, pH 7.5, 1 mM EDTA) at 70° C. for 4 min followed by brief sonication in Covaris. For the quantification of pulldown efficiency, MEFs, without crosslinking, were used and elution was done at 95° C. The elute was used for RNA isolation and RT-qPCR. When crosslinked MEFs were used, elute was subjected for proteinase-K treatment (50 mM Tris pH 7.5, 100 mM NaCl, 0.5% SDS, 10 μg proteiase K) for 1 hr at 55° C. RNA were isolated by Trizol and quantified with SYBR green qPCR. Input samples were used to make standard curve by 10 fold dilutions, to which the RNA we, pulldown efficiencies were compared and calculated. The efficiency of Xist pulldown was relatively lower after UV crosslinking, similar to (48, 49).

| | | SEQ |
|---|---|---|
| | | ID |
| | Sequence | NO: |
| TABLE 1A. Biotinylated Oligos used in Xist | | |
| interactome capture | | |
| X1 | CAGTTTAAGAGCAAAGTCGTTTTTC | 1 |
| X2 | AATATGTTTACATTACAGGTGGCAA | 2 |
| X3 | TAAAGACCAAGCAAAGATACTTGTC | 3 |
| X4 | ATGCTTCATATATTCAGTGGTTCAC | 4 |
| X5 | TGTATTAAGTGAAATTCCATGACCC | 5 |
| X6 | AACTTAGCAATTAATTCTGGGACTC | 6 |
| X7 | ATGCATATCTGTATGCATGCTTATT | 7 |
| X8 | CATATTACTTGGGGACTAAGGACTA | 8 |
| X9 | ATGGGCACTGCATTTTAGCAATA | 9 |

Table 1B. Primers used in qPCR

| | | |
|---|---|---|
| U1 snRNA-F | CCAGGGCGAGGCTTATCCATT | 10 |
| U1 snRNA-R | GCAGTCCCCCACTACCACAAAT | 11 |
| eGFP-F | GAC GTA AAC GGC CÁC AAG TT | 12 |
| eGFP-R | AAG TCG TG CTG CTT CAT GTG | 13 |
| U6 snRNA-F | CTC GCT TCG GCA GCA CA | 14 |
| U6 snRNA-R | AAC GCT TCA CGA ATT TGC GT | 15 |
| Smc1a-F | TCG GAC CAT TTC AGA GGT TTA CC | 16 |
| Smc1a-R | CAG GTG CTC CAT GTA TCA GGT | 17 |
| Smc3-F | CGA AGT TAC CGA GAC CAA ACA | 18 |
| Smc3-R | TCA CTG AGA ACA AAC TGG ATT GC | 19 |
| Rad21-F | ATG TTC TAC GCA CAT TTT GTC CT | 20 |
| Rad21-R | TGC ACT CAA ATA CAT GGG CTT T | 21 |
| Kif4-F | AGG TGA AGG GGA TTC CCG TAA | 22 |
| Kif4-R | AAA CAC GCC TTT TAT GAG TGG A | 23 |
| Pds5a-F | TTG GGA AAC TGA TGA CCA TAG C | 24 |
| Pds5a-R | ACA CAA ACG TCA GCC TGC TT | 25 |
| Aurkb-F | CAG AAG GAG AAC GCC TAC CC | 26 |
| Aurkb-R | GAG AGC AAG CGC AGA TGT C | 27 |
| Top2b-F | CTG ACC TGG GTG AAC AAT GCT | 28 |
| Top2b-R | TGG CTC CAC TGA TCC AAT GTA T | 29 |
| Top2a-F | GAG AGG CTA CGA CTC TGA CC | 30 |
| Top2a-R | CTC CAG GTA GGG GGA TGT TG | 31 |
| Top1-F | AAG ATC GAG AAC ACC GGC ATA | 32 |
| Top1-R | CTT TTC CTC CTT CGG TCT TTC C | 33 |
| Ctcf-F | GAT CCT ACC CTT CTC CAG ATG AA | 34 |
| Ctcf-R | GTA CCG TCA CAG GAA CAG GT | 35 |
| Smarca4-F | CAA AGA CAA GCA TAT CCT AGC CA | 36 |
| Smarca4-R | CAC GTA GTG TGT GTT AAG GAC C | 37 |
| Smarca5-F | GAC ACC GAG ATG GAG GAA GTA | 38 |
| Smarca5-R | CGA ACA GCT CTG TCT GCT TTA | 39 |
| Smarcc1-F | AGC TAG ATT CGG TGC GAG TCT | 40 |
| Smarcc1-R | CCA CCA GTC CAG CTA GTG TTT T | 41 |
| Smarcc2-F | GCT GCC TAC AAA TTC AAG AGT GA | 42 |
| Smarcc2-R | AGG AAA ATG TTA GGT CGT GAC AG | 43 |
| Smarcb1-F | TCC GAG GTG GGA AAC TAC CTG | 44 |
| Smarcb1-R | CAG AGT GAG GGG TAT CTC TTG T | 45 |
| Sun2-F | ATC CAG ACC TTC TAT TTC CAG GC | 46 |
| Sun2-R | CCC GGA AGC GGT AGA TAC AC | 47 |

### Quantitative Proteomics

Proteins co-enriched with Xist from female or male cells were quantitatively analyzed either using a label-free approach based on spectral-counting (21) or by multiplexed quantitative proteomics using tandem-mass tag (TMT) reagents (50, 51) on an Orbitrap Fusion mass spectrometer (Thermo Scientific). Disulfide bonds were reduced with ditheiothreitol (DTT) and free thiols alkylated with iodoacetamide as described previously (22). Proteins were then precipitated with tricholoracetic acid, resuspended in 50 mM HEPES (pH 8.5) and 1 M urea and digested first with endoproteinase Lys-C (Wako) for 17 hours at room temperature and then with sequencing-grade trypsin (Promega) for 6 hours at 37° C. Peptides were desalted over Sep-Pak C₁₈ solid-phase extraction (SPE) cartridges (Waters), the peptide concentration was determined using a BCA assay (Thermo Scientific). For the label-free analysis peptides were then dried and re-suspended in 5% formic acid (FA) and % acetonitrile (ACN) and 5 μg of peptides were analyzed by mass spectrometry as described below. For the multiplexed quantitative analysis a maximum of 50 μg of peptides were labeled with one out of the available TMT-10plex reagents (Thermo Scientific) (51). To achieve this, peptides were dried and resuspended in 50 μl of 200 mM HEPES (pH 8.5) and 30% (ACN) and 10 μg of the TMT in reagent in 5 μl of anhydrous ACN was added to the solution, which was incubated at room temperature (RT) for one hour. The reaction was then quenched by adding 6 μl of 5% (w/v) hydroxylamine in 200 mM HEPES (pH 8.5) and incubation for 15 min at RT. The labeled peptide mixture was then subjected to a fractionation using basic pH reversed phase liquid chromatography (bRPLC) on an Agilent 1260 Infinity HPLC system equipped with an Agilent Extend-C18 column (4.6×250 mm; particle size, 5 μm) basically as described previously (52). Peptides were fractionated using a gradient from 22-35% ACN in 10 mM ammonium bicarbonate over 58 min at a flowrate of 0.5 ml/min. Fractions of 0.3 ml were collected into a 96-well plate to then be pooled into a total twelve fractions (A1-A12, B1-B12, etc.) that were dried and re-suspended in 8 μl of 5% FA and 5% ACN, 3 of which were analyzed by microcapillary liquid chromatography tandem mass spectrometry on an Orbitrap Fusion mass spectrometer and using a recently introduced multistage (MS3) method to provide highly accurate quantification (53).

The mass spectrometer was equipped with an EASY-nLC 1000 integrated autosampler and HPLC pump system. Peptides were separated over a 100 μm inner diameter microcapillary column in-house packed with first 0.5 cm of Magic C4 resin (5 μm, 100 Å, Michrom Bioresources), then with 0.5 cm of Maccel C₁₈ resin (3 μm, 200 Å, Nest Group) and 29 cm of GP-C₁₈ resin (1.8 μm, 120 Å, Sepax Technologies). Peptides were eluted applying a gradient of 8-27% ACN in 0.125% formic acid over 60 min (label-free) and 165 min (TMT) at a flow rate of 300 nl/min. For label-free analyses we applied a tandem-MS method where a full-MS spectrum (MS1; m/z 375-1500; resolution 6×10⁴; AGC target, 5×10⁵; maximum injection time, 100 ms) was acquired using the Orbitrap after which the most abundant peptide ions where selected for linear ion trap CID-MS2 in an automated fashion. MS2 scans were done in the linear ion trap using the following settings: quadrupole isolation at an isolation width of 0.5 Th; fragmentation method, CID; AGC target, 1×10⁴; maximum injection time, 35 ms; normalized collision energy, 30%). The number of acquired MS2 spectra was defined by setting the maximum time of one experimental cycle of MS1 and MS2 spectra to 3 sec (Top Speed). To identify and quantify the TMT-labeled peptides we applied a synchronous precursor selection MS3 method (22, 53, 54) in a data dependent mode. The scan sequence was started with the acquisition of a full MS or MS1 one spectrum acquired in the Orbitrap (m/z range, 500-1200; other parameters were set as described above), and the most intense peptide ions from detected in the full MS spectrum were then subjected to MS2 and MS3 analysis, while the acquisition time was optimized in an automated fashion (Top Speed, 5 sec). MS2 scans were performed as described above. Using synchronous precursor selection the 10 most abundant fragment ions were selected for the MS3 experiment following each MS2 scan. The fragment ions were further fragmented using the HCD fragmentation (normalized collision energy, 50%) and the MS3 spectrum was acquired in the Orbitrap (resolution, 60,000; AGC target, 5×10⁴; maximum injection time, 250 ms).

Data analysis was performed on an on an in-house generated SEQUEST-based (55) software platform. RAW files were converted into the mzXML format using a modified version of ReAdW.exe. MS2 spectra were searched against a protein sequence database containing all protein sequences in the mouse UniProt database (downloaded 02/04/2014) as well as that of known contaminants such as porcine trypsin. This target component of the database was followed by a decoy component containing the same protein sequences but in flipped (or reversed) order (56). MS2 spectra were matched against peptide sequences with both termini consistent with trypsin specificity and allowing two missed trypsin cleavages. The precursor ion m/z tolerance was set to 50 ppm, TMT tags on the N-terminus and on lysine residues (229.162932 Da, only for TMT analyses) as well as carbamidomethylation (57.021464 Da) on cysteine residues were set as static modification, and oxidation (15.994915 Da) of methionines as variable modification. Using the target-decoy database search strategy (56) a spectra assignment false discovery rate of less than 1% was achieved through using linear discriminant analysis with a single discriminant score calculated from the following SEQUEST search score and peptide sequence properties: mass deviation, XCorr, dCn, number of missed trypsin cleavages, and peptide length (57). The probability of a peptide assignment to be correct was calculated using a posterior error histogram and the probabilities for all peptides assigned to a protein were combined to filter the data set for a protein FDR of less than 1%. Peptides with sequences that were contained in more than one protein sequence from the UniProt database were assigned to the protein with most matching peptides (57).

For a quantitative estimation of protein concentration using spectral-counts we simply counted the number of MS2 spectra assigned to a given protein (Tables 5-6). TMT reporter ion intensities were extracted as that of the most intense ion within a 0.03 Th window around the predicted reporter ion intensities in the collected MS3 spectra. Only MS3 with an average signal-to-noise value of larger than 28 per reporter ion as well as with an isolation specificity (22) of larger than 0.75 were considered for quantification. Reporter ions from all peptides assigned to a protein were summed to define the protein intensity. A two-step normalization of the protein TMT-intensities was performed by first normalizing the protein intensities over all acquired TMT channels for each protein based to the median average protein intensity calculated for all proteins. To correct for slight mixing errors of the peptide mixture from each sample a median of the normalized intensities was calculated from all protein intensities in each TMT channel and the protein intensities were normalized to the median value of these median intensities.

### Uv Rip

The protocol followed is similar to the one described in (18). Briefly, MEFs were crosslinked with UV light at 200 mJ and collected by scraping in PBS. Cell pellets were resuspended in CSKT-0.5% for 10 min at 4° C. followed by a spin. The nuclei were resuspended in the UV RIP buffer (PBS buffer containing 300 mM NaCl (total), 0.5% Nonidet-P 40, 0.5% sodium deoxycholate, and 1×protease inhibitors) with Turbo DNase I 30 U/IP for 30 min at 37° C. Supernatants were collected after a spin and incubated with 5 μg specific antibodies prebound to 40 μl protein-G magnetic beads (Invitrogen) at 4° C. overnight. Beads were washed three times with cold UV RIP buffer. The beads were resuspended in 200 μl Turbo DNase I buffer with 20 U Turbo DNase, SuperaseIN, 1× protease inhibitors) for 30 min at 37° C. The beads were resuspended and washed three more times in the UV RIP washing buffer containing 10 mM EDTA. The final 3 washes were given after three fold dilution of UV RIP washing buffer. The beads were resuspended in 200 μl proteinase-K buffer with 10 μg proteinase-K and incubated at 55° C. for 1 hr. RNA was isolated by Trizol [0.1 and pulldown efficiencies were calculated by SYBR qPCR using input for the standard curve.

Generation of Xi-TgGFP Clonal Fibroblasts Xi-TgGFP (68-5-11) tail-tip fibroblasts (TTF) were initially derived from a single female pup, a daughter of a cross between a M. castaneus male and a M. musculus female, homozygous for an X-linked GFP transgene driven by a strong, ubiquitous promoter (58). The fibroblasts were immortalized by SV40 transformation, and clonal lines were derived from individual GFP-negative cells selected by fluorescence-activated cell sorting. In our experience, occasional clones with undetectable GFP expression nevertheless have the transgene located on the active X chromosome. Thus, we confirmed the GFP transgene location on the inactive X for the particular clone used here, 68-5-11 (see FIG. 10).

### Generation of Stable KD of Xi-TgGFP TTF and 16.7 ES Cells

A cocktail of 3 shRNA viruses were used for infections (Table 2) followed with puromycin selection using standard methodology. In all the experiments, polyclonal knock down cells were used.

TABLE 2
Lentiviral shRNA constructs used for stable knockdowns of
candidate Xist interactors.

| RefSeq_shRNA viruses | Xist interacting candidates |
|---|---|
| TRCN0000011883 | Top1 |
| TRCN0000321370 | Ctcf |
| TRCN0000071385 | Smarca4 |
| TRCN0000295773 | Smarca5 |
| TRCN0000321371 | Ctcf |
| TRCN0000109008 | SMc3 |
| TRCN0000276847 | Rad21 |
| TRCN0000174832 | Rad21 |
| TRCN0000321718 | Aurkb |
| TRCN0000317702 | Smarcb1 |
| TRCN0000071383 | SMarca4 |
| TRCN0000325493 | Top2a |
| TRCN0000295713 | Smarca5 |
| TRCN0000309135 | Kif4 |
| TRCN0000321651 | Aurkb |
| TRCN0000109007 | Smc3 |
| TRCN0000090909 | Kif4 |
| TRCN0000321444 | Ctcf |
| TRCN0000071388 | Smarcc1 |
| TRCN0000288446 | Smarca5 |
| TRCN0000072181 | GFP |
| TRCN0000071389 | Smarcc1 |
| TRCN0000070988 | Top2b |
| TRCN0000011884 | Top1 |
| TRCN0000070990 | Top2b |
| TRCN0000229486 | Pds5a |
| TRCN0000011886 | Top1 |
| TRCN0000085541 | Smarcc2 |
| TRCN0000317622 | Smarcb1 |
| TRCN0000324673 | Smc1a |
| TRCN0000229484 | Pds5a |
| TRCN0000085540 | Smarcc2 |
| TRCN0000070987 | Top2a |
| TRCN0000071386 | Smarca4 |
| TRCN0000109009 | Smc3 |
| TRCN0000246806 | Sun2 |
| TRCN0000276903 | Rad21 |
| TRCN0000071391 | Smarcc1 |
| TRCN0000070992 | Top2b |
| TRCN0000317701 | Smarcb1 |
| TRCN0000085542 | Smarcc2 |
| TRCN0000321719 | Aurkb |
| TRCN0000246805 | Sun2 |
| TRCN0000246804 | Sun2 |
| TRCN0000217996 | Pds5a |
| TRCN0000090908 | Kif4 |
| TRCN0000324674 | Smc1a |
| TRCN0000324672 | Smc1a |
| TRCN0000353984 | Top2a |
| TRCN0000231782_pLKO_TRC021 | control |
| TRCN0000231782_pLKO_TRC021 | control |

Assay for the reactivation of Xi-TgGFP

Approximately, 125,000-150,000 Xi-TgGFP (68-5-11) cells were plated along with control (shNegative control, i.e., shNC) cells treated with DMSO or stable KD cells treated with 0.3 μM azacytidine and 0.3 μM Etoposide for 3 days in 6 well plates. RNA was isolated by Trizol twice, with an intermittent TurboDNase treatment after the first isolation for 30 min at 37° C. One μg RNA was used for each of the RT+ and RT− reactions (Superscript III, Invitrogen) followed by the SYBR green qPCR using the primers listed in Table 3, with annealing temperature of 60° C. for 45 cycles. The relative efficiency of Xi-TgGFP reactivations was calculated by comparing to U1 snRNA as the internal control.

TABLE 3
Primers used in PCR for generation
of Xi-TgGFP cell line

| | | SEQ |
|---|---|---|
| | | ID |
| | Sequence | NO: |
| MeCP2-F | ATGGTAGCTGGGATGTTAGGG | 48 |
| MeCP2-R | GAGCGAAAAGCTTTTCCCTGG | 49 |

### ImmunoFISH

Cells were grown on coverslips, rinsed in PBS, pre-extracted in 0.5% CSKT on ice, washed once in CSK, followed by fixation with 4% paraformaldehyde in PBS at room temperature. After blocking in 1% BSA in PBS for 20 min supplemented with 10 mM VRC (New England Biolabs) and RNase inhibitor (Roche), incubation was carried out with primary antibodies (Table 4) at room temperature for 1 hr. Cells were washed three times in PBST-0.02% Tween-20. After incubating with secondary antibody at room temperature for 30 min, cells were washed three times by PBS/0.02% Tween-20. Cells were fixed again in 4% paraformaldehyde and dehydrated in ethanol series. RNA FISH was performed using a pool of Cy3B or Alexa 568 labeled Xist oligonucleotides for 4-6 hours at 42° C. in a humid chamber. Cells were washed three times in 2×SSC and nuclei were counter-stained by Hoechst 33342. Cells were observed under Nikon 90i microscope equipped with 60×/1.4 N. A. objective lens, Orca ER CCD camera (Hamamatsu), and Volocity software (Perkin Elmer). Xist RNA FISH probes, a set of total 37 oligonucleotides with 5′ amine modification (IDT), were labeled with NHS-Cy3B (GE Healthcare) overnight at room temperature followed by ethanol precipitation. In the case of confirmation of Xi-TgGFP cells, probes were made by nick-translation of a GFP PCR product with Cy3-dUTP and of a plasmid containing the first exon of the mouse Xist gene, with FITC-dUTP.

TABLE 4
Antibodies

| Brand | Antibodies and Catalog # |
|---|---|
| NOVUS BIOLOGICALS INC | SMC3 antibody (NB100-207) |
| NOVUS BIOLOGICALS INC | SMC1 Antibody (A300-055A) |
| BETHYL LABORATORIES INC | TOP1 Antibody (A302-589A) |
| SIGMA-ALDRICH INC | ANTI-SUN2 antibody (HPA001209-100UL) |
| ABCAM INC | Anti-BRG1 antibody [EPNCIR111A] (ab110641) |
| PROTEINTECH GROUP INC | TOP2A-Specific Antibody (20233-1-AP) |
| ABCAM INC | Anti-Aurora B Kinase antibody (ab2254) |
| ABCAM INC | Anti-Rad21 antibody - ChIP Grade (ab992) |
| ACTIVE MOTIF | Histone H3K27me3 antibody (pAb) (39155) |
| PROTEINTECH GROUP INC | TOP2B Polyclonal Antibody (20549-1-AP) |
| CELL SIGNALING TECHNOLOGY | SMARCC2/BAF170 (D8O9V) Rabbit mAb (12760) |
| E M D MILLIPORE | Anti-CTCF Antibody (07-729) |

### Allelic ChIP-seq

Allele-specific ChIP-seq was performed according to the method of Kung et al (25), in two biological replicates. To increase available read depth, we pooled together two technical replicates for X^{ΔXist}/Xa^{WT} Rad21 replicate 1 sequenced on a 2×50 bp HiSeq2500 rapid run and we also pooled two technical replicates of wild-type Rad21 replicate 1, one sequenced on a HiSeq2×50 bp run and one on a MiSeq 2×50 bp run. All other libraries were sequenced on using 2×50 bp HiSeq2500 rapid runs. To visualize ChIP binding signal, we generated fpm-normalized bigWig files from the raw ChIP read counts for all reads (comp), mus-specific (mus) and cas-specific reads separately. For Smc1a, CTCF and Rad21, peaks were called using macs2 with default settings. To generate consensus peak sets for all three epitopes, peaks for the two wild-type and X^{ΔXist}/Xa^{WT} replicates were pooled and peaks present in at least two experiments were used as the common peak set. To make comparisons between allelic read counts between different experiments, we defined a scaling factor as the ratio of the total read numbers for the two experiments and multiplied the allelic reads for each peak in the larger sample by the scaling factor. We plotted the number of reads on Xi vs Xa in wild-type for all peaks on the X-chromosome to determine if there is a general bias towards binding to the Xa or the Xi. To evaluate allelic skew on an autosome, we generated plots of mus read counts vs cas read counts for all peaks on chromosome 5 from 1-140,000,000. We used this particular region of chromosome 5 because X^{ΔXist}/Xa^{WT} is not fully hybrid, and this is a large region of an autosome that is fully hybrid based on even numbers of read counts from input and from our Hi-Cs over this region in X^{ΔXist}/Xa^{WT} (data not shown). To identify peaks that are highly Xa-skewed in wild-type but bind substantially to the Xi in X^{ΔXist}/Xa^{WT} (restored peaks), for Xa-skewed peaks in wild-type, we plotted normalized read counts on Xi in X^{ΔXist}/Xa^{WT} versus read counts on Xa in wild-type. We defined restored peaks as peaks that are 1.) more than 3×Xa-skewed in wild-type 2.) have at least 5 allelic reads in wild-type 3.) exhibit normalized read counts on Xi in X^{ΔXist}/Xa^{WT} that are at least half the level of Xa in wild-type. This threshold ensures that all restored peaks have at least a 2×increase in binding to the Xi in X^{ΔXist}/Xa^{WT} relative to wild-type. We identified restored peaks using these criteria in both replicates of Smc1a and Rad21 ChIP separately, and to merge these calls into a consensus set for each epitope, we took all peaks that met criteria for restoration in at least one replicate and had at least 50% wild-type Xa read counts on Xi in Xi^{t}/Xa^{WT} in both replicates.

Allele specific RNA-seq

Xi-TgGFP TTFs (68-5-11) with the stable knock down of candidates were treated with 5′-azacytidine and etoposide at 0.3 μM each for 3 days. Strand-specific RNA-seq, the library preparation, deep sequencing, and data analysis was followed as described in (25). Two biological replicates of each drug treatment were produced. All libraries were sequenced with Illumina Hiseq 2000 or 2500 using 50 cycles to obtain paired end reads. To determine the allelic origin of each sequencing read from the hybrid cells, reads were first depleted of adaptors dimers and PCR duplicates, followed by the alignment to custom mus/129 and cas genomes to separate mus and cas reads. After removal of PCR duplicates, ˜90% of reads were mappable. Discordant pairs and multi-mapped reads were discarded. Reads were then mapped back to reference mm9 genome using Tophat v2.0.10 (-g 1-no-coverage-search-read-edit-dist 3-read-mismatches 3-read-gap-length 3-b2-very-sensitive-mate-inner-dist 50-mate-std-dev 50-library-type fr-firststrand), as previously described (59, 32, 25). Following alignment, gene expression levels within each library were quantified using Homer v4.7 (ma mm9-count genes-strand+-noadj−condenseGenes) (59) and the normalized differential expression analyses across samples were performed by using EdgeR (60).

### HiC Library Preparation and Analysis

Hi-C libraries were generated according to the protocol in Lieberman-Aiden et al., 2009 (61). Two biological replicate libraries were prepared for wild-type and X^{ΔXist}/Xa^{WT} fibroblasts each. We obtained 150-220 million 2×50 bp paired-end reads per library. The individual ends of the read-pairs were aligned to the mus and cas reference genomes separately using novoalign with default parameters for single-end alignments, and the quality score of the alignment was used to determine whether each end could be assigned to either the mus or the cas haplotype (62). The single-end alignments were merged into a Hi-C summary file using custom scripts. Reads were filtered for self-ligation events and short fragments (less than 1.5× the estimated insert length) likely to be random shears using Homer (59, 63). Hi-C contact maps were generated using Homer. “Comp” maps were made from all reads. “Xi” and “Xa” reads were from reads where at least one read-end could be assigned to either the mus or cas haplotype, respectively. A small fraction of reads (˜5% of all allelic reads) aligned such that one end aligned to mus, the other to cas. These “discordant” reads were excluded from further analysis, as they are likely to be noise arising due to random ligation events and/or improper SNP annotation (64, 46). All contact maps were normalized using the matrix balancing algorithm of Knight and Ruiz (65), similar to iterative correction (66, 46), using the MATLAB script provided at the end of their paper. We were able to generate robust contact maps using the comp reads in one replicate at 40kb resolution, but due to the fact that only ˜44% of reads align allele-specifically, we were only able to generate contact maps for the cas and mus haplotypes at 200kb. To increase our resolution, we pooled together both biological replicates and analyzed the comp contact map at 40kb resolution and the mus and cas contact maps at 100kb. We called TADs at 40kb on chrX, chr5 and chr13 using the method of Dixon et al. (27). specifically, we processed the normalized comp 40kb contact maps separately into a vector of directionality indices using DI_from matrix.pl with a bin size of 40000 and a window size of 200000. We used this vector of directionality indices as input for the HMM_calls.m script and following HMM generation, we processed the HMM and generated TAD calls by passing the HMM output to file_ends_cleaner.pl, converter 7col.pl, hmm probablity_correcter.pl, hmm-state_caller.pl and finally hmm-state_domains.p1. We used parameters of min=2, prob=0.99, binsize=40000 as input to the HMM probability correction script.

To create a general metric describing interaction frequencies within TADS at resolution available in the allele-specific interaction maps, for each TAD, on chrX and chr5 we averaged the normalized interaction scores for all bins within each TAD, excluding the main diagonal. To make comparisons between interaction frequency over TADS between the cas (Xa) and mus (Xi) haplotypes at the resolution available with our current sequencing depth, we defend the “fraction mus” as the average interaction score for a TAD in the mus contact map divided by the sum of the average interaction scores in the mus and cas contact maps.

To discover TADS that show significantly increased interaction frequency in X^{ΔXist}/Xa^{WT}, we generated a null distribution of changes in average normalized interaction scores for all TADS on chromosome 5, 1-140 Mb using the cas and mus contact maps. We reasoned that there would be few changes in interaction frequency on an autosome between the mus or cas contact maps for wild-type and X^{ΔXist}/Xa^{WT}, thus the distribution of fold changes in interaction score on an autosome constitutes a null distribution. Using this distribution of fold changes allowed us to calculate a threshold fold change for an empirical FDR of 0.05, and all TADS that had a greater increase in average normalized interaction score on Xi between wild-type and X^{ΔXist}/Xa^{WT} were considered restored TADs. We preformed this analysis of restored TADS separately in each biological replicate using the 200kb contact maps to generate interaction scores over TADs, and using the combined data at 100kb resolution.

### References for Materials and Methods Section Only

1. A. Castello et al., Insights into RNA biology from an atlas of mammalian mRNA-binding proteins. Cell 149, 1393-1406 (2012).

2. S. C. Kwon et al., The RNA-binding protein repertoire of embryonic stem cells. Nature structural & molecular biology 20, 1122-1130 (2013).

3. D. H. Lundgren, S. I. Hwang, L. Wu, D. K. Han, Role of spectral counting in quantitative proteomics. Expert review of proteomics 7, 39-53 (2010).

4. G. C. McAlister et al., Increasing the multiplexing capacity of TMTs using reporter ion isotopologues with isobaric masses. Analytical chemistry 84, 7469-7478 (2012).

5. A. Thompson et al., Tandem mass tags: a novel quantification strategy for comparative analysis of complex protein mixtures by MS/MS. Analytical chemistry 75, 1895-1904 (2003).

6. L. Ting, R. Rad, S. P. Gygi, W. Haas, MS3 eliminates ratio distortion in isobaric multiplexed quantitative proteomics. Nature methods 8, 937-940 (2011).

7. A. C. Tolonen, W. Haas, Quantitative proteomics using reductive dimethylation for stable isotope labeling. Journal of visualized experiments: JoVE, (2014).

8. G. C. McAlister et al., MultiNotch MS3 enables accurate, sensitive, and multiplexed detection of differential expression across cancer cell line proteomes. Analytical chemistry 86, 7150-7158 (2014).

9. M. P. Weekes et al., Quantitative temporal viromics: an approach to investigate host-pathogen interaction. Cell 157, 1460-1472 (2014).

10. J. K. Eng, A. L. McCormack, J. R. Yates, An approach to correlate tandem mass spectral data of peptides with amino acid sequences in a protein database. Journal of the American Society for Mass Spectrometry 5, 976-989 (1994).

11. J. E. Elias, S. P. Gygi, Target-decoy search strategy for increased confidence in large-scale protein identifications by mass spectrometry. Nature methods 4, 207-214 (2007).

12. E. L. Huttlin et al., A tissue-specific atlas of mouse protein phosphorylation and expression. Cell 143, 1174-1189 (2010).

13. Y. Jeon, J. T. Lee, YY1 tethers Xist RNA to the inactive X nucleation center. Cell 146, 119-133 (2011).

14. A. K. Hadjantonakis, L. L. Cox, P. P. Tam, A. Nagy, An X-linked GFP transgene reveals unexpected paternal X-chromosome activity in trophoblastic giant cells of the mouse placenta. Genesis 29, 133-140 (2001).

15. J. T. Kung et al., Locus-Specific Targeting to the X Chromosome Revealed by the RNA Interactome of CTCF. Molecular cell 57, 361-375 (2015).

16. J. T. Kung et al., Locus-specific targeting to the X chromosome revealed by the RNA interactome of CTCF. Molecular cell 57, 361-375 (2015).

17. S. F. Pinter et al., Spreading of X chromosome inactivation via a hierarchy of defined Polycomb stations. Genome research 22, 1864-1876 (2012).

18. S. Heinz et al., Simple combinations of lineage-determining transcription factors prime cis-regulatory elements required for macrophage and B cell identities. Molecular cell 38, 576-589 (2010).

19. M. D. Robinson, D. J. McCarthy, G. K. Smyth, edgeR: a Bioconductor package for differential expression analysis of digital gene expression data. Bioinformatics 26, 139-140 (2010).

20. E. Lieberman-Aiden et al., Comprehensive mapping of long-range interactions reveals folding principles of the human genome. Science (New York, N.Y.) 326, 289-293 (2009).

21. E. Yildirim, R. Sadreyev, S. Pinter, J. Lee, X-chromosome hyperactivation in mammals via nonlinear relationships between chromatin states and transcription. Nature structural & molecular biology 19, 56-61 (2012).

22. C. L. Yin et al., Global changes in the nuclear positioning of genes and intra- and interdomain genomic interactions that orchestrate B cell fate. Nature Immunology 13, 1196-1204 (2012).

23. H. Sven et al., Simple Combinations of Lineage-Determining Transcription Factors Prime cis-Regulatory Elements Required for Macrophage and B Cell Identities. Molecular Cell 38, (2010).

24. S. Selvaraj, J. R Dixon, V. Bansal, B. Ren, Whole-genome haplotype reconstruction using proximity-ligation and shotgun sequencing. Nature biotechnology, (2013).

25. S. S. Rao et al., A 3D map of the human genome at kilobase resolution reveals principles of chromatin looping. Cell 159, 1665-1680 (2014).

26. P. A. Knight, D. Ruiz, A fast algorithm for matrix balancing. IMA Journal of Numerical Analysis, (2012).

27. I. Maxim et al., Iterative correction of Hi-C data reveals hallmarks of chromosome organization. Nature Methods 9, 999-1003 (2012).

28. J. Dixon et al., Topological domains in mammalian genomes identified by analysis of chromatin interactions. Nature 485, 376-380 (2012).

### Example 1. iDRiP Identifies Multiple Classes of Xist-Interacting Proteins

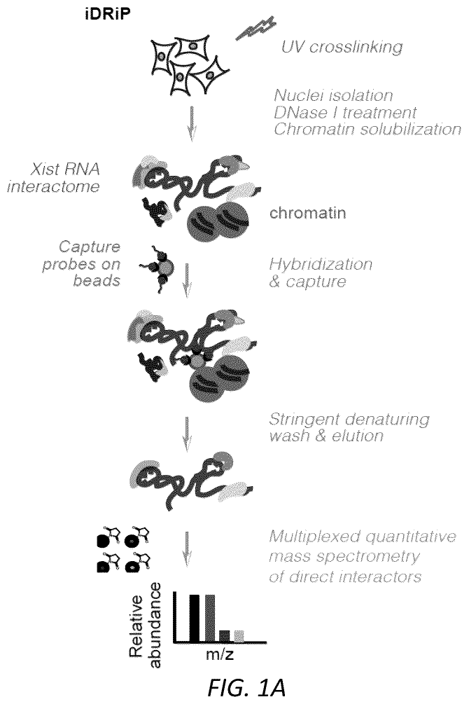

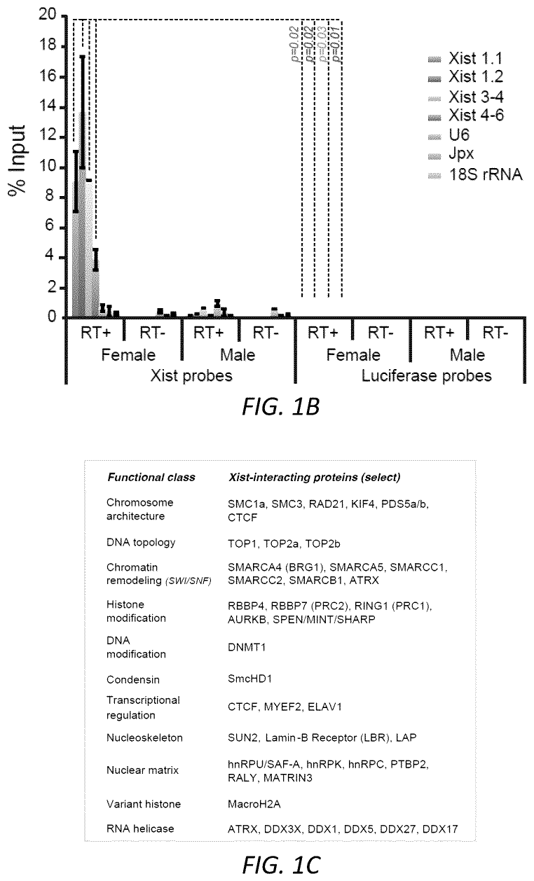

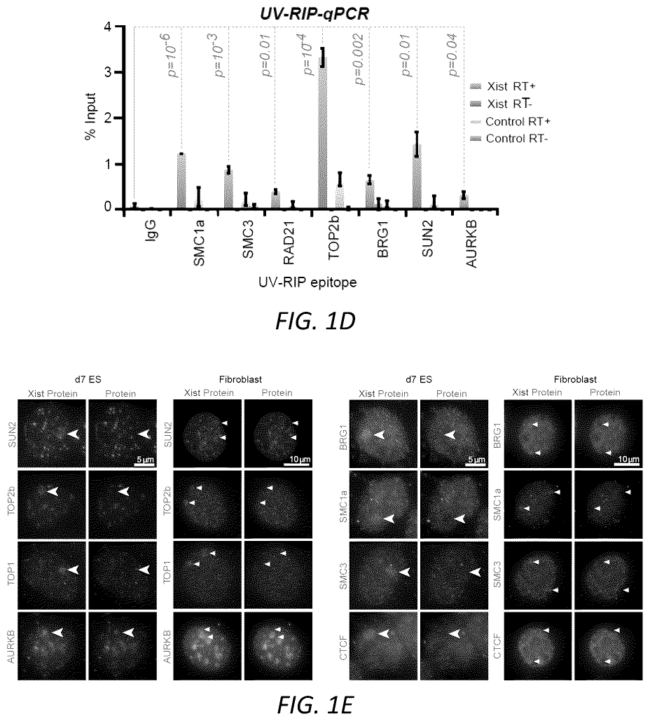

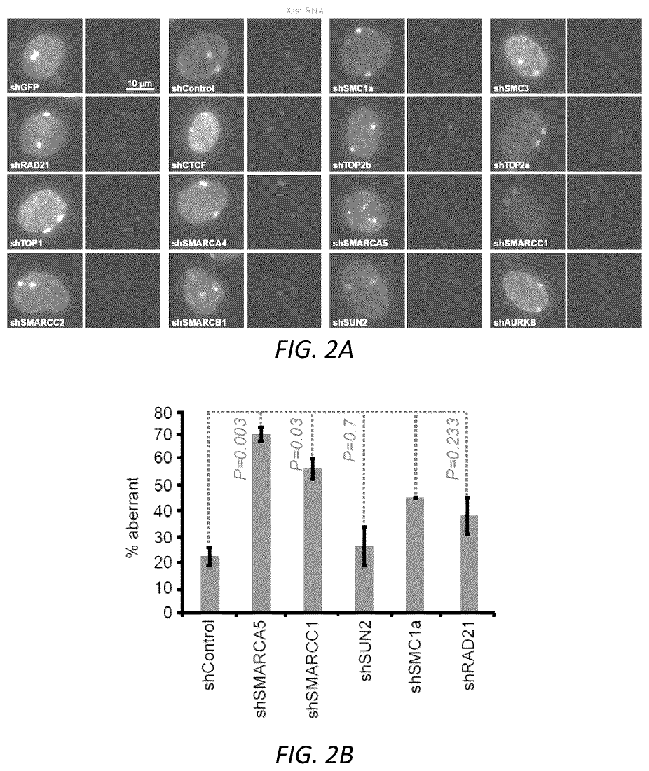

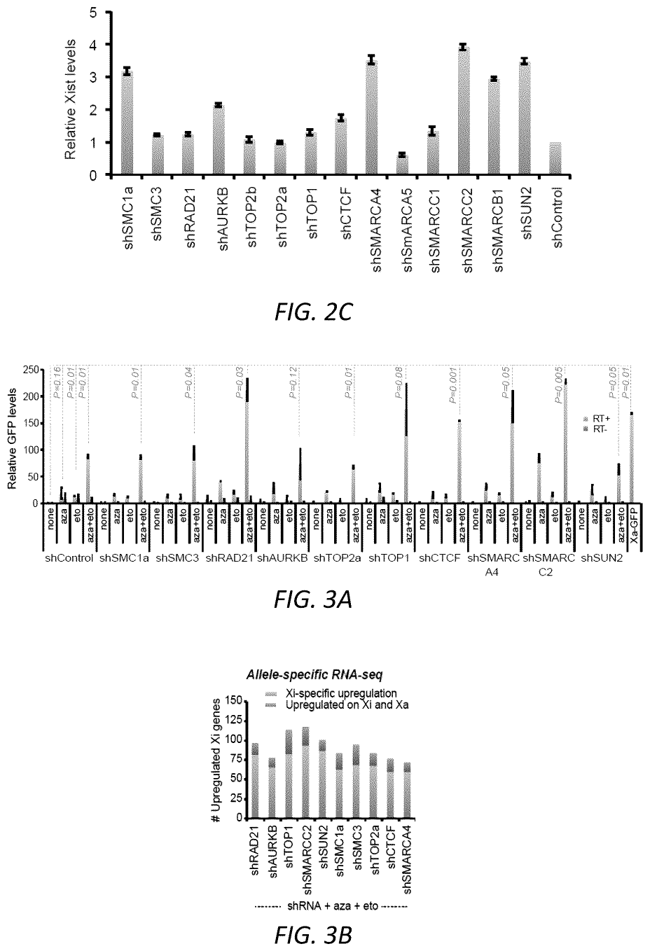

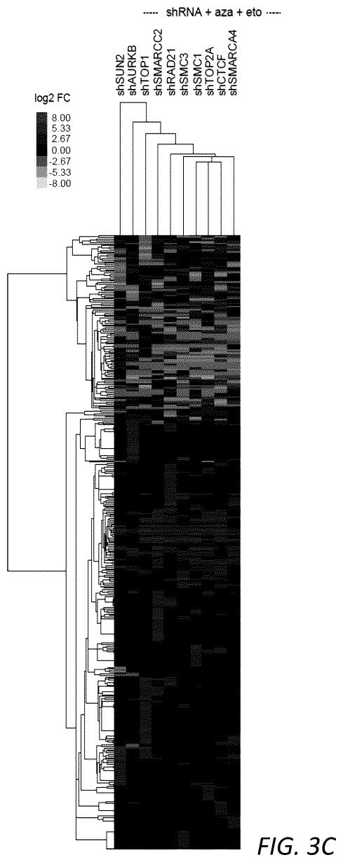

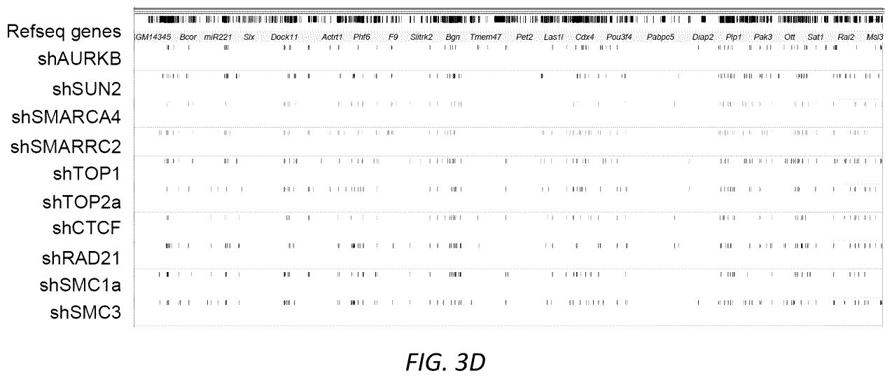

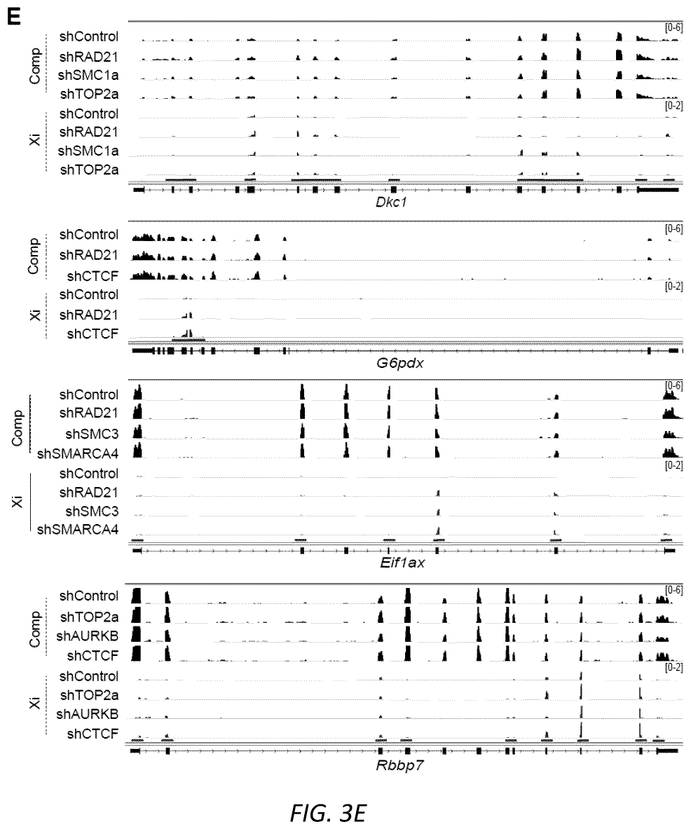

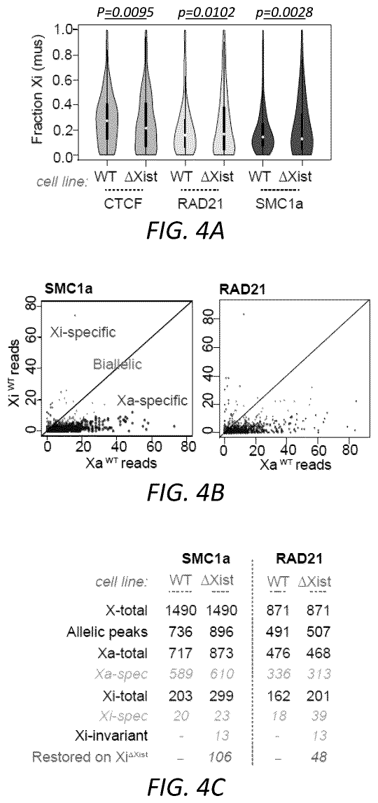

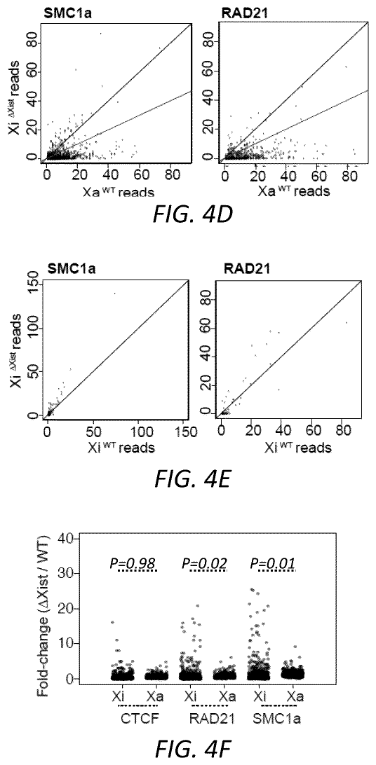

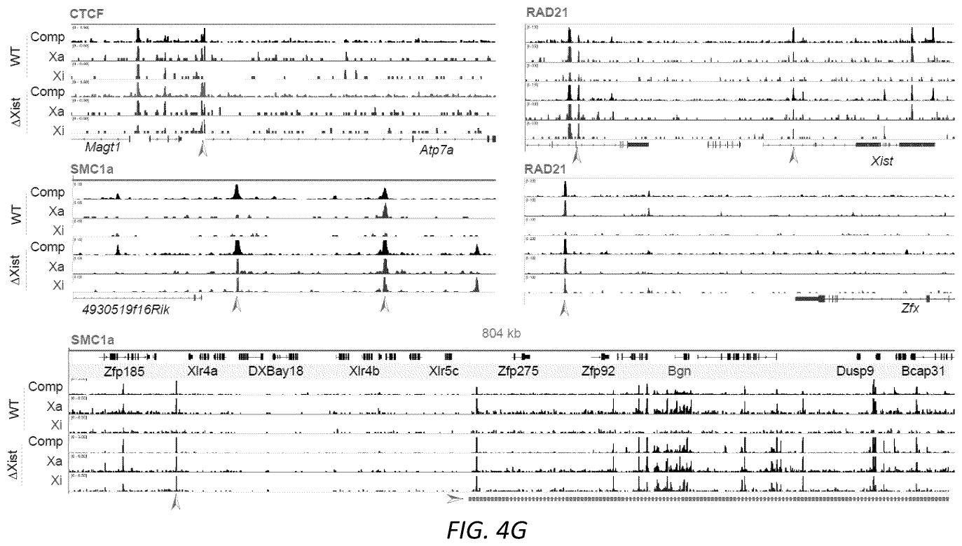

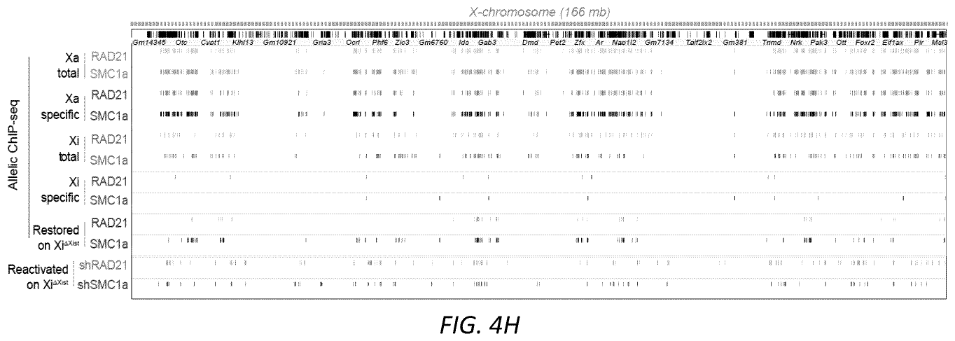

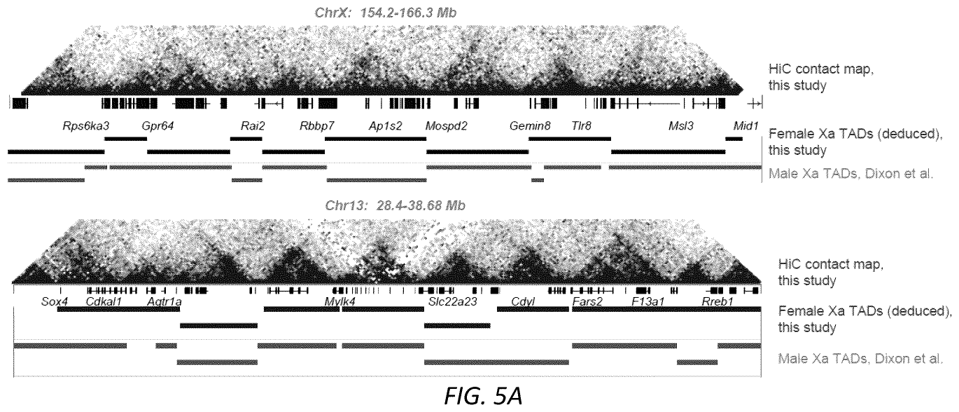

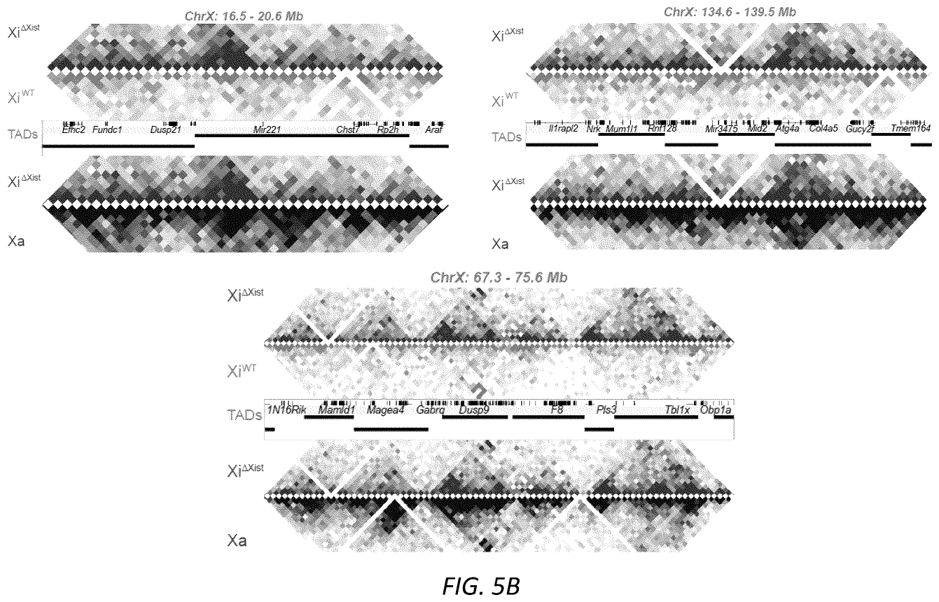

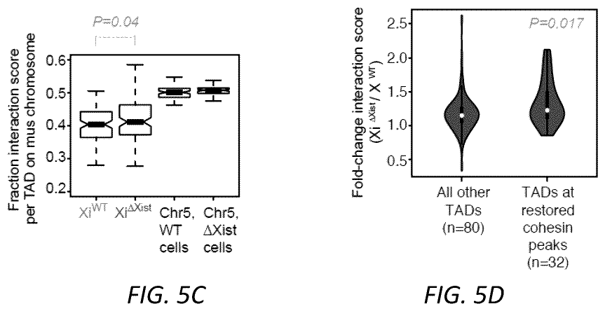

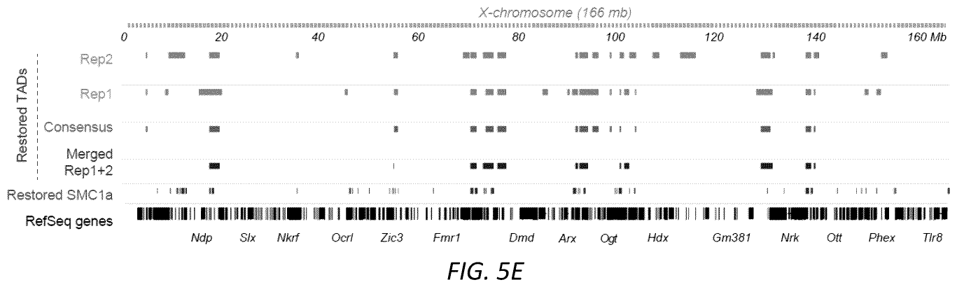

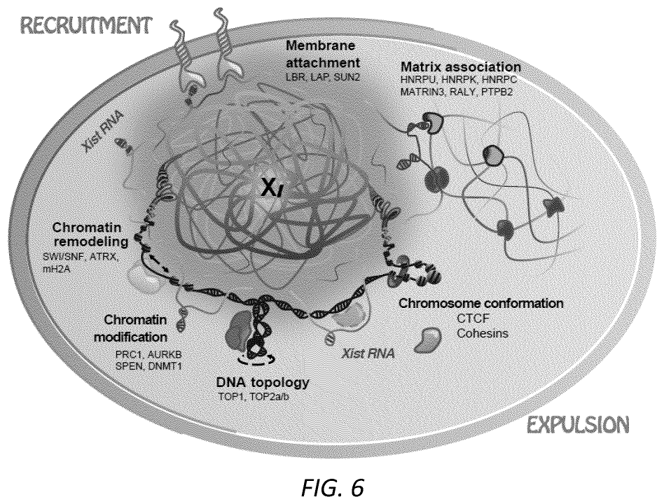

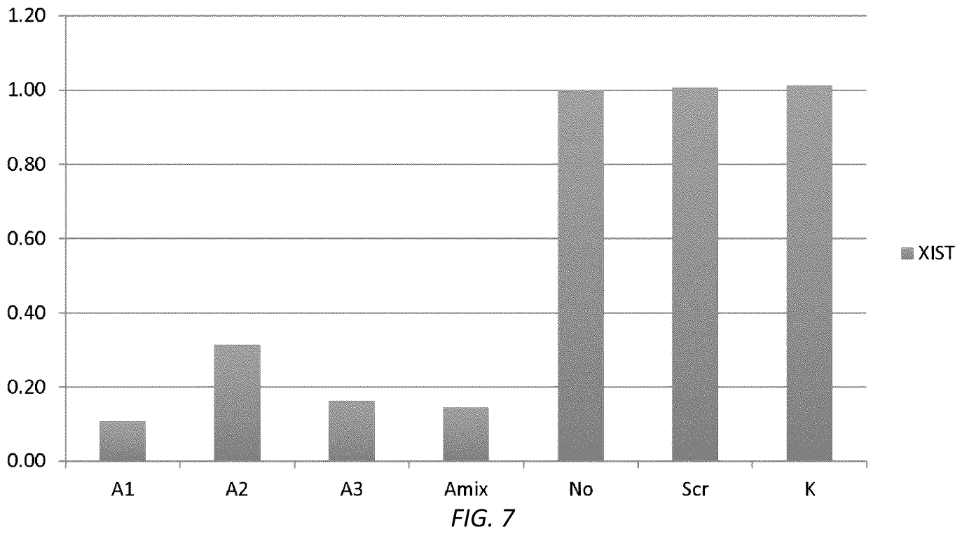

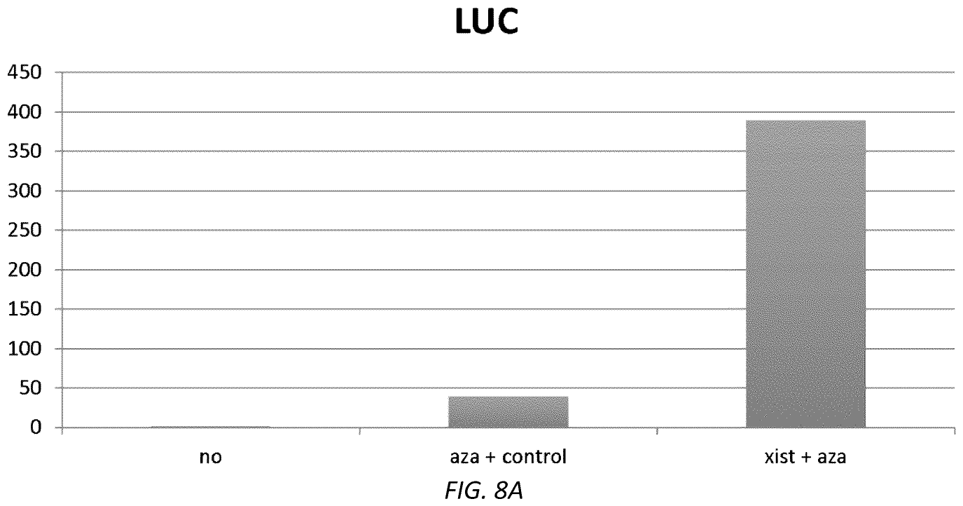

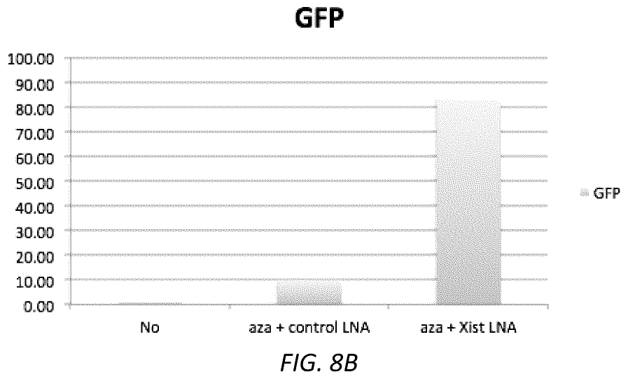

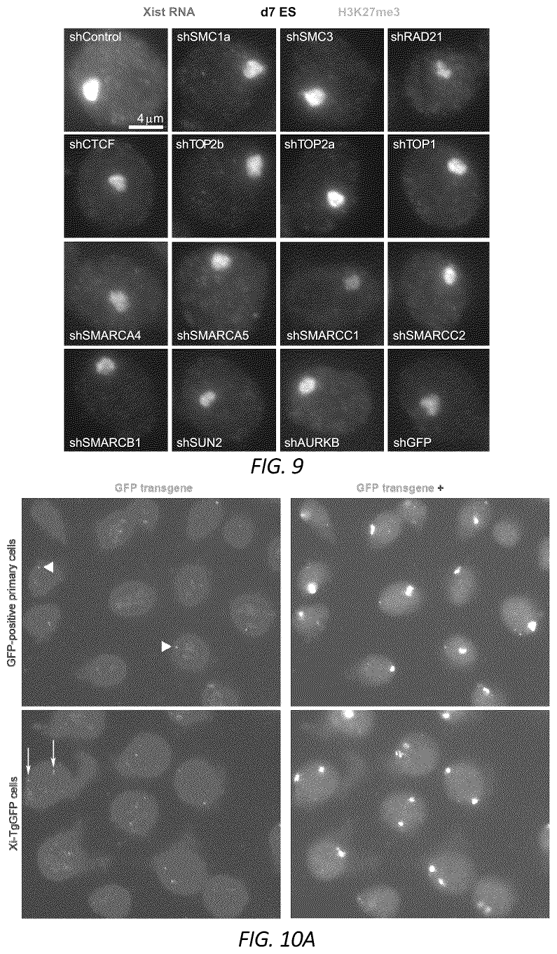

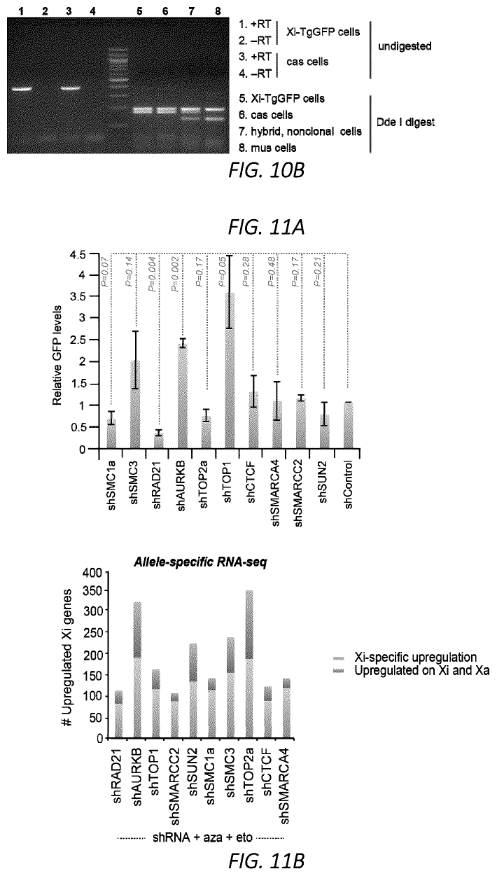

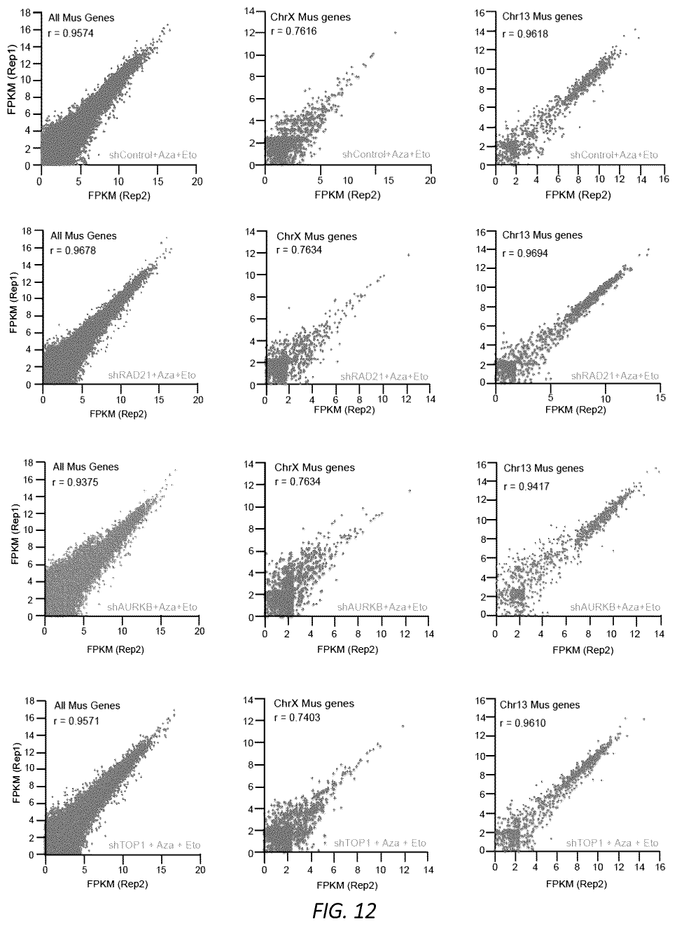

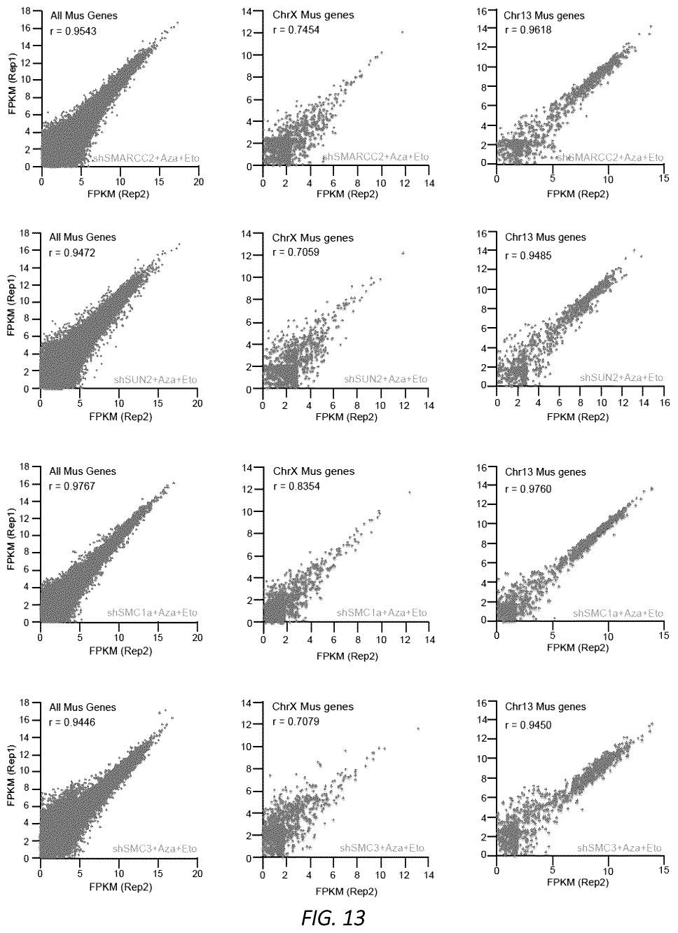

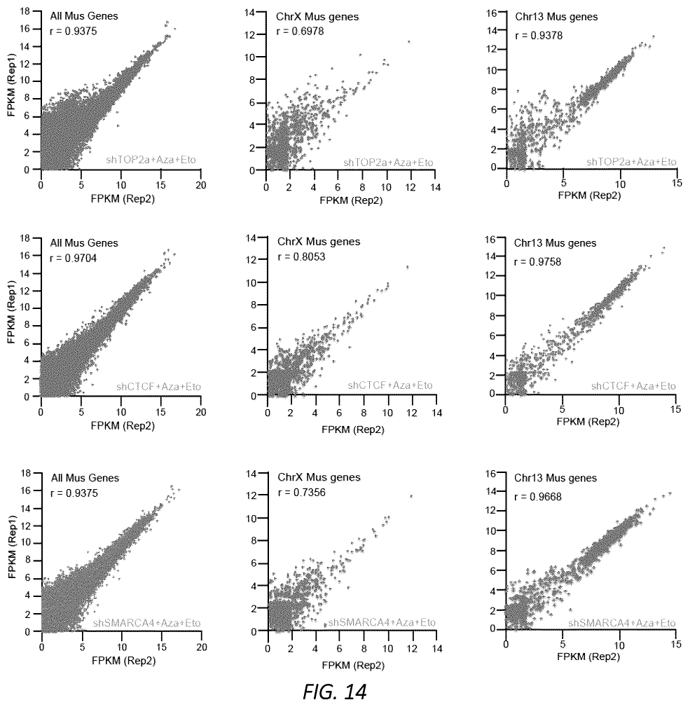

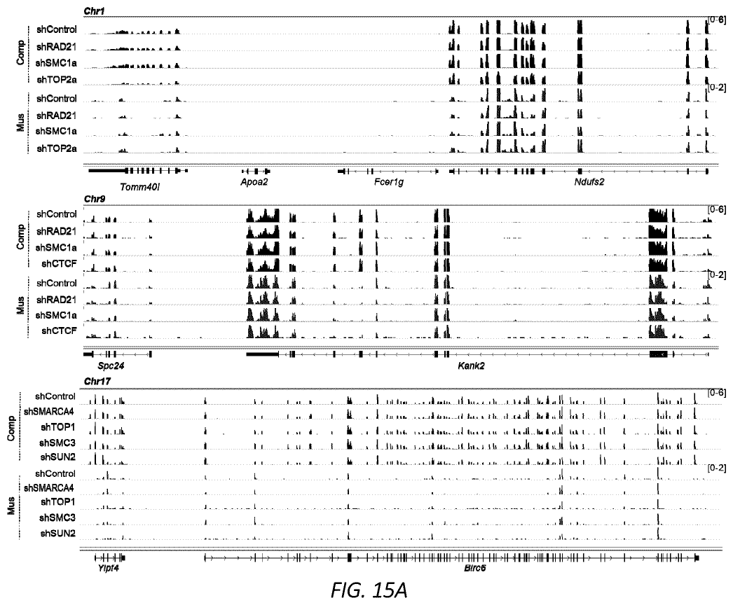

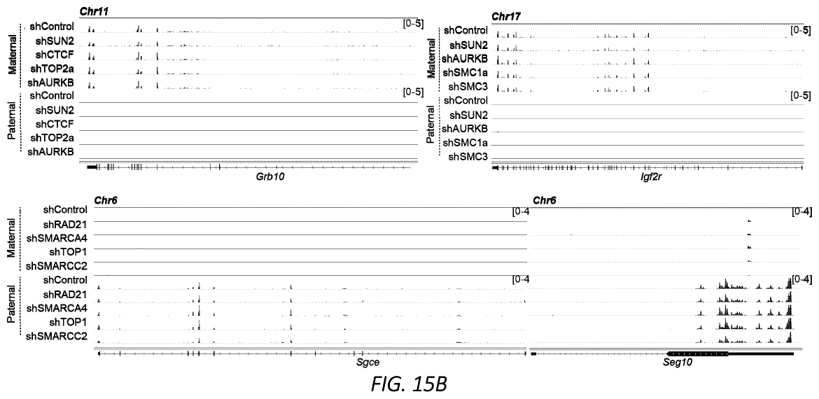

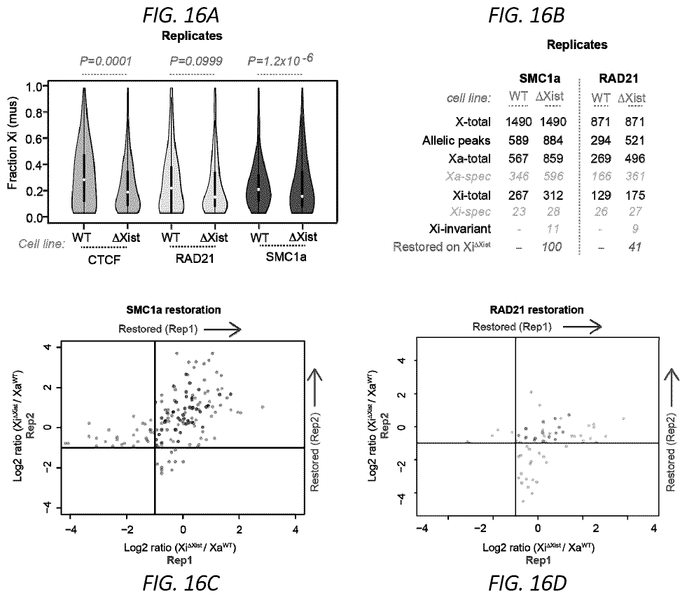

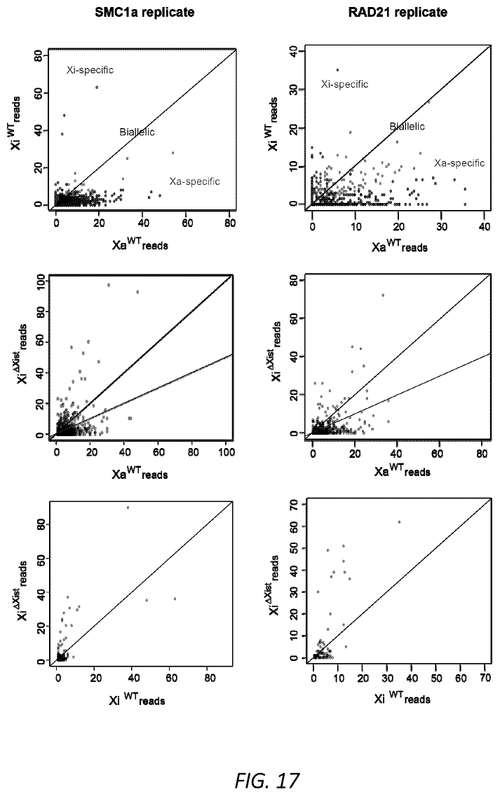

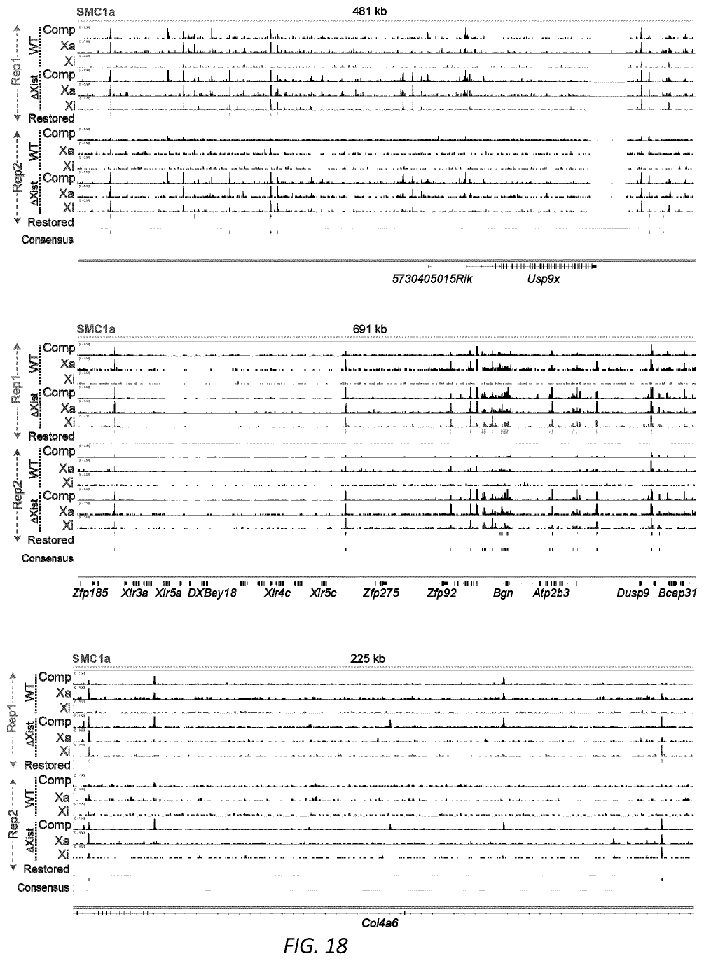

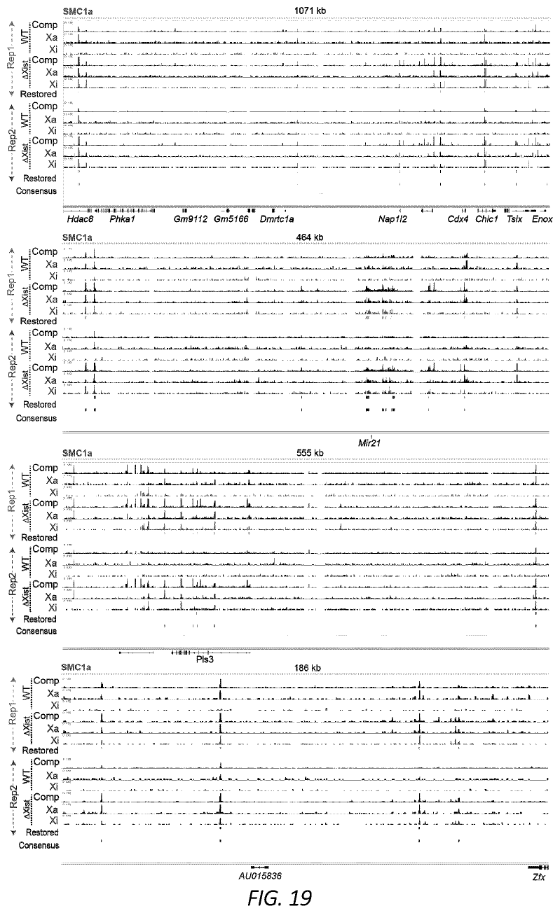

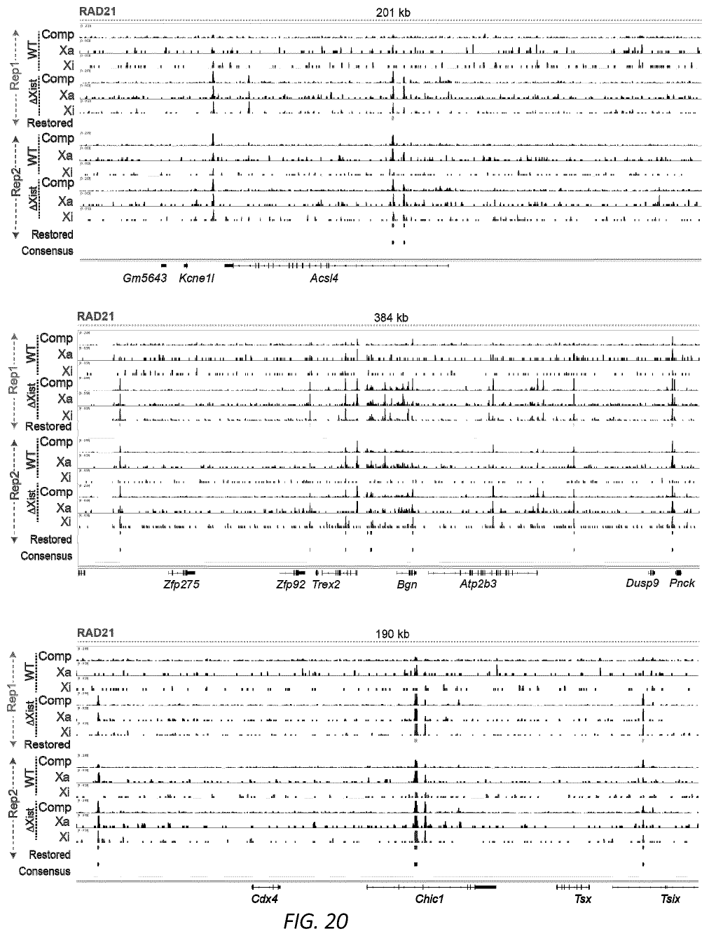

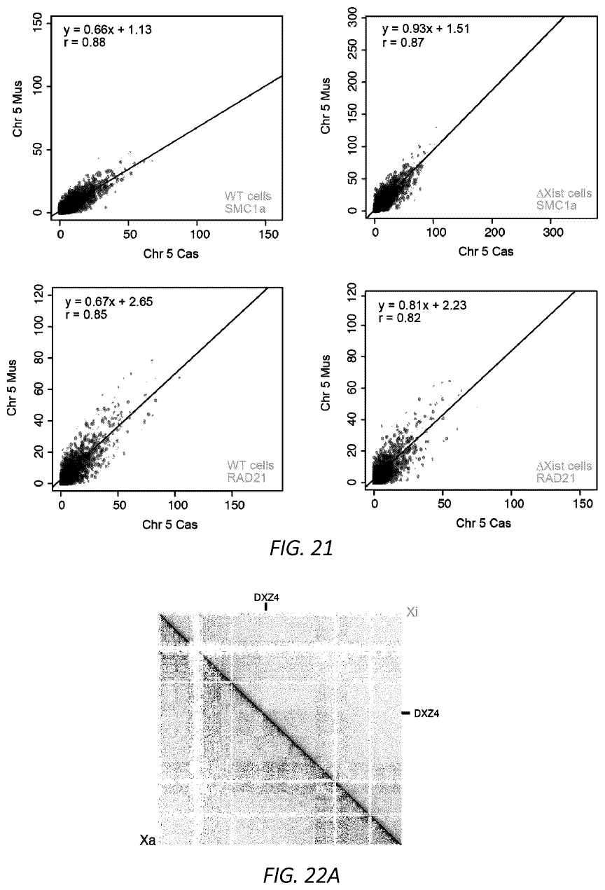

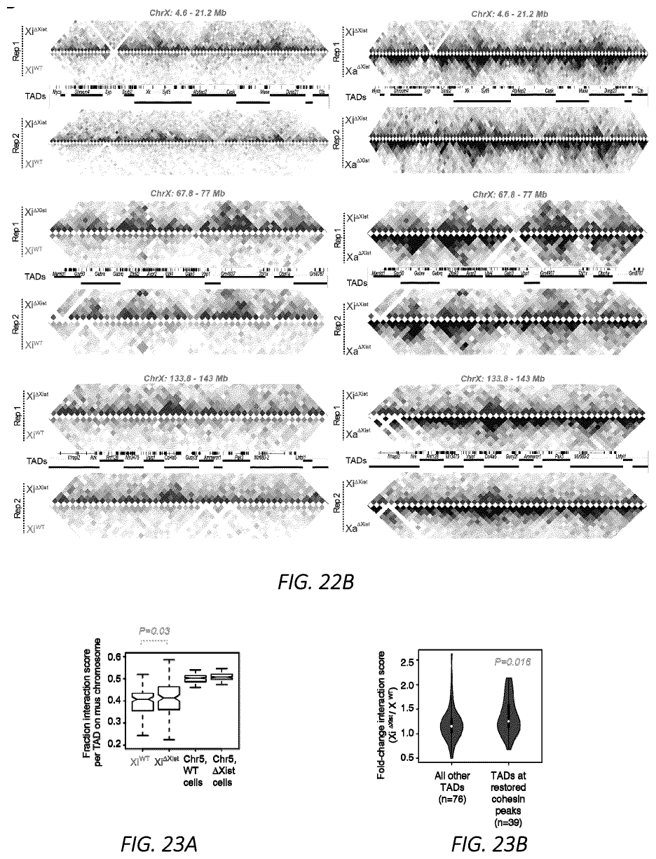

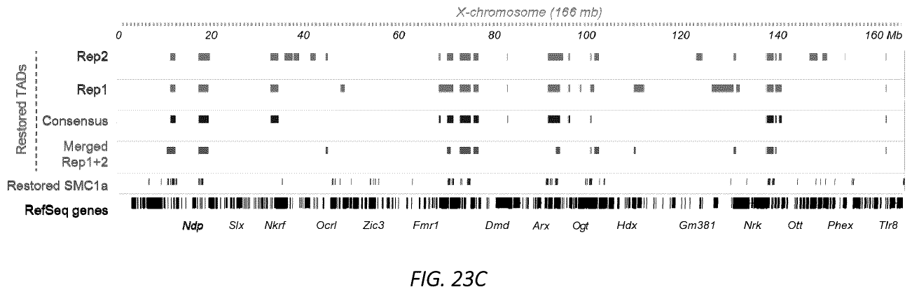

A systematic identification of interacting factors has been challenging because of Xist's large size, the expected complexity of the interactome, and the persistent problem of high background with existing biochemical approaches (20). A high background could be particularly problematic for chemical crosslinkers that create extensive covalent networks of proteins, which could in turn mask specific and direct interactions. We developed iDRiP (identification of direct RNA interacting proteins) using the zero-length crosslinker, UV light, to implement an unbiased screen of directly interacting proteins in female mouse fibroblasts expressing physiological levels of Xist RNA (FIG. 1A). We performed in vivo UV crosslinking, prepared nuclei, and solubilized chromatin by DNase I digestion. Xist-specific complexes were captured using 9 complementary oligonucleotide probes spaced across the 17-kb RNA, with a 25-nt probe length designed to maximize RNA capture while reducing non-specific hybridization. The complexes were washed under denaturing conditions to eliminate factors not covalently linked by UV to Xist RNA. To minimize background due to DNA-bound proteins, a key step was inclusion of DNase I treatment before elution of complexes. We observed significant enrichment of Xist RNA over highly abundant cytoplasmic and nuclear RNAs (U6, Jpx, 18S rRNA) in eluates of female fibroblasts (FIG. 1B). Enrichment was not observed in male eluates or with luciferase capture probes. Eluted proteins were subjected to quantitative mass spectrometry (MS), with spectral counting (21) and multiplexed quantitative proteomics (22) yielding similar enrichment sets (Tables 5-6).

From three independent replicates, iDRiP-MS revealed a large Xist protein interactome (FIG. 1C; Tables 5 and 6). Recovery of known Xist interactors PRC2 (RBBP4, RBBP7), ATRX, and HNRPU provided a first validation of the iDRiP technique. Also recovered were PRC1 (RING1), macrohistone H2A (mH2A) and the condensing component, SmcHD1, all of which proteins are known to be enriched on the Xi (23, 24, 19), but not previously shown to interact directly with Xist. More than 80 proteins were found to be ≥3-fold enriched over background; >200 proteins were ≥2-fold enriched (Tables 5-6). In many cases, multiple subunits of the epigenetic complex were identified, boosting our confidence in them as interactors. We verified select interactions by performing a test of reciprocity: By baiting with candidate proteins in an antibody capture, RIP-qPCR of UV-crosslinked cells reciprocally identified Xist RNA in the pulldowns (FIG. 1D). Called on the basis of high enrichment values, presence of multiple subunits within a candidate epigenetic complex, and tests of reciprocity, novel high-confidence interactors fell into several functional categories: (i) Cohesin complex proteins, SMC1a, SMC3, RAD21, WAPL, PDS5a/b, as well as CTCF (25), which are collectively implicated in chromosome looping and transcriptional regulation (26-28); (ii) histone modifiers such as aurora kinase B (AURKB), a serine/threonine kinase that phosphorylates histone H3 (29); RING1, the catalytic subunit of Polycomb repressive complex 1 (PRC1) for H2A-K119 ubiquitylation (23); and SPEN and RBM15, which associate with HDACs; (iii) SWI/SNF chromatin remodeling factors; (iv) topoisomerases, TOP2a, TOP2b, and TOP1, that relieve torsional stress during transcription and DNA replication; (v) miscellaneous transcriptional regulators, MYEF2 and ELAV1; (vi) nucleoskeletal proteins that anchor chromosomes to the nuclear envelope, SUN2, Lamin-B receptor (LBR), and LAP2; (vii) nuclear matrix proteins, hnRPU/SAF-A, hnRPK, and MATRIN3; and (viii) the DNA methyltransferase, DNMT1, known as a maintenance methylase for CpG dinucleotides (30).

To study their function, we first performed RNA immunoFISH of female cells and observed several patterns of Xi coverage relative to the surrounding nucleoplasm (FIG. 1E). Like PRC2, RING1 (PRC1) has been shown to be enriched on the Xi (23) and is therefore not pursued further. TOP1 and TOP2a/b appeared neither enriched nor depleted on the Xi (100%, n>50 nuclei). AURKB showed two patterns of localization—peri-centric enrichment (20%, n>50) and a more diffuse localization pattern (80%, data now shown), consistent with its cell-cycle dependent chromosomal localization (29). On the other hand, while SUN2 was depleted on the Xi (100%, n=52), it often appeared as pinpoints around the Xi in both day 7 differentiating female ES cells (establishment phase; 44%, n=307) and in fibroblasts (maintenance phase; 38.5%, n=52), consistent with SUN2's function in tethering telomeres to the nuclear envelope. Finally, the cohesins and SWI/SNF remodelers unexpectedly showed a depletion relative to the surrounding nucleoplasm (100%, n=50-100). These patterns suggest that the Xist interactors operate in different XCI pathways.

To ask if the factors intersect the PRC2 pathway, we stably knocked down (KD) top candidates using shRNAs (Table 2) and performed RNA immunoFISH to examine trimethylation of histone H3-lysine 27 (H3K27me3; FIGS. 2A,B). No major changes to Xist localization or H3K27me3 were evident in d7 ES cells (FIG. 9). There were, however, long-term effects in fibroblasts: The decreased in H3K27me3 enrichment in shSMARCC1 and shSMARCA5 cells (FIG. 2A,B) indicated that SWI/SNF interaction with Xist is required for proper maintenance of PRC2 function on the Xi. Steady state Xist levels did not change by more than 2-fold (FIG. 2C) and were therefore unlikely to be the cause of the Polycomb defect. Knockdowns of other factors (cohesins, topoisomerases, SUN2, AURKB) had no obvious effects on Xist localization and H3K27me3. Thus, whereas the SWI/SNF factors intersect the PRC2 pathway, other interactors do not overtly impact PRC2.

### Example 2. Xi-Reactivation Via Targeted Inhibition of Synergistic Interactors

Given the large number of interactors, we created a screen to analyze effects on Xi gene expression. We derived clonal fibroblast lines harboring a transgenic GFP reporter on the Xi (FIG. 10) and shRNAs against Xist interactors. Knockdown of any one interactor did not reactivate GFP by more than 4-fold (FIG. 3A, shControl+none; FIG. 11A). Suspecting synergistic repression, we targeted multiple pathways using a combination drugs. To target DNMT1, we employed the small molecule, 5′-azacytidine (aza)(30) at a nontoxic concentration of 0.3 μM (≤IC₅₀) which minimally reactivated GFP (FIG. 3A, shControl+aza). To target TOP2a/b (31), we employed etoposide (eto) at 0.3 μM (≤IC₅₀), which also minimally reactivated GFP (FIG. 3A, shControl+eto). Combining 0.3 μM aza+eto led to an 80-to 90-fold reactivation—a level that was almost half of GFP levels on the Xa (Xa-GFP, FIG. 3A), suggesting strong synergy between DNMT1 and TOP2 inhibitors. Using aza+eto as priming agents, we designed triple-drug combinations inclusive of shRNAs for proteins that have no specific small molecule inhibitors. In various shRNA+aza+eto combinations, we achieved up to 230-fold GFP reactivation—levels that equaled or exceeded Xa-GFP levels (FIG. 3A). Greatest effects were observed for combinations using shSMARCC2 (227×), shSMARCA4 (180×), and shRAD21 (211×). shTOP1 and shCTCF were also effective (175×, 154×). Combinations involving remaining interactors yielded 63× to 94× reactivation.

We then performed allele-specific RNA-seq to investigate native Xi genes. In an F1 hybrid fibroblast line in which the Xi is of Mus musculus (mus) origin and the Xa of Mus casteneus (cas) origin, >600,000 X-linked sequence polymorphisms enabled allele-specific calls (32). Two biological replicates of each of the most promising triple-drug treatments showed good correlation (FIG. 12-14). RNA-seq analysis showed reactivation of 75-100 Xi-specific genes in one replicate (FIG. 3B) and up to 200 in a second replicate (FIG. 11B), representing a large fraction of expressed X-linked genes, considering that only ˜210 X-linked genes have an FPKM≥1.0 in this hybrid fibroblast line. Heatmap analysis demonstrated that, for individual Xi genes, reactivation levels ranged from 2-˜80× for various combinatorial treatments (FIG. 3C). There was a net increase in expression level (ΔFPKM) from the Xi in the triple-drug treated samples relative to the shControl+aza+eto, whereas the Xa and autosomes showed no obvious net increase, thereby suggesting preferential effects on the Xi due to targeting synergistic components of the Xist interactome. Reactivation was not specific to any one Xi region (FIG. 3D). Most effective were shRAD21, shSMC3, shSMC1a, shSMARCA4, shTOP2a, and shAURKB drug combinations. Genic examination confirmed increased representation of mus-specific tags (red) relative to the shControl (FIG. 3E). Such allelic effects were not observed at imprinted loci and other autosomal genes (FIG. 14), further suggesting Xi-specific allelic effects. The set of reactivated genes varied among drug treatments, though some genes (Rbbp7, G6pdx, Fmr1, etc.) appeared more prone to reactivation. Thus, the Xi is maintained by multiple synergistic pathways and Xi genes can be reactivated preferentially by targeting two or more synergistic Xist interactors.

### Example 3. Xist Interaction Leads to Cohesin Repulsion

To investigate mechanism, we focused on one group of interactors—the cohesins—because they were among the highest-confidence hits and their knockdowns consistently destabilized Xi repression. To obtain Xa and Xi binding patterns, we performed allele-specific ChIP-seq for two cohesin subunits, SMC1a and RAD21, and for CTCF, which works together with cohesins (33, 34, 28, 35). In wildtype cells, CTCF binding was enriched on Xa (cas), but also showed a number of Xi (mus)-specific sites (FIG. 4A)(36, 25). Allelic ratios ranged from equal to nearly complete Xa or Xi skewing (FIG. 4A). For the cohesins, 1490 SMC1a and 871 RAD21 binding sites were mapped onto ChrX in total, of which allelic calls could be made on ˜50% of sites (FIG. 4B,C). While the Xa and Xi each showed significant cohesin binding, Xa-specific greatly outnumbered Xi-specific sites. For SMC1a, 717 sites were called on Xa, of which 589 were Xa-specific; 203 sites were called on Xi, of which 20 were Xi-specific. For RAD21, 476 sites were called on Xa, of which 336 were Xa-specific; 162 sites were called on Xi, of which 18 were Xi-specific. Biological replicates showed similar trends (FIGS. 16A,B).

Cohesin's Xa preference was unexpected in light of Xist's physical interaction with cohesins—an interaction suggesting that Xist might recruit cohesins to the Xi. We therefore conditionally ablated Xist from the Xi (Xi^{ΔXist}) and repeated ChIP-seq analysis in the Xi^{ΔXist}/Xa^{WT} fibroblasts (37). Surprisingly, Xi^{ΔXist} acquired 106 SMC1a and 48 RAD21 sites in cis, at positions that were previously Xa-specific (FIG. 4C,D). Biological replicates trended similarly (FIG. 16-17). In nearly all cases, acquired sites represented a restoration of Xa sites, rather than binding to random positions. By contrast, sites that were previously Xi-specific remained intact (FIG. 4C,E, 16B), suggesting that they do not require Xist for their maintenance. The changes in cohesin peak densities were Xi-specific and significant (FIG. 4F). Cohesin restoration occurred throughout Xi^{ΔXist}, resulting in domains of biallelic binding (FIG. 4G, 18-20), and often favored regions that harbor genes that escape XCI (e.g., Bgn)(38, 39). There were also shifts in CTCF binding, more noticeable at a locus-specific level than at a chromosomal level (FIG. 4A,G), suggesting that CTCF and cohesins do not necessarily track together on the Xi. The observed dynamics were ChrX-specific and were not observed on autosomes (FIG. 21). To determine whether there were restoration hotspots, we plotted restored SMC1a and RAD21 sites (FIG. 4H; purple) on Xi^{ΔXist} and observed clustering within gene-rich regions. We conclude that Xist does not recruit cohesins to the Xi-specific sites. Instead, Xist actively repels cohesins in cis to prevent establishment of the Xa pattern.

### Example 4. Xist RNA Directs an Xi-Specific Chromosome Conformation

Cohesins and CTCF have been shown to facilitate formation of large chromosomal domains called TADs (topologically associated domains)(27, 40, 34, 28, 35, 41, 42). The function of TADs is currently not understood, as TADs are largely invariant across development. However, X-linked domains are exceptions to this rule and are therefore compelling models to study function of topological structures (43-46). By carrying out allele-specific Hi-C, we asked whether cohesin restoration altered the chromosomal architecture of Xi^{ΔXist} First, we observed that, in wildtype cells, our TADs called on autosomal contact maps at 40-kb resolution resembled published composite (non-allelic) maps (27)(FIG. 5A, bottom). Our ChrX contact maps were also consistent, with TADs being less distinct due to a summation of Xa and Xi reads in the composite profiles (FIG. 5A, top). Using the 44% of reads with allelic information, our allelic analysis yielded high-quality contact maps at 100-kb resolution by combining replicates (FIG. 5B, 22A) or at 200-kb resolution with a single replicate. In wildtype cells, we deduced 112 TADs at 40-kb resolution on ChrX using the method of Dixon et al. (27). We attempted TAD calling for the Xi on the 100 kb contact map, but were unable to obtain obvious TADs, suggesting the 112 TADs are present only on the Xa. The Xi instead appeared to be partitioned into two megadomains at the DXZ4 region (FIG. 22A) (46). Thus, while the Xa is topologically organized into structured domains, the Xi is devoid of TADs across its full length.

When Xist was ablated, however, TADs were restored in cis and the Xi reverted to an Xa-like conformation (FIG. 5B, 22B). In mutant cells, ˜30 TADs were gained on Xi^{ΔXist} in each biological replicate. Where TADs were restored, Xi^{ΔXist} patterns (red) became nearly identical to those of the Xa (blue), with similar interaction frequencies. These Xi^{ΔXist} regions now bore little resemblance to the Xi of wildtype cells (Xi^{IT}, orange). Overall, the difference in the average interaction scores between Xi^{IT} and Xi^{ΔXist} was highly significant (FIG. 5C, 23A). Intersecting TADs with SMC1a sites on Xi^{ΔXist} revealed that 61 restored cohesin sites overlapped restored TADs (61 did not overlap). In general, restored cohesin sites occurred both within TADs and at TAD borders. TADs overlapping restored peaks had larger increases in interaction scores relative to all other TADs (FIG. 5D, 23B) and we observed an excellent correlation between the restored cohesin sites and the restored TADs (FIG. 5E, 23C), consistent with a role of cohesins in re-establishing TADs following Xist deletion. Taken together, these data uncover a role for RNA in establishing topological domains of mammalian chromosomes and demonstrate that Xist must actively and continually repulse cohesins from the Xi, even during the maintenance phase, to prevent formation of an Xa chromosomal architecture.

### Example 5. Xist Knockdown with an LNA Results in Increased Reactivation

To determine whether an LNA targeting XIST could also be used in addition to or as an alternative to an agent described herein, experiments were performed in the following cells: immortalized monoclonal MEFs with the reporter GFP (Bird) or LUC (Bedalov) fused to Mecp2, on the Xi or Xa, immortalized human fibroblasts from a 3 year old female with Rett syndrome (Coriell) and primary mouse cortical neurons.

The LNAs were designed with the Exiqon web tool. Xist LNA for mouse (TCTTGGTTACTAACAG; SEQ ID NO:50) targets exon 1 between rep C and rep D. The human Xist LNAs target the following sequences: A1: GAAGAAGCAGAGAACA; SEQ ID NO:51; A2: AGTAGCTCGGTGGAT; SEQ ID NO:52; A3: TGAGTCTTGAGGAGAA; SEQ ID NO:53. The LNAs were delivered into the cells (0.5 105/ml) with Lipofectamine LTX with Plus (Life Technologies), and incubated for 3 days. 5-azadeoxycitidine (in DMSO) was added to a final concentration of 0.5 μM (except in the titration experiment 0.1-2.5 μM). Synergistic reactivation could be observed with AzadC or EED knockdown.

qPCR was performed with Sybr chemistry (SybrGreen supermix Bio-Rad), with the primers shown in Table 9. RNA for these experiments was extracted with Triazol (Ambion), DNAse treated (Turbo DNAse kit from Ambion) and reverse transcribed with Superscript 111

TABLE 9

| | | SEQ |
|---|---|---|
| | | ID |
| Target | Sequence | NO: |
| Xist F | GCTGGTTCGTCTATCTTGTGGG | 54 |
| Xist R | CAGAGTAGCGAGGACTTGAAGAG | 55 |
| GapdH F | ATGAATACGGCTACAGCAACAGG | 56 |
| GapdH R | CTCTTGCTCAGTGTCCTTGCTG | 57 |
| Luc F | TCTAAGGAAGTCGGGGAAGC | 58 |
| Luc R | CCCTCGGGTGTAATCAGAAT | 59 |
| TBP F | ACGGACAACTGCGTTGATTTT | 60 |
| TBP R | ACTTAGCTGGGAAGCCCAAC | 61 |
| GFP F | ACCATCTTCTTCAAGGACGA | 62 |
| GFP R | GGCTGTTGTAGTTGTACTCC | 63 |
| hXist F | TAGGCTCCTCTTGGACATT | 64 |
| hXist R | GCAACCCATCCAAGTAGATT | 65 |

FIG. 7 shows the results of experiments in the Mecp2-GFP fusion Xi cell line, after treatment for 3 days with 20 nM Xist LNA administered with lipofectamine LTX with Plus reagent. qPCR analysis of XIST expression using the primers above showed that the LNAs produced a significant reduction in XIST levels.

Luciferase experiments were performed on a Microbeta2 LumiJet with a luciferase assay system (Promega). Mecp2-Luc fusion Xi and Xa cell lines (0.5 10⁵ cells/ml) were contacted with 20 nM Xist LNA administered with Lipofectamine LTX with Plus reagent, with or without 5-aza-deoxycitidine 0.5 μM, for three days. Afterwards, the cells were trypsinized, washed, and lysed using cell culture lysis reagent. Normalized measurements were performed in 96 well plates, during 10 seconds after a 2 second incubation period. Table 11 shows the results of the luciferase screen, demonstrating a significant level of reactivation with an XIST LNA plus Aza.

TABLE 11

| | 20 uM LNA, 0.5 uM aza | | 20 uM LNA, 0.5 uM aza | | 20 uM LNA, 0.5 uM aza | |
|---|---|---|---|---|---|---|
| | 3 days, | | 3 days, | | 3 days, | |
| | New 1 10\hat{ }5 | | NEW 0.5 10\hat{ }5 | | new 0.5 10\hat{ }5 | |
| | cells/ml | | cells/ml | | cells/ml | |
| | 24-well | | 6-well | | 6-well | |
| | trial 1 | | trial 3 | | trial 6 | |
| | LCPS | raw CPS | LCPS | raw CPS | LCPS | raw CPS |
| buffer | 0.0/0.0 | 39.4/ | 0/0 | 25.4/19.2 | 0/0 | 32.8/24.4 |
| | | 26.6 | | | | |
| xa | | | 656.4 | 65947.8 | | |
| No | 0 | 35.6 | | | 0 | 30 |
| ctrl | 0 | 31 | | | | |
| ctrl + aza | | | 1.1 | 140.6 | 0.8 | 130 |
| xist | 0 | 29.8 | | | | |
| xist + aza | 67.4 | 7187.6 | 44.7 | 4518.4 | 26.1 | 2814.2 |
| smchd1 | 0 | 29.2 | | | | |
| smchd1 + aza | 2.2 | 273.4 | | | | |
| ctcf + aza | | | | | 0.3 | 78.4 |
| xist + ctcf + aza | | | 6.8 | 718 | | |
| eed + aza | | | 1.7 | 207 | 1.6 | 213.6 |
| eed + xist + aza | | | 28.9 | 2933.8 | | |
| dxz + aza | | | | | 0.7 | 122.8 |
| xist + dxz4 | 0 | 27 | | | | |
| xist + dxz4 + aza | 32.9 | 3536.6 | | | | |
| firre + aza | | | | | 0.5 | 98.6 |
| firre + xist | | | | | 0 | 24.2 |

Reactivation of Mecp2 was measured in the immortalized monoclonal MEFs with the reporter GFP (Bird) or LUC (Bedalov) fused to Mecp2 on the Xi; as shown in FIGS. 8A and 8B, significant levels of reactivation of Mecp2 expression were obtained in both LUC (8A) and GFP (8B) test models after treatment with Aza plus an XIST-targeted LNA.

### REFERENCES

1. J. Starmer, T. Magnuson, Development 136, 1 (2009).

2. C. M. Disteche, Annual review of genetics 46, 537 (2012).

3. A. Wutz, R. Agrelo, Dev Cell 23, 680 (2012).

4. C. J. Brown et al., Cell 71, 527 (1992).

5. J. Wang et al., Nat Genet 28, 371 (2001).

6. A. Kohlmaier et al., PLoS Biol 2, E171 (2004).

7. K. Plath et al., J Cell Biol 167, 1025 (2004).

8. J. Zhao et al., Molecular cell 40, 939 (2010).

9. Y. Marahrens, et al., Genes Dev 11, 156 (1997).

10. E. Yildirim et al., Cell 152, 727 (2013).

11. C. J. Brown, H. F. Willard, Nature 368, 154 (1994).

12. G. Csankovszki, A. Nagy, R. Jaenisch, J Cell Biol 153, 773 (2001).

13. S. Bhatnagar et al., Proc Nat Acad Sci US A 111, 12591 (2014).

14. W. Mak et al., Science 303, 666 (2004).

15. M. Sugimoto, K. Abe, PLoS Genet 3, el 16 (2007).

16. J. Zhao, B. K. Sun, J. A. Erwin, J. J. Song, J. T. Lee, Science 322, 750 (2008).

17. K. Sarma et al., Cell 159, 869 (2014).

18. Y. Jeon, J. T. Lee, Cell 146, 119 (2011).

19. Y. Hasegawa et al., Dev Cell 19, 469 (2010).

20. A. Wutz, Nat Rev Genet 12, 542 (2011).

21. D. H. Lundgren, et al., Expert Rev Proteomics 7, 39 (2010).

22. L. Ting, R. Rad, S. P. Gygi, W. Haas, Nat Methods 8, 937 (2011).

23. S. Schoeftner et al., The EMBO journal 25, 3110 (2006).

24. M. E. Blewitt et al., Nat Genet 40, 663 (2008).

25. J. T. Kung et al., Molecular cell 57, 361 (2015).

26. M. H. Kagey et al., Nature 467, 430 (2010).

27. J. R. Dixon et al., Nature 485, 376 (2012).

28. M. Merkenschlager, D. T. Odom, Cell 152, 1285 (2013).

29. L. L. Hall, M. Byron, G. Pageau, J. B. Lawrence, J Cell Biol 186, 491 (2009).

30. V. Singh, P. Sharma, N. Capalash, Current cancer drug targets 13, 379 (2013).

31. M. E. Ashour, et al, Nature reviews. Cancer 15, 137 (2015).

32. S. F. Pinter et al., Genome Res 22, 1864 (2012).

33. S. Lin, et al., Molecular and cellular biology 31, 3094 (2011).

34. W. Li et al., Nature 498, 516 (2013).

35. J. M. Dowen et al., Cell 159, 374 (2014).

36. J. M. Calabrese et al., Cell 151, 951 (2012).

37. L. F. Zhang, K. D. Huynh, J. T. Lee, Cell 129, 693 (2007).

38. L. Carrel, H. F. Willard, Nature 434, 400 (2005).

39. J. B. Berletch et al., Human genetics 130, 237 (2011).

40. C. Feig, D. T. Odom, The EMBO journal 32, 3114 (2013).

41. C. T. Ong, V. G. Corces, Nat Rev Genet 15, 234 (2014).

42. M. Vietri Rudan et al., Cell reports 10, 1297 (2015).

43. E. Splinter et al., Genes Dev 25, 1371 (2011).

44. E. P. Nora et al., Nature 485, 381 (2012).

45. T. Nagano et al., Nature 502, 59 (2013).

46. S. S. Rao et al., Cell 159, 1665 (2014).

47. S. Sun et al., Cell 153, 1537 (2013).

48. A. Castello et al., Cell 149, 1393 (2012).

49. S. C. Kwon et al., Nature structural & molecular biology 20, 1122 (2013).

50. A. Thompson et al., Anal Chem 75, 1895 (2003).

51. G. C. McAlister et al., Anal Chem 84, 7469 (2012).

52. A. C. Tolonen, W. Haas, J Vis Exp, (2014).

53. G. C. McAlister et al., Anal Chem 86, 7150 (2014).

54. M. P. Weekes et al., Cell 157, 1460 (2014).

55. J. K. Eng, et al., J Am Soc Mass Spectrom 5, 976 (1994).

56. J. E. Elias, S. P. Gygi, Nat Methods 4, 207 (2007).

57. E. L. Huttlin et al., Cell 143, 1174 (2010).

58. A. K. Hadjantonakis, L. L. Cox, P. P. Tam, A. Nagy, Genesis 29, 133 (2001).

59. S. Heinz et al., Molecular cell 38, 576 (2010).

60. M. D. Robinson, D. J. McCarthy, G. K. Smyth, Bioinformatics 26, 139 (2010).

61. E. Lieberman-Aiden et al., Science 326, 289 (2009).

62. E. Yildirim, et al., Nat Struct Mol Biol 19, 56 (2012).

63. Y. C. Lin et al., Nat Immunol 13, 1196 (2012).

64. S. Selvaraj, R. D. J, V. Bansal, B. Ren, Nat Biotechnol 31, 1111 (2013).

65. P. A. Knight, D. Ruiz, IMA Journal of Numerical Analysis 33, 1029 (2012).

66. M. Imakaev et al., Nat Methods 9, 999 (2012).

TABLE 5
iDRiP proteomics results-Spectral counts of proteins pulled down by iDRiP and identified by mass spectrometry.

| UniProt Entry | Human | Human | Human Gene | | |
|---|---|---|---|---|---|
| Name | Gene ID | Protein | symbol | Gene Synonyms | Accession numbers |
| PLIN1_MOUSE | 5346 | PLIN1 | PLIN1 | PLIN; FPLD4; PERI; perilipin | NM_001145311; NM_002666; XM_005254934; |
| Q3UJB0_MOUSE | 10992 | SF3B2 | SF3B2 | SF3b1; Cus1; SF3b150; SAP145; | XM_005273726; XM_011544740; NM_006842 |
| | | | | SF3B145 | NM_003292; XM_011509955; |
| TPR_MOUSE | 7175 | TPR | TPR | GUITHDRAFT_135836 | NM_003292; XM_011509955; |
| PLIN4_MOUSE | 729359 | PLIN4 | PLIN4 | KIAA1881; S3-12; | XM_011528237; XM_006722866; |
| | | | | MDA_GLEAN10011097 | XM_011528235; XM_006722868; |
| | | | | | NM_001080400; XM_011528233; |
| | | | | | XM_011528236; XM_011528234 |
| NB5R3_MOUSE | 1727 | NBR5 | CYB5R3 | B5R; DIA1; CB5R | NM_007326; NM_000398; NM_001129819; |
| | | | | | NM_001171660; NM_001171661; |
| ATRX_MOUSE | 546 | ATRX | ATRX | ATR2; SFM1; ZNF-HX; SHS; XH2; | XM_005262155; XM_005262154; |
| | | | | RAD54; JMS; MRXHF1; RAD54L; | XM_006724667; XM_006724668; XM_000489; |
| | | | | XNP; ATIG8600; CHR20; | XM_005262156; XM_005261253; |
| | | | | F22O13.8; F22O13_8 | XR_938400; ; NM_138270; XM_005262157; |
| | | | | | NM_138271; XM_006724666 |
| MPP10_MOUSE | 10199 | MPP10 | MPHOSPH10 | CT90; PPP1R106; MPP10P; | NM_005791 |
| | | | | MPP10; PANDA_013440 | |
| RFA1_MOUSE | 6117 | RFA1 | RPA1 | P1CST_79093; LMJF_28_1820; | NM_002945 |
| | | | | LINJ_28_1940; | |
| | | | | GUITHDRAFT_166372; REPA1; | |
| | | | | RF-A; RP-A; MST075; HSSB; | |
| | | | | RPA70; PHATRDRAFT_14457; | |
| | | | | NGA_0366300; LPMP_28_1930; | |
| | | | | CHLREDRAFT_176094; | |
| | | | | LBRM_28_1990; | |
| | | | | THAPSDRAFT_40884; | |
| | | | | GUITHDRAFT_79993 | |
| DDX50_MOUSE | 79009 | DDX50 | DDX50 | DDX21; PAL_GLEAN10020554; | NM_024045; XM_005270148; XM_011540143; |
| | | | | RH-1I/GuB; mcdrh; GU2; GUB | XM_011540144 |
| RFC1_MOUSE | 5981 | RFC1 | RFC1 | YOR217W; CDC44; CaO19.14180; | NM_001204747; XM_011513730; XM_002913; |
| | | | | GUITHDRAFT_100231; | XM_011513731 |
| | | | | GUITHDRAFT_160531; RFC140; | |
| | | | | PO-GA; RECC1; A1; MHCBFB; | |
| | | | | RFC; CHLREDRAFT_150793; | |
| | | | | AtRFC1; replication factor C1; | |
| | | | | AT5G22010; replication factor C 1; | |
| | | | | EMIHUDRAFT_558179; | |
| | | | | CaO19.6891 | |
| HP1B3_MOUSE | 50809 | HP1B3 | HP1BP3 | HP1BP74; HP1-BP74; Anapl_13059 | XM_005245875; XM_005245879; |
| | | | | | XM_005245876; XM_005245878; |
| | | | | | XM_005245877; NM_016287; XM_011541535; |
| | | | | | XM_011541532; XM_011541533; |
| | | | | | XM_011541534 |
| TOP2B_MOUSE | 7155 | TOP2B | TOP2B | top2bets; TOPIIB | XR_940497; NM_001068; XM_005265427; |
| | | | | | XM_011534057 |
| RIF1_MOUSE | 55183 | RIF1 | RIF1 | PICST_28386; YBR275C | XR_922954; NM_001177663; XM_005246665; |
| | | | | | XR_922957; XR_022055; XR_922956; |
| | | | | | XM_011511393; NM_001177664; |
| | | | | | NM_001177665; XM_011511394; NM_018151; |
| | | | | | XM_011511395 |
| EPIPL_MOUSE | 83481 | EPIPL | EPPK1 | EPIPL1; EPIPL | XM_011517325; NM_031308; |
| PSPC1_MOUSE | 55269 | PSPC1 | PSPC1 | PANDA_015253; | XM_006719844; XM_011535140; XR_941619; |
| | | | | MDA_GLEAN10004221; PSP1 | XM_011535142; XM_011535139; |
| | | | | | XM_011535137; XR_941616; ; NM_001042414; |
| | | | | | XR_941617; XM011535138; XM_011535141; |
| | | | | | XM_011535143; NR_003272; NR_044998 |
| HNRLL_MOUSE | 92906 | HNRLL | HNRNPLL | HNRPLL; SRRF | XM_005264640; XM_011533165; |
| | | | | | XM_005264639; XR_939744; NM_138934; |
| | | | | | XM_011533166; NM_001142650 |
| RRBP1_MOUSE | — | — | | | |
| RL14_MOUSE | 9045 | RL14 | RPL14 | OSTLU_9318; CAG-ISL-7; L14; | NM_001034996; NM_003973 |
| | | | | CTG-B33; RL14; hRL14; | |
| | | | | CHLREDRAFT_145271 | |
| SMC1A_MOUSE | 8243 | SMC1A | SMC1A | SMC1; PANDA_016538; SMC1L1; | ; NM_006306; NM_001281463 |
| | | | | SMCB; SB1.8; SMC1alpha; | |
| | | | | DXS423E; CDLS2; SMC-1A | |
| NOC2L_MOUSE | 26155 | NOC2L | NOC2L | NIR; PPP1R112; NET15; NET7 | NM_015658 |
| A2AJ72_MOUSE | 8939 | FUBP3 | FUBP3 | FBP3 | XM_011519172; XM_006717314; |
| | | | | | XM_005272232; XM_006717312; |
| | | | | | XM_011519173; XM_006717313; NM_003934; |
| | | | | | XM_011519174; XM_011519171; XR_929871 |
| DNJB6_MOUSE | 10049 | DNJ | DNAJB6 | DJ4; HHDJ1; LGMDIE; MRJ; | ; XM_005249515; XM_005249516; |
| | | | | MSJ-1; HSJ2; HSJ-2; DnaJ; | XM_058246; NM_005494; XM_006715823; |
| | | | | LGMID1D | XM_011515704 |
| KIF4_MOUSE | 24137 | KIF4A | KIF4A | PANDA_006442; | XM_01130893; ; NM_012310 |
| | | | | MDA_GLEAN10002731; | |
| | | | | PAL_GLEAN10005701; KIF4; | |
| | | | | KIF4G1; MRX100 | |
| 1433T_MOUSE | 10971 | 1433T | YWHAQ | IC5; 14-3-3; HSI;; | NM_006826 |
| | | | | TREES_T100010476 | |
| SURF6_MOUSE | 6838 | SURF6 | SURF6 | RRP14; EGK_07243 | NR_103874; NM_006753; NM_001278942 |
| KI20A_MOUSE | 10112 | KI20A | KIF20A | MDA_GLEAN10012479; | NM_005733; XR_948224 |
| | | | | Anap_l14151; PANDA_011785; | |
| | | | | PAL_GLEAN10016825; | |
| | | | | RAB6KIFL; MKLP2 | |
| PDS5B_MOUSE | 23047 | PDS5B | PDS5B | APRIN; AS3; CG008 | XM_011535002; XM_005266298; |
| | | | | | XM_011535001; NM_015032; NM_015928; |
| | | | | | XM_011534999; XM_011535000; |
| ZN638_MOUSE | 27332 | ZN638 | ZNF638 | ZFML; Zfp638; NP220 | XM_011532767; XR_939678; NM_001014972; |
| | | | | | NM_001252613; XM_006711989; |
| | | | | | XM_011532769; XM_011523768; |
| | | | | | NM_001252612; NM_014497; XM_005264263 |
| RAD21_MOUSE | 5885 | RAD21 | RAD21 | HRAD21; SCC1; MCD1; NXP1; | NM_006265 |
| | | | | CDLS4; HR21; hHR21; | |
| | | | | PANDA_018369; | |
| | | | | PAL_GLEAN10021417; | |
| | | | | MDA_GLEAN10024618 | |
| SMHD1_MOUSE | 23347 | SMHD1 | SMCHD1 | — | XM_011525645; NM_015295; XM_011525646; |
| | | | | | ; XM_011525643; XM_011525644; XR_935054; |
| | | | | | XM_011525642; XM_011525647; XR_935055; |
| | | | | | XR_430039 |
| DDX10_MOUSE | 1662 | DDX10 | DDX10 | HRH-J8 | XM_011542646; NM_004398 |
| PDIP3_MOUSE | 84271 | PDIP3 | POLDIP3 | SKAR; PDIP46 | XM_011530457; NM_032311; NM_178136; |
| | | | | | NM_001278657; XR_937942; NR_103820 |
| K0020_MOUSE | 9933 | K0020 | KIAA0020 | PUF6; HA-8; HLA-HA8; PEN; | NM_001031691; NM_014878 |
| | | | | XTP5; PUF-A | |
| CPSF7_MOUSE | 79869 | CPFS7 | CPSF7 | CFIm59; PAL_GLEAN10011510; | XM_011545257; XM_011545263; |
| | | | | UY3_12626 | XM_005274303; NM_001142565; |
| | | | | | XM_011545258; XM_011545262; |
| | | | | | XM_005274299; XM_011545260; NM_024811; |
| | | | | | XM_011545261; NM_001136040; |
| | | | | | XM_005274298; XM_011545259 |
| ELYS_MOUSE | 25909 | ELYS | AHCTF1 | MSTP108; MST108; ELYS; | XM_006711758; XR_949137; NM_015446; |
| | | | | TMBS62 | XM_011544156; XR_426916; XM_006711759; |
| | | | | | XM_011544157; XR_949136 |
| APE_HMOUSE | 327 | ACPH | APEH | AARE; D3S48E; D3F15S2; ACPH; | XM_005265097; XM_011533658; |
| | | | | DNF15S2; APH; OPH; | XM_005265098; XM_011533656; |
| | | | | CB1_000145050; | XM_011533660; XM_011533657; |
| | | | | PAL_GLEAN10009189; AAP | XM_011533659; XM_011533662; ; |
| | | | | | XM_011533661; XM_011533663; NM_001640 |
| TDIF2_MOUSE | 30836 | TDIF2 | DNTTIP2 | LPTS-RP2; ERBP; FCF2; | NM_014597 |
| | | | | HSU15552; TdIF2; | |
| | | | | MDA_GLEAN10013834 | |
| NXF1_MOUSE | 10482 | NXF1 | NXF1 | TREES_T100020891; MEX67; | NM_001081491; NM_006362 |
| | | | | TAP; PAL_GLEAN10011461 | |
| PRP19_MOUSE | 27339 | PRP19 | PRPF19 | hPSO4; PSO4; UBOX4; PRP19; | NM_014502 |
| | | | | SNEV; NMP200; | |
| | | | | TREES_T100002308; EGK_06157; | |
| | | | | CB_1002300027; nmp-200 | |
| SF3A3_MOUSE | 10946 | SF3A3 | SF3A3 | TREES_T100000917; GB11549; | NM_006802; XM_005270390 |
| | | | | PRP9; PRPF9; SAP61; SF3a60; | |
| | | | | NGZ_0471300 | |
| PSA1_MOUSE | 5682 | B4E0X6 | PSMA1 | CC2; NU; HC2; HEL-S-275; | NM_001143937; NM_002786; NM_148976 |
| | | | | PROS30 | |
| WDR46_MOUSE | 9277 | WDR46 | WDR46 | PANDA002273; C6orf11; FP221; | XM_011547332; XM_011548316; |
| | | | | BING4; UTP7; | XM_011548317; XM_011514993; |
| | | | | PAL_GLEAN10007103 | XM_011547730; XM_011547729; NM_005452; |
| | | | | | XM_011547333; XM_011514992; |
| | | | | | XM_011548119; XM_0111548118; |
| | | | | | NM_001164267 |
| RED_MOUSE | 3550 | RED | IK | RED; CSA2 | NM_006083 |
| SNUT1_MOUSE | 9092 | SNUT1 | SART1 | Snu66; SART1259; SNRNP110; | XM_011535345; XM_011535344; XR_950099; |
| | | | | Ara1; HOMS1 | NM_005146 |
| Q0VBL3_MOUSE | 64783 | RBM15 | RBM15 | SPEN; OTT; OTT1 | XM_011541967; NM_001201545; |
| | | | | | XM_011541965; XM_011541966; |
| | | | | | XM_011541964; XM_011541969; NM_022768; |
| | | | | | XM_011541968 |
| Q8BK35_MOUSE | 29997 | GSCR2 | GLTSCR2 | P1CT1; P1CT-1 | NM_015710 |
| TPX2_MOUSE | 22974 | TPX2 | TPX2 | MDA_GLEAN10014018; | XM_011528697; XM_011528698; |
| | | | | AacL_AAEL004112; DIL-2; | NM_0121112; XM_011528700; |
| | | | | REPP86; C20orf1; p100; | XM_011528699 |
| | | | | GD:C20orf1; C20orf2; DIL2; | |
| | | | | FLS353; HCA519; HCTP4; | |
| | | | | AT1G03780; targeting protein for | |
| | | | | XKLP2; F21M11_31; F21M11.31; | |
| | | | | PAL_GLEAN10024200; | |
| | | | | AgaP_AGAP011054; | |
| | | | | ENSANGG00000017293; | |
| | | | | AgaP_ENSANGG00000017293; | |
| | | | | F12P19_13; thioredocin-dependent | |
| | | | | peroxidase 2; AT1G65970; | |
| | | | | PROXIREDOXIN TPX2; | |
| | | | | F12P19.13; | |
| | | | | ARALYDRAFT475704 | |
| LAS1L_MOUSE | 81887 | LAS1L | LAS1L | Las1-like; dJ475B7.2; LAS1-like | XM_005262304; XM_005262305; |
| | | | | | NM_001170649; NM_001170650; |
| | | | | | XM_005262306; XR_430522; XM_011531045; |
| | | | | | XM_005262301; XM_005262307; |
| | | | | | XM_011531046; NM_031206; XR_244504; ; |
| | | | | | XR_938411; XR_938412 |
| ZFR_MOUSE | 51663 | ZFR | ZFR | SPG71; ZFR1; | XR_427659; NM_016107 |
| | | | | PAL_GLEAN10014079 | |
| AMY1_MOUSE | | | — | | — |
| RL27A_MOUSE | 6157 | RL27A | RPL27A | L27A; RPL27; YHR010W | NM_032650; NM_000990 |
| UBF1_MOUSE | 7343 | UBF1 | UBTF | NPOR-90; UBF-1; UBF2; UBF1; | XM_006722061; NM_014233; XM_006722059; |
| | | | | UNF | XM_006722060; XM_011525177; |
| | | | | | NM_001076683; NM_001076684; NR_045058 |
| VP26A_MOUSE | 9559 | VP26A | VPS26A | MDA_GLEAN10020826; | NM_001035260; NM_004896; XM_011540378 |
| | | | | GUITHDRAFT_135609; MNC6_7; | |
| | | | | AT5G53530; MNC6.7; vacuolar | |
| | | | | protein sorting 26A; Hbeta58; | |
| | | | | HB58; PEP8A; VPS26 | |
| ALDH2_MOUSE | 217 | ALDH2 | ALDH2 | LINJ_25_1160; LMJF_25_1120; | NM_001204889; NM_000690; |
| | | | | PAL_GLEAN10008876; ALDH1; | |
| | | | | ALDH-E2; ALDM; | |
| | | | | EMIHUDRAFT_350230; | |
| | | | | LPMP_251150 | |
| DHB4_MOUSE | 3295 | DHB4 | HSD17B4 | MFE-2; PRLTS1; SCR8C1; DBP; | NM_001292028; NM_001292027; |
| | | | | MPF-2 | NM_001199292; ; NM_001199291; NM_000414 |
| IMA1_MOUSE | 3838 | IMA1 | KPNA2 | UY3_02579; IPOA1; QIP2; | XM_011524783; NM_002266 |
| | | | | SRP1alpha; RCH1; Anapl_03182; | |
| | | | | PANDA_014057; | |
| | | | | PAL_GLEAN10014864 | |
| SPB5_MOUSE | 5268 | SPB5 | SERPINB5 | maspin; P15 | NM_002639; XM_006722483 |
| TIAR_MOUSE | 7073 | TIAR | TIAL1 | TIAR; TCBP | XM_005270108; XR_428715; |
| | | | | | XM_005270109; ; XM_005270110; XR_945808; |
| | | | | | NM_003252; NM_001033925 |
| SMRC1_MOUSE | 6599 | SMRC1 | SMARCC1 | BAF155; Rsc8; CRACC1; SW13; | XM_011534034; XM_011534035; NM_003074 |
| | | | | SRG3 | |
| LARP7_MOUSE | 51574 | LARP7 | LARP7 | UY3_01935; ALAZS; P1P7S; | ; NM_015454; NM_016648; NR_049768; |
| | | | | HDCMA18P | NM_001267039 |
| NSUN2_MOUSE | 54888 | NSUN2 | NSUN2 | TRM4; SALI; MRT5; M1SU | NM_017755; ; NM_001193455; NR_037947 |
| NOL8_MOUSE | 55035 | NOL8 | NOL8 | C9orf34; NOP132; bA62C3.4; | XM_006717169; XM_006717170; |
| | | | | bA62C3.3 | XM_011518824; XM_011518828; NR_046106; |
| | | | | | XM_006717168; XM_006717173; |
| | | | | | XM_011518825; XM_006717166; |
| | | | | | XM_011518826; XM_011518827; NM_017948; |
| | | | | | XM_006717172; XR_929816; XM_006717167; |
| | | | | | NM_001256394 |
| ERMP1_MOUSE | 79956 | ERMP1 | ERMP1 | FXNA; KIAA1815; bA207C16.3; | XR_9293338; NM_024896; XM_011518034; |
| | | | | PAL_GLEAN10021042 | XR_428431; XM_005251587; XR_929337; |
| | | | | | XR_929340 |
| NPA1P_MOUSE | 9875 | NPA1P | URB1 | C21orf108; NPA1; YKL014C | NM_014825 |
| UTP20_MOUSE | 27340 | UTP20 | UTP20 | P1CST_74252; CaO19.9301; | NM_014503; XM_006719343 |
| | | | | CaO19.10668; DRIM; YBL004W; | |
| | | | | CaO19.1733; MICPUN_107415; | |
| | | | | CaO19.3159; | |
| | | | | PAL_GLEAN10015492 | |
| LAP2A_MOUSE | 7112 | LAP2B | TMPO | LAP2beta; LAP2; CMD1T; | ; NM_001032284; XM_005269132; |
| | | | | LEMD4; TP; PRO0868 | XM_005269130; NM_001032283; NM_003276 |
| REQU_MOUSE | 5977 | REQU | DPF2 | REQ; MDA_GLEAN10017910; | XR_950008; XM_005274149; NM_006268 |
| | | | | UB1D4; ubi-d4; | |
| | | | | PAL_GLEAN10011379 | |
| PLSL_MOUSE | 3936 | PLSL | LCP1 | plastin-2; CP64; LC64P; L- | XM_005266374; NM_002298 |
| | | | | PLASTIN; LPL; PLS2; HEL-S-37; | |
| | | | | LCP-1; EGK_09301; Plastibn-2 | |
| SCAF8_MOUSE | 22828 | SCAF8 | SCAF8 | RBM16 | NM_014892; NM_001286194; NM_001286189; |
| | | | | | NM_001286199; NM_001286188 |
| ABCF1_MOUSE | 23 | ABCF1 | ABCF1 | D1CPUDRAFT_157052; | NM_001025091; NM_001090 |
| | | | | PAL_GLEAN10001332; ABC27; | |
| | | | | ABC50; LMJF_03_0160; | |
| | | | | LINJ_03_0150 | |
| DCA13_MOUSE | 25879 | DCA13 | DCAF13 | WDSOF1; HSPC064; GM83 | NM_015420 |
| SMRC2_MOUSE | 6601 | SMRC2 | SMARCC2 | CRACC2; BAF170; Rsc8 | NM_139067; NM_001130420; XM_005269101; |
| | | | | | XM_005269104; XM_005269102; |
| | | | | | XM_011538693; XM_005269103; |
| | | | | | XM_011538694; NM_003075 |
| TRA2A_MOUSE | 29896 | TRA2A | TRA2A | AWMS1; HSU53209 | NM_013293; NM_001282757; NM_001282759; |
| | | | | | XM_005249725; XM_011515331; |
| | | | | | XM_006715713; NM_001282758 |
| POGZ_MOUSE | 23126 | POGZ | POGZ | ZNF635; ZNF635m; ZNF280E; | XM_011509331; NM_015100; XM_005244999; |
| | | | | PANDA_007985 | XR_921760; NM_001194938; XM_005245006; |
| | | | | | XM_011509330; XM_145796; NM_207171; |
| | | | | | XM_005245000; XM_005245001; |
| | | | | | XM_005245005; XM_001194937 |
| CHERP_MOUSE | 10523 | CHERP | CHERP | MDA_GLEAN10007202; SCAF6; | NM_006387 |
| | | | | SRA1; DAN16 | |
| RBM12_MOUSE | 10137 | RBM12 | RBM12 | CPNE1; Anapl_04462; | NM_001198838; NM_001198840; NM_152838; |
| | | | | AS27_09836; EGK_02457; SWAN; | NM_006047 |
| | | | | HR1HFB2091; PANDA_004540; | |
| | | | | TREES_T100008592 | |
| PHIP_MOUSE | 55023 | PHIP | PHIP | WDR11; DCAF14; BRWD2; ndrp | XM_011535919; NM_017934; XM_005248729; |
| | | | | | XM_011535917; XM_011535918; XR_942499 |
| ATPG_MOUSE | 509 | ATPG; | ATP5C1 | ATP5CL1; ATP5C | NM_005174; NM_001001973; XM_011519490 |
| | | Q8TAS0 | | | |
| LRC59_MOUSE | 55379 | LRC59 | LRRC59 | p34; PRO1855; | NM_018509 |
| | | | | PAL_GLEAN10019724; | |
| | | | | UY3_00259; TREES_T100015351 | |
| MFAP1_MOUSE | 4236 | MFAP1 | MFAP1 | AMF; PAL_GLEAN10023540; | NM_005926 |
| | | | | PANDA_001004; EGK_17436 | |
| SNW1_MOUSE | 22938 | SNW1 | SNW1 | SKIIP; SKIP; PRPF45; Prp45; | NM_012245; XM_005267414; XM_005267413 |
| | | | | Bx42; NCOA-62; NGA_0680000 | |
| RAVR1_MOUSE | 125950 | RAVR1 | RAVER1 | — | NM_133452; XM_011527671; XM_011527672 |
| EMC4_MOUSE | 51234 | EMC4 | EMC4 | PIG17; TMEM85; EGK_17318; | NM_001286420; NM_016454 |
| | | | | PAL_GLEAN10023658; | |
| | | | | PANDA_014713; YGL231C | |
| BRX1_MOUSE | 55299 | BRX1 | BRIX1 | BXDC2; BRIX; PANDA_008108; | NM_018321 |
| | | | | PAL_GLEAN10001729 | |
| DAZP1_MOUSE | 26528 | DAZP1 | DAZAP1 | — | XM_005259535; XM_005259536; NM_170711; |
| | | | | | XM_011527906; XM_011527904; |
| | | | | | XM_011527908; XM_005259534; |
| | | | | | XM_011527909; NM_018959; XM_005259531; |
| | | | | | ; XM_011527907; XM_011527910; |
| | | | | | XM_011527905 |
| WDR12_MOUSE | 55759 | Q53T99; | WDR12 | PAL_GLEAN10026133; YTM1; | XM_011511469; NM_018256 |
| | | WDR12 | | MDA_GLEAN10017295 | |
| CELF2_MOUSE | 10659 | CELF2 | CELF2 | CUGBP2; NAPOR; BRUNOL3; | NM_001083591; NM_006561; XM_006717373; |
| | | | | ETR-3; ETR3; | XM_011519294; XM_011519295; |
| | | | | PAL_GLEAN10015786 | XM_011519297; XM_011519298; |
| | | | | | XM_005252534; XM_006717371; |
| | | | | | NM_001025076; XM_006717374; |
| | | | | | XM_006717375; XM_011519299; |
| | | | | | NM_001025077; XM_005252357; |
| | | | | | XM_005252358; XM_006717369; |
| | | | | | XM_011519296; XM_006717370 |
| ADNP_MOUSE | 23394 | ADNP | ADNP | EGK_02296; MED28; ADNP1; | ; NM_181442; NM_001282531; |
| | | | | PANDA_000791 | NM_001282532; NM_015339; XM_011528747; |
| | | | | | XM_011528748 |
| B9EJ54_MOUSE | 23165 | NU205 | NUP205 | C7orf14 | XM_005250235; NM_015135 |
| E9PW12_MOUSE | — | | | | |
| Q3TA68_MOUSE | 134430 | WDR36 | WDR36 | TA-WDRP; GLC1G; UTP21; | NM_139281; XM_011543163; |
| | | | | TAWDRP | |
| DEGS1_MOUSE | 8560 | DEGS1 | DEGS1 | DES1; MLD; DEGS-1; Des-1; | XM_011544317; NM_003676; XM_011544318; |
| | | | | MIG15; DEGS; FADS7 | NM_144780 |
| RPA1_MOUSE | 25885 | RPA1 | POLR1A | A190; RPO14; RPA194; RPA1; | XM_006711983; NM_015425 |
| | | | | RPO1-4 | |
| PTRF_MOUSE | 284119 | PTRF | PTRF | PANDA_011158; cavin-1; CAVIN; | ; NM_012232; XM_005257242 |
| | | | | CAVIN1; CGL4; FKSG13 | |
| COPB2_MOUSE | 9276 | COPB2 | COPB2 | beta′-COP; | NM_004766; XM_011513317; NR_023350 |
| | | | | CHLREDRAFT_154280; | |
| | | | | PAL_GLEAN10015932; Beta′-COP | |
| SPT5H_MOUSE | 6829 | SPT5H | SUPT5H | Tat_CT1; SPT5H; SPT5; | NM_003169; XM_005259183; NM_001111020; |
| | | | | PAL_GLEAN10001502; | NM_001130824; NM_001130825; |
| | | | | CB1_000338026 | XM_006723337 |
| AURKB_MOUSE | 9212 | AURKB | AURKB | STK5; aurkb-sv2; AurB; ARK2; | XM_011524070; XR_934118; NM_001256834; |
| | | | | PPP1R48; aurkb-sv1; AIM-1; A1K2; | XM_011524071; XR_934117; NM_001284526; |
| | | | | IPL1; A1M1; STK12; STK-1; ARK- | NM_004217; XM_011524072 |
| | | | | 2 | |
| PSA3_MOUSE | 5684 | PSA3 | PSMA3 | EGK_18227; PSC3; HC8; | NM_152132; NM_002788; NR_038123 |
| | | | | NGA_0516100 | |
| ACTN3_MOUSE | 49860 | CRNN | CRNN | DRC1; SEP53; C1orf10; PDRC1 | NM_016190 |
| AATM_MOUSE | 2806 | AATM | GOT2 | mitAAT; KAT1V; KAT4; FABPpm; | NM_001286220; NM_002080 |
| | | | | mAspAT; FABP-1; | |
| | | | | PAL_GLEAN10016182 | |
| CATL1_MOUSE | 1515 | CATL2 | CTSV | CTSL1; CTSL; CTSL2; | NM_001333; NM_001201575 |
| | | | | PANDA_020645; CATL2; CTSU | |
| TRFL_MOUSE | 4057 | TRFL | LTF | LF; PLF; Lf; HEL110; HLF2; | ; NM_002343; NM_001199149 |
| | | | | GIG12 | |
| SODC_MOUSE | 6647 | V9HWC9 | SOD 1 | YJR104C; CRS4; SOD1L1; | ; NM_000454 |
| | | ; SODC | | DKFZP469M1833; hSod1; HEL-S- | |
| | | | | 44; ALS1; 1POA; ALS; SOD; | |
| | | | | homodimer; | |
| | | | | EMIHUDRAFT_96386; | |
| | | | | PHATRDRAFT_12583; | |
| | | | | SPAPADRAFT_146717; | |
| | | | | PICST_89018; CU/ZN-SOD | |
| HSPB1_MOUSE | 3315 | HSPB1 | HSPB1 | Hsp25; HEL-S-102; SRP27; | NM_001540; |
| | | | | HS.76067; HSP27; CMT2F; HSP28; | |
| | | | | HMN28; PAL_GLEAN10012025; | |
| | | | | UY3_14010 | |
| SBP1_MOUSE | 8991 | SBP1 | SELENBP1 | SBP; SBP56; SP56; HEL-S-134P; | XM_011510110; XM_011510111; |
| | | | | hSBP; LPSB | NM_001258288; XR_921993; NM_001258289; |
| | | | | | NM_003944 |
| RL13A_MOUSE | 23521 | RL13A | RPL13A | YDL082W; TSTA1; L13A | NR_073024; NM_001270491; NM_012423 |
| HEXB_MOUSE | 3074 | HEXB; | HEXB | ENC-1AS; HEL-248; | ; NM_001292004; NM_000521 |
| | | A0A024R | | PAL_GLEAN10024890; | |
| | | AJ6 | | EGK_16586 | |
| PNPH_MOUSE | 4860 | PNPH; | PNP | NP; PRO1837; PUNP; | NM_000270; |
| | | V9HWH6 | | CB1_001481042 | |
| H2AX_MOUSE | 3014 | H2AX | H2AFX | H2A/X; H2A.X; H2AX; | NM_002105 |
| | | | | EGK_06977 | |
| ACADM_MOUSE | 34 | ACADM | ACADM | ACAD1; MCAD; MCADH | NM_001127328; NM_001286042; |
| | | | | | NM_001286043; ; NM_000016; |
| | | | | | NM_001286044; NR_022013 |
| EXOSX_MOUSE | 5394 | EXOSX | EXOSC10 | Rrp6p; p4; PMSCL2; PM-Scl; | XM_005263475; NM_002685; XM_005263476; |
| | | | | PMSCL; p2; PM/Scl-100l RRP6; p3 | NM_001001998; XM_011541595 |
| PAXB1_MOUSE | 94104 | PAXB1 | PAXBP1 | GCFC1; GCFC; FSAP105; | XM_006724066; XM_011529804; |
| | | | | C21orf66; BM020 | XM_011529805; NM_016631; NR_027873; |
| | | | | | NM_013329; NM_145328; XM_006724067; |
| | | | | | NM_058191 |
| CSRN3_MOUSE | 80034 | CSRN3 | CSRNP3 | FAM130A2; PA1P-2; TA1P2; | NM_024969; XM_005246865; NM_001172173 |
| | | | | PPP1R73 | |
| NUP43_MOUSE | 348995 | NUP43 | NUP43 | p42; bA350J20.1 | XM_011535799; XM_005266961; |
| | | | | | XM_011535798; NM_198887; XM_005266960; |
| | | | | | XM_005266962; XR_942420; NM_024647; |
| | | | | | NR_104456 |
| KDM2A_MOUSE | 22992 | KDM2A | KDM2A | CXXC8; FBL11; FBL7; JHDM1A; | NR_027473; NM_012308; XM_011544860; |
| | | | | FBXL11; LILINA | XM_006718479; XM_006718480; |
| | | | | | XM_011544861; XM_011544862; |
| | | | | | NM_001256405 |
| SUMO2_MOUSE | 6613 | SUMO2; | SUMO2 | Smt3A; HSMT3; SMT3H2; | NM_001005849; NM_006937 |
| | | A0A024R | | SMT3B; SUMO3 | |
| | | 8S3 | | | |
| RUXE_MOUSE | 6635 | RUXE | SNRPE | SME; Sm-E; B-raf; HYPT11 | NM_001304464; NR_130746; NM_003094 |
| RS30_MOUSE | 2197 | UB1M | FAU | FAU1; MNSFbeta; RPS30l Fub1; | NM_001997 |
| | | | | Fubi; S30; asr1 | |
| RL32_MOUSE | 6161 | RL32 | RPL32 | L32; PP9932 | NM_000994; NM_001007073; NM_001007074 |
| PP1G_MOUSE | 5501 | PP1G; | PPP1CC | PP-1G; PPP1G; PP1C | ; XM_011538505; XM_011538504; |
| | | A0A024R | | | NM_001244974; NM_002710 |
| | | BP2 | | | |
| CRNL1_MOUSE | 51340 | CRNL1 | CRNKL1 | HCRN; CLF; CRN; MSTP021; | NM_001278627; NM_001278626; |
| | | | | Clf1; SYF3 | NM_001278628; NM_001278625; NM_016652 |
| IMB1_MOUSE | 3837 | IMB1 | KPNB1 | NTF97; IMB1; IPO1; IPOB; Impnb | NM_002265; NM_001276453 |
| PEBP1_MOUSE | 5037 | PEBP1 | PEBP1 | HCNP; HEL-S-34; HCNPpp; PBP; | NM_002567 |
| | | | | PEBP-1; HEL-210; PEBP; RKIP | |
| TP53B_MOUSE | 7158 | TP53B | TP53BP1 | p202; 53BP1 | XM_011521986; XR_931898; XR_931899; |
| | | | | | NM_001141980; XM_011521985; NM_005657; |
| | | | | | XM_011521984; NM_001141979; |
| | | | | | XM_005254635 |
| RL19_MOUSE | 6143 | RL19; | RPL19 | L19 | NM_000981; XM_005257564 |
| | | J3KTE4 | | | |
| CO1A2_MOUSE | 1278 | CO1A2 | COL1A2 | OI4 | ; NM_000089 |
| SSRP1_MOUSE | 6749 | SSRP1 | SSRP1 | FACT80; FACT; T160 | NM_003146; XM_005274194; XM_011545218 |
| SMCA4_MOUSE | 6597 | SMCA4 | SMARCA4 | BAF190; RTPS2; SNF2; hSNF2b; | NM_001128844; ; XM_005260031; |
| | | | | SW12; BAF190A; MRD16; | XM_005260033; XM_005260034; |
| | | | | SNF2LB; BRG1; SNF2L4 | NM_001128846; XM_005260032; |
| | | | | | XM_005260035; XM_006722847; |
| | | | | | NM_001128845; NM_001128848; NM_003072; |
| | | | | | XM_006722845; XM_006722846; |
| | | | | | NM_001128849; XM_005260028; |
| | | | | | XM_005260030; XM_011528198; |
| | | | | | NM_001128847 |
| CAPR1_MOUSE | 4076 | CAPR1 | CAPRIN1 | RNG105; GPIP137; GRIP137; | XR_0930869; NM_005898; NM_203364 |
| | | | | M11S1; GPIAP1; p137GPI | |
| SYHC_MOUSE | 3035 | SYHC | HARS | USH3B; HRS | ; NM_001258042; NM_001289093; |
| | | | | | NM_001258040; NM_001289092; |
| | | | | | NM_001289094; NM_002109; NM_001258041 |
| CTCF_MOUSE | 10664 | CTCF | CTCF | MRD21 | NM_006565; XM_005255775; ; NM_001191022 |
| HCFC1_MOUSE | 3054 | HCFC1 | HCFC1 | HCF1; HCF1; PPP1R89; VCAF; | XM_006724816; XM_011531147; ; |
| | | | | MRX3; CFF; HCF; HCF-1 | XM_011531144; XM_011531146; |
| | | | | | XM_011531150; XM_011531148; NM_005334; |
| | | | | | XM_006724815; XM_011531149; |
| | | | | | XM_011531145 |
| BAP31_MOUSE | 10134 | BAP31 | BCAP31 | CDM; DXS1357E; 6C6-AG; | NM_001139441; NM_001256447; |
| | | | | BAP31; DDCH | NM_001129457; NM_005745 |
| CBX5_MOUSE | 23468 | CBX5 | CBX5 | HEL25; HP1; HP1A | NM_001127321; NM_001127322; NM_012117 |
| CLH1_MOUSE | 1213 | CLH1; | CLTC | CLTCL2; CHC17; CLH-17; Hc; | XM_011524279; XM_011524280; |
| | | A0A087 | | CHC | XM_01152481; XM_005257012; |
| | | WVQ6 | | | NM_001288653; NM_004859 |
| PDS5A_MOUSE | 23244 | PDS5A | PDS5A | PIG54; SCC112; SCC-112 | NM_001100400; XM_011513673; |
| | | | | | XM_011513674; NM_015200; |
| | | | | | NM_001100399; XM_011513672 |
| TPM4_MOUSE | 9169 | SCAFB | SCAF11 | SRSF21P; SFRS21P; CASP11; SIP1; | XM_011538985; NM_004719; XM_011538986; |
| | | | | SRRP129 | XM_006719692; XM_011538984; |
| | | | | | XM_005269230; XM_011538983; |
| | | | | | XM_011538987 |
| REXO4_MOUSE | 57109 | REXO4 | REXO4 | XPMC2H; XPMC2; REX4q | NM_001279350; NR_103996; NM_020385; |
| | | | | | NM_001279351; NR_103995; NM_001279349 |
| CNFN_MOUSE | 84518 | CNFN | CNFN | PLAC8L2 | XM_005259332; XM_011527396; NM_032488; |
| | | | | | XM_011527397 |
| RS9_MOUSE | 6203 | RS9 | RPS9 | S9 | XM_011547987; XM_011548358; |
| | | | | | XM_011548624; XR_431025; XR_431068; |
| | | | | | XR_953069; NM_001013; XM_005278288; |
| | | | | | XM_006726201; XM_006726202; |
| | | | | | XM_011547988; XM_011548623; XR_254260; |
| | | | | | XR_254311; XR_431090; XR_952765; |
| | | | | | XR_952994; XM_011547789; XM_011547790; |
| | | | | | XR_431067; XR_952920; XR_952995; |
| | | | | | XR_953155; XR_254518; XR_953156; |
| | | | | | XM_005277274; XM_006725965; XR_431057; |
| | | | | | XR_431069; XR_952922; XR_952996; |
| | | | | | XR_953068; XM_005278287; XM_011548167; |
| | | | | | XR_254517; XR_952766; XR_953070; |
| | | | | | XR_953157; XM_005277315; XM_011548359; |
| | | | | | XR_431058; XR_952764; XR_952919; |
| | | | | | XM_005277084; XM_005277085; |
| | | | | | XM_011548166; XR_430207; XR_431099 |
| RPA34_MOUSE | 10849 | RPA34 | CD3EAP | CAST; PAF49; ASE-1; ASE1 | NM_001297590; NM_012099 |
| LC7L2_MOUSE | 51631 | LC7L | LUC7L | CGI-74; LUC7B2; CGI-59 | ; NM_001244585; NM_016019; NM_001270643 |
| DHX33_MOUSE | 56919 | DHX33 | DHX33 | DDX33 | XR_934069; NM_001199699; NM_020162 |
| TNPO1_MOUSE | 3842 | TNPO1 | TNPO1 | MIP, IPO2; MIP1; TRN; KPNB2 | XM_005248500; NM_153188; XR_948249; |
| | | | | | NM_002270; XM_005248501 |
| MAK16_MOUSE | 84549 | MAK16 | MAK16 | MAK16L; RBM13 | NM_032509 |
| NU107_MOUSE | 57122 | NU107 | NUP107 | NUP84 | XM_005269037; NM_020401; XM_011538576 |
| WDR3_MOUSE | 10885 | WDR3 | WDR3 | UTP12; DIP2 | NM_006784 |
| BOREA_MOUSE | 55143 | BOREA | CDCA8 | DasraB; BOR; MESRGP; | NM_018101; NM_001256875 |
| | | | | BOREALIN | |
| MAL2_MOUSE | 114569 | MAL2 | MAL2 | — | NM_052886; XM_011516807 |
| CARF_MOUSE | 55602 | CARF | CDKN2AIP | CARF | XM_005263118; NM_017632 |
| NUP93_MOUSE | 9688 | NUP93 | NUP93 | NIC96 | NM_001242795; XM_005256263; NM_014669; |
| | | | | | NM_001242796 |
| NKRF_MOUSE | 55922 | NKRF | NKRF | NRF; ITBA4 | XM_011531365; NM_001173488; |
| | | | | | NM_001173487; NM_017544; |
| RBM34_MOUSE | 23029 | RBM34 | RBM34 | — | XM_011544134; NM_015014; NM_001161533; |
| | | | | | XM_011544133; NR_027762 |
| UTP15_MOUSE | 84135 | UTP15 | UTP15 | NET21 | NM_001284431; XM_011543680; |
| | | | | | NM_001284430; NM_032175 |
| EMC1_MOUSE | 23065 | EMC1 | EMC1 | KIAA0090 | XM_005245788; ; XM_005245787; |
| | | | | | NM_001271429; NM_001271427; |
| | | | | | NM_001271428; NM_015047 |
| ELOA1_MOUSE | 6924 | ELOA1 | TCEB3 | TCEB3A; SIII; EloA; SIII_p110 | NM_003198 |
| P66A_MOUSE | 54815 | P66A | GATAD2A | p66alpha | XM_005259956; XM_011528104; |
| | | | | | XM_005259962; XM_006722780; |
| | | | | | XM_011528106; XM_011528107; NM_017660; |
| | | | | | XM_005259957; XM_005259961; |
| | | | | | NM_001300946; XM_005259959; |
| | | | | | XM_005259960; XM_011528105; |
| | | | | | XM_011528108 |
| SPF45_MOUSE | 84991 | SPF45 | RBM17 | SPF45 | NM_032905; NM_001145547 |
| SF3A1_MOUSE | 10291 | SF3A1 | SF3A1 | PRPF21; PRP21; SF3A120; SAP114 | ; NM_005877; NM_001005409 |
| NU133_MOUSE | 55746 | NU133 | NUP133 | hNUP133 | ; NM_018230 |
| THOC1_MOUSE | 9984 | THOC1 | THOC1 | HPR1; P84N5; P84 | XM_011525773; XM_011525774; NM_005131; |
| | | | | | XM_011525772 |
| NOL6_MOUSE | 65083 | NOL6 | NOL6 | NRAP; bA311H10.1; UTP22 | NM_022917; NM_139235; NM_130793 |
| NDC1_MOUSE | 55706 | NDC1 | NDC1 | NET3, TMEM48 | XM_011541766; NR_033142; XM_006710762; |
| | | | | | NM_018087; NM_001168551 |
| CCAR2_MOUSE | 57805 | CCAR2 | CCAR2 | p30 DBC; DBC1; KIAA1967; | XM_011544604; NM_199205; NR_033902; |
| | | | | NET35; p30DBC; DBC-1 | XM_011544603; NM_021174 |
| LEGL_MOUSE | 29094 | LEGL | LGALSL | GRP; HSPC159 | NM_014181 |
| P66B_MOUSE | 57459 | P66B | GATAD2B | MRD18; P66beta; p68 | XM_005245364; XM_011509808; NM_020699; |
| | | | | | XM_006711469 |
| FLNC_MOUSE | 2318 | FLNC | FLNC | ABP-280; ABPA; MPD4; ABPL; | ; NM_001127487; NM_001458 |
| | | | | MFM5; ABP280A; FLN2 | |
| DDX1_MOUSE | 1653 | DDX1 | DDX1 | DBP-RB; UKVH5d | NM_004939 |
| DNJC9_MOUSE | 23234 | DNJC9 | DNAJC9 | JDD1; HDJC9; SB73 | NM_015190 |
| PTBP2_MOUSE | 58155 | PTBP2 | PTBP2 | nPTB; PTBLP; brPTB | XR_946723; XT946722; NM001300987; |
| | | | | | NR_125357; XM_011541876; XM_011541875; |
| | | | | | XR_946720; NM_001300986; NM_001300988; |
| | | | | | NM_02190; NM_001300990; NR_125356; |
| | | | | | XM_011541874; XR_946721; NM_001300985; |
| | | | | | NM_001300989 |
| SMC6_MOUSE | 79677 | SMC6 | SMC6 | hSMC6; SMC-6; SMC6L1 | XR_939716; NM_001142286; XM_011533107; |
| | | | | | XM_011533108; NM_024624 |
| SFXN1_MOUSE | 94081 | SFXN1 | SFXN1 | — | XM_005266102; NM_022754 |
| RLP24_MOUSE | 51187 | RLP24 | RSL24D1 | HRP-L30-iso; TVAS3; RLP24; | NM_016304 |
| | | | | C15orf15; L30; RPL24; RPL24L | |
| RTCB_MOUSE | 51493 | RTCB | RTCB | HSPC117; C22orf28; DJ149A16.6; | NM_014306 |
| | | | | FAAP | |
| CPSF5_MOUSE | 11051 | CPSF5 | NUDT21 | CFIM25; CPSF5 | NM_007006 |
| LSM7_MOUSE | 51690 | LSM7 | LSM7 | YNL147W | XM_011528061; NM_016199 |
| RER1_MOUSE | 11079 | RER1 | RER1 | — | XM_005244713; XM_011540543; NM_007033; |
| | | | | | XM_011540542; XM_006710306; |
| NSA2_MOUSE | 10412 | NSA2 | NSA2 | CDK105, TINP1; HUSSY-29; | XM_011543098; NM_001271665; XR_948227; |
| | | | | HUSSY29; HCLG1; HCL-G1 | NM_014886; NR_073403 |
| RRP15_MOUSE | 51018 | RRP15 | RRP15 | CGI-115; KIAA0507 | XM_011509597; NM_016052 |
| CISY_MOUSE | 1431 | A0A024R | CS | — | NM_004077; NM_198324 |
| | | B75; | | | |
| | | CISY | | | |
| RFC5_MOUSE | 5985 | RFC5 | RFC5 | RFC36 | XM_011538645; NM_001130112; |
| | | | | | NM_001130113; NM_007370; NM_001206801; |
| | | | | | XM_011538643; XM_011538644; NM_181578 |
| SYRC_MOUSE | 5917 | SYRC | PARS | HLD9; DALRD1; ArgRS | NM_002887; |
| PHF6_MOUSE | 84295 | PHF6 | PHF6 | BFLS; BORJ; CENP-31 | NM_001015877; NM_032335; ; NM_032458 |
| SUN1_MOUSE | 23353 | SUN1 | SUN1 | UNC84A | NM_001171945; NM_001130965; |
| | | | | | NM_001171944; NM_025154; NM_001171946 |
| CALL3_MOUSE | 810 | CALL3 | CLAML3 | CLP | NM_005185 |
| TGM5_MOUSE | 9333 | TGM5 | TGM5 | TGASE5; TGM6; TGX; PSS2; | XM_011522229l XR_931948; NM_201631; |
| | | | | TGMX; TGASEX | NM_004245; XM_011522230 |
| CPNS2_MOUSE | 84290 | CPNS2 | CAPNS2 | — | NM_032330 |
| FIP1_MOUSE | 81608 | FIP1 | FIP1L1 | FIP1; Rhc; hFip1 | XM_005265770; NM_001134937; |
| | | | | | XM_005265768; XM_005265781; NM_030917; |
| | | | | | XM_005265769; XM_005265773; |
| | | | | | XM_005265774; XM_005265778; |
| | | | | | XM_005265779; ; XM_005265771; |
| | | | | | NM_001134938; XM_005265780; |
| | | | | | XM_005265782; XM_005265776; |
| | | | | | XM_005265777; XM_005265772; |
| | | | | | XM_005265775 |
| EVPL_MOUSE | 2125 | EVPL | EVPL | EVPK | XM_011524516; NM_001988 |
| SNAA_MOUSE | 8775 | SNAA | NAPA | SNAPA | XM_011537437; NR_038457; NM_003827; |
| | | | | | XM_011527436; NR_039456 |
| RRP8_MOUSE | 23378 | RRP8 | RRP8 | NML; KIAA0409 | XR_930858; XM_011519955; XR_930859; |
| | | | | | NM_015324; XR_930860 |
| XRN2_MOUSE | 22803 | XRN2 | XRN2 | — | XM_011529184; NM_012255 |
| NDUA9_MOUSE | 4704 | NDUA9 | NDUFA9 | CI-39k; CI39k; CC6; NDUFFS2L; | ; NM_005002 |
| | | | | SDR22E1 | |
| CPSF1_MOUSE | 29894 | CPSF1 | CPSF1 | CPFS160; P/c1.18; HSU37012 | XM_006716548; XM_011516999; NM_013291; |
| | | | | | XM_006716550; XM_011516998; |
| | | | | | XM_011516997; XM_006716549 |
| AR6P4_MOUSE | 51329 | AR6P4 | ARL6IP4 | SRrp37; SR-25; SFRS20; SRp25 | NR_103512; NM_001002252; NM_001278380; |
| | | | | | NM_018694; NM_001278378; NM_001278379; |
| | | | | | NM_001002251; NM_016638 |
| CAF1A_MOUSE | 10036 | CAF1A | CHAF1A | CAF-1; CAF1B; CAF1; CAF1P150; | XR_936135; XM_011527607; XM_011527605; |
| | | | | P150 | XM_011527606; NM_005483 |
| INCE_MOUSE | 3619 | INCE | ICNENP | — | XM_011544998; XM_011544995; |
| | | | | | XM_011544997; XM_006718533; |
| | | | | | XM_011544996; NM_001040694; NM_020238 |
| RFC2_MOUSE | 5982 | RFC2 | RFC2 | RFC40 | XR_927506; NM_001278792; NM_001278793; |
| | | | | | NM_002914; NM_181471; ; NM_001278791; |
| | | | | | XM_006716080 |
| SNF5_MOUSE | 6598 | SNF5 | SMARCB1 | MRD15; Snr1; INI1; RDT; RTPS1; | ; XM_011546908; XM_011546909; |
| | | | | SWNTS1; PPP1R144; SNF5; Sth1p; | NM_001007468; NM_003073; XM_011530346; |
| | | | | SNF5L1; BAF47; hSNFS | XM_011530345 |
| HNRPC_MOUSE | 3183 | HNRPC | HNRNPC | HNRNP; SNRPC; C1; C2; HNRPC | NM_031314; XM_011536708; XM_006720125; |
| | | | | | XM_011536710; NM_001077442; |
| | | | | | XM_011536709; ; NM_004500; |
| | | | | | NM_001077443; XM_011536711; |
| | | | | | XM_011536712 |
| B0LM42_MOUSE | 29028 | ATAD2 | ATAD2 | PRO2000; CT137; ANCCA | XM_011516995; XM_011516996; XR_928326; |
| | | | | | XM_011516994; NM_014109 |
| D3YUU6_MOUSE | 64794 | DDX31 | DDX31 | PPP1R25 | XM_011518923; XM_005272206; |
| | | | | | XM_011518921; XM_011518924; NM_138620; |
| | | | | | XR_246600; XR_929836; XM_006717236; |
| | | | | | NM_022779; XM_005272207; XM_011518922 |
| E9PWW9_MOUSE | 57466 | SFR15 | SCAF4 | SRA4; SFRS15 | NM_001145445; XM_006724036; |
| | | | | | NM_001145444; XM_005261017; |
| | | | | | XM_006724035; NM_020706 |
| E9PZM8_MOUSE | — | | | | |
| G3X963_MOUSE | 5646 | TYR3 | PRSS3 | PRSS4; TRY4; TRY3; MTG; T9 | ; NM_001197098; NM_007343; |
| | | | | | NM_001197097; XM_011517965; NM_002771 |
| Q3TWW8_MOUSE | — | | | | |
| Q6NZQ2_MOUSE | 10180 | RBM6 | RBM6 | DEF-3; HLC-11; 3G2; g16; NY-LU- | NM_005777; XM_005264787; XM_005264786; |
| | | | | 12; DEF3 | XM_005264785; XM_005264788; |
| | | | | | NM_001167582; XM_005264784; |
| | | | | | XM_006712916; XR_940359; XR_940360 |
| Q6PFF0_MOUSE | 4288 | K167 | MK167 | KIA; MIB-1; MIB-; PPP1R105 | NM_002417; NM_001145966; XM_006717864; |
| | | | | | XM_011539818 |
| Q9ZIR9_MOUSE | 56252 | YLPM1 | YLPM1 | PPP1R169; ZQP3; C14orf170; | XM_005267860; XM_011536966; |
| | | | | ZAP113 | XM_011536967; NM_019589; XR_943494 |
| S4R1W5_MOUSE | 142 | PARP1 | PARP1 | PARP; PARP-1; ADPRT1; PPOL; | NM_001618 |
| | | | | pADPRT-1; ADPRT; ADPRT 1; | |
| | | | | ARTD1 | |
| E9PVX6_MOUSE | 9790 | BMS1 | BMS1 | ACC; BMS1L | XR_428728; XM_005271846; XM_005271849; |
| | | | | | XM_006718081; XM_014753; XM_005271848; |
| | | | | | XR_246522; XM_005271847; XM_011540403; |
| | | | | | XM_011540402 |
| D3YWX2_MOUSE | 10940 | PQP1 | PQP1 | — | NM_001145860; NM_01145861; NM_015029; |
| | | | | | XM_011516800; XM_011516801 |
| Q921K2_MOUSE | 9416 | DDX23 | DDX23 | prp28; SNRNP100; PRPF28; U5- | NM_004818 |
| | | | | 100K; U5-100KD | |
| SUN2_MOUSE | 25777 | SUN2 | SUN2 | UNC84B | NM_015374; XM_011530105; XM_011530104; |
| | | | | | NM_001199580; NM_01199579 |
| SAFB1_MOUSE | 6294 | SAFB1 | SAFB | HAP; HET; SAF-B1; SAFB1 | XM_006722839; NR_037699; NM_001201340; |
| | | | | | NM_001201339; NM_001201338; NM_002967 |
| HNRL2_MOUSE | 221092 | HNRL2 | HNRNPUL2 | HNRPUL2; SAF-A2 | NM_001079559 |
| CHD4_MOUSE | 1108 | CHD4 | CHD4 | Mi2-BETA; Mi-2b; CHD-4 | XM_006718958; NM_001273; XM_006718962; |
| | | | | | XM_006718960; XM_006718959; |
| | | | | | XM_005253668; XM_006718961; |
| | | | | | NM_001297553 |
| TCOF_MOUSE | 6949 | TCOF | TCOF1 | treacle; MFD1; TCS1; TCS | NM_001008656; XM_005268504; |
| | | | | | XM_005268505; NM_001135243; |
| | | | | | XM_005268509; NM_000356; NM_001008657; |
| | | | | | NM_001135245; XM_011537678; XR_427780; |
| | | | | | XM_005268502; XM_005268507; XR_427778; |
| | | | | | XM_005268506; XM_005268508; ; |
| | | | | | NM_001135244; XM_005268503; XR_427779; |
| | | | | | NM_001195141 |
| RRP1B_MOUSE | 23076 | RRP1B | RRP1B | PPP1R136; KIAA0179; NNP1L; | NM_015056 |
| | | | | Nnp1; RRP1 | |
| LA_MOUSE | 6741 | LA | SSB | La; La/SSB; LARP3 | NM_003142; NM_001294145; |
| Q6PGF5_MOUSE | 3187 | HNRH1 | HNRNPH1 | HNRPH1; hnRNPH; HNRPH | XM_006714862; XM_005265895; |
| | | | | | XM_006714863; XM_011534541; |
| | | | | | XM_005265901; XM_005265896; |
| | | | | | XM_011534542; XM_011534543; |
| | | | | | XM_011534544; NM_001257293; NM_005520; |
| | | | | | XM_011534547; XM_005265902; |
| | | | | | XM_011534545; XM_011534546 |
| Q8K205_MOUSE | 9221 | NOLC1 | NOLC1 | NOPP130; NOPP140; P130; | XM_005270273; NM_004741; NM_001284389; |
| | | | | NS5ATP13 | NM_001284388 |
| HMGB2_MOUSE | 3148 | HMGB2 | HMGB2 | HMG2 | NM_002129; NM_001130688; NM_001130689 |
| HNRH2_MOUSE | 3188 | HNRH2 | HNRNPH2 | FTP3; HNRPH′; HNRPH2; | ; NM_019597; NM_001032393 |
| | | | | hnRNPH′ | |
| TR150_MOUSE | 9967 | TR150 | THRAP3 | TRAP150 | XM_005271371; XR_246308; NM_005119 |
| SNR40_MOUSE | 9410 | SNR40 | SNRNP40 | PRPF8BP; 40K; SPF38; WDR57; | NM_004814 |
| | | | | HPRP8BP; PRP8BP | |
| MTA2_MOUSE | 9219 | MTA2 | MTA2 | MTA1L1; PID | NM_004739 |
| RRP5_MOUSE | 22984 | RRP5 | PDCD11 | NFBP; RRP5; ALG-4; ALG4 | NM_014976; XM_011539538; XM_011539540; |
| | | | | | XM_005269647; XM_011539539 |
| CO1A1_MOUSE | 1277 | CO1A1 | COL1A1 | O14 | NM_000088; ; XM_005257059; |
| | | | | | XM_005257058; XM_011524341 |
| CATA_MOUSE | 847 | CATA | CAT | — | ; NM_001752 |
| PSA2_MOUSE | 5683 | A0A024R | PSMA2 | OSMA2; HC3; MU; PSC2 | NM_002787 |
| | | A52; | | | |
| | | PSA2 | | | |
| PUF60_MOUSE | 22827 | PUF60 | PUF60 | SIAHBP1; RoBPI; FIR; VRJS | NM_001271096; NM_001271097; |
| | | | | | NM_001136033; NM_014281; ; |
| | | | | | NM_001271100; NM_078480; XM_011516929; |
| | | | | | NM_001271098; XM_011516930; |
| | | | | | NM_001271099 |
| SF01_MOUSE | 7536 | SF01 | SF1 | MBBP; D11S636; ZCCHC25; BBP; | NM_001178031; NR_033649; NR_033650; |
| | | | | ZFM1; ZNF162 | NM_001178030; XM_011545247; NM_201995; |
| | | | | | NM_201998; XM_011545245; ; NM_004630; |
| | | | | | XM_011545244; XM_011545248; NM_201997; |
| | | | | | XM_011545246 |
| IMMT_MOUSE | — | | | | |
| DDX54_MOUSE | 79039 | DDX54 | DDX54 | DP97 | NM_001111322; NM_024072 |
| RBM19_MOUSE | 9904 | RBM19 | RBM19 | — | XM_011539038; XR_944848; NM_016196; |
| | | | | | NM_001146698; NM_001146699 |
| SMCA5_MOUSE | 8467 | SMCA5 | SMARCA5 | ISWI; SNF2H; hISWI; WCRF135; | NM_003601; XM_011532361 |
| | | | | hSNF2H | |
| GLYR1_MOUSE | 84656 | GLYR1 | GLRY1 | BM045; N-PAC; NP60; HIBDL | XM_005255638; XM_011522717; XR_932954; |
| | | | | | XM_005255640; NM_032569; XM_005255639; |
| | | | | | XM_011522716; XM_011522718; |
| | | | | | XM_005255637; XR_243321 |
| PSIP1_MOUSE | 11168 | PSIP1 | PSIP1 | PSIP2; p52; DFS70; LEDGF; p75; | XM_005251358; XM_011517698; |
| | | | | PAIP | NM_001128217; NM_033222; XM_011517697; |
| | | | | | XM_011517700; NM_021144; XM_011517699 |
| NOG1_MOUSE | 23560 | NOG1; | GTPBP4 | CRFG; NGB; NOG1 | NM_012341 |
| | | D2CFK9 | | | |
| PSA6_MOUSE | 5687 | PSA6 | PSMA6 | IOTA; p27K; PROS27 | ; NM_001282234; NM_002791; |
| | | | | | NM_001282232; NM_001292233; NR_104110 |
| D3Z0M9_MOUSE | 9295 | SRS11 | SRSF11 | dJ677H15.2; p54; SFRS11; NET2 | XM_005271339; XM_011542429; NM_004768; |
| | | | | | XM_011542430; NM_001190987; |
| | | | | | XM_005271338; XM_006711037; |
| | | | | | XM_011542432; XM_006711038; |
| | | | | | XM_011542433; XR_426640; XM_011542428; |
| | | | | | XM_006711039 |
| DHX9_MOUSE | 1660 | DHX9 | DHX9 | DDX9; LKP; NHD2; NDHII; RHA | ; NM_001357; NM_030588; NR_033302 |
| DHX15_MOUSE | 1665 | DHX15 | DHX15 | PRPF43; HRH2; PRP43; DBP1; | XR_925314; NM_001358 |
| | | | | DDX15; PrPp43p | |
| ELAV1_MOUSE | 1994 | ELAV1 | ELAVL1 | ELAV1; Me1G; Hua; HUR | XM_011527777; NM_001419 |
| CDC5L_MOUSE | 988 | CDC5L | CDC5L | PCDC5RP; CDC50LIKE; | XM_006715289; NM_001253; XR_926346 |
| | | | | dJ319D22.1; CEF1; CDC5 | |
| NUP98_MOUSE | 10236 | HNPRP | HNRNPR | hnRNP-R; HNRPR | XM_011540473; XM_005245711; |
| | | | | | XM_011540472; NM_001102399; |
| | | | | | NM_001102397; XM_011540474; |
| | | | | | XM_011540476; NM_001297621; |
| | | | | | NM_001297622; XM_011540471; |
| | | | | | XM_011540475; XM_011540477; |
| | | | | | NM_001102398; NM_001297620; NM_005826 |
| RBM28_MOUSE | 55131 | RBM28 | RBM28 | ANES | XM_011516370; XM_011516371; NM_018077; |
| | | | | | NM_001166135; XR_927487; |
| Q8C2Q7_MOUSE | 79026 | AHNK | AHNAK | AHNAKRS | XM_005274240; XM_005274242; |
| | | | | | XM_005274243; XM_011545250; |
| | | | | | XM_005274241; XM_005274244; NM_024060; |
| | | | | | XM_005274245; XM_011545249; NM_001620 |
| PRP8_MOUSE | 10594 | PRP8 | PRPF8 | SNRNP220; HPRP8; PRPC8; PRP8; | NM_006445; |
| | | | | RP13 | |
| U520_MOUSE | 23020 | U520 | SNRNP200 | ASCC3L1; BRR2; RP33; U5- | ; NM_014014 |
| | | | | 200KD; HELIC2 | |
| BAZIB_MOUSE | 9031 | BAZIB | BAZIB | WBSCR9; WBSR10; WSTF | NM_032408; NM_023005; XM_005250683; |
| UST48_MOUSE | 1650 | A0A024R | DDOST | OST; OST48; AGER1; OKSWc145; | ; NM_005216 |
| | | AD5; | | CDG1R; WBP1 | |
| | | OST48 | | | |
| P53_MOUSE | 7157 | H2EHT1; | TP54 | TRP53; BCC7; P53; LFS1 | NM_001126112; NM_001276697; |
| | | K7PPA8; | | | NM_01126115; ; NM_01126114; |
| | | P53; | | | NM_001276698; NM_001276761; |
| | | A0A087 | | | NM_001126118; NM_001126113; |
| | | WXZ1; | | | NM_001126117; NM_001276695; |
| | | A0A087X | | | NM_001276699; NM_001276760; NM_000546; |
| | | 1Q1; | | | NM_001126116; NM_001276696 |
| | | Q53GA5; | | | |
| | | A0A087 | | | |
| | | WT22 | | | |
| LYZ1_MOUSE | 1E+08 | XP32 | C1orf68 | XP32; LEP7 | NM_001024679 |
| H2A1_MOUSE | 5725 | PTBP1 | PTBP1 | pPTB; PTB3; HNRNP-1; PTB; | XR_244034; NM_002819; XR_244035; |
| | | | | HNRNPI; PTB-T; PTB2; HNRP1; | XM_005259597; NM_031991; NM_175847; |
| | | | | PTB-1; PTB4 | XM_005259598; NM_031990 |
| RL27_MOUSE | 6155 | A0A024R | RPL27 | L27 | NM_000988 |
| | | 1V4; | | | |
| | | RL27 | | | |
| RS6_MOUSE | — | | | | |
| RBBP6_MOUSE | 5930 | RBBP6 | RBBP6 | P2P-R; MY038; RBQ-1; SNAMA; | XM_005255461; NM_018703; XM_005255462; |
| | | | | PACT | NM_006910; NM_032626 |
| LYAR_MOUSE | 55646 | LYAR | LYAR | ZC2HC2; ZYLAR | XM_011513505; NM_001145725; NM_017816; |
| | | | | | XM_011513506 |
| PSA_MOUSE | 9520 | PSA | NPEPPS | PSA; AAP-S; MP100 | XM_011525496; NM_006310 |
| RRP12_MOUSE | 23223 | RRP12 | RRP12 | KIAA0690 | NM_015179; XM_011539556; XM_011539557; |
| | | | | | XM_011539555; NM_001145114; |
| | | | | | NM_001284337 |
| WDR43_MOUSE | 23160 | WDR43 | WDR43 | NET12; UTP5 | NM_015131 |
| RS27_MOUSE | 6232 | RS27 | RPS27 | MPS-1; S27; MPS1 | NM_001030 |
| RL24_MOUSE | 6152 | RL24 | RPL24 | HEL-S-310; L24 | NM_000986 |
| RFOX2_MOUSE | 23543 | RFOX2 | RBFOX2 | FOX2; Fox-2; HNRBP2; HRNBP2; | XM_006724190; XM_006724193; |
| | | | | RBM9; RTA; fxh; dJ106I20.3 | XM_006724185; XM_006724187; |
| | | | | | XM_011530036; NM_001031695; |
| | | | | | NM_001082577; XM_005261428; |
| | | | | | XM_005261430; XM_005261431; |
| | | | | | XM_005261432; XM_005261433; |
| | | | | | XM_005261437; NM_001082579; |
| | | | | | XM_005261429; XM_006724186; |
| | | | | | XM_006724194; XM_006724192; |
| | | | | | NM_001082578; NM_014309; NM_001082576; |
| | | | | | XM_005261435; XM_006724188; |
| | | | | | XM_006724189; XM_006724191 |
| MYEF2_MOUSE | 50804 | MYEF2 | MYEF2 | myEF-2; MSTP156; HsT18564; | XM_005254424; NM_006720553; |
| | | | | MEF-2; MST156 | XM_005254422; XM_005254425; |
| | | | | | NM_001301210; NM_016132; XM_005254427; |
| | | | | | XM_011521657; NR_125408 |
| MATR3_MOUSE | 9782 | MATR3 | MATR3 | MPD2; ALS21; VCPDM | NM_001282278; NM_018834; NM_001194956; |
| | | | | | NM_199189; ; NM_01194954; NM_001194955 |
| RBM39_MOUSE | 9584 | RBM39 | RBM39 | CAPERalpha; FSAP59; CAPER; | XM_011529110; NM_184237; XM_006723891; |
| | | | | HCC1; RNPC2 | XM_006723893; NM_001242599; NM_184234; |
| | | | | | ; NM_001242600; NR_040722; XM_006723890; |
| | | | | | XM_01152911; NM_004902; NR_040723; |
| | | | | | NM_184241; NR_040724; NM_184244 |
| PRP6_MOUSE | 24148 | PRP6 | PRPF6 | TOM; ANT-1; Prp6; hPrp6; | XM_006723769; ; NM_012469 |
| | | | | C20orf14; RP60; ANT1; | |
| | | | | SNRNP102; U5-102K | |
| SSF1_MOUSE | 56342 | SSF1 | PPAN | SSF-1; SSF1; BXDC3; SSF; SSF2 | NM_020230 |
| ILF2_MOUSE | 3608 | ILF2 | ILF2 | NF45; PRO3063 | NM_001267809; NM_004515 |
| TMM43_MOUSE | 79188 | TMM43 | TMEM43 | LUMA; ARVC5; ARVD5; ADMD7 | XM_011534109; ; NM_024334 |
| PK1IP_MOUSE | 55003 | PK1IP1 | PAK1IP1 | bA421M1.5; PIP1; hPIP1; MAK11; | XM_005249204; XM_011514720; |
| | | | | WDR84 | XM_006715129; XM_011514721; NM_017906 |
| GSDMA_MOUSE | 284110 | GSDMA | GSDMA | FKSG9; GSDM; GSDM1 | XM_006721832; XM_011524651; NM_178171 |
| SON_MOUSE | 6651 | SON | SON | NREBP; BASS1; DBP-5; C21orf50; | NR_103797; NM_138927; NM_001291412; |
| | | | | SON3 | NM_003103; NR_103798; NM_001291411; |
| | | | | | NM_032195; NM_138925; NR_103796 |
| E9Q5C9_MOUSE | — | | | | |
| E9Q6E5_MOUSE | — | | | | |
| Q8VHM5_MOUSE | — | | | | |
| TOP2A_MOUSE | 7153 | TOP2A | TOP2A | TOP2; TP2A | XM_005257632; XM_011525165; NM_001067; |
| FINC_MOUSE | 2335 | FINC | FN1 | FNZ; GFND; C1G; ED-B; GFND2; | XM_005246416; ; XM_005246413; |
| | | | | MSF; FINC; FN; LETS | NM_212476; XM_005246407; XM_005246410; |
| | | | | | XM_005246414; NM_212474; XM_005246402; |
| | | | | | XM_005246408; XM_005246409; |
| | | | | | XM_005246399; NM_054034; XM_005246400; |
| | | | | | XM_005246403; XM_005246405; |
| | | | | | XM_005246406; XM_005246415; NM_002026; |
| | | | | | XM_005246398; XM_005246401; |
| | | | | | XM_005246404; XM_005246412; |
| | | | | | XM_005246417; XM_005246397; |
| | | | | | XM_005246411; NM_212478; NM_212482; |
| | | | | | NM_212475 |
| RASK_MOUSE | 3845 | RASK | KRAS | KI-RAS; NS; K-RAS4B; K-RAS4A; | XM_011520653; NM_004985; ; |
| | | | | RASK2; CFC2; K-RAS2B; KRAS2; | XM_006719069; NM_033360 |
| | | | | KRAS1; C-K-RAS; K-RAS2A; NS3 | |
| HNRPQ_MOUSE | 10492 | HNRPQ | SYNCRIP | GRY-RBP; HNRPQ1; PP68; | XM_005248636; XM_005248637; |
| | | | | hnRNP-Q; GRYRBP; NSAP1; | NM_001159676; ; NM_001159673; |
| | | | | HNRNPQ | NM_001159674; NM_001159677; |
| | | | | | NM_001159675; NM_001253771; NM_006372; |
| | | | | | XM_005248635 |
| MYH10_MOUSE | 4628 | MYH10 | MYH10 | NMMHC-IIB; NMMHCB | NM_001256095; XM_011523875; |
| | | | | | XM_011523877; XM_011523879; |
| | | | | | XM_011523880; XM_011523876; |
| | | | | | XM_005256651; NM_005964; XM_011523878; |
| | | | | | NM_001256012 |
| DDX51_MOUSE | 317781 | DDX51 | DDX51 | — | XM_011538256; NM_175066 |
| DEK_MOUSE | 7913 | DEK | DEK | D6S231E | XM_011514889; NM_001134709; XR_926307; |
| | | | | | NM_003472 |
| NOP16_MOUSE | 51491 | NOP16 | NOP16 | HSPC185; HSPC111 | NM_001291306; NM_016391; NM_001256539; |
| | | | | | NM_001256540; NM_001291305; |
| | | | | | XM_011534567; NM_001291308; |
| | | | | | XM_011534566; NM_001291307 |
| RBM14_MOUSE | 10432 | RBM14 | RBM14 | COAA; TMEM137; SIP; SYTIP1; | NM_001198837; ; NM_001198836; |
| | | | | PSP2 | NM_006328; NM_032886 |
| RL4_MOUSE | 6124 | RL4 | RPL4 | L4 | NM_000968 |
| ADT1_MOUSE | 291 | SDT1 | SLC25A4 | AAC1; ANT; ANT1; PEO2; PEO3; | NM_001151; |
| | | | | 1; ANT 1; MTDPS12; T1 | |
| HNRPL_MOUSE | 3191 | HNRPL | HNRNPL | HNRPL; hnRNP-L; P/OKc1.14 | XM_011526887; XR_243927; XM_011526886; |
| | | | | | XM_011526889; NM_001533; NM_001005335; |
| | | | | | XM_011526888; XM_011526890 |
| NONO_MOUSE | 4841 | NONO | NONO | P54; PPP1R114; NMT55; NRB54; | NM_001145410; NM_007363; NM_001145409; |
| | | | | P54NRB | NM_001145408 |
| DNMT1_MOUSE | 1786 | I6L9H2; | DNMT1 | AIM; CXXC9; DNMT; MCMT; | XM_011527773; ; NM_001130823; |
| | | DNMT1 | | ADCADN; HSN1I | NM_001379; XM_011527772; XM_011527774 |
| E9Q616_MOUSE | — | | | | |
| HNRPM_MOUSE | 4670 | HNRPM | HNRNPM | HTGR1; NAGR1; hnRNP M; | NM_005968; XM_005272478; XM_005272480; |
| | | | | HNRPM; CEAR; HNRNPM4; | XM_005272483; XM_005272479; |
| | | | | HNRPM4 | XM_005272481; NM_001297418; NM_031203; |
| FBX50_MOUSE | 342897 | FBX50 | NCCRP1 | NCCRP-1; FBXO50 | NM_001001414; XM_011526906 |
| PSB1_MOUSE | 5689 | PSB1 | PSMB1 | PSC5; PMSB1; HC5 | NM_002793 |
| SRSF5_MOUSE | 6430 | SRSF5 | SRSF5 | HRS; SRP40; SFRS5 | XM_005267999; XR_943505; NM_006925; |
| | | | | | XM_005267998; XR_943506; NM_001039465; |
| | | | | | XM_005268000; XM_011537077 |
| CAN1_MOUSE | 823 | CAN1 | CAPN1 | muCL; CANPL1; muCANP; CANP; | NM_001198868; NR_040008; XM_006718698; |
| | | | | CANP1 | XM_011545292; NM_005186; NM_001198869 |
| ZN326_MOUSE | 284695 | ZN326 | ZNF36 | Zfp326; ZAN75; dJ871E2.1; ZIRD | NM_181781; XM_005270780; XM_005270779; |
| | | | | | XM_011541288; XM_011541289; |
| | | | | | XM_011541290; NM_182975; NM_182976 |
| CASPE_MOUSE | 23581 | CASPE | CAP14 | — | NM012114; XM011527861 |
| COX2_MOUSE | 4513 | COX2; | COX2 | COII; MTCO2 | |
| | | U5Z487 | | | |
| MAOX_MOUSE | 4199 | MAOX | MEI | HUMNDME; MES | XM_011535836; NM_002395 |
| RL7_MOUSE | 6129 | RL7 | RPL7 | L7; humL7-1 | XM_006716463; NM_000971 |
| NDKA_MOUSE | 4830 | NDKA | NME1 | GAAD; NB; AWD; NBS; NDPK-A; | ; NM_198175; NM_000269 |
| | | | | NDPKA; NDKA; NM23; NM23-H1 | |
| TPM3_MOUSE | 7170 | TPM3 | TPM3 | NEM1; HEL-189; OK/SW-c1.5; | XM_006711520; XM_006711521; |
| | | | | TM30nm; TM-5; TH5; CAPM1; | XM006711523; NR_103461; XM_006711517; |
| | | | | TM3; TM30; CFTD; hscp30; | NM_001043353; XM_006711522; |
| | | | | TPMsk3; HEL-S-82p; TRK | XM_006711519; XM_011509950; |
| | | | | | XM_011509953; NM_001278190; NM_152263; |
| | | | | | XM_011509952; NM_153649; XM_006711515; |
| | | | | | XM_011509954; NM_001278189; |
| | | | | | XM_011509951; NM_001278188; |
| | | | | | NM_001278191; XM_006711518; |
| | | | | | NM_001043351; NM_001043352; NR_103460 |
| RS2_MOUSE | 6187 | RS2 | RPS2 | LLREP3; S2 | NM_002952 |
| RL12_MOUSE | 6136 | RL12 | RPL12 | L12 | NM_000976 |
| H11_MOUSE | 3024 | H11 | HISTH1A | H1.1; HIST1; H1A; H1F12 | NM_005325 |
| CAPZB_MOUSE | 832 | CAPBZ | CAPBZ | CAPB; CAPZ; CAPPB | XM_011542229; NM_001206541; NM_004930; |
| | | | | | XM_006710938; XM_011542230; |
| | | | | | NM_001206540; XM_011542228; |
| | | | | | NM_001282162 |
| LIS1_MOUSE | 5048 | LIS1 | PAFAH1B1 | LIS1; LIS2; MDCR; PAFAH; MDS | XM_011523902; XM_011523903; |
| | | | | | XM_011523904; NM_000430; XM_011523901; |
| HMGB1_MOUSE | 3146 | HNGB1 | HNGB1 | HNG3; SBP-1; HNG1 | XM_005266368; XM_011535056; |
| | | | | | XM_011535055; XR_941568; NM_002128; |
| | | | | | XM_005266363; XM_005266365 |
| RS10_MOUSE | 1.01E+08 | S4R435 | RPS10- | — | NM_001202470 |
| | | | NUDT3 | | |
| PHB_MOUSE | 5245 | PHB | PHB | HEL-S-54e; PHB1; HEL-215 | ; NM_002634; NM_001281715; |
| | — | | | | NM_001281496; NM_001281497 |
| NACAM_MOUSE | | | | | |
| PHF5A_MOUSE | 84844 | PHF5A | PHF5A | DAP14b; INI; Rds3; bK223H9.2; | NM_032758 |
| | | | | SF3B7; SF3b14b | |
| RS3A_MOUSE | 6189 | RS3A; | RPS3A | S3A; MFTL; FTE1 | NM_001267699; NM_001006 |
| | | B7Z3M5 | | | |
| ZCH18_MOUSE | 124245 | ZCH18 | ZC3H18 | NHN1 | XM_011522864; XM_011522863; |
| | | | | | XM_011522865; XM_011522862; |
| | | | | | NM_001294340; NM_144604 |
| FUBP2_MOUSE | 8570 | FUBP2 | KHSRP | FUBP2; FBP2; KSRP | XM_005259668; NM_003685; XM_011528395 |
| DDX17_MOUSE | 10521 | DDX17 | DDX17 | RH70; P72 | NM_001098505; NM_030881; NM_001098504; |
| | | | | | ; NM_006386 |
| LC7L3_MOUSE | 51747 | LC7L3 | LUC7L3 | hLuc7A; CRA; CREAP-1; CROP; | XM_005257448; NM_006107; XM_005257449; |
| | | | | LUC7A; OA48018 | XM_006721943; XM_005257455; NM_016424; |
| | | | | | XM_005257454; XM_005257452; |
| | | | | | XM_005257450 |
| EWS_MOUSE | 2130 | EWS | EWSR1 | EWS; bK984G1.4 | XM_005261389; XM_011529999; |
| | | | | | XM_011530001; NM_013986; XM_011529995; |
| | | | | | XM_011529997; XM_011529996; ; |
| | | | | | NM_001163285; NM_001163286; |
| | | | | | XM_005261390; XM_011529998; |
| | | | | | NM_001163287; XM_011530000; |
| | | | | | XM_011530002; NM_005243 |
| UT14A_MOUSE | 10813 | UT14A | UTP14A | NYCO16; dJ537K23.3; SDCCAG16 | XM_011531264; NM_001166221; NM_006649; |
| | | | | | XM_005262363 |
| PWP2_MOUSE | 5822 | PWP2 | PWP2 | EHOC-17; UTP1; PWP2H | XM_011529667; NM_005049 |
| CPNE1_MOUSE | 8904 | CPNE1 | CPNE1 | COPN1; CPN1 | NM_152931; NM_152927; NM_152930; |
| | | | | | NM_152925; NR_037188; NM_003915; |
| | | | | | NM_152926; NM_001198863; NM_152928 |
| H2AW_MOUSE | 55506 | A0A024Q | H2AFY2 | macroH2A2 | NM_018649 |
| | | ZP6; | | | |
| | | H2AW | | | |
| SLTM_MOUSE | 79811 | SLTM | SLTM | Met | XM_011522027; XM_011522030; |
| | | | | | XM_011522023; XM_011522032; XR_931906; |
| | | | | | NM_017968; XM_011522024; XM_011522026; |
| | | | | | NM_001013843; XM_011522022; |
| | | | | | XM_011522028; XM_006720690; |
| | | | | | XM_011522029; NM_024755; XM_006720686; |
| | | | | | XM_011522025; XM_011522031 |
| GNL3_MOUSE | 27354 | GNL3 | GNL3 | C77032; E21G3; NNP47; NS | NM_206826; NM_014366; NM_206825 |
| PYGB_MOUSE | 5834 | PYGB | PYGB | GPBB | NM_002862 |
| NAT10_MOUSE | 55226 | NAT10 | NAT10 | NET43; ALP | XM_011520197; NM_001144030; NM_024662 |
| DDX52_MOUSE | 11056 | DDX52 | DDX52 | HUSSY19; ROK1 | XM_011546776; NM_007010; XR_951954; |
| | | | | | NM_001291476; XM_011524232; |
| | | | | | XM_011546775; XM_011524233; NM_152300 |
| PRAF3_MOUSE | 10550 | PRAF3 | ARL61P5 | jmw; HSPC127; DERP11; JWA; | NM_006407 |
| | | | | PRAF3; addicsin; GTRAP3-18; | |
| | | | | hp22 | |
| SRSF4_MOUSE | 6429 | SRSF4 | SRSF4 | SFRS4; SRP75 | XM_011541951; NM_005626 |
| SP16H_MOUSE | 11198 | SP16H | SUPT16H | FACTP140; SPT16; CDC68; | NM_007192; ; XM_011536381 |
| | | | | SPT16/CDC68 | |
| TADBP_MOUSE | 23435 | TADBP | TARDBP | ALS10; TDP-43 | NM_007375; XR_946596; ; XR_946597 |
| SF3B1_MOUSE | 34251 | SF3B1 | SF3B1 | PRPF10; SAP155; MDS; SF3b155; | XR_241302; NM_001005526; XR_241300; |
| | | | | Hsh155; PRP10 | NM_012433; XM_011510867; ; XM_011510868 |
| NU155_MOUSE | 9631 | NU155 | NUP155 | ATFB15; N155 | XM_011514166; XM_011514164; |
| | | | | | NM_00178312; XM_011514165; NM_004298; |
| | | | | | NM_153485 |
| SMC3_MOUSE | 9126 | SMC3 | SMC3 | BAM; HCAP; SMC3L1; CSPG6; | ; NM_005445 |
| | | | | CDLS3; BMH | |
| ROA0_MOUSE | 10949 | ROA0 | HNRNPA0 | HNRPA0 | NM_006805 |
| SSRA_MOUSE | 6745 | SSRA | SSR1 | TRAPA | NM_003144; NM_001292008; NR_120448 |
| NH2L1_MOUSE | 4809 | NH2L1 | NHP2L1 | NHPX; SSFA1; FA-1; FA1; | XM_011530201; NM_005008; NM_001003796 |
| | | | | SNU13; SNRNP15-5; 15.5K; | |
| | | | | SPAG12; OTK27 | |
| S10AE_MOUSE | 57402 | S10AE | S100A14 | BCMP84; S100A15 | XM_005245362; NM_020672 |
| NOP56_MOUSE | 10528 | NOP56 | NOP56 | SCA36; NOL5A | NR_027700; ; NM_006392 |
| RPN2_MOUSE | 6185 | RPN2 | RPN2 | SWP1; RPNII; RPN-II; RIBIIR | XM_006723850; NM_002951; XM_006723851; |
| | | | | | XM_005260491; XM_006723849; |
| | | | | | NM_001135771; XM_00672852 |
| RBP2_MOUSE | 5903 | RBP2 | RANBP2 | ANE1; TRP1; TRP2; ADANE; | XM_011511576; NM_006267; XM_005264002; |
| | | | | NUP358; HAE3 | XM_005264004; XM_011511575; |
| | | | | | XM_005264003; XM_005264007; |
| | | | | | XM_011511577; XM_005264005; |
| | | | | | XM_011511578; |
| DKC1_MOUSE | 1736 | DKC1 | DKC1 | DKC; XAP10; NAP57; NOLA4; | ; NR_110021; NM_001288747; NR_110023; |
| | | | | CBF5; DKCX | NM_001363; NR_110022; NM_001142463 |
| IDE_MOUSE | 3416 | IDE | IDE | INSULYSIN | XM_005269769; ; XM_005269766; XR_945727; |
| | | | | | NM_004969; NM_001165946 |
| SAS10_MOUSE | 57050 | SAS10 | UTP3 | SAS10; CRL1; CRLZ1 | NM_020368 |
| AL9A1_MOUSE | 223 | AL9A1 | ALDH9A1 | E3; ALDH7; ALDH9; TMABADH; | NM_000696; ; XM_011509294 |
| | | | | ALDH4 | |
| PSA7_MOUSE | 5688 | PSA7 | PSMA7 | RC6-1; HSPC; XAPC7; C6 | NM_002792; NM_152255 |
| G5E8Z3_MOUSE | — | | | | |
| Q8BGJ5_MOUSE | — | | | | |
| Q9QUK9_MOUSE | — | | | | |
| FBRL_MOUSE | 2091 | FBRL | FBL | FIB; FLRN; RNU31P1 | XM_011548799; XM_011526623; |
| | | | | | XM_011548798; XM_005258651; NM_001436 |
| CEBPZ_MOUSE | 10153 | CEBPZ | CEBPZ | HSP-CBF; CBF2; BOC1; CBF | NM_005760 |
| ACTN4_MOUSE | 81 | ACTN4 | ACTN4 | FSGS; FSGS1; ACTININ-4 | XM_006723406; NM_004924; XM_005259282; |
| | | | | | ; XM_005259281 |
| DDX21_MOUSE | 9188 | DDX21 | DDX21 | GURDB; GUA; RH-II/GuAl RH- | NM_004728; NM_001256910; XM_011540336 |
| | | | | II/GU | |
| Q8BVY0_MOUSE | 26156 | RL1D1 | RSL1D1 | PBK1; L12; UTP30; CS1G | NM_015659 |
| PLEC_MOUSE | — | — | — | — | — |
| E9Q7G0_MOUSE | 4926 | NUMA1 | NUMA1 | NMP-22; NUMA | XM_011545059; XM_011545066; |
| | | | | | NM_001286561; XM_011545054; |
| | | | | | XM_011545060; XM_011545064; |
| | | | | | XM_011545062; XM_011545065; NR_104476; |
| | | | | | XM_011545063; XM_011545055; |
| | | | | | XM_011545061; NM_006185; XM_011545057; |
| | | | | | XM_011545058; XM_006718564; |
| | | | | | XM_011545056 |
| ADT2_MOUSE | 292 | ADT2 | SLC25A5 | ANT2; T2; AAC2; T3; 2F1 | ; NM_001152 |
| LAP2B_MOUSE | 7112 | LAP2B; | TMPO | LAP2; CMD1T; LEMD4; TP; | ; NM_001032284; XM_005269132; |
| | | LAP2A | | PRO0868 | XM_005269130; NM_001032283; NM_003276 |
| NOP58_MOUSE | 51602 | NOP58 | NOP58 | NOP5/NOP58; NOP5; HSPC120 | NM_015934 |
| SRSF1_MOUSE | — | | | | |
| TGM1_MOUSE | — | | | | |
| ILF3_MOUSE | — | | | | |
| H2B1F_MOUSE | 8340; | — | — | — | — |
| | 8341 | | | | |
| E1QN31_MOUSE | 4839 | NOP2 | NOP2 | NSUN1; p120; NOP120; NOL1 | XM_005253691; NM_006170; NM_001033714; |
| | | | | | NM_001258310; NM_001258308; |
| | | | | | NM_001258309; XM_011520962 |
| PRDX4_MOUSE | 10549 | PRDX4 | PRDX4 | AOE37-2; PRX-4; HEL-S-97n; | NM_006406; XM_005274438; |
| | | | | AOE372 | |
| PSB5_MOUSE | 5693 | PSB5 | PSMB5 | MB1; X; LMPX | XM_005267871; NM_002797; NM_001144932; |
| | | | | | NM_001130725 |
| PDIA1_MOUSE | 5034 | A0A024R | P4HB | PHDB; P4Hbeta; PO4DB; PROHB; | ; NM_000918 |
| | | 8S5; | | ERBA2L; GIT; DSI; PDI; PDIA1; | |
| | | PDIA1 | | PO4HB | |
| NUCL_MOUSE | 4691 | NUCL | NCL | C23 | NM_005381 |
| THIO_MOUSE | 7295 | H9ZYJ2; | TXN | TRDX; TRX1; TRX | NM_003329; NM_001244938 |
| | | THIO | | | |
| DDX3L_MOUSE | 8653 | DDX3Y | DDX3Y | DBY | ; XM_006724878; NM_001122665; |
| | | | | | NM_001302552; NM_004600; XM_011531471 |
| TPIS_MOUSE | 7167 | V9HWK1; | TP11 | TPID; HEL-D-49; TPI; TIM | NM_001159287; NM_000365; NM_001258026; |
| | | Q53HE2; | | | |
| | | TPIS | | | |
| RL18_MOUSE | — | | | | |
| RL6_MOUSE | 6128 | A0A024R | RPL6 | TXREB1l TAXREB107; SHUJUN- | XM_006719548; XM_006719546; NM_000970; |
| | | BK3; | | 2; L6 | NM_001024662; XM_006719547; |
| | | Q8TBK5; | | | XM_006719549; XM_011538647; |
| | | RL6 | | | XM_011538646 |
| SAHH_MOUSE | 191 | SAHH | AHCY | SAHH; adoHcyase | XM_005260317; ; XM_005260316; |
| | | | | | XM_011528660; XM_011528657; |
| | | | | | XM_011528658; XM_011528659; NM_000687; |
| | | | | | NM_001161766; XM_011528656 |
| KPYM_MOUSE | 5315 | A0A024R | PKM | CTHBP; HEL-S-30; PK3; OIP3; | NM_001206796; NM_001206797; |
| | | 5Z9; | | TCB; THBP1; PKM2 | XM_011521673; XM_005254445; |
| | | V9HWB8; | | | XM_011521670; XM_011521672; NM_002654; |
| | | B4DNK4; | | | NM_001206798; XM_005254443; |
| | | KPYM | | | XM_006720570; XM_011521671; |
| | | | | | NM_001206799; NM_182470; NM_182471 |
| CALM_MOUSE | — | | | | |
| RUXF_MOUSE | 6636 | RUXF | SNRPF | Sm-F; snRNP-F; SMF | NM_003095 |
| SMD2_MOUSE | 6633 | SMD2 | SNRPD2 | SMD2; SNRPD1; Sm-D2 | NM_004597; NM_177542; XM_005259180 |
| TOP1_MOUSE | 7150 | TOP1 | TOP1 | TOP1 | XM_011529033; ; XM_011529032; NM_003286 |
| HNRPD_MOUSE | — | | | | |
| VDAC1_MOUSE | — | | | | |
| ARGI1_MOUSE | 383 | ARGI1 | ARG1 | — | NM_000045; NM_001244438; ; XM_011535801 |
| RALY_MOUSE | — | | | | |
| CPNE_MOUSE | 8895 | CPNE3 | CPNE3 | CPN3; PRO1071 | XM_005251093; NM_003909 |
| DDX18_MOUSE | 8886 | DDX18 | DDX18 | MrDb | NM_006773 |
| DDX27_MOUSE | 55661 | DDX27 | DDX27 | HSPC259; Drs1p; dJ686N3.1; | NM_017895; XM_011528888 |
| | | | | PP3241; DRS1; RHLP | |
| ROAA_MOUSE | 3182 | ROAA | HNRNPAB | HNRPAB; ABBP1 | NM_004499; NM_031266 |
| NOG2_MOUSE | 29889 | NOG2 | GNL2 | Hug2; Ngp-1; Nog2; NGP1; | XM_011541300; NM_013285 |
| | | | | HUMAUANT1G | |
| RL17_MOUSE | — | | | | |
| GGCT_MOUSE | 79017 | GGCT | GGCT | C7orf24; GGC; CRF21; GCTG | NM_001199817; NM_024051; NM_001199815; |
| | | | | | NM_001199816; NR_037669 |
| NVL_MOUSE | 4931 | NVL | NVL | — | XM_011544199; NM_001243146; |
| | | | | | XM_011544202; XM_011544198; |
| | | | | | XM_011544201; NM_206840; XM_011544196; |
| | | | | | XM_011544197; XM_011544200; ; |
| | | | | | NM_001243147; NM_002533 |
| PSB3_MOUSE | 5691 | PSB3 | PSMB3 | HC10-II | NR_104195; NM_002795; NR_104194 |
| LOXE3_MOUSE | 59344 | LOXE3 | ALOXE3 | ARCI3; eLOX3; E-LOX3; eLOX-3 | NM_001165960; ; NM_021628 |
| D3YWT1_MOUSE | 3189 | HNRH3 | HNRNPH3 | HNRPH3; 2H9 | XM_005269753; XM_005269748; |
| | | | | | XM_005269752; XM_006717816; |
| | | | | | XM_005269751; XM_011539743; |
| | | | | | XM_006717817; XM_005269749; |
| | | | | | XM_005269754; NM_012207; NM_021644; |
| | | | | | XM_011539742 |
| HNRPF_MOUSE | — | | | | |
| FILA2_MOUSE | — | | | | |
| DSG1A_MOUSE | 1828 | DSG1 | DSG1 | CDHF4; DSG; PPKS1; EPKHIA; | ; NM_001942 |
| | | | | SPPK1; DG1; EPKHE | |
| SRSF7_MOUSE | — | | | | |
| MYH9_MOUSE | 4627 | MYH9 | MYH9 | BDPLT6; DFNA17; FTNS; | XM_011530197; ; NM_002473 |
| | | | | NMMHCA; EPSTS; NMHC-II-A; | |
| | | | | MHA; NMMHC-11A | |
| SON_MOUSE | — | | | | |
| RBM25_MOUSE | 58517 | RBM25 | RBM25 | Snu71; NET52; RED120; RNPC7; | XR_943501; NM_021239; XM_011537044; |
| | | | | S164; fSAP94 | XM_011537045 |
| PCNA_MOUSE | 5111 | PCNA | PCNA | ATDL2 | NM_002592; NM_182649 |
| TRA2B_MOUSE | 6434 | TRA2B | TRA2B | PPP1R156; SFRS10; TRAN2B; | XM_011513072; XM_006713724; NM_004593; |
| | | | | SRFS10; TRA2-BETA; Htra2-beta | ; NM_001243879; XM_005247703 |
| DDX5_MOUSE | — | | | | |
| EFTU_MOUSE | 7284 | EFTU | TUFM | COXPD4; EFTU; P43; EF-TuMT | ; NM_003321; XM_011545928 |
| UHRF1_MOUSE | — | | | | |
| SFPQ_MOUSE | 6421 | SFPQ | SFPQ | PPP1R140; PSF; POMP100 | XM_005271113; XM_005271115; |
| | | | | | XM_011541950; XM_005271112; NM_005066 |
| DDX24_MOUSE | 57062 | DDX24 | DDX24 | — | NM_020414 |
| HNRDL_MOUSE | 9987 | HNRDL | HNRNPDL | LGMD1G; HNRNP; HNRPDL; | NM_031372; ; NM_005463; NM_001207000; |
| | | | | JKTBP2; JKTBP; 1aAUF1 | NR_003249 |
| HNRPC_MOUSE | — | | | | |
| U2AF2_MOUSE | 11338 | U2AF2 | U2AF2 | U2AF65 | XM_006722994; NM_001012478; ; |
| | | | | | NM_007279; XM_011526410 |
| H13_MOUSE | 3007 | H13 | HISTIH1D | H1.3; H1s-2; H1F3; H1D | NM_005320 |
| HNRPK_MOUSE | — | | | | |
| RS27A_MOUSE | 6233 | RS27A | RPS27A | UBC; UBCEP80; S27A; UBCEP1; | NM_002954; NM_001177413; ; NM_001135592 |
| | | | | CEP80; CEL112; UBA80 | |
| TBB5_MOUSE | 203068 | TBB5 | TUBB | M40; TUBB1; CDCBM6; OK/SW- | ; NM_001293213; NM_001293214; |
| | | | | c1.56; TUBB5 | NM_001293212; NR_120608; NM_001293215; |
| | | | | | NM_001293216; NM_178014 |
| FUBP1_MOUSE | — | | | | |
| G3X9B1_MOUSE | 55127 | HETA1 | HEATR1 | UTP10; BAP28 | NM_018072; NM_011544219 |
| HNRPU_MOUSE | 3192 | HNRPU | HNRNPU | HNRPU; SAF-A; U21.1; hmRNP U | NM_004501; NM_031844 |
| HSP7C_MOUSE | 3312 | HSP7C | HSPA8 | LAP1; LAP-1; HSC70; HSPA10; | ; XM_011542798; NM_153201; NM_006597 |
| | | | | HEL-33; HDC54; HSC71; HSP71; | |
| | | | | HSP73; HEL-S-72p; NIP71 | |
| SRRM2_MOUSE | — | | | | |
| HS71B_MOUSE | 3304; | — | — | — | — |
| | 3303 | | | | |
| ROA1_MOUSE | 3178; | — | — | — | — |
| | 144983 | | | | |
| MBB1A_MOUSE | 10514 | MBB1A | MYBBP1A | PAP2; P160 | NM_001105538; NM_014520; XM_011523616 |
| NPM3_MOUSE | 10360 | NPM3 | NPM3 | TMEM123; PORMIN | NM_006993 |
| MDHM_MOUSE | 4191 | A0A024R | MDH2 | MGC:3559; M-MDH; MOR1; MDH | NR_104165; NM_001282403; NM_001282404; |
| | | 4K3; | | | NM_005918 |
| | | MDHM; | | | |
| | | B3KTM1; | | | |
| | | G3XAL0 | | | |
| H14_MOUSE | 3008 | H14 | HIST1H1E | H1F4; dJ221C16.5; H1.4; H1E; | NM_005321 |
| | | | | H1s04 | |
| ATPB_MOUSE | 506 | V9HW31; | ATP5B | ATPMB; HEL-S-271; ATPSB | NM_001686 |
| | | ATPB | | | |
| H2AY_MOUSE | 9555 | H2AY | H2AFY | H2AFJ; H2A.y; H2AF12M; H2A/y; | NM_138609; XM_011543731; XR_948308; |
| | | | | mH2A1; macroH2A1.2; | NM_004893; XM_005272132; XM_005272134; |
| | | | | MACROH2A1.1 | XM_011543735; XR_948310; XM_011543728; |
| | | | | | XR_948306; XR_948307; XM_005272135; |
| | | | | | XM_011543730; XM_011543733; XR_948309; |
| | | | | | NM_138610; XM_011543729; XM_011543732; |
| | | | | | NM_001040158; XM_011543734; XR_948311 |
| DESP_MOUSE | 1832 | DESP | DSP | DCWHKTA; DP; DP1; DPI1 | ; XM_011514323; NM_001008844; NM_004415 |
| ANXA2_MOUSE | 302 | A0A024R | ANXA2 | LPC2; ANX2L4; LIP2; LPC2D; | NM_004039; XM_011521475; XM_011521476; |
| | | 5Z7; | | PAP-IV; ANX2; P36; HEL-S-270; | NM_001002858; NM_001002857; |
| | | ANXA2 | | CAL1H | NM_001136015; XM_011521477 |
| VIME_MOUSE | 7431 | VIME | VIM | CTRCT30; HEL113 | XM_011519649; XM_006717500; NM_003380 |
| ROA2_MOUSE | 3181 | ROA2 | HNRNPA2B1 | HNRPA2; RNPA2; SNRPB1; | XR_242076; XR_242077; NM_002137; |
| | | | | HNRNPA2; HNRNPB1; IBMPFD2; | XR_428077; XR_428078; XM_006715714; |
| | | | | HNRPA2B1; HNRPB1 | NM_031243; XM_005249729 |
| ATPA_MOUSE | 498 | ATPA; | ATP5A1 | hATP1; ATP5A; HEL-S-123m; | NM_001257334; ; NM_001001937; |
| | | V9HW26 | | MOM2; COXPD22; OMR; ATPM; | XM_011526018; NM_001001935; |
| | | | | MC5DN4; ORM; ATP5AL2 | XM_001257335; NM_004046 |
| NPM_MOUSE | 4869 | NPM | NPM1 | B23; NPM | XM_005265920; ; NM_001037738; |
| | | | | | NM_002520; NM_199185; XM_011534564 |
| LMNA_MOUSE | 4000 | LMNA | LMNA | LMN1; LMNL1; EMD2; FPL; IDC; | XR_921781; NM_005572; NM_170707; |
| | | | | CDCD1; LMNC; CDDC; CMD1A; | NM_170708; ; NM_001282624; |
| | | | | FPLD; PRO1; LFP; LGMD1B; | NM_001282626; NM_001282625; |
| | | | | CMT2B1; FPLD2; HGPS; LDP1 | XM_011509534; NM_001257374; |
| | | | | | XM_0115909533 |
| MUP17_MOUSE | — | | | | |
| THOC4_MOUSE | 10189 | THOC4 | ALYREF | ALY/REF; THOC4; BEF; ALY; | NM_005782; XR_933919 |
| | | | | REF | |
| U5S1_MOUSE | 9343 | U5S1 | EFTUD2 | MFDGA; Snrp116; Snu114; | NM_001258353; NM_001142605; XR_934602; |
| | | | | SNRNP116; U5-116KD; MFDM | NM_001258354; NM_004247; |
| HDAC1_MOUSE | 3065 | Q6IT96; | HDAC1 | RPD3; GON-10; HD1; RPD3L1 | XM_011541309; NM_004964 |
| | | HDAC1 | | | |
| NEP1_MOUSE | 10436 | NEP1 | EMG1 | C2F; Grcc2f; NEP1 | ; XM_011520907; NM_006331 |
| | 7528 | YY1 | YY1 | NF-EI; INO80S; UCRBP; DELTA; | NM_003403 |
| | | | | YIN-YANG-1 | |
| | 23429 | RYBP | RYBP | AAP1; DEDAF; YEAF1 | XM_011548867; XM_011548866; NM_012234 |
| | 2146 | EZH2 | EZH2 | KMT6; KMT6A; WVS; EZH2b; | XM_011515896; XM_011515897; |
| | | | | ENX-1; EZHI; ENX1; WVS2 | XM_011515901; NM_001203249; |
| | | | | | XM_005249964; XM_011515884; |
| | | | | | XM_011515890; XM_011515894; |
| | | | | | XM_011515899; NM_004456; XM_011515886; |
| | | | | | XM_011515892; XM_011515900; NM_152998; |
| | | | | | XM_011515888; XM_011515889; |
| | | | | | XM_011515902; ; NM_001203247; |
| | | | | | NM_001203248; XM_005249962; |
| | | | | | XM_011515895; XM_011515883; |
| | | | | | XM_005249963; XM_011515885; |
| | | | | | XM_011515887; XM_011515898; |
| | | | | | XM_011515891; XM_011515893 |
| | 8726 | EED | EED | HEED; WAIT1 | XM_011545330; XM_005274373; |
| | | | | | XM_011545331; XM_011535329; |
| | | | | | XR_247215; ; |
| | | | | | NM_003797l NM_152991 |
| | 3720 | JARID2 | JARID2 | JMJ | XM_011514578; NM_004973; XM_011514580; |
| | | | | | NM_001267040; NM_011514581; |
| | | | | | XM_011514579; XM_011514584; |
| | | | | | XM_011514583; XM_005249089; |
| | | | | | XM_011514582 |
| | 23512 | SUZ12 | SUZ12 | CHET9; JJAZ1 | XM_005257954; XM_011524578; NM_015355; |
| | | | | | XM_006721794; XM_011524576; |
| | | | | | XM_011524577; |
| | 84733 | CBX2 | CBX2 | CDCA6; SRXY5; M33 | XM_011525382; XM_011525383; NM_032647; |
| | | | | | NM_005189; |
| | 8535 | CBX4 | CBX4 | NBP16; PC2 | XM_011525399; NM_003655 |
| | 23468 | CBX5 | CBX5 | HEL25; HP1; HP1A | NM_001127321; NM_001127322; NM_012117 |
| | 23466 | CBX6 | CBX6 | — | NM_001127321; NM_001127322; NM_012117 |
| | 23492 | CBX7 | CBX7 | — | XM_006724178; XM_006724174; |
| | | | | | XM_006724176; NM_175709; XM_006724175; |
| | | | | | XM_011530025; XM_005261413; |
| | | | | | XM_006724177 |
| | 57332 | CBX8 | CBX8 | RC1; PC3 | NM_020649 |
| | 6015 | RING1 | RING1 | RING1A; RNF1; | XM_008581826; XM_002914334; |
| | | | | MDA_GLEAN10006855; | XM_011282270; XM_004711741; |
| | | | | AT5G10380; ATRING1; | XM_010994566; NM_001114959; |
| | | | | F12B17.270; F12B17_270; | XM_008263251; XM_008160095; |
| | | | | PAL_GLEAN10007107 | XM_006144236; XM_003789083; |
| | | | | | XM_003768961; XM_003421045; |
| | | | | | XM_003340366; XM_006882095; |
| | | | | | XM_004673367; XM_004043802; |
| | | | | | NM_001081482; XM_009450849; |
| | | | | | XM_007939317; XM_004817207; |
| | | | | | XM_004817208; XM_004770597; |
| | | | | | XM_004770598; XM_006105473; |
| | | | | | XM_007972961; XM_010848711; |
| | | | | | XM_005891414; XM_007460823; |
| | | | | | XM_003808593; NM_001048128; |
| | | | | | XM_006738062; XM_004479796; |
| | | | | | XM_001493382; XM_005603802; |
| | | | | | NM_001190235; XM_002746424; |
| | | | | | XM_007093228; XM_003897435; |
| | | | | | XM_008693443; XM_002809147; NM_002931; |
| | | | | | XM_010357860; XM_004617672; |
| | | | | | XM_005867940; XM_005867939; |
| | | | | | XM_005867938; XM_008060264; |
| | | | | | XM_010949097; XM_006769154; |
| | | | | | XM_006769153; XM_006769152; |
| | | | | | XM_004389800; XM_004389799; |
| | | | | | XM_006202134; XM_006860350; NM_121076; |
| | | | | | XM_009398851; XM_008501211; |
| | | | | | XM_008507909; XM_008507908; |
| | | | | | XM_007186905; XM_006180513; |
| | | | | | XM_005553330; XM_003923131; |
| | | | | | XM_011373441; XM_006050304; |
| | | | | | XM_004267748; XM_003271891; |
| | | | | | XM_007527425; XM_006907387; |
| | | | | | NM_001105051; XM_004018744; |
| | | | | | XM_005979771; XM_004407693; |
| | | | | | XM_004326287; XM_004424282; |
| | | | | | XM_005696449; XM_004590264 |
| | 6045 | RNF2 | RNF2 | UY3_04118; RING1B; | XM_007056701; NM_001133961; |
| | | | | Anapl_15990; | XM_009240017; XM_010010379; |
| | | | | PAL_GLEAN10017658; RING2; | XM_005030750; XM_005030748; |
| | | | | BAP-1; DING; HIPI3; BAP1; | XM_005030749; XM_005622432; XM_537164; |
| | | | | TREES_T100002675; AS27_08110 | XM_003785791; XM_005232734; |
| | | | | | XM_004313674; XM_011588767; |
| | | | | | XM_011588768; XM_011588769; |
| | | | | | XM_008066104; XM_007166956; |
| | | | | | XM_007166955; XM_008249917; |
| | | | | | XM_002722443; XM_010184804; |
| | | | | | XM_010131884; XM_009940829; |
| | | | | | XM_009582679; XM_009967773; |
| | | | | | XM_008588367; XM_006872706; |
| | | | | | XM_004808116; XM_004808117; |
| | | | | | XM_004767958; XM_004767956; |
| | | | | | XM_004767957; XM_009636901; |
| | | | | | XM_003264459; XM_004088945; |
| | | | | | XM_004613710; XM_005856664; |
| | | | | | XM_009902653; XM_006907738; |
| | | | | | XM_005667822; XM_005667824; |
| | | | | | XM_005667826; XM_005667821; |
| | | | | | XM_003130379; XM_005667823; |
| | | | | | XM_005667825; XM_006135799; |
| | | | | | XM_010853086; XM_009979151; |
| | | | | | XM_009191712; XM_002893395; XM_514507; |
| | | | | | XM_003308638; XM_009439610; |
| | | | | | XM_009439605; XM_007937694; |
| | | | | | XM_005531309; XM_006267565; |
| | | | | | XM_008945965; XM_009919202; |
| | | | | | XM_011227014; XM_002920849; |
| | | | | | XM_006089858; XM_005049912; |
| | | | | | XM_005049913; XM_001516642; |
| | | | | | XM_007668980; XM_006037004; |
| | | | | | XM_005146513; XM_005893101; |
| | | | | | XM_005893100; XM_004372913; |
| | | | | | XM_010406351; XM_010580692; |
| | | | | | XM_011509852; NM_007212; XM_005245413; |
| | | | | | XM_011509851; XM_010155175; |
| | | | | | XM_009074584; XM_007434510; |
| | | | | | XM_005963396; XM_005963397; |
| | | | | | XM_010213372; XM_004468429; |
| | | | | | XM_004468430; XM_004468431; |
| | | | | | XM_005487039; XM_009463539; |
| | | | | | XM_011364545; XM_004688568; |
| | | | | | XM_004688569; XM_009997849; |
| | | | | | XM_005506534; XM_004943287; XM_422295; |
| | | | | | XM_004943285; XM_004943286; |
| | | | | | XM_003208502; XM_010715546; |
| | | | | | XM_010715547; XM_009088429; |
| | | | | | XM_009481357; XM_001490007; |
| | | | | | XM_008534655; XM_006772947; |
| | | | | | XM_006772948; XM_006185546; |
| | | | | | XM_003925286; XM_004424928; |
| | | | | | XM_005690974; XM_008968563; |
| | | | | | XM_003815602; XM_008145953; |
| | | | | | XM_006143866; XM_005443319; |
| | | | | | XM_009865895; XM_010204370; |
| | | | | | XM_006060513; XM_006060510; |
| | | | | | XM_006060512; XM_006060511; |
| | | | | | XM_002190830; XM_009275831; |
| | | | | | XM_011291005; XM_004001340; |
| | | | | | XM_007989177; XM_007989176; |
| | | | | | XM_007989179; XM_010368958; |
| | | | | | XM_010368957; XM_004275227; |
| | | | | | XM_007523806; XM_005540227; |
| | | | | | XM_005540230; XM_005540228; |
| | | | | | XM_005540229; XM_005540231; |
| | | | | | NM_001101203; XM_004028047; |
| | | | | | XM_004028046; XM_004578826; |
| | | | | | XM_010084954; XM_009556366; |
| | | | | | XM_006198790; XM_009506697; |
| | | | | | XM_010313112; XM_009324640; |
| | | | | | XM_003767510; XM_010591103; |
| | | | | | XM_002760258; XM_008984832; |
| | | | | | XM_008984833; XM_007451138; |
| | | | | | XM_007451139; XM_010992086; |
| | | | | | XM_010992085; XM_007096230; |
| | | | | | XM_004013869; XM_009884364; |
| | | | | | XM_009672662; XM_008635395; |
| | | | | | XM_008635394; XM_005290711; |
| | | | | | XM_010116863; XM_009947654; |
| | | | | | XM_010165133; XM_004405742; |
| | | | | | XM_006732908; XM_006732907; |
| | | | | | XM_009808327; XM_010296946; |
| | | | | | XM_008494822; XM_005997271; |
| | | | | | XM_001366864; XM_004706673; |
| | | | | | XM_004706674; XM_010973308; |
| | | | | | XM_010973311; XM_008930821; |
| | | | | | XM_005423737; XM_008697076; |
| | | | | | XM_008697084; XM_008697091; |
| | | | | | XM_009695548 |

TABLE 6
iDRiP proteomics results - Multiplexed quantitation of proteins pulled down by iDRiP and identified by mass spectrometry.

| UniProt Entry | Human | Human | Human Gene | | |
|---|---|---|---|---|---|
| Name | Gene ID | Protein | Symbol | Gene Synonyms | Accession numbers |
| MINT_MOUSE | 23013 | MINT | SPEN | HIAA0929; | NM_015001 |
| | | | | MINT; SHARP; | |
| | | | | RBM15C | |
| FIBB_MOUSE | 2244 | FIBB | FGB | HEL-S-78p | NM_005141; ; NM_001184741 |
| CO1A2_MOUSE | 1278 | CO1A2 | COL1A2 | OI4 | NM_000089 |
| IKIP_MOUSE | 121457 | IKIP | IKBIP | IKIP | NM_153687; NM_201613; NM_201612 |
| RGAP1_MOUSE | 29127 | RGAP1 | RACGAP1 | CYK4; | NM_001126104; XM_005268814; XM_011538235; |
| | | | | MgcRacGAP; | XM_011538242; XM_005268813; XM_011538240; |
| | | | | ID-GAP; | NM_013277; XM_006719359; XM_011538241; |
| | | | | HsCYK-4 | XM_011538243; NM_001126103; XM_005268815; |
| | | | | | XM_011538236; XM_005268812; XM_011538237; |
| | | | | | XM_011538238; XM_011538239 |
| RFC1_MOUSE | 5981 | RFC1 | RFC1 | RFC140; PO- | NM_001204747; XM_011513730; NM_002913; |
| | | | | GA; RECC1; A1; | XM_011513731 |
| | | | | MHCBFB; RFC | |
| COCA1_MOUSE | 1303 | COCA1 | COL12A1 | BA209D8.1; | XM_011535436; NM_004370; XM_011535435; |
| | | | | COL12A1L; | NM_080645; XM_011535434 |
| | | | | DJ234P15.1 | |
| NEP_MOUSE | 4311 | NEP | MME | CALLA; NEP; | XM_006713647; NM_007289; XM_011512856; |
| | | | | CD10; SFE | NM_007287; XM_006713646; NM_007288; |
| | | | | | XM_011512855; XM_011512858; XM_011512857; |
| | | | | | NM_000902 |
| NUP88_MOUSE | 4927 | NUP88 | NUP88 | — | XM_011523893; XM_005256659; NM_002532 |
| UHRF1_MOUSE | 29128 | UHRF1 | UHRF1 | RNF106; | NM_001290052; XM_011527942; ; NM_001290051; |
| | | | | ICBP90; Np95; | NM_001048201; NM_001290050; NM_013282 |
| | | | | hNP95; | |
| | | | | hUHRF1; | |
| | | | | huNp95 | |
| WAPL_MOUSE | 23063 | WAPL | WAPAL | KIAA0261; | XM_011539547; XM_011539548; XM_006717729; |
| | | | | WAPL; FOE | NM_015045 |
| ZFR_MOUSE | 51663 | ZFR | ZFR | SPG71; ZFR1 | XR_427659; NM_016107 |
| BAK_MOUSE | 578 | BAK | BAK1 | BAK; BAK- | XM_011514779; XM_011514780; NM_001188 |
| | | | | LIKE; CDN1; | |
| | | | | BCL2L7 | |
| NU133_MOUSE | 55746 | NU133 | NUP133 | hNUP133 | NM_018230 |
| Q8BVY0_MOUSE | — | — | — | — | — |
| CO1A1_MOUSE | 1277 | CO1A1 | COL1A1 | OI4 | NM_000088; ; XM_005257059; XM_005257058; |
| | | | | | XM_011524341 |
| NHP2_MOUSE | 55651 | NHP2 | NHP2 | DKCB2; | NM_001034833; NM_017838 |
| | | | | NHP2P; NOLA2 | |
| HELLS_MOUSE | 3070 | HELLS | HELLS | PASG; LSH; | NM_001289067; NM_001289071; NM_001289073; |
| | | | | Nbla10143; | NM_001289074; NM_001289075; NM_001289068; |
| | | | | SMARCA6 | NM_001289070; NM_001289069; NM_001289072; |
| | | | | | NM_018063 |
| HNRPU_MOUSE | 3192 | HNRPU | HNRNPU | HNRPU; SAF-A; | NM_004501; NM_031844 |
| | | | | U21.1; hnRNP U | |
| LRWD1_MOUSE | 222229 | LRWD1 | LRWD1 | CENP-33; | XM_005250204; NM_152892 |
| | | | | ORCA | |
| RCC1_MOUSE | 1104 | RCC1 | RCC1 | CHC1; SNHG3- | NM_001048199; NM_001269; NM_001048195; |
| | | | | RCC1; RCC1-I | NR_030725; NR_030726; NM_001048194 |
| MBB1A_MOUSE | 10514 | MBB1A | MYBBP1A | PAP2; P160 | NM_001105538; NM_014520; XM_011523616 |
| MYEF2_MOUSE | 50804 | MYEF2 | MYEF2 | myEF-2; | XM_005254424; XM_006720553; XM_005254422; |
| | | | | MSTP156; | XM_005254425; NM_001301210; NM_016132; |
| | | | | HsT18564; | XM_005254427; XM_011521657; NR_125408 |
| | | | | MEF-2; MST156 | |
| LRP1_MOUSE | 4035 | LRP1 | LRP1 | CD91; | NM_002332; |
| | | | | IGFBP3R; | |
| | | | | A2MR; LRP1A; | |
| | | | | APOER; APR; | |
| | | | | LRP; TGFBR5 | |
| NXF1_MOUSE | 10482 | NXF1 | NXF1 | MEX67; TAP | NM_001081491; NM_006362 |
| RL7L_MOUSE | 285855 | RL7L | RPL7L1 | dJ475N16.4 | XM_005249026; NM_198486 |
| HXA5_MOUSE | 3202 | HXA5 | HOXA5 | HOX1.3; HOX1; | NM_019102 |
| | | | | HOX1C | |
| SMHD1_MOUSE | 23347 | SMHD1 | SMCHD1 | — | XM_011525645; NM_015295; XM_011525646; ; |
| | | | | | XM_011525643; XM_011525644; XR_935054; |
| | | | | | XM_011525642; XM_011525647; XR_935055; |
| | | | | | XR_430039 |
| NFIC_MOUSE | 4782 | NFIC | NFIC | NFI; NF-I; CTF; | NM_001245005; NM_005597; XM_005259563; |
| | | | | CTF5 | XM_006722759; NM_205843; NM_001245002; |
| | | | | | NM_001245004; XM_005259564 |
| P53_MOUSE | 7157 | H2EHT1 | TP53 | TRP53; BCC7; | NM_001126112; NM_001276697; NM_001126115; ; |
| | | | | P53; LFS1 | NM_001126114; NM_001276698; NM_001276761; |
| | | | | | NM_001126118; NM_001126113; NM_001126117; |
| | | | | | NM_001276695; NM_001276699; NM_001276760; |
| | | | | | NM_000546; NM_001126116; NM_001276696 |
| CELF2_MOUSE | 10659 | CELF2 | CELF2 | CUGBP2; | NM_001083591; NM_006561; XM_006717373; |
| | | | | NAPOR; | XM_011519294; XM_011519295; XM_011519297; |
| | | | | BRUNOL3; | XM_011519298; XM_005252354; XM_006717371; |
| | | | | ETR-3; ETR3 | NM_001025076; XM_006717374; XM_006717375; |
| | | | | | XM_011519299; NM_001025077; XM_005252357; |
| | | | | | XM_005252358; XM_006717369; XM_011519296; |
| | | | | | XM_006717370 |
| XPO5_MOUSE | 57510 | XPO5 | XPO5 | exp5 | NM_020750 |
| GAPR1_MOUSE | 152007 | GAPR1 | GLIPR2 | C9orf19; GAPR- | NM_001287012; NM_001287014; NR_104638; |
| | | | | 1; GAPR1 | NM_001287011; NR_104640; NR_104641; NR_104637; |
| | | | | | NR_104639; XM_011517714; NM_001287013; |
| | | | | | NM_022343; NM_001287010 |
| MSH2_MOUSE | 4436 | MSH2 | MSH2 | HNPCC; | NM_000251; XM_005264332; NM_001258281; |
| | | | | HNPCC1; FCC1; | XR_939685; ; XM_011532867 |
| | | | | COCA1; LCFS2 | |
| PNO1_MOUSE | 56902 | PNO1 | PNO1 | KHRBP1; | NM_020143 |
| | | | | RRP20 | |
| TSP1_MOUSE | 7057 | TSP1 | THBS1 | TSP; TSP1; | XM_011521970; XR_931897; XM_011521971; |
| | | | | THBS; THBS-1; | NM_003246 |
| | | | | TSP-1 | |
| LBR_MOUSE | 3930 | LBR | LBR | PHA; | XM_011544187; NM_002296; XM_011544185; |
| | | | | DHCR14B; | XM_011544186; NM_194442; XM_005273125 |
| | | | | TDRD18; | |
| | | | | LMN2R | |
| PGS1_MOUSE | 633 | PGS1 | BGN | PG-S1; DSPG1; | NM_001711 |
| | | | | SLRR1A; PGI | |
| PCOC1_MOUSE | 5118 | PCOC1 | PCOLCE | PCPE-1; PCPE1; | NM_002593 |
| | | | | PCPE | |
| RING1_MOUSE | 6015 | RING1 | RING1 | RING1A; RNF1 | NM_002931 |
| ROA0_MOUSE | 10949 | ROA0 | HNRNPA0 | HNRPA0 | NM_006805 |
| RB15B_MOUSE | 29890 | RB15B | RBM15B | HUMAGCGB; | NM_013286 |
| | | | | OTT3 | |
| FBLN4_MOUSE | 30008 | FBLN4 | EFEMP2 | UPH1; FBLN4; | NM_016938; ; NR_037718 |
| | | | | ARCL1B; MBP1 | |
| HNRL2_MOUSE | 221092 | HNRL2 | HNRNPUL2 | HNRPUL2; | NM_001079559 |
| | | | | SAF-A2 | |
| NIP7_MOUSE | 51388 | NIP7 | NIP7 | HSPC031; | NM_001199434; NM_016101 |
| | | | | CGI-37; KD93 | |
| J3QQ16_MOUSE | — | | — | — | — |
| RRP1B_MOUSE | 23076 | RRP1B | RRP1B | PPP1R136; | NM_015056 |
| | | | | KIAA0179; | |
| | | | | NNP1L; Nnp1; | |
| | | | | RRP1 | |
| DCLK1_MOUSE | 9201 | DCLK1 | DCLK1 | CL1; CLICK1; | XM_006719893; XM_005266592; NM_001195430; |
| | | | | DCDC3A; | NM_001195416; NM_001195415; NM_004734 |
| | | | | DCAMKL1; | |
| | | | | DCLK | |
| ACADS_MOUSE | 35 | ACADS | ACADS | ACAD3; SCAD | NM_000017; NM_001302554 |
| MD1L1_MOUSE | 8379 | MD1L1 | MAD1L1 | TXBP181; | XM_011515570; XM_005249877; XM_011515567; |
| | | | | TP53I9; MAD1; | XM_011515571; NM_001013837; NM_001304525; |
| | | | | PIG9 | XM_011515568; ; NM_001013836; NM_001304523; |
| | | | | | NM_003550; XM_011515569; NM_001304524 |
| XRN2_MOUSE | 22803 | XRN2 | XRN2 | — | XM_011529184; NM_012255 |
| CO6A2_MOUSE | 1292 | CO6A2 | COL6A2 | PP3610 | XR_937439; NM_058175; NM_058174; XR_937438; |
| | | | | | NM_001849; ; XM_011529452; XM_011529451 |
| TADBP_MOUSE | 23435 | TADBP | TARDBP | ALS10; TDP-43 | NM_007375; XR_946596; ; XR_946597 |
| MYOF_MOUSE | 26509 | MYOF | MYOF | FER1L3 | XM_006717760; NM_133337; XM_005269693; |
| | | | | | XM_011539632; XM_011539633; NM_013451; |
| | | | | | XM_005269694 |
| NID2_MOUSE | 22795 | NID2 | NID2 | NID-2 | XM_005267405; XM_005267406; XM_005267407; |
| | | | | | NM_007361 |
| MGN2_MOUSE | 55110 | MGN2 | MAGOHB | mago; MGN2; | NM_018048; XM_005253402; NM_001300739; |
| | | | | magoh | XM_011520718 |
| SNTB2_MOUSE | 6645 | SNTB2 | SNTB2 | SNT2B2; SNT3; | NM_006750; NM_130845 |
| | | | | SNTL; | |
| | | | | D16S2531E; | |
| | | | | EST25263 | |
| H3BJG4_MOUSE | — | | — | — | — |
| KDM2A_MOUSE | 22992 | KDM2A | KDM2A | CXXC8; FBL11; | NR_027473; NM_012308; XM_011544860; |
| | | | | FBL7; JHDM1A; | XM_006718479; XM_006718480; XM_011544861; |
| | | | | FBXL11; | XM_011544862; NM_001256405 |
| | | | | LILINA | |
| DJC10_MOUSE | 54431 | DJC10 | DNAJC10 | ERdj5; MTHr; | NM_001271581; NM_018981; NR_073367; NR_073366; |
| | | | | JPDI; PDIA19 | NR_073365 |
| MAOM_MOUSE | 4200 | MAOM | ME2 | ODS1 | NM_002396; XR_935223; ; NM_001168335 |
| SUN2_MOUSE | 25777 | SUN2 | SUN2 | UNC84B | NM_015374; XM_011530105; XM_011530104; |
| | | | | | NM_001199580; NM_001199579 |
| Q921K2_MOUSE | — | | — | — | — |
| GPX1_MOUSE | 2876 | GPX1 | GPX1 | GSHPX1; GPXD | NM_000581; NM_201397; |
| DYR_MOUSE | 1719 | DYR | DHFR | DHFRP1; DYR | NM_000791; NM_001290357; ; NM_001290354; |
| | | | | | NR_110936 |
| G5E924_MOUSE | — | | — | — | — |
| LEG8_MOUSE | 3964 | LEG8 | LGALS8 | Po66-CBP; | NM_201544; XM_011544188; NM_201543; |
| | | | | PCTA-1; Gal-8; | NM_006499; NM_201545 |
| | | | | PCTA1 | |
| LYOX_MOUSE | 4015 | LYOX | LOX | — | NM_001178102; ; NM_002317 |
| EIF2A_MOUSE | 83939 | EIF2A | EIF2A | EIF-2A; | XM_011513224; XM_011513223; NM_032025 |
| | | | | MST089; | |
| | | | | CDA02; | |
| | | | | MSTP004; | |
| | | | | MSTP089 | |
| PTBP2_MOUSE | 58155 | PTBP2 | PTBP2 | nPTB; PTBLP; | XR_946723; XR_946722; NM_001300987; NR_125357; |
| | | | | brPTB | XM_011541876; XM_011541875; XR_946720; |
| | | | | | NM_001300986; NM_001300988; NM_021190; |
| | | | | | NM_001300990; NR_125356; XM_011541874; |
| | | | | | XR_946721; NM_001300985; NM_001300989 |
| STT3B_MOUSE | 201595 | STT3B | STT3B | SIMP; CDG1X; | XM_011533465; NM_178862 |
| | | | | STT3-B | |
| HNRPM_MOUSE | 4670 | HNRPM | HNRNPM | HTGR1; | NM_005968; XM_005272478; XM_005272480; |
| | | | | NAGR1; hnRNPM; | XM_005272483; XM_005272479; XM_005272481; |
| | | | | HNRPM; | NM_001297418; NM_031203; |
| | | | | CEAR; | |
| | | | | HNRNPM4; | |
| | | | | HNRPM4 | |
| FARP1_MOUSE | 10160 | FARP1 | FARP1 | CDEP; FARP1- | NM_001001715; NM_001286839; XM_011521046; |
| | | | | IT1; PPP1R75; | NM_005766 |
| | | | | PLEKHC2 | |
| ERH_MOUSE | 2079 | A0A024R6D4 | ERH | DROER | NM_004450 |
| SMD2_MOUSE | 6633 | SMD2 | SNRPD2 | SMD2; | NM_004597; NM_177542; XM_005259180 |
| | | | | SNRPD1; Sm- | |
| | | | | D2 | |
| PTPRS_MOUSE | 5802 | PTPRS | PTPRS | PTPSIGMA | XM_006722809; XM_006722810; XM_006722820; |
| | | | | | NM_002850; XM_005259606; XM_005259607; |
| | | | | | XM_006722808; XM_006722815; NM_130854; |
| | | | | | XM_011528157; NM_130855; XM_005259610; |
| | | | | | XM_006722812; XM_006722819; NM_130853; |
| | | | | | XM_005259600; XM_006722817; XM_006722818; |
| | | | | | XM_011528158; ; XM_006722814; XM_005259601; |
| | | | | | XM_005259609; XM_006722811 |
| MYO1D_MOUSE | 4642 | MYO1D | MYO1D | myr4; PPP1R108 | XR_934470; NM_001303280; NM_001303279; |
| | | | | | NM_015194 |
| NB5R3_MOUSE | 1727 | NB5R3 | CYB5R3 | B5R; DIA1 | NM_007326; NM_000398; NM_001129819; |
| | | | | | NM_001171660; NM_001171661; |
| RM46_MOUSE | 26589 | RM46 | MRPL46 | P2ECSL; | NM_022163 |
| | | | | LIECG2; | |
| | | | | C15orf4 | |
| NEDD4_MOUSE | 4734 | NEDD4 | NEDD4 | RPF1; NEDD4-1 | NM_001284339; XM_011521626; XM_011521624; |
| | | | | | NM_006154; NR_104302; XM_011521627; |
| | | | | | NM_001284338; NM_198400; XM_011521625; |
| | | | | | NM_001284340 |
| FBRL_MOUSE | 2091 | FBRL | FBL | FIB; FLRN; | XM_011548799; XM_011526623; XM_011548798; |
| | | | | RNU3IP1 | XM_005258651; NM_001436 |
| LXN_MOUSE | 56925 | LXN | LXN | TCI; ECI | NM_020169 |
| RAB9A_MOUSE | 9367 | RAB9A | RAB9A | RAB9 | NM_004251; NM_001195328 |
| HMGCL_MOUSE | 3155 | HMGCL | HMGCL | HL | NM_000191; NM_001166059 |
| Q8VHM5_MOUSE | — | | — | — | — |
| ITPR3_MOUSE | 3710 | ITPR3 | ITPR3 | IP3R; IP3R3 | XM_011514577; ; NM_002224; XM_011514576 |
| DHB12_MOUSE | 51144 | DHB12 | HSD17B12 | SDR12C1; KAR | XM_011520156; NM_016142 |
| PHIP_MOUSE | 55023 | PHIP | PHIP | DCAF14; | XM_011535919; NM_017934; XM_005248729; |
| | | | | WDR11; | XM_011535917; XM_011535918; XR_942499 |
| | | | | BRWD2; ndrp | |
| PTBP3_MOUSE | 9991 | PTBP3 | PTBP3 | ROD1 | XM_006717346; XM_005252324; XM_011519267; |
| | | | | | NM_001244897; NM_005156; XM_006717343; |
| | | | | | XM_011519266; NM_001163788; NM_001244898; |
| | | | | | NM_001163790; XM_011519265; NM_001244896 |
| NUP43_MOUSE | 348995 | NUP43 | NUP43 | p42; bA350J20.1 | XM_011535799; XM_005266961; XM_011535798; |
| | | | | | NM_198887; XM_005266960; XM_005266962; |
| | | | | | XR_942420; NM_024647; NR_104456 |
| ROAA_MOUSE | 3182 | ROAA | HNRNPAB | HNRPAB; | NM_004499; NM_031266 |
| | | | | ABBP1 | |
| KAD3_MOUSE | 50808 | Q7Z4Y4; | AK3 | AK3L1; | NM_001199855; NM_001199853; NM_016282; |
| | | KAD3 | | AKL3L1; AK6; | NM_001199854; NM_001199852; NM_001199856 |
| | | | | AKL3L; FIX | |
| RBM14_MOUSE | 10432 | RBM14 | RBM14 | COAA; | NM_001198837; ; NM_001198836; NM_006328; |
| | | | | TMEM137; SIP; | NM_032886 |
| | | | | SYTIP1; PSP2 | |
| MYH1_MOUSE | 4619 | MYH1 | MYH1 | HEL71; MyHC- | NM_005963 |
| | | | | 2x; MYHSA1; | |
| | | | | MYHa; MyHC- | |
| | | | | 2X/D | |
| RBBP6_MOUSE | 5930 | RBBP6 | RBBP6 | P2P-R; MY038; | XM_005255461; NM_018703; XM_005255462; |
| | | | | RBQ-1; | NM_006910; NM_032626 |
| | | | | SNAMA; PACT | |
| RFC2_MOUSE | 5982 | RFC2 | RFC2 | RFC40 | XR_927506; NM_001278792; NM_001278793; |
| | | | | | NM_002914; NM_181471; ; NM_001278791; |
| | | | | | XM_006716080 |
| Q0VBL3_MOUSE | — | | — | — | — |
| E9Q5G3_MOUSE | — | | — | — | — |
| RALY_MOUSE | 22913 | RALY | RALY | P542; HNRPCL2 | XM_005260336; XM_011528694; NM_007367; |
| | | | | | NM_016732; XM_011528695; XM_005260334 |
| STA5A_MOUSE | 6776 | STA5A; | STAT5A | MGF; STAT5 | NM_001288720; NM_001288719; XM_005257624; |
| | | Q59GY7; | | | NM_001288718; NM_003152 |
| | | A8K6I5; | | | |
| | | K7EK35 | | | |
| PHF5A_MOUSE | 84844 | PHF5A | PHF5A | SAP14b; INI; | NM_032758 |
| | | | | Rds3; | |
| | | | | bK223H9.2; | |
| | | | | SF3B7; SF3b14b | |
| ADRO_MOUSE | 2232 | ADRO | FDXR | ADXR | XM_006721772; XM_011524532; NM_001258015; |
| | | | | | XM_011524528; XM_011524531; NM_001258016; |
| | | | | | XM_011524527; XM_011524530; XM_011524533; |
| | | | | | NM_004110; NR_047576; NM_001258013; |
| | | | | | NM_001258014; XM_011524529; NM_001258012; |
| | | | | | NM_024417 |
| RT11_MOUSE | 64963 | RT11 | MRPS11 | HCC-2 | NM_176805; XM_011521946; XM_005254978; |
| | | | | | XM_011521947; NM_022839; XM_005254977 |
| BAZ1B_MOUSE | 9031 | BAZ1B | BAZ1B | WBSCR9; | NM_032408; NM_023005; XM_005250683; |
| | | | | WBSCR10; | |
| | | | | WSTF | |
| RAVR1_MOUSE | 125950 | RAVR1 | RAVER1 | — | NM_133452; XM_011527671; XM_011527672 |
| E41L2_MOUSE | 2037 | E41L2 | EPB41L2 | 4.1G; 4.1-G | XM_006715362; XM_011535523; NM_001431; |
| | | | | | XM_011535527; XR_942326; XR_942328; |
| | | | | | NM_001135554; XM_006715356; XM_011535531; |
| | | | | | XM_011535535; NM_001252660; XM_005266840; |
| | | | | | XM_011535522; XM_11535526; XM_011535530; |
| | | | | | XM_011535534; XM_011535521; XM_011535525; |
| | | | | | XM_011535528; XM_011535529; XM_011535532; |
| | | | | | NM_001199389; NM_001135555; NM_001199388; |
| | | | | | XM_005266841; XM_011535524; XM_011535533; |
| | | | | | XM_011535536 |
| DCA13_MOUSE | 25879 | DCA13 | DCAF13 | HSPC064; | NM_015420 |
| | | | | WDSOF1; | |
| | | | | GM83 | |
| Q3TIX6_MOUSE | — | | — | — | — |
| CLK3_MOUSE | 1198 | CLK3 | CLK3 | PHCLK3/152; | XM_005254153; XM_011521210; XM_011521206; |
| | | | | PHCLK3 | XM_011521209; XM_011521208; NM_003992; |
| | | | | | XM_005254151; XM_006720384; XM_011521205; |
| | | | | | XR_931746; NM_001292; XM_011521207; |
| | | | | | NM_001130028 |
| LAP2_MOUSE | 55914 | LAP2 | ERBB2IP | HEL-S-78; | XM_011543514; NM_001253698; NM_018695; ; |
| | | | | LAP2; ERBIN | XM_005248554; XM_005248555; NM_001006600; |
| | | | | | NM_001253699; XM_006714660; NM_001253697; |
| | | | | | NM_001253701 |
| WDR33_MOUSE | 55339 | WDR33 | WDR33 | WDC146; | XM_005263697; NM_001006623; NM_018383; |
| | | | | NET14 | XM_011511436; NM_001006622 |
| SMC3_MOUSE | 9126 | SMC3 | SMC3 | BAM; HCAP; | NM_005445 |
| | | | | SMC3L1; | |
| | | | | CSPG6; CDLS3; | |
| | | | | BMH | |
| GULP1_MOUSE | 51454 | GULP1 | GULP1 | CED6; CED-6; | XM_006712583; XM_006712585; XM_006712589; |
| | | | | GULP | XM_011511327; XM_011511332; NM_001252668; |
| | | | | | NM_001252669; XM_011511328; XM_011511329; |
| | | | | | XM_006712590; XM_011511331; XM_011511334; |
| | | | | | NM_016315; NR_045563; XM_006712581; |
| | | | | | XM_011511333; XM_011511335; XM_006712580; |
| | | | | | XM_006712582; XM_006712584; NR_045562; |
| | | | | | XM_011511330 |
| LS14A_MOUSE | 26065 | LS14A | LSM14A | C19orf13; | XM_011547018; NM_015578; XM_011526708; |
| | | | | RAP55A; | XM_005276949; XM_005258719; XM_005258720; |
| | | | | RAP55; | XM_005258721; XM_005276948; NM_001114093; |
| | | | | FAM61A | XM_005276950 |
| MCU_MOUSE | 90550 | MCU | MCU | C10orf42; | NR_073062; NM_138357; NM_001270679; |
| | | | | CCDC109A | NM_001270680 |
| KANK2_MOUSE | 25959 | KANK2 | KANK2 | PPKWH; SIP; | NM_001136191; NM_015493 |
| | | | | ANKRD25; | |
| | | | | MXRA3 | |
| ALDH2_MOUSE | 217 | ALDH2 | ALDH2 | ALDHI; ALDH- | NM_001204889; NM_000690; |
| | | | | E2; ALDM | |
| CBR2_MOUSE | — | | — | — | — |
| MAAI_MOUSE | 2954 | MAAI | GSTZ1 | GSTZ1-1; | XM_011536671; NM_001513; XM_005267559; |
| | | | | MAAI; MAI | NM_145871; NM_145870; XM_011536670 |
| TRA2A_MOUSE | 29896 | TRA2A | TRA2A | AWMS1; | NM_013293; NM_001282757; NM_001282759; |
| | | | | HSU53209 | XM_005249725; XM_011515331; XM_006715713; |
| | | | | | NM_001282758 |
| TENC1_MOUSE | 23371 | TENC1 | TNS2 | C1TEN; TENC1; | XM_006719303; NM_015319; XM_006719304; |
| | | | | C1-TEN | XM_011538079; NM_170754; XM_006719302; |
| | | | | | NM_198316 |
| ACSF2_MOUSE | 80221 | ACSF2 | ACSF2 | AVYV493; | XR_934566; XR_934563; XR_934564; NM_025149; |
| | | | | ACSMW | XR_429924; NM_001288970; XM_006722110; |
| | | | | | XM_011525294; XR_934567; NM_001288968; |
| | | | | | NM_001288969; NM_001288971; NM_001288972; |
| | | | | | XR_934565; NR_110232 |
| PRP19_MOUSE | 27339 | PRP19 | PRPF19 | hPSO4; PSO4; | NM_014502 |
| | | | | UBOX4; PRP19; | |
| | | | | SNEV; NMP200 | |
| ENV1_MOUSE | — | | — | — | — |
| PR38A_MOUSE | 84950 | PR38A | PRPF38A | Prp38 | NM_032864; XM_011542315; NM_032284 |
| RRP5_MOUSE | 22984 | RRP5 | PDCD11 | NFBP; RRP5; | NM_014976; XM_011539538; XM_011539540; |
| | | | | ALG-4; ALG4 | XM_005269647; XM_011539539 |
| SQRD_MOUSE | 58472 | SQRD | SQRDL | CGI-44; | NM_001271213; NM_021199 |
| | | | | PRO1975; | |
| | | | | SQOR | |
| THOC3_MOUSE | 84321 | THOC3 | THOC3 | hTREX45; | XM_011534668; XM_011534666; NM_032361; |
| | | | | THO3 | XM_011534667 |
| THIKA_MOUSE | 30 | THIK | ACAA1 | ACAA; THIO; | NM_001130410; XM_006713122; NR_024024; |
| | | | | PTHIO | NM_001607; XM_011533650; ; XM_006713123 |
| P5CR2_MOUSE | 29920 | P5CR2 | PYCR2 | P5CR2 | NM_001271681; NM_013328 |
| PDK3_MOUSE | 5165 | PDK3 | PDK3 | CMTX6; GS1- | ; NM_001142386; NM_005391 |
| | | | | 358P8.4 | |
| Q8BGJ5_MOUSE | — | | — | — | — |
| S12A2_MOUSE | 6558 | S12A2 | SLC12A2 | BSC2; NKCC1; | NM_001256461; NM_001046; XM_011543588; |
| | | | | BSC; PPP1R141 | NR_046207 |
| RRMS2_MOUSE | 5939 | RBMS2 | RBMS2 | SCR3 | XM_005269059; NM_002898; XM_006719543; |
| | | | | | XM_011538639; XM_005269060; XM_011538640; |
| | | | | | XM_006719541; XM_006719542; XM_006719544; |
| | | | | | XM_011538637; XM_005269061; XM_011538642; |
| | | | | | XM_005269066; XM_011538638; XM_011538641 |
| PLRG1_MOUSE | 5356 | PLRG1 | PLRG1 | PRPF46; PRL1; | NM_002669; NM_001201564 |
| | | | | PRP46; Cwc1; | |
| | | | | TANGO4 | |
| RINI_MOUSE | 6050 | RINI | RNH1 | RAI; RNH | XM_011520263; XM_011546605; XM_011520257; |
| | | | | | XM_011546603; XM_011546606; NM_203383; |
| | | | | | NM_203389; XM_011520261; XM_011546604; |
| | | | | | XM_011546609; XM_011546607; XM_011546608; |
| | | | | | NM_203386; NM_203388; XM_011520259; |
| | | | | | XM_011520262; XM_011546602; XM_011520260; |
| | | | | | XM_011546610; XM_011520256; NM_002939; |
| | | | | | NM_203385; NM_203387; XM_011520255; |
| | | | | | XM_011520258; NM_203384 |
| CDK4_MOUSE | 1019 | CDK4 | CDK4 | PSK-J3; CMM3 | NM_052984; NM_000075 |
| ACADM_MOUSE | 34 | ACADM | ACADM | ACAD1; | NM_001127328; NM_001286042; NM_001286043; ; |
| | | | | MCAD; | NM_000016; NM_001286044; NR_022013 |
| | | | | MCADH | |
| HNRPK_MOUSE | 3190 | HNRPK | HNRNPK | TUNP; CSBP; | XM_011518616; NM_002140; NM_031262; |
| | | | | HNRPK | XM_005251965; ; XM_005251960; XM_005251961; |
| | | | | | NM_031263; XM_005251964; XM_005251966; |
| | | | | | XM_005251963 |
| GPX41_MOUSE | 2879 | Q6PI42 | GPX4 | GPx-4; MCSP; | NM_002085; NM_001039847; NM_001039848 |
| | | | | snPHGPx; | |
| | | | | PHGPx; GSHPx- | |
| | | | | 4; snGPx | |
| RBM3_MOUSE | 5935 | RBM3 | RBM3 | IS1-RNPL; | NM_001017430; XM_011543939; NM_001017431; |
| | | | | RNPL | NM_006743; XM_011543938 |
| SNR40_MOUSE | 9410 | SNR40 | SNRNP40 | PRPF8BP; 40K; | NM_004814 |
| | | | | SPF38; WDR57; | |
| | | | | HPRP8BP; | |
| | | | | PRP8BP | |
| KHDR1_MOUSE | 10657 | KHDR1 | KHDRBS1 | Sam68; p62; p68 | NR_073498; NR_073499; NM_001271878; NM_006559 |
| ILK_MOUSE | 3611 | ILK | ILK | HEL-S-28; | XM_005252904; NM_001278441; ; XM_011520065; |
| | | | | p59ILK; ILK-1; | XM_005252905; NM_001014795; NM_001278442; |
| | | | | ILK-2; P59 | NM_001014794; NM_004517 |
| GAR1_MOUSE | 54433 | GAR1 | GAR1 | NOLA1 | NM_032993; NM_018983 |
| CSTF1_MOUSE | 1477 | CSTF1 | CSTF1 | CstFp50; CstF- | NM_001033522; NM_001033521; NM_001324; |
| | | | | 50 | XM_011528600 |
| UGGG1_MOUSE | 56886 | UGGG1 | UGGT1 | UGCGL1; | XM_006712635; XR_922969; NM_020120; |
| | | | | HUGT1; UGT1 | NM_001025777; NR_027671; XM_006712634; |
| | | | | | XM_006712636 |
| CPSF4_MOUSE | 10898 | CPSF4 | CPSF4 | CPSF30; NAR; | XM_011515755; XM_011515756; NM_006693; |
| | | | | NEB1 | XM_011515757; NM_001081559; XM_011515758; |
| | | | | | XM_011515759 |
| IF4A3_MOUSE | 9775 | IF4A3 | EIF4A3 | MUK34; | XM_011525522; NM_014740 |
| | | | | NMP265; | |
| | | | | NUK34; | |
| | | | | eIF4AIII; RCPS; | |
| | | | | DDX48 | |
| PCBP2_MOUSE | 5094 | PCBP2 | PCBP2 | HNRNPE2; | NM_001128912; NM_001128911; NM_001128914; |
| | | | | HNRPE2; | NM_001098620; NM_031989; NM_001128913; |
| | | | | hnRNP-E2 | NM_005016 |
| QKI_MOUSE | 9444 | QKI | QKI | Hqk; QK; QK3; | XM_011536259; XM_011536260; XR_942633; ; |
| | | | | hqkI; QK1 | XM_011536258; NM_206853; NM_001301085; |
| | | | | | NM_006775; XM_011536261; NM_206854; |
| | | | | | XR_245557; NM_206855 |
| ACADV_MOUSE | 37 | ACADV | ACADVL | ACAD6; | XM_011523829; XR_934023; NM_001270447; |
| | | | | LCACD; | XR_934021; NM_001270448; NM_000018; |
| | | | | VLCAD | XM_006721516; ; XM_011523830; XR_934022; |
| | | | | | NM_001033859 |
| ELAV1_MOUSE | 1994 | ELAV1 | ELAVL1 | ELAV1; MelG; | XM_011527777; NM_001419 |
| | | | | Hua; HUR | |
| FINC_MOUSE | 2335 | FINC | FN1 | FNZ; GFND; | XM_005246416; ; XM_005246413; NM_212476; |
| | | | | CIG; ED-B; | XM_005246407; XM_005246410; XM_005246414; |
| | | | | GFND2; MSF; | NM_212474; XM_005246402; XM_005246408; |
| | | | | FINC; FN; LETS | XM_005246409; XM_005246399; NM_054034; |
| | | | | | XM_005246400; XM_005246403; XM_005246405; |
| | | | | | XM_005246406; XM_005246415; NM_002026; |
| | | | | | XM_005246398; XM_005246401; XM_005246404; |
| | | | | | XM_005246412; XM_005246417; XM_005246397; |
| | | | | | XM_005246411; NM_212478; NM_212482; |
| | | | | | NM_212475 |
| WDR3_MOUSE | 10885 | WDR3 | WDR3 | UTP12; DIP2 | NM_006784 |
| SRSF9_MOUSE | 8683 | SRSF9 | SRSF9 | SFRS9; SRp30c | NM_003769 |
| NPM_MOUSE | 4869 | NPM | NPM1 | B23; NPM | XM_005265920; ; NM_001037738; NM_002520; |
| | | | | | NM_199185; XM_011534564 |
| FUBP2_MOUSE | 8570 | FUBP2 | KHSRP | FUBP2; FBP2; | XM_005259668; NM_003685; XM_011528395 |
| | | | | KSRP | |
| HNRPD_MOUSE | 3184 | HNRPD | HNRNPD | P37; AUF1; | ; NM_002138; NM_001003810; NM_031370; |
| | | | | AUF1A; | NM_031369 |
| | | | | HNRPD; | |
| | | | | hnRNPD0 | |
| UTP15_MOUSE | 84135 | UTP15 | UTP15 | NET21 | NM_001284431; XM_011543680; NM_001284430; |
| | | | | | NM_032175 |
| IMMT_MOUSE | — | | — | — | — |
| CD2A1_MOUSE | 1029 | CD2A2 | CDKN2A | P16INK4A; | XM_011517676; XR_929166; ; NM_058197; |
| | | | | CMM2; P14; | NM_058196; XR_929165; NM_001195132; XR_929162; |
| | | | | P16INK4; P19; | NM_058195; XM_011517675; XM_011517678; |
| | | | | P19ARF; | XM_011517679; XR_929159; NM_000077; |
| | | | | CDKN2; INK4; | XM_011517677; XR_929161; XR_929163; |
| | | | | TP16; MTS1; | XM_005251343; XR_929164 |
| | | | | INK4A; | |
| | | | | P14ARF; ARF; | |
| | | | | MTS-1; P16- | |
| | | | | INK4A; CDK4I; | |
| | | | | MLM; P16 | |
| RSMB_MOUSE | 6628 | Q66K91 | SNRPB | CCMS; COD; | NM_198216; NM_003091; |
| | | | | Sm-B/B′; | |
| | | | | SmB/SmB′; | |
| | | | | snRNP-B; | |
| | | | | SNRPB1; | |
| | | | | SmB/B′ | |
| IMA1_MOUSE | 3838 | IMA1 | KPNA2 | IPOA1; QIP2; | XM_011524783; NM_002266 |
| | | | | SRP1alpha; | |
| | | | | RCH1 | |
| THIL_MOUSE | 38 | THIL | ACAT1 | ACAT; MAT; | XM_006718834; XM_006718835; NM_000019; |
| | | | | T2; THIL | |
| RT07_MOUSE | 51081 | RT07 | MRPS7 | S7mt; bMRP27a; | NM_015971 |
| | | | | MRP-S7; | |
| | | | | RPMS7; RP-S7; | |
| | | | | MRP-S | |
| MEN1_MOUSE | 4221 | MEN1 | MEN1 | MEAI; SCG2 | NM_130800; NM_130802; NM_130799; |
| | | | | | XM_011545041; NM_130804; NM_000244; |
| | | | | | XM_005274001; NM_130801; NM_130803; |
| | | | | | XM_011545040; ; XM_011545042 |
| HNRPF_MOUSE | 3185 | HNRPF | HNRNPF | HNRPF; | NM_001098207; NM_001098208; NM_001098204; |
| | | | | OK/SW-cl.23; | NM_001098205; NM_001098206; NM_004966 |
| | | | | mcs94-1 | |
| ROA3_MOUSE | 220988 | ROA3 | HNRNPA3 | 2610510D13Rik; | NM_194247; XM_005246380; XM_006712365; |
| | | | | D10S102; | XM_005246381 |
| | | | | HNRPA3; | |
| | | | | FBRNP | |
| NCOA5_MOUSE | 57727 | NCOA5 | NCOA5 | bA465L10.6; | NM_020967; XM_011528951; XM_005260474 |
| | | | | CIA | |
| KIF4_MOUSE | 24137 | KIF4A | KIF4A | KIF4; KIF4G1; | XM_011530893; ; NM_012310 |
| | | | | MRX100 | |
| FBLN1_MOUSE | 2192 | Q8NBH6 | FBLN1 | FBLN; FIBL1 | NM_006486; NM_001996; ; NM_006485; NM_006487 |
| SYWM_MOUSE | 10352 | SYWM | WARS2 | TrpRS | XM_006710283; NM_015836; NM_201263; |
| | | | | | XM_011540493; XM_005270350; XM_011540495; |
| | | | | | XM_011540494 |
| GELS_MOUSE | 2934 | GELS | GSN | AGEL; ADF | XM_006717075; XM_011518587; NM_198252; |
| | | | | | XM_005251940; XM_005251945; XM_011518584; |
| | | | | | XM_011518594; XM_005251943; XM_005251944; |
| | | | | | XM_011518586; XM_011518592; NM_001127666; |
| | | | | | XM_006717079; XM_0115118589; NM_001127664; |
| | | | | | XM_011518585; XM_011518588; XM_011518590; |
| | | | | | XM_011518593; ; NM_000177; NM_001127662; |
| | | | | | NM_001127663; NM_001127667; XM_011518591; |
| | | | | | NM_001258029; NM_001127665; NM_001258030 |
| UTP20_MOUSE | 27340 | UTP20 | UTP20 | DRIM | NM_014503; XM_006719343 |
| TENA_MOUSE | 3371 | TENA | TNC | 150-225; | XM_011518624; XM_011518627; ; XM_005251974; |
| | | | | GMEM; JI; GP; | XM_011518629; XM_006717100; XM_011518622; |
| | | | | TN; TN-C; | XM_011518623; XM_011518626; XM_005251972; |
| | | | | DFNA56; HXB | XM_006717097; XM_005251975; XM_011518625; |
| | | | | | XM_006717096; XM_011518628; XM_011518630; |
| | | | | | NM_002160; XM_005251973; XM_006717098; |
| | | | | | XM_006717101 |
| SENP3_MOUSE | 26168 | SENP3 | SENP3 | Ulp1; SMT3IP1; | NM_015670 |
| | | | | SSP3 | |
| CPT2_MOUSE | 1376 | CPT2 | CPT2 | CPTASE; CPT1; | ; XM_005270484; NM_000098 |
| | | | | IIAE4 | |
| RBBP7_MOUSE | 5931 | RBBP7 | RBBP7 | RbAp46 | XM_011545553; NM_001198719; XM_011545554; |
| | | | | | NM_002893 |
| AOFA_MOUSE | 4128 | AOFA | MAOA | MAO-A | NM_000240; NM_001270458; |
| ECHB_MOUSE | 3032 | ECHB | HADHB | ECHB; | NM_001281513; NM_000183; XM_011532803; ; |
| | | | | MSTP029; | NM_001281512; XM_011532804 |
| | | | | MTPB; TP- | |
| | | | | BETA | |
| E9QNN1_MOUSE | — | | — | — | — |
| Q91VA7_MOUSE | 3420 | A0A087WZN1 | IDH3B | RP46; H-IDHB | XM_005260716; XR_937066; ; NM_174856; |
| | | | | | NM_174855; NM_001258384; NM_006899 |
| PYC_MOUSE | 5091 | A0A024R5C5 | PC | PCB | XM_006718577; ; NM_001040716; XM_011545086; |
| | | | | | XM_005274031; XM_005274032; XM_006718578; |
| | | | | | XM_006718579; NM_000920; XM_011545087; |
| | | | | | NM_022172; XM_011545085; XM_011545088 |
| DNMT1_MOUSE | 1786 | I6L9H2 | DNMT1 | AIM; CXXC9; | XM_011527773; ; NM_001130823; NM_001379; |
| | | | | DNMT; MCMT; | XM_011527772; XM_011527774 |
| | | | | ADCADN; | |
| | | | | HSN1E | |
| ROA2_MOUSE | 3181 | ROA2 | HNRNPA2B1 | HNRPA2; | XR_242076; XR_242077; NM_002137; ; XR_428077; |
| | | | | RNPA2; | XR_428078; XM_006715714; NM_031243; |
| | | | | SNRPB1; | XM_005249729 |
| | | | | HNRNPA2; | |
| | | | | HNRNPB1; | |
| | | | | IBMPFD2; | |
| | | | | HNRPA2B1; | |
| | | | | HNRPB1 | |
| LARP7_MOUSE | 51574 | LARP7 | LARP7 | ALAZS; PIP7S; | NM_015454; NM_016648; NR_049768; NM_001267039 |
| | | | | HDCMA18P | |
| PREP_MOUSE | 10531 | PREP | PITRM1 | PreP; MP1 | XM_005252345; XM_011519292; NM_014968; |
| | | | | | NM_001242307; NM_001242309; XM_006717362; |
| | | | | | NM_014889 |
| EDC4_MOUSE | 23644 | EDC4 | EDC4 | RCD-8; HEDLS; | NM_014329 |
| | | | | Ge-1; RCD8; | |
| | | | | GE1; HEDL5 | |
| RFOX2_MOUSE | 23543 | RFOX2 | RBFOX2 | FOX2; Fox-2; | XM_006724190; XM_006724193; ; XM_006724185; |
| | | | | HNRBP2; | XM_006724187; XM_011530036; NM_001031695; |
| | | | | HRNBP2; | NM_001082577; XM_005261428; XM_005261430; |
| | | | | RBM9; RTA; | XM_005261431; XM_005261432; XM_005261433; |
| | | | | fxh; dJ106I20.3 | XM_005261437; NM_001082579; XM_005261429; |
| | | | | | XM_006724186; XM_006724194; XM_006724192; |
| | | | | | NM_001082578; NM_014309; NM_001082576; |
| | | | | | XM_005261435; XM_006724188; XM_006724189; |
| | | | | | XM_006724191 |
| SMD3_MOUSE | 6634 | SMD3 | SNRPD3 | SMD3; Sm-D3 | NM_001278656; NR 103819; NM_004175 |
| ODBA_MOUSE | 593 | ODBA | BCKDHA | MSU; MSUD1; | ; NM_000709; NM_001164783 |
| | | | | BCKDE1A; | |
| | | | | OVD1A | |
| RT23_MOUSE | 51649 | RT23 | MRPS23 | CGI-138; | NM_016070 |
| | | | | HSPC329; MRP- | |
| | | | | S23 | |
| RBP2_MOUSE | 5903 | RBP2 | RANBP2 | ANE1; TRP1; | XM_011511576; NM_006267; XM_005264002; |
| | | | | TRP2; ADANE; | XM_005264004; XM_011511575; XM_005264003; |
| | | | | NUP358; IIAE3 | XM_005264007; XM_011511577; XM_005264005; |
| | | | | | XM_011511578; |
| NIPA_MOUSE | 51530 | NIPA | ZC3HC1 | HSPC216; NIPA | NM_001282190; XM_005250403; NM_001282191; |
| | | | | | XM_011516288; XM_011516289; XM_011516290; |
| | | | | | NM_016478 |
| KAD1_MOUSE | 203 | Q6FGX9 | AK1 | HTL-S-58j | XM_005251786; ; XM_011518348; XM_011518349; |
| | | | | | NM_000476 |
| SUCB2_MOUSE | 8801 | SUCB2 | SUCLG2 | GBETA | XR_940506; XR_245062; NM_001177599; NM_003848 |
| PRP8_MOUSE | 10594 | PRP8 | PRPF8 | SNRNP220; | NM_006445; |
| | | | | HPRP8; PRPC8; | |
| | | | | PRP8; RP13 | |
| NCPR_MOUSE | 5447 | NCPR | POR | P450R; CPR; | ; NM_000941 |
| | | | | CYPOR | |
| LMNB1_MOUSE | 4001 | LMNB1 | LMNB1 | LMN2; LMNB; | NM_001198557; XR_948250; ; NM_005573 |
| | | | | LMN; ADLD | |
| SF3B4_MOUSE | 10262 | SF3B4 | SF3B4 | SF3b49; Hsh49; | ; NM_005850 |
| | | | | AFD1; SAP49 | |
| A2ANY6_MOUSE | 23195 | MDN1 | MDN1 | — | XM_011535635; XR_942362; XM_005248700; |
| | | | | | XM_006715405; XM_011535636; ; NM_014611 |
| LAP2B_MOUSE | 7112 | LAP2B; | TMPO | LAP2; CMD1T; | ; NM_001032284; XM_005269132; XM_005269130; |
| | | LAP2A | | LEMD4; TP; | NM_001032283; NM_603276 |
| | | | | PRO0868 | |
| GNL3_MOUSE | 26354 | GNL3 | GNL3 | C77032; E21G3; | NM_206826; ; NM_014366; NM_206825 |
| | | | | NNP47; NS | |
| RL6_MOUSE | 6128 | A0A024RBK3; | RPL6 | TXREB1; | XM_006719548; XM_006719546; NM_000970; |
| | | Q8TBK5; RL6 | | TAXREB107; | NM_001024662; XM_006719547; XM_006719549; |
| | | | | SHUJUN-2; L6 | XM_011538647; XM_011538646 |
| RBM22_MOUSE | 55696 | RBM22 | RBM22 | Cwc2; ZC3H16; | NM_018047 |
| | | | | fSAP47 | |
| MYO5A_MOUSE | 4644 | MYO5A | MYO5A | GS1; MYO5; | XM_011521610; XM_011521611; NM_001142495; |
| | | | | MYH12; | XM_011521607; ; NM_000259; XM_005254398; |
| | | | | MYR12 | XM_011521606; XM_005254397; XM_011521609; |
| | | | | | XM_011521612; XM_011521608 |
| HYOU1_ MOUSE | 10525 | HYOU1 | HYOU1 | HSP12A; ORP- | XM_005271392; XM_011548779; NM_001130991; |
| | | | | 150; Grp170; | XM_011548780; XM_011548781; XM_011548782; |
| | | | | ORP150; GRP- | NM_006389; XM_011542557; XM_005271394; |
| | | | | 170 | XR_947790; XR_953214; XM_005271393; |
| | | | | | XM_011542558; XM_011548778 |
| ACDSB_MOUSE | 36 | ACDSB | ACADSB | 2-MEBCAD; | ; NM_001609 |
| | | | | ACAD7; | |
| | | | | SBCAD | |
| NOL11_MOUSE | 25926 | NOL11 | NOL11 | — | NM_015462; NM_001303272 |
| HEMH_MOUSE | 2235 | HEMH; | FECH | FCE; EPP | NM_000140; XM_011525882; NM_001012515; ; |
| | | Q7KZA3 | | | XM_011525881 |
| SNUT2_MOUSE | 10713 | SNUT2 | USP39 | SNRNP65; | NM_006590; NM_001256726; NM_001256728; |
| | | | | HSPC332; 65K; | NR_046347; XM_011532488; XR_939653; |
| | | | | SAD1; CGI-21 | NM_001256725; NM_001256727; XM_006711922; |
| | | | | | XR_939652; XM_006711923; XM_011532487 |
| NOG1_MOUSE | 23560 | NOG1; | GTPBP4 | CRFG; NGB; | NM_012341 |
| | | D2CFK9 | | NOG1 | |
| NEP1_MOUSE | 10436 | NEP1 | EMG1 | C2F; Grcc2f; | ; XM_011520907; NM_006331 |
| | | | | NEP1 | |
| WDR61_MOUSE | 80349 | WDR61 | WDR61 | REC14; SKI8 | NM_001303248; NM_001303247; XM_011522094; |
| | | | | | XR_931918; NM_025234 |
| RFC3_MOUSE | 5983 | RFC3 | RFC3 | RFC38 | XM_011535174; NM_002915; NM_181558; |
| | | | | | XM_011535173; XM_011535175; XM_011535172; |
| | | | | | XM_011535176 |
| Q3TWW8_MOUSE | 6431 | SRSF6 | SRSF6 | SRP55; B52; | ; NR_034009; XR_936608; NM_006275 |
| | | | | HEL-S-91; | |
| | | | | SFRS6 | |
| PPIL2_MOUSE | 23759 | PPIL2 | PPIL2 | CYP60; Cyp-60; | XM_011530047; XM_011530051; XM_011530041; |
| | | | | CYC4; UBOX7; | XM_011530045; NM_148175; XM_011530046; |
| | | | | hCyP-60 | XM_011530048; XM_011530050; XM_005261447; |
| | | | | | XM_011530043; NM_014337; XM_005261448; |
| | | | | | XM_011530042; XM_011530044; XM_011530049; |
| | | | | | NM_148176 |
| HDAC1_MOUSE | 3065 | Q6IT96; | HDAC1 | RPD3; GON-10; | XM_011541309; NM_004964 |
| | | HDAC1 | | HD1; RPD3L1 | |
| PAPD1_MOUSE | 55149 | PAPD1 | MTPAP | PAPD1; SPAX4 | ; NM_018109 |
| MCM3_MOUSE | 4172 | MCM3 | MCM3 | P1-MCM3; P1.h; | NM_002388; NM_001270472 |
| | | | | HCC5; RLFB | |
| SRSF7_MOUSE | 6432 | SRSF7 | SRSF7 | SFRS7; 9G8; | XM_011533032; XR_939708; XR_426994; |
| | | | | AAG3 | NM_001195446; XR_939711; NM_001031684; |
| | | | | | XM_005264484; XM_005264485; XR_939709; |
| | | | | | XR_939710; NM_006276 |
| THIM_MOUSE | 10449 | THIM | ACAA2 | DSAEC | NM_006111 |
| PKIIP_MOUSE | 55003 | PKIIP | PAKIIP1 | bA421M1.5; | XM_005249204; XM_011514720; XM_006715129; |
| | | | | PIP1; hPIP1; | XM_011514721; NM_017906 |
| | | | | MAK11; | |
| | | | | WDR84 | |
| ATAD1_MOUSE | 84896 | ATAD1 | ATAD1 | THORASE; | XM_005270251; XM_011540302; XM_005270253; |
| | | | | FNP001; AFDC1 | XR_945847; NM_032810; XM_005276252; |
| | | | | | XM_011540303; XM_011540304 |
| Q3U821_MOUSE | — | — | — | — | — |
| SYYM_MOUSE | 51067 | SYYM | YARS2 | MT-TYRRS; | XR_931297; XR_931299; ; XR_242892; XR_429036; |
| | | | | TYRRS; | XR_931298; XR_242891; NM_001040436; XR_931296; |
| | | | | MLASA2; CGI- | NM_015936 |
| | | | | 04 | |
| RU17_MOUSE | 6625 | RU17 | SNRNP70 | U1-70K; Snp1; | XM_011527241; NM_001009820; NM_001301069; |
| | | | | U170K; | NM_003089; XM_005259178; XM_011527240 |
| | | | | SNRP70; U1AP; | |
| | | | | U1RNP; RPU1; | |
| | | | | RNPU1Z | |
| NUP85_MOUSE | 79902 | NUP85 | NUP85 | FROUNT; | XR_429921; NM_024844; XR_243683; XM_005257690; |
| | | | | Nup75 | XM_011525267; NM_001303276; XM_005257693; |
| | | | | | XM_005257692; XM_006722094; XM_011525268; |
| | | | | | XR_934552 |
| E9Q5F4_MOUSE | — | | | | |
| POGZ_MOUSE | 23126 | POGZ | POGZ | ZNF635; | XM_011509331; NM_015100; XM_005244999; |
| | | | | ZNF635m; | XR_921760; NM_001194938; XM_005245006; |
| | | | | ZNF280E | XM_011509330; NM_145796; NM_207171; |
| | | | | | XM_005245000; XM_005245001; XM_005245005; |
| | | | | | NM_001194937 |
| WDR12_MOUSE | 55759 | Q53T99; | WDR12 | YTM1 | XM_011511469; NM_018256 |
| | | WDR12 | | | |
| RL12_MOUSE | 6136 | RL12 | RPL12 | L12 | NM_000976 |
| ARL2_MOUSE | 402 | ARL2 | ARL2 | ARFL2 | NM_001667; NM_001199745 |
| RPAB3_MOUSE | 5437 | RPAB3 | POLR2H | RPABC3; RPB8; | XM_006713667; XM_006713666; XM_006713670; |
| | | | | RPB17 | NM_001278700; NM_001278714; XM_005247541; |
| | | | | | NM_001278698; XM_006713668; NM_001278699; |
| | | | | | NM_001278715; NM_006232 |
| CALX_MOUSE | 821 | CALX | CANX | P90; IP90; CNX | XM_011534664; XM_011534665; NM_001024649; |
| | | | | | NM_001746 |
| AP2A2_MOUSE | 161 | AP2A2 | AP2A2 | HIP9; HYPJ; | XM_011519928; NM_012305; NM_001242837; |
| | | | | ADTAB; | XM_011519930; XR_930847; XM_011519929 |
| | | | | CLAPA2; HIP-9 | |
| EFGM_MOUSE | 85476 | E5KND5; | GFM1 | EGF1; COXPD1; | ; NM_024996; XM_006713795; XM_011513247 |
| | | EFGM | | GFM; EFG; | |
| | | | | hEFG1; EFG1; | |
| | | | | EFGM | |
| CELF1_MOUSE | 10658 | CELF1 | CELF1 | CUGBP1; | XM_011519847; XM_011519853; XM_011519856; |
| | | | | NAB50; hNab50; | XM_011519855; XM_011519859; NM_001172640; |
| | | | | CUG-BP; | NM_006560; XM_011519849; XM_011519854; |
| | | | | CUGBP; | XM_011519851; XM_011519852; NM_001172639; |
| | | | | BRUNOL2; | XM_011519850; XM_011519857; NM_198700; |
| | | | | NAPOR; EDEN- | XM_011519848; XM_011519858; NM_001025596 |
| | | | | BP | |
| ARAF_MOUSE | 369 | ARAF | ARAF | A-RAF; ARAF1; | XM_011543909; XM_011543907; XM_011543906; ; |
| | | | | PKS2; RAFA1 | XM_006724529; NM_001256196; XM_011543908; |
| | | | | | NM_001256197; NM_001654 |
| HNRPC_MOUSE | — | | | | |
| SMCA5_MOUSE | 8467 | SMCA5 | SMARCA5 | ISWI; SNF2H; | NM_003601; XM_011532361 |
| | | | | hISWI; | |
| | | | | WCRF135; | |
| | | | | hSNF2H | |
| HNRH1_MOUSE | 3187 | HNRH1 | HNRNPH1 | HNRPH1; | XM_006714862; XM_005265895; XM_006714863; |
| | | | | hnRNPH; | XM_011534541; XM_005265901; XM_005265896; |
| | | | | HNRPH | XM_011534542; XM_011534543; XM_011534544; |
| | | | | | NM_001257293; NM_005520; XM_011534547; |
| | | | | | XM_005265902; XM_011534545; XM_011534546 |
| RBM4B_MOUSE | 83759 | RBM4B | RBM4B | ZCCHC21B; | XR_247214; NM_001286135; XR_247213; |
| | | | | ZCRB3B; | XM_011545297; NM_031492 |
| | | | | RBM4L; | |
| | | | | ZCCHC15; | |
| | | | | RBM30 | |
| MTMR5_MOUSE | 6305 | MTMR5 | SBF1 | CMT4B3; | XM_005261931; XM_005261935; XM_011530709; |
| | | | | MTMR5; | XM_011530710; XM_011530707; NM_002972; |
| | | | | DENND7A | XR_938344; ; XM_011530708; XM_011530711 |
| RL23_MOUSE | 9349 | RL23 | RPL23 | rpL17; L23 | NM_000978 |
| DDX3X_MOUSE | 1654 | DDX3X | DDX3X | DBX; DDX14; | ; NM_024005; NM_001356; NR_126093; |
| | | | | DDX3; CAP-Rf; | XM_011543892; NM_001193417; NM_001193416; |
| | | | | HLP2 | NR_126094 |
| NMRL1_MOUSE | 57407 | NMRL1 | NMRAL1 | HSCARG; | XM_006720905; NM_020677; XM_006720906; |
| | | | | SDR48A1 | XM_006725239; XM_011522566; XM_005255447; |
| | | | | | XM_006725238; NM_001305141; XM_005255446; |
| | | | | | XM_006725236; XM_011546747; XM_006725237; |
| | | | | | XM_011522567; XM_011546748; NM_001305142 |
| TR150_MOUSE | 9967 | TR150 | THRAP3 | TRAP150 | XM_005271371; XR_246308; NM_005119 |
| NAT10_MOUSE | 55226 | NAT10 | NAT10 | NET43; ALP | XM_011520197; NM_001144030; NM_024662 |
| ODPB_MOUSE | 5162 | ODPB | PDHB | PHE1B; PDHE1- | XM_011533828; NM_000925; NR_033384; |
| | | | | B; PDHBD | NM_001173468; |
| DDX1_MOUSE | 1653 | DDX1 | DDX1 | DBP-RB; | NM_004939 |
| | | | | UKVH5d | |
| ECHA_MOUSE | 3030 | ECHA | HADHA | MTPA; LCHAD; | NM_000182; |
| | | | | ECHA; GBP; | |
| | | | | TP-ALPHA; | |
| | | | | HADH; LCEH | |
| PREB_MOUSE | 10113 | PREB | PREB | SEC12 | XM_011532471; XM_011532472; XR_939649; |
| | | | | | XM_006711914; XR_939648; NM_013388 |
| LA_MOUSE | 6741 | LA | SSB | La; La/SSB; | NM_003142; NM_001294145; |
| | | | | LARP3 | |
| PDIP2_MOUSE | 26073 | PDIP2 | POLDIP2 | POLD4; p38; | NM_001290145; NM_015584 |
| | | | | PDIP38 | |
| AGAP3_MOUSE | 116988 | AGAP3 | AGAP3 | CRAG; cnt-g3; | NM_001281300; XM_005249942; XM_005249943; |
| | | | | AGAP-3; | XM_011515780; NM_001042535; NM_031946 |
| | | | | CENTG3; | |
| | | | | MRIP-1 | |
| CO6A1_MOUSE | 1291 | CO6A1 | COL6A1 | OPLL | NM_001848; |
| CRNL1_MOUSE | 51340 | CRNL1 | CRNKL1 | HCRN; CLF; | NM_001278627; NM_001278626; NM_001278628; |
| | | | | CRN; MSTP021; | NM_001278625; NM_016652 |
| | | | | Clf1; SYF3 | |
| MATR3_MOUSE | 9782 | MATR3 | MATR3 | MPD2; ALS21; | NM_001282278; NM_018834; NM_001194956; |
| | | | | VCPDM | NM_199189; ; NM_001194954; NM_001194955 |
| PRP17_MOUSE | 51362 | PRP17 | CDC40 | PRP17; PRPF17; | NM_015891; XM_011535880 |
| | | | | EHB3 | |
| RL7_MOUSE | 6129 | RL7 | RPL7 | L7; humL7-1 | XM_006716463; NM_000971 |
| NUCL_MOUSE | 4691 | NUCL | NCL | C23 | NM_005381 |
| RS9_MOUSE | 6203 | RS9 | RPS9 | S9 | XM_011547987; XM_011548358; XM_011548624; |
| | | | | | XR_431025; XR_431068; XR_953069; NM_001013; |
| | | | | | XM_005278288; XM_006726201; XM_006726202; |
| | | | | | XM_011547988; XM_011548623; XR_254260; |
| | | | | | XR_254311; XR_431090; XR_952765; XR_952994; |
| | | | | | XM_011547789; XM_011547790; XR_431067; |
| | | | | | XR_952920; XR_952995; XR_953155; XR_254518; |
| | | | | | XR_953156; XM_005277274; XM_006725965; |
| | | | | | XR_431057; XR_431069; XR_952922; XR_952996; |
| | | | | | XR_953068; XM_005278287; XM_011548167; |
| | | | | | XR_254517; XR_952766; XR_953070; XR_953157; |
| | | | | | XM_005277315; XM_011548359; XR_431058; |
| | | | | | XR_952764; XR_952919; XM_005277084; |
| | | | | | XM_005277085; XM_011548166; XR_430207; |
| | | | | | XR_431099 |
| HTRA2_MOUSE | 27429 | HTRA2 | HTRA2 | PARK13; OMI; | ; NM_145074; XM_005264266; NM_013247 |
| | | | | PRSS25 | |
| E9Q7G0_MOUSE | — | | | | |
| LRC59_MOUSE | 55379 | LRC59 | LRRC59 | p34; PRO1855 | NM_018509 |
| THOC2_MOUSE | 57187 | THOC2 | THOC2 | THO2; CXorf3; | XM_005262447; XM_011531369; XM_011531372; ; |
| | | | | hTREX120; | XM_011531368; XM_011531374; XR_938550; |
| | | | | dJ506G2.1 | XR_938552; NM_001081550; XM_011531373; |
| | | | | | XR_938551; XM_005262450; XR_938553; NM_020449; |
| | | | | | XM_011531367; XM_011531370; XM_011531371 |
| ERLN2_MOUSE | 11160 | ERLN2 | ERLIN2 | NET32; SPFH2; | XM_005273392; XM_006716280; NM_001003790; |
| | | | | Erlin-2; SPG18; | NM_007175; ; NM_001003791 |
| | | | | C8orf2 | |
| GALK1_MOUSE | 2584 | GALK1 | GALK1 | GALK; HEL-S- | ; NM_000154 |
| | | | | 19; GK1 | |
| SAFB1_MOUSE | 6294 | SAFB1 | SAFB | HAP; HET; | XM_006722839; NR_037699; NM_001201340; |
| | | | | SAF-B1; SAFB1 | NM_001201339; NM_001201338; NM_002967 |
| RL28_MOUSE | 6158 | RL28 | RPL28 | L28 | NM_001136135; NM_001136137; NM_001136136; |
| | | | | | NM_001136134; XM_005259132; NM_000991 |
| MYO1C_MOUSE | 4641 | MYO1C | MYO1C | myr2; MMI-beta; | NM_033375; NM_001080950; NM_001080779 |
| | | | | MMIb; NMI | |
| SRS10_MOUSE | 10772 | SRS10 | SRSF10 | PPP1R149; | NM_001191009; NM_001191006; NM_001191007; |
| | | | | SFRS13A; | NM_001300937; NM_054016; NR_034035; |
| | | | | TASR2; | NM_001191005; NM_006625; NM_001300936 |
| | | | | SFRS13; TASR; | |
| | | | | TASR1; FUSIP1; | |
| | | | | FUSIP2; NSSR; | |
| | | | | SRp38; SRrp40 | |
| E9PYF4_MOUSE | — | | | | |
| ACAD9_MOUSE | 28976 | ACAD9 | ACAD9 | NPD002 | NR_033426; XR_427367; XM_011512742; ; |
| | | | | | NM_014049 |
| KIF2A_MOUSE | 3796 | B0AZS5; | KIF2A | KIF2; CDCBM3; | NM_004520; NM_001243952; NM_001098511; |
| | | KIF2A | | HK2 | NM_001243953 |
| IDH3A_MOUSE | 3419 | B4DJB4; | IDH3A | — | XM_005254334; NM_005530; XM_005254337; |
| | | IDH3A | | | XM_005254336 |
| PWP2_MOUSE | 5822 | PWP2 | PWP2 | EHOC-17; | XM_011529667; NM_005049 |
| | | | | UTP1; PWP2H | |
| CPSF7_MOUSE | 79869 | CPSF7 | CPSF7 | CFIm59 | XM_011545257; XM_011545263; XM_005274303; |
| | | | | | NM_001142565; XM_011545258; XM_011545262; |
| | | | | | XM_005274299; XM_011545260; NM_024811; |
| | | | | | XM_011545261; NM_001136040; XM_005274298; |
| | | | | | XM_011545259 |
| Q6PGF5_MOUSE | — | | | | |
| NUP93_MOUSE | 9688 | NUP93 | NUP93 | NIC96 | NM_001242795; XM_005256263; NM_014669; |
| | | | | | NM_001242796 |
| H14_MOUSE | 3008 | H14 | HIST1H1E | H1F4; | NM_005321 |
| | | | | dJ221C16.5; | |
| | | | | H1.4; H1E; H1s-4 | |
| FUND2_MOUSE | 65991 | FUND2 | FUNDC2 | HCBP6; DC44; | NM_023934 |
| | | | | PD03104; HCC3 | |
| APT_MOUSE | 353 | APT | APRT | APRTD; AMP | NM_000485; ; NM_001030018 |
| MCM5_MOUSE | 4174 | B1AHB0; | MCM5 | CDC46; P1- | XM_006724242; NM_006739 |
| | | MCM5 | | CDC46 | |
| CLPX_MOUSE | 10845 | CLPX | CLPX | — | XR_931743; XM_011521164; NM_006660 |
| RBM8A_MOUSE | 9939 | RBM8A; | RBM8A | BOV-1C; BOV- | ; NM_005105 |
| | | A0A023T787 | | 1B; DEL1q21.1; | |
| | | | | ZRNP1; TAR; | |
| | | | | BOV-1A; | |
| | | | | C1DELq21.1; | |
| | | | | RBM8B; | |
| | | | | MDS014; | |
| | | | | RBM8; Y14; | |
| | | | | ZNRP | |
| L2GL1_MOUSE | 3996 | L2GL1 | LLGL1 | HUGL-1; | XM_011523851; XM_011523853; XM_011523854; |
| | | | | HUGL1; HUGL; | XM_011523856; XM_011523850; XM_011523855; |
| | | | | DLG4; LLGL | NM_004140; XM_011523852; XM_011523849 |
| SMC5_MOUSE | 23137 | SMC5 | SMC5 | SMC5L1 | NM_015110; XM_005251837; XM_005251839; |
| | | | | | XM_005251838 |
| NAA15_MOUSE | 80155 | NAA15 | NAA15 | TBDN100; | XM_005263236; NM_057175 |
| | | | | NATH; NAT1P; | |
| | | | | Ga19; NARG1; | |
| | | | | TBDN | |
| RS11_MOUSE | 6205 | RS11 | RPS11 | S11 | NM_001015 |
| ATAD3_MOUSE | 83858; | — | — | — | — |
| | 55210 | | | | |
| TIAR_MOUSE | 7073 | TIAR | TIAL1 | TIAR; TCBP | XM_005270108; XR_428715; XM_005270109; ; |
| | | | | | XM_005270110; XR_945808; NM_003252; |
| | | | | | NM_001033925 |
| RL9_MOUSE | 6133 | RL9 | RPL9 | L9; NPC-A-16 | NM_000661; NM_001024921; XM_005262661 |
| ACO13_MOUSE | 55856 | ACO13 | ACOT13 | HT012; PNAS- | NM_001160094; NM_018473 |
| | | | | 27; THEM2 | |
| WDR82_MOUSE | 80335 | WDR82 | WDR82 | PRO2730; | XM_011534136; XM_011534137; NM_025222 |
| | | | | WDR82A; | |
| | | | | MSTP107; | |
| | | | | SWD2; MST107; | |
| | | | | PRO34047; | |
| | | | | TMEM113 | |
| PTRF_MOUSE | 284119 | PTRF | PTRF | cavin-1; CAVIN; | ; NM_012232; XM_005257242 |
| | | | | CAVIN1; CGL4; | |
| | | | | FKSG13 | |
| DDX5_MOUSE | 1655 | DDX5 | DDX5 | p68; HUMP68; | XM_006721738; XM_011524456; XM_011524457; |
| | | | | HLR1; G17P1 | NM_004396; XM_005257111 |
| WDR5_MOUSE | 11091 | WDR5 | WDR5 | CFAP89; SWD3; | NM_017588; NM_052821; XM_005272163 |
| | | | | BIG-3 | |
| CDC73_MOUSE | 79577 | CDC73 | CDC73 | HRPT2; HYX; | XM_006711537; ; NM_024529 |
| | | | | C1orf28; FIHP; | |
| | | | | HRPT1; HPTJT | |
| RM03_MOUSE | 11222 | RM03 | MRPL3 | RPML3; MRL3; | ; NM_007208 |
| | | | | COXPD9 | |
| THOC6_MOUSE | 79228 | THOC6 | THOC6 | BBIS; fSAP35; | NM_024339; NM_001142350 |
| | | | | WDR58 | |
| RL13A_MOUSE | 23521 | RL13A | RPL13A | TSTA1; L13A | NR_073024; NM_001270491; NM_012423 |
| RL22_MOUSE | 6146 | RL22 | RPL22 | EAP; HBP15; | NM_000983 |
| | | | | L22; HBP15/L22 | |
| DAZP1_MOUSE | 26528 | DAZP1 | DAZAP1 | — | XM_005259535; XM_005259536; NM_170711; |
| | | | | | XM_011527906; XM_011527904; XM_011527908; |
| | | | | | XM_005259534; XM_011527909; NM_018959; |
| | | | | | XM_005259531; ; XM_011527907; XM_011527910; |
| | | | | | XM_011527905 |
| E41L3_MOUSE | 23136 | E41L3 | EPB41L3 | 4.1B; DAL-1; | XM_011525619; XM_011525620; XM_011525611; |
| | | | | DAL1 | XM_011525625; XM_011525626; XM_011525635; |
| | | | | | XM_011525609; XM_011525612; XM_011525613; |
| | | | | | XM_011525614; XM_011525615; XM_011525628; |
| | | | | | XM_011525631; NM_001281535; XM_011525607; |
| | | | | | XM_011525616; XM_011525621; XM_011525624; |
| | | | | | XM_011525630; NM_001281533; XM_011525610; |
| | | | | | XM_011525623; XM_011525627; NM_001281534; |
| | | | | | XM_011525606; XM_011525617; XM_011525618; |
| | | | | | XM_011525622; XM_011525629; XM_011525632; |
| | | | | | XM_011525637; XM_011525633; XM_011525636; |
| | | | | | NM_012307; XM_011525608; XM_011525634 |
| RBMX_MOUSE | 27316 | RBMX | RBMX | RBMXP1; | NR_028477; NR_028476; NM_001164803; ; |
| | | | | HNRNPG; | NM_002139 |
| | | | | hnRNP-G; | |
| | | | | RBMXRT; | |
| | | | | HNRPG; RNMX | |
| IDHP_MOUSE | 3418 | IDHP | IDH2 | IDP; IDPM; | ; NM_001289910; NM_002168; NM_001290114 |
| | | | | mNADP-IDH; | |
| | | | | IDH; IDHM; | |
| | | | | D2HGA2; ICD-M | |
| DDX27_MOUSE | 55661 | DDX27 | DDX27 | HSPC259; | NM_017895; XM_011528888 |
| | | | | Drs1p; | |
| | | | | dJ686N3.1; | |
| | | | | PP3241; DRS1; | |
| | | | | RHLP | |
| NTKL_MOUSE | 57410 | NTKL | SCYL1 | GKLP; TAPK; | NM_020680; XM_005274120; XM_005274118; |
| | | | | TRAP; HT019; | NM_001048218; XM_005274121 |
| | | | | NKTL; NTKL; | |
| | | | | P105; TEIF | |
| RL22L_MOUSE | 200916 | RL22L | RPL22L1 | — | NM_001099645; XM_005247205 |
| RBM10_MOUSE | 8241 | RBM10 | RBM10 | GPATC9; | ; NM_152856; XM_005272678; XM_005272679; |
| | | | | GPATCH9; | NM_001204467; NM_005676; NM_001204466; |
| | | | | DXS8237E; | XM_011543989; NM_001204468; XM_006724563; |
| | | | | TARPS; | XM_005272677 |
| | | | | ZRANB5; S1-1 | |
| TBL3_MOUSE | 10607 | TBL3 | TBL3 | UTP13; SAZD | NM_006453 |
| Q99N15_MOUSE | — | | | | |
| RL3_MOUSE | 6122 | RL3 | RPL3 | ASC-1; TARBP- | NM_000967; NM_001033853 |
| | | | | B; L3 | |
| HNRDL_MOUSE | 9987 | HNRDL | HNRNPDL | LGMD1G; | NM_031372; ; NM_005463; NM_001207000; |
| | | | | HNRNP; | NR_003249 |
| | | | | HNRPDL; | |
| | | | | JKTBP2; | |
| | | | | JKTBP; laAUF1 | |
| B1B0C7_MOUSE | — | | | | |
| TIM44_MOUSE | 10469 | TIM44 | TIMM44 | TIM44 | NM_006351 |
| TOP2A_MOUSE | 7153 | TOP2A | TOP2A | TOP2; TP2A | XM_005257632; XM_011525165; NM_001067; |
| FBLN2_MOUSE | 2199 | FBLN2 | FBLN2 | — | XM_006713026; NM_001004019; NM_001165035; |
| | | | | | NM_001998 |
| ILF2_MOUSE | 3608 | ILF2 | ILF2 | NF45; PRO3063 | NM_001267809; NM_004515 |
| U2AF2_MOUSE | 11338 | U2AF2 | U2AF2 | U2AF65 | XM_006722994; NM_001012478; ; NM_007279; |
| | | | | | XM_011526410 |
| CDC5L_MOUSE | 988 | CDC5L | CDC5L | PCDC5RP; | XM_006715289; NM_001253; XR_926346 |
| | | | | CDC5-LIKE; | |
| | | | | dJ319D22.1; | |
| | | | | CEF1; CDC5 | |
| SND1_MOUSE | 27044 | SND1 | SND1 | TDRD11; p100 | NM_014390; XM_011516051 |
| ETFB_MOUSE | 2109 | ETFB | ETFB | FP585; MADD | NM_001014763; ; NM_001985 |
| SMC2_MOUSE | 10592 | B7ZLZ7; | SMC2 | SMC-2; CAP-E; | XM_011518150; XM_011518149; XM_011518151; |
| | | A8K984; | | SMC2L1; CAPE | XM_011518153; NM_006444; XM_011518148; |
| | | B3KMB1; | | | NM_001042550; XM_006716933; XM_011518152; |
| | | SMC2; | | | NM_001265602; XM_011518154; NM_001042551 |
| | | A0A024R158 | | | |
| DDX54_MOUSE | 79039 | DDX54 | DDX54 | DP97 | NM_001111322; NM_024072 |
| RAI14_MOUSE | 26064 | RAI14 | RAI14 | NORPEG; | XM_011514022; XM_011514024; XM_011514016; |
| | | | | RAI13 | XM_011514019; NM_001145520; XM_011514025; |
| | | | | | NM_001145521; NM_001145525; NM_001145522; |
| | | | | | XM_006714469; XM_011514018; XM_011514021; |
| | | | | | XM_011514017; NM_001145523; NM_015577; |
| | | | | | XM_011514020; XM_011514023 |
| PCNA_MOUSE | 5111 | PCNA | PCNA | ATLD2 | NM_002592; NM_182649 |
| CNOT1_MOUSE | 23019 | CNOT1 | CNOT1 | NOT1; AD-005; | NM_206999; NM_001265612; NR_049763; NM_016284 |
| | | | | CDC39; NOT1H | |
| CPSF3_MOUSE | 51692 | CPSF3 | CPSF3 | CPSF-73; | XM_005246167; XM_011510362; NM_016207; |
| | | | | CPSF73 | XM_005246168 |
| RS2_MOUSE | 6187 | RS2 | RPS2 | LLREP3; S2 | NM_002952 |
| PPIL4_MOUSE | 85313 | PPIL4 | PPIL4 | HDCME13P | NM_139126 |
| FXR1_MOUSE | 8087 | FXR1 | FXR1 | FXR1P | XM_005247816; NM_001013438; XM_005247814; |
| | | | | | XM_011513216; XM_005247815; XM_006713775; |
| | | | | | XM_011513215; XM_011513217; NM_005087; |
| | | | | | NM_001013439; XM_005247813 |
| COR1C_MOUSE | 23603 | A0A024RBI5; | CORO1C | HCRNN4 | XM_011538124; NM_014325; XM_011538125; |
| | | COR1C | | | NM_001105237; XR_944514; NM_001276471 |
| DNLI1_MOUSE | 3978 | DNLI1; | LIG1 | — | NR_110296; NM_001289064; XM_006723215; |
| | | B4DM52; | | | XR_430200; NM_000234; NM_001289063; XR_243932; |
| | | F5GZ28 | | | ; XM_005258934; XM_006723216 |
| RM22_MOUSE | 29093 | RM22 | MRPL22 | MRP-L25; | NM_014180; NM_001014990 |
| | | | | RPML25; | |
| | | | | HSPC158; | |
| | | | | L22mt; MRP- | |
| | | | | L22 | |
| RBM5_MOUSE | 10181 | RBM5 | RBM5 | RMB5; G15; | XM_006712917; ; XM_011533261; XM_011533262; |
| | | | | H37; LUCA15 | NM_005778; NR_03627; XM_006712919; XR_427245; |
| U520_MOUSE | 23020 | U520 | SNRNP200 | ASCC3L1; | NM_014014 |
| | | | | BRR2; RP33; | |
| | | | | U5-200KD; | |
| | | | | HELIC2 | |
| MCM6_MOUSE | 4175 | MCM6 | MCM6 | MCG40308; | ; NM_005915 |
| | | | | Mis5; P105MCM | |
| CPSF2_MOUSE | 53981 | CPSF2 | CPSF2 | CPSF100 | XM_005267767; NM_017437 |
| FXR2_MOUSE | 9513 | FXR2 | FXR2 | FMR1L2; | XR_243572; ; NM_004860 |
| | | | | FXR2P | |
| CPSF5_MOUSE | 11051 | CPSF5 | NUDT21 | CFIM25; CPSF5 | NM_007006 |
| RL14_MOUSE | 9045 | RL14 | RPL14 | CAG-ISL-7; | NM_001034996; NM_003973 |
| | | | | L14; CTG-B33; | |
| | | | | RL14; hRL14 | |
| TRA2B_MOUSE | 6434 | TRA2B | TRA2B | PPP1R156; | XM_011513072; XM_006713724; NM_004593; ; |
| | | | | SFRS10; | NM_001243879; XM_005247703 |
| | | | | TRAN2B; | |
| | | | | SRFS10; TRA2- | |
| | | | | BETA; Htra2- | |
| | | | | beta | |
| VWA8_MOUSE | 23078 | VWA8 | VWA8 | KIAA0564 | NM_001009814; XM_011535006; NM_015058; |
| | | | | | XM_006719791; XM_011535007 |
| NAA38_MOUSE | 51691 | LSM8 | LSM8 | NAA38 | NM_016200 |
| HNRPQ_MOUSE | — | | | | |
| TRAP1_MOUSE | 10131 | TRAP1 | TRAP1 | TRAP-1; | NM_001272049; ; XM_011522345; NM_016292 |
| | | | | HSP90L; HSP | |
| | | | | 75; HSP75 | |
| STAG1_MOUSE | 10274 | STAG1 | STAG1 | SCC3A; SA1 | XM_011512332; XM_011512331; NM_005862; |
| | | | | | XM_011512333; XM_011512329; XM_011512330 |
| DDX17_MOUSE | 10521 | DDX17 | DDX17 | RH70; P72 | NM_001098505; NM_030881; NM_001098504; ; |
| | | | | | NM_006386 |
| ERD21_MOUSE | 10945 | ERD21 | KDELR1 | HDEL; PM23; | XM_011526358; NM_006801 |
| | | | | ERD2; ERD2.1 | |
| RL18A_MOUSE | 6142 | RL18A | RPL18A | L18A | NM_000980 |
| UBXN1_MOUSE | 51035 | UBXN1 | UBXN1 | SAKS1; | XM_011545090; NM_001286077; XM_005274033; |
| | | | | UBXD10; 2B28 | NM_015853; NM_001286078 |
| EPDR1_MOUSE | 54749 | EPDR1 | EPDR1 | MERP-1; | NM_001242946; NM_001242948; NM_017549 |
| | | | | MERP1; EPDR; | |
| | | | | UCC1 | |
| KAP0_MOUSE | 5573 | KAP0 | PRKAR1A | ACRDYS1; | XM_011524985; ; NM_212471; NM_001278433; |
| | | | | CAR; CNC; | NM_001276290; XM_011524984; NM_001276289; |
| | | | | PPNAD1; | NM_212472; XM_011524983; NM_002734 |
| | | | | ADOHR; CNC1; | |
| | | | | PRKAR1; TSE1; | |
| | | | | PKR1 | |
| CBR4_MOUSE | 84869 | CBR4 | CBR4 | SDR45C1 | XR_938789; XM_005263315; XM_006714392; |
| | | | | | XM_011532386; XM_006714391; NM_032783; |
| | | | | | XM_011532385; XM_005263316 |
| RL13_MOUSE | 6137 | RL13; | RPL13 | D16S444E; L13; | NM_001243130; NM_033251; NM_000977; |
| | | A8K4C8 | | D16S44E; BBC1 | NM_001243131 |
| SFPQ_MOUSE | 6421 | SFPQ | SFPQ | PPP1R140; PSF; | XM_005271113; XM_005271115; XM_011541950; |
| | | | | POMP100 | XM_005271112; NM_005066 |
| PDS5B_MOUSE | 23047 | PDS5B | PDS5B | AS3; CG008; | XM_011535002; XM_005266298; XM_011535001; |
| | | | | APRIN | NM_015032; NM_015928; XM_011534999; |
| | | | | | XM_011535000; |
| KPCI_MOUSE | 5584 | KPCI | PRKCI | PKCI; | NM_002740 |
| | | | | DXS1179E; | |
| | | | | nPKC-iota | |
| THOC4_MOUSE | 10189 | THOC4 | ALYREF | ALY/REF; | NM_005782; XR_933919 |
| | | | | THOC4; BEF; | |
| | | | | ALY; REF | |
| SF3B3_MOUSE | 23450 | SF3B3 | SF3B3 | SAP130; RSE1; | NM_012426 |
| | | | | STAF130; | |
| | | | | SF3b130 | |
| E9QN31_MOUSE | — | | | | |
| AKT1_MOUSE | 207 | AKT1 | AKT1 | AKT; PKB- | NM_005163; XM_011536544; NM_001014431; |
| | | | | ALPHA; RAC; | XM_005267401; XM_011536543; NM_001014432; |
| | | | | PRKBA; RAC- | |
| | | | | ALPHA; CWS6; | |
| | | | | PKB | |
| NOP56_MOUSE | 10528 | NOP56 | NOP56 | SCA36; NOL5A | NR_027700; ; NM_006392 |
| SMU1_MOUSE | 55234 | SMU1 | SMU1 | SMU-1; BWD; | XM_005251503; NM_018225 |
| | | | | fSAP57 | |
| MTA1_MOUSE | 9112 | MTA1 | MTA1 | — | XM_011537305; XM_011537309; XM_011537301; |
| | | | | | XM_011537304; XM_011537311; XM_011537315; ; |
| | | | | | XM_011537306; XM_011537308; XM_011537314; |
| | | | | | XM_011537310; XM_011537302; XM_011537303; |
| | | | | | XM_011537307; NM_004689; NM_001203258; |
| | | | | | XM_011537312; XM_011537313 |
| BUB3_MOUSE | 9184 | BUB3 | BUB3 | BUB3L; hBUB3 | NM_004725; ; NM_001007793 |
| RPF2_MOUSE | 84154 | RPF2 | RPF2 | bA397G5.4; | NM_001289111; NM_032194 |
| | | | | BXDC1 | |
| ATLA3_MOUSE | 25923 | ATLA3 | ATL3 | HSN1F | ; NM_015459; XM_006718493; XM_006718494; |
| | | | | | XM_011544902; NM_001290048 |
| NSA2_MOUSE | 10412 | NSA2 | NSA2 | CDK105; | XM_011543098; NM_001271665; XR_948227; |
| | | | | TINP1; HUSSY- | NM_014886; NR_073403 |
| | | | | 29; HUSSY29; | |
| | | | | HCLG1; HCL- | |
| | | | | G1 | |
| ACON_MOUSE | 50 | ACON | ACO2 | ACONM; ICRD | ; NM_001098 |
| DNJC3_MOUSE | 5611 | DNJC3 | DNAJC3 | PRKRI; HP58; | XM_011521105; NM_006260; XM_011521104; |
| | | | | P58; ERdj6; | |
| | | | | P58IPK; ACPHD | |
| RPB2_MOUSE | 5431 | RPB2; | POLR2B | POL2RB; | NM_001303269; NM_000938; NM_001303268 |
| | | B4DH29; | | hRPB140; RPB2 | |
| | | C9J4M6; | | | |
| | | B4DHJ3; | | | |
| | | C9J2Y9 | | | |
| RL11_MOUSE | 6135 | RL11 | RPL11 | L11; DBA7; | NM_000975; NM_001199802; |
| | | | | GIG34 | |
| PRP6_MOUSE | 24148 | PRP6 | PRPF6 | TOM; ANT-1; | XM_006723769; ; NM_012469 |
| | | | | Prp6; hPrp6; | |
| | | | | C20orf14; RP60; | |
| | | | | ANT1; | |
| | | | | SNRNP102; U5- | |
| | | | | 102K | |
| LSM2_MOUSE | 57819 | LSM2 | LSM2 | YBL026W; | NM_021177 |
| | | | | C6orf28; G7B; | |
| | | | | snRNP | |
| RS28_MOUSE | — | | | | |
| K6PF_MOUSE | 5213 | A0A024R0Y5; | PFKM | PFKA; PFK1; | NM_001166688; NM_001166687; NM_001166686; |
| | | PFKAM | | PFK-1; PFKX; | XM_005268976; XM_005268978; ; XM_005268977; |
| | | | | PPP1R122; ATP- | XM_011538487; XM_005268974; XM_005268975; |
| | | | | PFK; GSD7 | XM_005268979; XM_011538488; NM_000289 |
| NU155_MOUSE | 9631 | NU155 | NUP155 | ATFB15; N155 | XM_011514166; ; XM_011514164; NM_001278312; |
| | | | | | XM_011514165; NM_004298; NM_153485 |
| PTH2_MOUSE | 51651 | PTH2 | PTRH2 | 2; CFAP37; | XM_011524886; NM_001015509; XM_005257447; |
| | | | | PTH2; CGI-147; | XM_011524887; NM_016077 |
| | | | | IMNEPD; PTH; | |
| | | | | BIT1; PTH 2 | |
| FLOT1_MOUSE | 10211 | FLOT1 | FLOT1 | — | XM_005275502; XM_005275503; XM_005272759; |
| | | | | | XM_005272760; XM_006725672; XM_006726072; |
| | | | | | XM_005248780; XM_005274909; XM_005275335; |
| | | | | | XM_005248781; XM_005274910; XM_006714947; |
| | | | | | XM_006725971; XM_005275336; XM_006725465; |
| | | | | | NM_005803 |
| NIPS2_MOUSE | 2631 | NIPS2 | GBAS | NIPSNAP2 | NM_001483; NM_001202469 |
| PUF60_MOUSE | 22827 | PUF60 | PUF60 | SIAHBP1; | NM_001271096; NM_001271097; NM_001136033; |
| | | | | RoBPI; FIR; | NM_014281; ; NM_001271100; NM_078480; |
| | | | | VRJS | XM_011516929; NM_001271098; XM_011516930; |
| | | | | | NM_001271099 |
| SMAL1_MOUSE | 50485 | SMAL1 | SMARCAL1 | HHARP; HARP | ; XM_006712557; NM_014140; NM_001127207; |
| | | | | | XM_005246632; XM_005246631 |
| MPPB_MOUSE | 9512 | MPPB | PMPCB | P-52; MPPB; | XM_005250717; XM_006716181; XR_242267; |
| | | | | Beta-MPP; | NM_004279 |
| | | | | MPP11; MPPP52 | |
| RBM39_MOUSE | 9584 | RBM39 | RBM39 | CAPERalpha; | XM_011529110; NM_184237; XM_006723891; |
| | | | | FSAP59; | XM_006723893; NM_001242599; NM_184234; ; |
| | | | | CAPER; HCC1; | NM_001242600; NR_040722; XM_006723890; |
| | | | | RNPC2 | XM_011529111; NM_004902; NR_040723; |
| | | | | | NM_184241; NR_040724; NM_184244 |
| SNX3_MOUSE | 8724 | SNX3 | SNX3 | Grd19; | NM_001300929; NM_001300928; ; NM_003795; |
| | | | | MCOPS8; SDP3 | NM_152828; NM_152827 |
| RBBP4_MOUSE | 5928 | RBBP4 | RBBP4 | lin-53; RBAP48; | NM_005610; NM_001135255; NM_001135256 |
| | | | | NURF55 | |
| AL4A1_MOUSE | 8659 | AL4A1 | ALDH4A1 | P5CD; P5CDh; | XR_946786; XM_011542353; NM_003748; |
| | | | | ALDH4 | NM_170726; XM_011542352; NM_001161504; |
| SMC1A_MOUSE | 8243 | G8JLG1; | SMC1A | SMCB; SB1.8; | ; NM_006306; NM_001281463 |
| | | SMC1A | | SMC1alpha; | |
| | | | | DXS423E; | |
| | | | | CDLS2; SMC1; | |
| | | | | SMC1L1 | |
| ILF3_MOUSE | 3609 | ILF3 | ILF3 | MMP4; | ; XM_005259895; XM_011527984; XM_006722742; |
| | | | | DRBP76; MPP4; | XM_011527987; XM_011527986; NM_004516; |
| | | | | NFAR2; NF-AT- | NM_012218; NM_017620; XM_011527985; |
| | | | | 90; NF110b; | NM_001137673; NM_153464 |
| | | | | MPHOSPH4; | |
| | | | | DRBF; NF90a; | |
| | | | | NF90b; NFAR; | |
| | | | | NFAR-1; | |
| | | | | TCP110; NF90; | |
| | | | | CBTF; NF110; | |
| | | | | TCP80 | |
| SERPH_MOUSE | 871 | SERPH | SERPINH1 | PPROM; RA- | ; NM_001235; XM_006718729; XM_011545327; |
| | | | | A47; CBP2; | NM_001207014; XM_011545326 |
| | | | | PIG14; CBP1; | |
| | | | | gp46; AsTP3; | |
| | | | | HSP47; OI10; | |
| | | | | SERPINH2 | |
| AP2A1_MOUSE | 160 | AP2A1 | AP2A1 | ADTAA; AP2- | NM_014203; XM_011526556; XM_011526557; |
| | | | | ALPHA; | NM_130787 |
| | | | | CLAPA1 | |
| CCAR2_MOUSE | 57805 | CCAR2 | CCAR2 | p30 DBC; | XM_011544604; NM_199205; NR_033902; |
| | | | | DBC1; | XM_011544603; NM_021174 |
| | | | | KIAA1967; | |
| | | | | NET35; | |
| | | | | p30DBC; DBC-1 | |
| SUCB1_MOUSE | 8803 | SUCB1; | SUCLA2 | SCS-betaA; | XM_011535293; NM_003850; ; XM_011535292; |
| | | E5KS60 | | MTDPS5; A- | XR_941688 |
| | | | | BETA | |
| RM14_MOUSE | 64928 | RM14 | MRPL14 | L32mt; | XM_005249301; NM_032111; XM_011514814; |
| | | | | MRPL32; MRP- | XM_005249300; XM_005249299 |
| | | | | L32; L14mt; | |
| | | | | MRP-L14; | |
| | | | | RMPL32; | |
| | | | | RPML32 | |
| RPB1_MOUSE | 5430 | RPB1 | POLR2A | RPB1; RPO2; | ; NM_000937 |
| | | | | RpIILS; POLR2; | |
| | | | | RPBh1; POLRA; | |
| | | | | hRPB220; | |
| | | | | hsRPB1; RPOL2 | |
| AGK_MOUSE | 55750 | AGK | AGK | MULK; | XM_011516397; XM_005250023; NM_018238; |
| | | | | MTDPS10; | |
| | | | | CATC5; | |
| | | | | CTRCT38 | |
| CSDE1_MOUSE | 7812 | CSDE1 | CSDE1 | UNR; D1S155E | NM_001007553; NM_001242892; NM_007158; |
| | | | | | NM_001242893; NM_001130523; NM_001242891 |
| PDLI7_MOUSE | 9260 | PDLI7 | PDLIM7 | LMP3; LMP1 | XM_011534699; NR_103804; XM_011534697; |
| | | | | | XM_011534700; XM_011534698; XM_011534696; |
| | | | | | NM_213636; NM_005451; NM_203352; NM_203353 |
| RB6I2_MOUSE | 23085 | RB6I2 | ERC1 | ELKS; ERC-1; | XM_011520940; NM_178039; NR_027948; |
| | | | | RAB6IP2; Cast2 | NM_001301248; XM_011520938; XM_011520942; |
| | | | | | XR_931510; XM_011520943; XR_931509; |
| | | | | | XM_011520936; NR_027949; XM_011520937; |
| | | | | | NM_178040; XM_011520939; XM_011520941; |
| | | | | | XM_011520944; XR_931508; NR_027946 |
| CHD4_MOUSE | 1108 | CHD4 | CHD4 | Mi2-BETA; Mi- | XM_006718958; NM_001273; XM_006718962; |
| | | | | 2b; CHD-4 | XM_006718960; XM_006718959; XM_005253668; |
| | | | | | XM_006718961; NM_001297553 |
| PRDX3_MOUSE | 10935 | PRDX3 | PRDX3 | AOP-1; SP-22; | NR_126105; NM_014098; NM_006793; NR_126103; |
| | | | | AOP1; MER5; | NM_001302272; NR_126102; NR_126106 |
| | | | | prx-III; HBC189; | |
| | | | | PRO1748 | |
| AP2M1_MOUSE | 1173 | AP2M1 | AP2M1 | AP50; mu2; | NM_004068; NM_001025205 |
| | | | | CLAPM1 | |
| LIMA1_MOUSE | 51474 | LIMA1 | LIMA1 | SREBP3; EPLIN | NM_001243775; XM_011538455; NM_001113547; ; |
| | | | | | NM_001113546; NM_016357 |
| GOLI4_MOUSE | 27333 | GOLI4 | GOLIM4 | GPP130; | XM_005247365; XM_005247364; NM_014498; |
| | | | | GIMPC; P138; | XM_005247366 |
| | | | | GOLPH4 | |
| HCFC1_MOUSE | 3054 | HCFC1 | HCFC1 | HCF1; HFC1; | XM_006724816; XM_011531147; ; XM_011531144; |
| | | | | PPP1R89; | XM_11011531146; XM_011531150; XM_011531148; |
| | | | | VCAF; MRX3; | NM_005334; XM_006724815; XM_011531149; |
| | | | | CFF; HCF; HCF-1 | XM_011531145 |
| E41L1_MOUSE | 2036 | E41L1 | EPB41L1 | MRD11; 4.1N | XM_011528669; XM_011528677; XM_011528681; |
| | | | | | XM_011528684; XM_011528670; XM_011528674; |
| | | | | | XM_011528686; XM_011528666; ; NM_001258331; |
| | | | | | XM_011528675; XM_011528676; XM_011528679; |
| | | | | | XM_011528680; NM_001258329; NM_012156; |
| | | | | | XM_011528667; XM_011528668; XM_011528671; |
| | | | | | XM_011528672; XM_011528685; NM_001258330; |
| | | | | | XM_011528664; XM_011528665; XM_011528682; |
| | | | | | XM_011528683; NM_177996; XM_011528673; |
| | | | | | XM_011528678 |
| TMM65_MOUSE | 157378 | TMM65 | TMEM65 | — | XM_011516847; NM_194291 |
| SMD1_MOUSE | 6632 | SMD1 | SNRPD1 | HsT2456; | NM_006938; NM_001291916 |
| | | | | SMD1; SNRPD; | |
| | | | | Sm-D1 | |
| RT05_MOUSE | 64969 | RT05 | MRPS5 | MRP-S5; S5mt | XM_006712694; XR_922989; NM_031902 |
| DHX15_MOUSE | 1665 | DHX15 | DHX15 | PRPF43; HRH2; | XR_925314; NM_001358 |
| | | | | PRP43; DBP1; | |
| | | | | DDX15; | |
| | | | | PrPp43p | |
| MK03_MOUSE | 5595 | MK03; | MAPK3 | P44ERK1; | NM_001040056; XR_243293; NM_001109891; |
| | | L7RXH5 | | P44MAPK; | NM_002746; |
| | | | | ERK-1; PRKM3; | |
| | | | | ERT2; | |
| | | | | HUMKER1A; | |
| | | | | p44-ERK1; p44- | |
| | | | | MAPK; ERK1; | |
| | | | | HS44KDAP | |
| CPSF1_MOUSE | 29894 | CPSF1 | CPSF1 | CPSF160; | XM_006716548; XM_011516999; NM_013291; |
| | | | | P/cl.18; | XM_006716550; XM_011516998; XM_011516997; |
| | | | | HSU37012 | XM_006716549 |
| SYMC_MOUSE | 4141 | SYMC | MARS | MRS; SPG70; | XM_006719398; NM_004990; XM_011538353; |
| | | | | MTRNS; | |
| | | | | METRS | |
| LPPRC_MOUSE | 10128 | LPPRC | LRPPRC | CLONE-23970; | XM_011532474; ; XM_006711915; XM_006711916; |
| | | | | LRP130; LSFC; | XM_011532473; NM_133259 |
| | | | | GP130 | |
| RL27A_MOUSE | 6157 | RL27A | RPL27A | L27A | NM_032650; NM_000990 |
| SRSF1_MOUSE | 6426 | SRSF1 | SRSF1 | SFRS1; SRp30a; | NR_034041; XM_006722012; XR_429911; XR_429912; |
| | | | | ASF; SF2; | NM_001078166; NM_006924 |
| | | | | SF2p33 | |
| BOP1_MOUSE | 23246 | BOP1 | BOP1 | — | ; NM_015201 |
| IMDH2_MOUSE | 3615 | IMDH2 | IMPDH2 | IMPD2; IMPDH- | XM_006713128; ; NM_000884 |
| | | | | II | |
| H31_MOUSE | 8353; | — | — | — | — |
| | 8358; | | | | |
| | 8357; | | | | |
| | 8968; | | | | |
| | 8350; | | | | |
| | 8351; | | | | |
| | 8355; | | | | |
| | 8354; | | | | |
| | 8356; | | | | |
| | 8352 | | | | |
| AACS_MOUSE | 65985 | AACS | AACS | ACSF1; SUR-5 | XM_005253611; XR_242960; NM_023928; |
| | | | | | XM_005253609; XM_005253610; XM_011538692 |
| PDS5A_MOUSE | 23244 | PDS5A | PDS5A | PIG54; SCC112; | NM_001100400; XM_011513673; XM_011513674; |
| | | | | SCC-112 | NM_015200; ; NM_001100399; XM_011513672 |
| PP1G_MOUSE | 5501 | PP1G; | PPP1CC | PP-1G; PPP1G; | ; XM_011538505; XM_011538504; NM_001244974; |
| | | A0A024RBP2 | | PP1C | |
| PCH2_MOUSE | 9319 | PCH2 | TRIP13 | 16E1BP | NM_001166260; NM_004237; XM_011514163 |
| DX39A_MOUSE | 10212 | DX39A | DDX39A | URH49; BAT1; | NM_001204057; NR 038336; NM_005804; NM_138998 |
| | | | | DDXL; BAT1L; | NR_046366; XM_006722606; XM_011527620; |
| | | | | DDX39 | XM_011527621; |
| AKAP8_MOUSE | 10270 | AKAP8 | AKAP8 | AKAP 95; | XM_011527624; XM_011527625; XR 244062; |
| | | | | AKAP-8; | NM_005858 |
| | | | | AKAP-95; | |
| | | | | AKAP95 | |
| LAR4B_MOUSE | 23185 | LAR4B | LARP4B | LARP5; | XM_005252431; XM_011519434; NM_015155; |
| | | | | KIAA0217 | XM_011519435; XM_011519436; XM_005252432; |
| | | | | | XM_005252435 |
| ARI1A_MOUSE | 8289 | ARI1A | ARID1A | B120; BAF250a; | NM_018450; ;NM_139135; NM_006015 |
| | | | | C1orf4; ELD; | |
| | | | | OSA1; P270; | |
| | | | | SMARCF1; | |
| | | | | hELD; hOSA1; | |
| | | | | BAF250; | |
| | | | | BM029; 1VIRD14 | |
| RUXE_MOUSE | 6635 | RUXE | SNRPE | SME; Sm-E; B- | NM_001304464; NR_130746; NM_003094 |
| | | | | raf; HYPT11 | |
| PNPT1_MOUSE | 87178 | PNPT1 | PNPT1 | OLD35; old-35; | XM_005264629; NM_033109; XM_011533142; |
| | | | | DFNB70; | |
| | | | | PNPASE; | |
| | | | | COXPD13 | |
| BAZ1A_MOUSE | 11177 | BAZ1A | BAZ1A | WALp1; | XM_011536376; XR_943381; NM_013448; |
| | | | | WCRF180; | XM_011536374; XM_011536375; NM_182648 |
| | | | | hACF1; ACF1 | |
| ACSF3_MOUSE | 197322 | ACSF3 | ACSF3 | — | XM_011522943; XR_933238; XR_933240; |
| | | | | | NM_001127214; XR_933239; XM_011522944; |
| | | | | | NR_104293; NM_001284316; XM_011522942; |
| | | | | | XR_933241; ; NM_174917; XM_005256293; |
| | | | | | NM_001243279; NR_045667; NR_045666 |
| RS23_MOUSE | 6228 | RS23 | RPS23 | S23 | NM_001025 |
| CHERP_MOUSE | 10523 | CHERP | CHERP | SCAF6; SRA1; | NM_006387 |
| | | | | DAN16 | |
| RL38_MOUSE | 6169 | RL38 | RPL38 | L38 | NM_000999; NM_001035258 |
| NOC3L_MOUSE | 64318 | NOC3L | NOC3L | C10orf117; | XM_005270048; NM_022451; XM_011540067; |
| | | | | FAD24; AD24 | XR_945799 |
| TBB6_MOUSE | 84617 | TBB6 | TUBB6 | HsT1601; | NM_ 001303530; NM_001303524; NM_ 001303528; |
| | | | | TUBB-5 | NM_ 001303525; NM_001303526; NM_ 001303529; |
| | | | | | NM_ 001303527; NM_032525 |
| PDIP3_MOUSE | 84271 | PDIP3 | POLDIP3 | SKAR; PDIP46 | XM_ 011530457; NM_032311; NM_178136; |
| | | | | | NM_001278657; XR_937942; NR 103820 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
(i) a DNMT Inhibitor selected from-decitabine and RG108;
(ii) an inhibitory nucleic acid targeting XIST RNA.

2. A method of activating an inactive X-linked allele in a cell, preferably a cell of a female heterozygous subject or male hemizygous subject, the method comprising administering to the cell
(i) a DNA methyltransferase (DNMT) inhibitor selected from decitabine and RG108; and
(ii) an inhibitory nucleic acid targeting XIST RNA, wherein the inhibitory nucleic acid is complementary to at least 8 consecutive nucleotides of XIST RNA.

3. The method of claim 2, wherein the X-linked allele is an epigenetically silenced or hypomorphic allele on the active X-chromosome.

4. The method of claim 2, wherein the DNMT inhibitor is decitabine.

5. The method of claim 2 wherein the DNMT inhibitor is RG 108.

6.-7. (canceled)

8. The method of claim 2, wherein the inactive X-linked allele is associated with an X-linked disorder, and the DNMT Inhibitor and the inhibitory nucleic acid targeting XIST RNA are administered in a therapeutically effective amount.

9. The method of claim 2, wherein the active X-linked allele is associated with an X-linked disorder, and the DNMT Inhibitor and the inhibitory nucleic acid targeting XIST RNA are administered in a therapeutically effective amount.

10. The method of claim 2, wherein the cell is in a living subject.

11.-13. (canceled)

14. The method of claim 2, wherein the inhibitory nucleic acid does not comprise three or more consecutive guanosine nucleotides or does not comprise four or more consecutive guanosine nucleotides.

15. The method of claim 2, wherein the inhibitory nucleic acid is 8 to 30 nucleotides in length.

16. The method of claim 2, wherein at least one nucleotide of the inhibitory nucleic acid is a nucleotide analogue.

17. The method of claim 2, wherein at least one nucleotide of the inhibitory nucleic acid comprises a 2′ O-methyl, or wherein each nucleotide of the inhibitory nucleic acid comprises a 2′ O-methyl.

18. The method of claim 2, wherein the inhibitory nucleic acid comprises at least one ribonucleotide, at least one deoxyribonucleotide, or at least one bridged nucleotide.

19. The method of claim 18, wherein the bridged nucleotide is a LNA nucleotide, a cEt nucleotide or a ENA modified nucleotide.

20. The method of claim 2, wherein each nucleotide of the inhibitory nucleic acid is a LNA nucleotide.

21. The method of claim 2, wherein one or more of the nucleotides of the inhibitory nucleic acid comprise 2′-fluoro-deoxyribonucleotides and/or 2′ O-methyl nucleotides.

22. The method of claim 2, wherein one or more of the nucleotides of the inhibitory nucleic acid comprise one of both of ENA nucleotide analogues or LNA nucleotides.

23. The method of claim 2, wherein the nucleotides of the inhibitory nucleic acid comprise phosphorothioate internucleotide linkages between at least two nucleotides, or between all nucleotides.

24. The method of claim 2, wherein the inhibitory nucleic acid is a gapmer or a mixmer.

25.-31. (canceled)

32. The composition of claim 1, wherein the inhibitory nucleic acid is 8 to 30 nucleotides in length.
